(19) United States
(12) Patent Application Publication
(10) Pub. No.: US 2023/0407261 A1
(43) Pub. Date: Dec. 21, 2023

(54) DIRECT CONVERSION OF HUMAN MESENCHYMAL STEM CELLS TO HUMAN CARDIOMYOCYTES

(71) Applicant: UNIVERSITY OF FLORIDA RESEARCH FOUNDATION, INCORPORATED, Gainesville, FL (US)

(72) Inventors: David Tran, La Cañada Flintridge, CA (US); Son Bang Le, South Pasadena, CA (US); Nathan Thai, Alhambra, CA (US)

(21) Appl. No.: 18/331,891

(22) Filed: Jun. 8, 2023

Related U.S. Application Data
(60) Provisional application No. 63/351,108, filed on Jun. 10, 2022.
(60) Provisional application No. 63/352,178, filed on Jun. 14, 2022.

(51) Int. Cl. C12N 5/077; C12N 15/86

(52) U.S. Cl. CPC C12N 5/0657; C12N 15/86; C12N 2501/60; C12N 2506/1346

(57) ABSTRACT

The invention provides compositions comprising a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B. The compositions are useful in the treatment of cardiac disorders and in reprogramming a mesenchymal stem cell (MSC) to an autologous induced cardiomyocyte (iCM).

### CROSS REFERENCE TO RELATED APPLICATIONS

This application claims the benefit of U.S. Provisional Application No. 63/351,108, filed Jun. 10, 2022, and U.S. Provisional Application No. 63/352,178, filed Jun. 14, 2022, each of which is incorporated by reference in its entirety for all purposes.

### REFERENCE TO A SEQUENCE LISTING

This application includes an electronic sequence listing in a file named 596175SEQLST.XML, created on Jun. 7, 2023 and containing 142,971 bytes, which is hereby incorporated by reference in its entirety for all purposes.

### BACKGROUND

As cardiomyocytes (CMs) are terminally differentiated cells, a reliable and abundant source of CMs is critical for regenerative applications for cardiac failure. Historically, cellular transdifferentiation has relied on the highly inefficient and time consuming induced pluripotent stem cell (iPSC) intermediary. More recently, direct CM conversion has been studied extensively since the first generation of CMs from mouse embryonic fibroblasts without having to transit through iPSC. However, mass production of autologous CMs remains the main obstacle to making transdifferentiation-sourced autologous cell transplantation a clinical reality.

### SUMMARY OF THE INVENTION

In one aspect, the invention provides a composition for treating a subject with a cardiac disorder, comprising a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B.

In another aspect, the invention provides a composition for reprogramming a mesenchymal stem cell (MSC) to an autologous induced cardiomyocyte (iCM), comprising a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B.

In another aspect, the invention provides a method of treating a subject with a cardiac disorder by administering a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B to said subject.

In another aspect, the invention provides a method of reprogramming a mesenchymal stem cell (MSC) to an autologous induced cardiomyocyte (iCM), by introducing a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B into the MSC.

In another aspect, the invention provides a method of treating a subject with a cardiac disorder by administering an autologous mesenchymal stem cell that has been introduced with a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B to said subject.

In some compositions and methods, the ribonucleotide or ribonucleotides or deoxyribonucleotide or deoxyribonucleotides encode at three cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B. In some compositions and methods, the ribonucleotide or ribonucleotides or deoxyribonucleotide or deoxyribonucleotides encode at least four cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B. In some compositions and methods, the ribonucleotide or ribonucleotides or deoxyribonucleotide or deoxyribonucleotides encode at least five cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B.

In some compositions and methods, the ribonucleotide or ribonucleotides or deoxyribonucleotide or deoxyribonucleotides encode POU2F1, HAND1, GATA4, NACA2, and TSHZ2. In some compositions and methods, the ribonucleotide or ribonucleotides or deoxyribonucleotide or deoxyribonucleotides encode GATA4, IKZF4, NACA2, and TSHZ2. In some compositions and methods, the ribonucleotide or ribonucleotides or deoxyribonucleotide or deoxyribonucleotides encode POU2F1, HAND1, GATA4, and HAND2. In some compositions and methods, the ribonucleotide or ribonucleotides or deoxyribonucleotide or deoxyribonucleotides encode GATA4, HAND2, and IKZF4. In some compositions and methods, the ribonucleotide or ribonucleotides or deoxyribonucleotide or deoxyribonucleotides encode POU2F1, GATA4, and TSHZ2. In some compositions and methods, the ribonucleotide or ribonucleotides or deoxyribonucleotide or deoxyribonucleotides encode HAND1, GATA4, IKZF4, and NACA2. In some compositions and methods, the ribonucleotide or ribonucleotides or deoxyribonucleotide or deoxyribonucleotides encode HAND1, GATA4, and NACA2. In some compositions and methods, the ribonucleotide or ribonucleotides or deoxyribonucleotide or deoxyribonucleotides encode POU2F1, HAND1, GATA4, IKZF4, and NACA2. In some compositions and methods, the ribonucleotide or ribonucleotides or deoxyribonucleotide or deoxyribonucleotides encode POU2F1, HAND1, GATA4, JUP, and TSHZ2. In some compositions and methods, the ribonucleotide or ribonucleotides or deoxyribonucleotide or deoxyribonucleotides encode ACTN2, POU2F1, HAND1, and GATA4.

In some compositions and methods, the ribonucleotide or ribonucleotides or deoxyribonucleotide or deoxyribonucleotides encode HAND1 and at least one of PBX2, ACTN2, POU2F1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B. In some compositions and methods, the ribonucleotide or ribonucleotides or deoxyribonucleotide or deoxyribonucleotides encode HAND2 and at least one of PBX2, ACTN2, POU2F1, HAND1, TRIM24, GATA4, PBX1, ZBTB39, IKZF4, NROB2, NACA2, SMYD1, JUP, NEUROD1, CKMT2, TSHZ2, MITF, MYOCD, and PPARGC1B. In some compositions and methods, the ribonucleotide or ribonucleotides or deoxyribonucleotide or deoxyribonucleotides encode HAND1, GATA4, and at least one of PBX2, ACTN2, POU2F1, TRIM24, PBX1, ZBTB39, HAND2, IKZF4, NROB2, NACA2, SMYD1, JUP, NEUROD1, CKMT2, TSHZ2, MITF, MYOCD, and PPARGC1B.

In some compositions and methods, the ribonucleotide or ribonucleotides or deoxyribonucleotide or deoxyribonucleotides encode HAND2, GATA4, and at least one of PBX2, ACTN2, POU2F1, HAND1, TRIM24, PBX1, ZBTB39, IKZF4, NROB2, NACA2, SMYD1, JUP, NEUROD1, CKMT2, TSHZ2, MITF, MYOCD, and PPARGC1B. In some compositions and methods, the ribonucleotide or ribonucleotides or deoxyribonucleotide or deoxyribonucleotides encode HAND1, HAND2, and at least one of PBX2, ACTN2, POU2F1, TRIM24, GATA4, PBX1, ZBTB39, IKZF4, NROB2, NACA2, SMYD1, JUP, NEUROD1, CKMT2, TSHZ2, MITF, MYOCD, and PPARGC1B.

In some compositions and methods, the ribonucleotide or ribonucleotides or deoxyribonucleotide or deoxyribonucleotides encode HAND1, HAND2, and GATA4. In some compositions and methods, the ribonucleotide or ribonucleotides or deoxyribonucleotide or deoxyribonucleotides encode HAND1, HAND2, GATA4, and at least one of PBX2, ACTN2, POU2F1, TRIM24, PBX1, ZBTB39, IKZF4, NROB2, NACA2, SMYD1, JUP, NEUROD1, CKMT2, TSHZ2, MITF, MYOCD, and PPARGC1B.

In some compositions and methods, the cardiac disorder is selected from the group consisting of myocardial infarction, coronary artery disease, ischemic cardiomyopathy, cardiac fibrosis, congestive heart failure (CHF), end-stage heart failure, cardiomyopathy, dilated cardiomyopathy, restrictive cardiomyopathy, and hypertrophic cardiomyopathy, viral cardiomyopathy, myocarditis, chemical-induced cardiomyopathy, post-partum cardiomyopathy, cardiomyopathy due to endocrine disorders, high cholesterol diseases, hemochromatosis and sarcoidosis.

In another aspect, the invention provides vector(s) comprising any of the ribonucleotide or ribonucleotides or deoxyribonucleotide or deoxyribonucleotides disclosed herein.

Some vector(s) are viral vectors including retroviral systems such as MMLV, HIV-1, and ALV, adenoviral vectors, adeno-associated virus vectors, lentiviral vectors such as those based on HIV or FIV gag sequences; the poxvirus family such as vaccinia virus and the avian pox viruses, the alpha virus genus such as those derived from Sindbis and Semliki Forest Viruses, Venezuelan equine encephalitis virus, rhabdoviruses such as vesicular stomatitis virus, papillomaviruses, and baculoviruses, or nonviral vectors such as lipid-based vectors, polymeric vectors, dendrimer vectors, polypeptide vectors, and nanoparticles.

Some vector(s) are viral vectors including retroviral systems such as MMLV, HIV-1, and ALV, adenoviral vectors, adeno-associated virus vectors, lentiviral vectors such as those based on HIV or FIV gag sequences, the poxvirus family such as vaccinia virus and the avian pox viruses, the alpha virus genus such as those derived from Sindbis and Semliki Forest Viruses, Venezuelan equine encephalitis virus; rhabdoviruses such as vesicular stomatitis virus; papillomaviruses; or baculoviruses. Some vector(s) are retroviral vectors including retroviral systems such as MMLV, HIV-1, and ALV.

In some methods, the ribonucleotide or ribonucleotides or deoxyribonucleotide or deoxyribonucleotides are introduced by a vector or vectors. In some methods, the vector(s) are viral vectors including retroviral systems such as MMLV, HIV-1, and ALV, adenoviral vectors, adeno-associated virus vectors, lentiviral vectors such as those based on HIV or FIV gag sequences, the poxvirus family such as vaccinia virus and the avian pox viruses, the alpha virus genus such as those derived from Sindbis and Semliki Forest Viruses, Venezuelan equine encephalitis virus, rhabdoviruses such as vesicular stomatitis virus, papillomaviruses, and baculoviruses, or nonviral vectors such as lipid-based vectors, polymeric vectors, dendrimer vectors, polypeptide vectors, and nanoparticles.

In some methods, the vector(s) are viral vectors including retroviral systems such as MMLV, HIV-1, and ALV; adenoviral vectors; adeno-associated virus vectors, lentiviral vectors such as those based on HIV or FIV gag sequences; the poxvirus family such as vaccinia virus and the avian pox viruses; the alpha virus genus such as those derived from Sindbis and Semliki Forest Viruses; Venezuelan equine encephalitis virus; rhabdoviruses such as vesicular stomatitis virus; papillomaviruses; or baculoviruses. In some methods, the vector(s) are retroviral vectors including retroviral systems such as MMLV, HIV-1, and ALV.

A further understanding of the nature and advantages of the present invention may be realized by reference to the remaining portions of the specification and claims.

### BRIEF DESCRIPTION OF THE DRAWINGS

The patent application file contains at least one drawing executed in color. Copies of this patent application with color drawing(s) will be provided by the Office upon request and payment of the necessary fee.

FIG. 1 is a schematic of General Workflow to identify CFDs.

FIG. 2 depicts CFD Combination Screen Schema.

FIG. 3 is a schematic of parallel optimization/screening plan.

FIG. 4 depicts 3D-UMAP of top 200 reprogrammed MSCs and CMs (A) vs. top 200 reprogrammed MSCs and the CM center (B).

FIG. 5 depicts Fractions of top 200 reprogrammed MSCs containing an exogene.

FIG. 6 depicts UMAP 3D slingshot pseudotime lineages in 5 MSC lines with similar end points

FIGS. 7A-D depict Expression of representative exogenous (Exo) and endogenous (Endo) CFDs within lineages created by slingshot

FIG. 8 depicts Immunocytochemistry (ICC) anti-MYH6 confocal microscopy images of MSCs transduced with GFP or indicated CFD combinations.

FIG. 9 is a schematic showing reprogramming of mesenchymal stem cells with 3 to 5 transcription factors to cardiomyocytes.

FIGS. 10A and 10B depict 3D t-distributed stochastic neighbor embedding (t-SNE). (10A) UMAP of all cells. (10B) UMAP of top 200 reprogrammed cells and cardiomyocytes closest to the cardio center.

FIG. 11 depicts Expression of exogenous genes in top 200 reprogrammed cells. y-axis shows name of exogenous gene.

FIGS. 12-56 depict results of tradeSeq with PCA 100 slingshot. FIGS. 12A-C show Cell line 1B mitochondria genes, FIGS. 13A-C show Cell line 2G mitochondria genes. FIGS. 14A-C show Cell line 1W mitochondria genes. FIGS. 15A-C show Cell line 2R mitochondria genes. FIGS. 16A-C show Cell line 3Y mitochondria genes. Results for indicated genes are depicted in: GATA4 FIGS. 17 (exogenous) and 18 (endogenous); HAND1 FIGS. 19 (exogenous) and 20 (endogenous); HAND2 FIGS. 21 (exogenous) and 22 (endogenous); NACA2 FIGS. 23 (exogenous) and 24 (endogenous); ACTN2 FIGS. 25 (exogenous) and 26 (endogenous); CKMT2 FIGS. 27 (exogenous) and 28 (endogenous); IKXF4 FIGS. 29 (exogenous) and 30 (endogenous); JUP FIGS. 31 (exogenous) and 32 (endogenous); MITF FIGS. 33 (exogenous) and 34 (endogenous); MYOCD FIGS. 35 (exogenous) and 36 (endogenous); NEUROD1 FIGS. 37 (exogenous) and 38 (endogenous); NROB2 FIGS. 39 (exogenous) and 40 (endogenous); PBX1 FIGS. 41 (exogenous) and 42 (endogenous); PBX2 FIGS. 43 (exogenous) and 44 (endogenous); POU2F1 FIGS. 45 (exogenous) and 46 (endogenous); PPARGC1B FIGS. 47 (exogenous) and 48 (endogenous); SMYD FIGS. 49 (exogenous) and 50 (endogenous); TRIM24 FIGS. 51 (exogenous) and 52 (endogenous); TSHX2 FIGS. 53 (exogenous) and 54 (endogenous); ZBT39 FIGS. 55 (exogenous) and 56 (endogenous).

FIGS. 57-101 depict results of tradeSeq with UMAP 3D slingshot. FIGS. 57A-C show Cell line 1B mitochondria genes, FIGS. 58A-C show Cell line 2G mitochondria genes. FIGS. 59A-C show Cell line 1W mitochondria genes. FIGS. 60A-C show Cell line 2R mitochondria genes. FIGS. 61A-C show Cell line 3Y mitochondria genes. Results for indicated genes are depicted in: GATA4 FIGS. 62 (exogenous) and 63 (endogenous); HAND1 FIGS. 64 (exogenous) and 65 (endogenous); HAND2 FIGS. 66 (exogenous) and 67 (endogenous); NACA2 FIGS. 68 (exogenous) and 69 (endogenous); ACTN2 FIGS. 70 (exogenous) and 71 (endogenous); CKMT2 FIGS. 72 (exogenous) and 73 (endogenous); IKXF4 FIGS. 74 (exogenous) and 75 (endogenous); JUP FIGS. 76 (exogenous) and 77 (endogenous); MITF FIGS. 78 (exogenous) and 79 (endogenous); MYOCD FIGS. 80 (exogenous) and 81 (endogenous); NEUROD1 FIGS. 82 (exogenous) and 83 (endogenous); NROB2 FIGS. 84 (exogenous) and 85 (endogenous); PBX1 FIGS. 86 (exogenous) and 87 (endogenous); PBX2 FIGS. 88 (exogenous) and 89 (endogenous); POU2F1 FIGS. 90 (exogenous) and 91 (endogenous); PPARGC1B FIGS. 92 (exogenous) and 93 (endogenous); SMYD FIGS. 94 (exogenous) and 95 (endogenous); TRIM24 FIGS. 96 (exogenous) and 97 (endogenous); TSHX2 FIGS. 98 (exogenous) and 99 (endogenous); ZBT39 FIGS. 100 (exogenous) and 101 (endogenous).

FIGS. 102-109 depict results of immunocytochemistry confocal microscopy studies of cells treated with indicated CFD combinations or GFP control (FIG. 102). COM1 (FIG. 103); COM2 (FIG. 104); COM3 (FIG. 105); COM4 (FIG. 106); COM6 (FIG. 107); COM7 (FIG. 108); COM8 (FIG. 109).

### BRIEF DESCRIPTION OF THE SEQUENCES

SEQ ID NO:1 sets forth the nucleotide sequence of Homo sapiens PBX2 (C1) NM_002586.5.

SEQ ID NO:2 sets forth the amino acid sequence of Homo sapiens PBX2 NP_002577.2.

SEQ ID NO:3 sets forth the nucleotide sequence of Homo sapiens ACTN2 (C2) V1: NM_001103.4.

SEQ ID NO:4 sets forth the amino acid sequence of Homo sapiens ACTN2 I1: NP_001094.1.

SEQ ID NO:5 sets forth the nucleotide sequence of Homo sapiens ACTN2 V2: NM_001278343.2.

SEQ ID NO:6 sets forth the amino acid sequence of Homo sapiens ACTN2 I2: NP_001265272.1.

SEQ ID NO:7 sets forth the nucleotide sequence of Homo sapiens ACTN2 V3: NM_001278344.2.

SEQ ID NO:8 sets forth the amino acid sequence of Homo sapiens ACTN2 I3: NP_001265273.1.

SEQ ID NO:9 sets forth the nucleotide sequence of Homo sapiens POU2F1 (C3) V1: NM_002697.4.

SEQ ID NO: 10 sets forth the amino acid sequence of Homo sapiens POU2F1 I1: NP_002688.3.

SEQ ID NO:11 sets forth the nucleotide sequence of Homo sapiens POU2F1 V2: NM_001198783.2.

SEQ ID NO:12 sets forth the amino acid sequence of Homo sapiens POU2F1 I2:NP_001185712.1.

SEQ ID NO:13 sets forth the nucleotide sequence of Homo sapiens POU2F1 V3: NM_001198786.2.

SEQ ID NO:14 sets forth the amino acid sequence of Homo sapiens POU2F1 I3:NP_001185715.1.

SEQ ID NO:15 sets forth the nucleotide sequence of Homo sapiens POU2F1 V6: NM_001365849.1 and of the nucleotide sequence of Homo sapiens POU2F1 V5: NM_001365848.1

SEQ ID NO:16 sets forth the amino acid sequence of Homo sapiens POU2F1 I4: NP_001352778.1.

SEQ ID NO:17 sets forth the nucleotide sequence of Homo sapiens HAND1 (C4) NM_004821.3.

SEQ ID NO:18 sets forth the amino acid sequence of Homo sapiens HAND1 NP_004812.1.

SEQ ID NO:19 sets forth the nucleotide sequence of Homo sapiens HAND1 XM_005268531.2.

SEQ ID NO:20 sets forth the amino acid sequence of Homo sapiens HAND1 XP_005268588.1.

SEQ ID NO:21 sets forth the nucleotide sequence of Homo sapiens TRIM24 (C5) V2: NM_003852.4.

SEQ ID NO: 22 sets forth the amino acid sequence of Homo sapiens TRIM24 Ib: NP_003843.3.

SEQ ID NO:23 sets forth the nucleotide sequence of Homo sapiens TRIM24 V1: NM_015905.3.

SEQ ID NO:24 sets forth the amino acid sequence of Homo sapiens TRIM24 Ia: NP_056989.2.

SEQ ID NO:25 sets forth the nucleotide sequence of Homo sapiens GATA4 (C6) V2: NM_002052.5.

SEQ ID NO:26 sets forth the amino acid sequence of Homo sapiens GATA4 I2: NP_002043.2.

SEQ ID NO:27 sets forth the nucleotide sequence of Homo sapiens GATA4 V1: NM_001308093.3.

SEQ ID NO:28 sets forth the amino acid sequence of Homo sapiens GATA4 IL: NP_001295022.1.

SEQ ID NO: 29 sets forth the nucleotide sequence of Homo sapiens GATA4 V3: NM_001308094.2 and of the nucleotide sequence of Homo sapiens GATA4 V4: NM_001374273.1.

SEQ ID NO:30 sets forth the amino acid sequence of Homo sapiens GATA4 I3: NP_001295023.1 and of the amino acid sequence of Homo sapiens GATA4 I3: NP_001361202.1.

SEQ ID NO:31 sets forth the nucleotide sequence of Homo sapiens GATA4 V5: NM_001374274.1.

SEQ ID NO:32 sets forth the amino acid sequence of Homo sapiens GATA4 I4: NP_001361203.1.

SEQ ID NO:33 sets forth the nucleotide sequence of Homo sapiens PBX1 (C7) XM_005245229.4.

SEQ ID NO:34 sets forth the amino acid sequence of Homo sapiens PBX1 XP_005245286.1.

SEQ ID NO:35 sets forth the nucleotide sequence of Homo sapiens ZBTB39 (C8) NM_014830.3.

SEQ ID NO:36 sets forth the amino acid sequence of Homo sapiens ZBTB39 NP_055645.1.

SEQ ID NO:37 sets forth the nucleotide sequence of Homo sapiens HAND2 (C9) NM_021973.3.

SEQ ID NO:38 sets forth the amino acid sequence of Homo sapiens HAND2 NP_068808.1.

SEQ ID NO:39 sets forth the nucleotide sequence of Homo sapiens IKZF4 (C10) NM_001351091.2.

SEQ ID NO:40 sets forth the amino acid sequence of Homo sapiens IKZF4 NP_001338020.1.

SEQ ID NO:41 sets forth the nucleotide sequence of Homo sapiens NROB2 (C11) NM_021969.3.

SEQ ID NO:42 sets forth the amino acid sequence of Homo sapiens NROB2 NP_068804.1.

SEQ ID NO: 43 sets forth the nucleotide sequence of Homo sapiens NACA2 (C12) NM_199290.4.

SEQ ID NO:44 sets forth the amino acid sequence of Homo sapiens NACA2 NP_954984.1.

SEQ ID NO:45 sets forth the nucleotide sequence of Homo sapiens SMYD1 (C13) V1: NM_198274.4.

SEQ ID NO:46 sets forth the amino acid sequence of Homo sapiens SMYD1 I1: NP_938015.1.

SEQ ID NO:47 sets forth the nucleotide sequence of Homo sapiens SMYD1 V2: NM_001330364.2.

SEQ ID NO:48 sets forth the amino acid sequence of Homo sapiens SMYD 1 I2: NP_001317293.1.

SEQ ID NO:49 sets forth the nucleotide sequence of Homo sapiens JUP (C14) NM_021991.4.

SEQ ID NO:50 sets forth the amino acid sequence of Homo sapiens JUP NP_068831.1.

SEQ ID NO:51 sets forth the nucleotide sequence of Homo sapiens NEUROD1 (C15) NM_002500.5.

SEQ ID NO:52 sets forth the amino acid sequence of Homo sapiens NEUROD1 NP_002491.3.

SEQ ID NO:53 sets forth the nucleotide sequence of Homo sapiens CKMT2 (C16) NM_001099736.2.

SEQ ID NO:54 sets forth the amino acid sequence of Homo sapiens CKMT2 NP_001093206.1.

SEQ ID NO:55 sets forth the nucleotide sequence of Homo sapiens TSHZ2 (C17) V1: NM_173485.6.

SEQ ID NO:56 sets forth the amino acid sequence of Homo sapiens TSHZ2 I1: NP_775756.3.

SEQ ID NO:57 sets forth the nucleotide sequence of Homo sapiens TSHZ2 V2: NM_001193421.2.

SEQ ID NO:58 sets forth the amino acid sequence of Homo sapiens TSHZ2 I2: NP_001180350.1.

SEQ ID NO:59 sets forth the nucleotide sequence of Homo sapiens MITF (C18) NM_198159.3.

SEQ ID NO:60 sets forth the amino acid sequence of Homo sapiens MITF NP_937802.1.

SEQ ID NO: 61 sets forth the nucleotide sequence of Homo sapiens MYOCD (C19) V1: NM_001146312.3.

SEQ ID NO:62 sets forth the amino acid sequence of Homo sapiens MYOCD I1: NP_001139784.1.

SEQ ID NO:63 sets forth the nucleotide sequence of Homo sapiens MYOCD V2: NM_153604.4.

SEQ ID NO:64 sets forth the amino acid sequence of Homo sapiens MYOCD I2: NP_705832.1.

SEQ ID NO:65 sets forth the nucleotide sequence of Homo sapiens MYOCD V3: NM_001378306.1.

SEQ ID NO:66 sets forth the amino acid sequence of Homo sapiens MYOCD I3: NP_001365235.1.

SEQ ID NO:67 sets forth the nucleotide sequence of Homo sapiens PPARGC1B (C20) NM_133263.4.

SEQ ID NO:68 sets forth the amino acid sequence of Homo sapiens PPARGC1B NP_573570.3.

### Definitions

The terms “protein,” “polypeptide,” and “peptide,” used interchangeably herein, refer to polymeric forms of amino acids of any length, including coded and non-coded amino acids and chemically or biochemically modified or derivatized amino acids. The terms include polymers that have been modified, such as polypeptides having modified peptide backbones. The terms include natural full length proteins, fragments and synthetic peptides.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

A “gene” refers to a transcriptional unit including a promoter and sequence to be expressed from it as an RNA or protein. The sequence to be expressed can be genomic or cDNA or one or more non-coding RNAs including siRNAs or microRNAs among other possibilities. Other elements, such as introns, and other regulatory sequences may or may not be present.

The term “naked polynucleotide” refers to a polynucleotide not complexed with colloidal materials. Naked polynucleotides are sometimes cloned in a plasmid vector.

The term “vector” or “DNA vector” or “gene transfer vector” refers to a polynucleotide that is used to perform a “carrying” function for another polynucleotide. For example, vectors are often used to allow a polynucleotide to be propagated within a living cell, or to allow a polynucleotide to be packaged for delivery into a cell, or to allow a polynucleotide to be integrated into the genomic DNA of a cell. A vector may further comprise additional functional elements, for example it may comprise a transposon.

“Codon optimization” refers to a process of modifying a nucleic acid sequence for enhanced expression in particular host cells by replacing at least one codon of the native sequence with a codon that is more frequently or most frequently used in the genes of the host cell while maintaining the native amino acid sequence. For example, a polynucleotide encoding a fusion polypeptide can be modified to substitute codons having a higher frequency of usage in a given host cell as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally Ausubel et al., supra). One example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (NCBI) website. Typically, default program parameters can be used to perform the sequence comparison, although customized parameters can also be used. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89, 10915 (1989)).

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

For purposes of classifying amino acids substitutions as conservative or non-conservative, amino acids are grouped as follows: Group I (hydrophobic sidechains): norleucine, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

A “homologous” sequence (e.g., nucleic acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “fragment” when referring to a polypeptide means a polypeptide that is shorter or has fewer amino acids than the full-length polypeptide. The term “fragment” when referring to a polynucleotide means a polynucleotide that is shorter or has fewer nucleotides than the full-length polynucleotide. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The term “variant” as used herein includes modifications, derivatives, or chemical equivalents of the amino acid and nucleic acid sequences disclosed herein that perform substantially the same function as the polypeptides or nucleic acid molecules disclosed herein in substantially the same way. For instance, the variants have the same function of being able to act as a CFD. In one embodiment, variants of polypeptides disclosed herein include, without limitation, conservative amino acid substitutions. Variants of polypeptides also include additions and deletions to the polypeptide sequences disclosed herein. In addition, variant nucleotide sequences and polypeptide sequences include analogs and derivatives thereof.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others.

The term “pharmaceutically acceptable” means that the carrier, diluent, excipient, or auxiliary is compatible with the other ingredients of the formulation and not substantially deleterious to the recipient thereof.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease as perceived by the subject. A “sign” refers to objective evidence of a disease as observed by a physician.

Therapeutic agents of the invention are typically substantially pure from undesired contaminant. This means that an agent is typically at least about 50% w/w (weight/weight) purity, as well as being substantially free from interfering proteins, interfering polynucleotides, and contaminants. Sometimes the agents are at least about 80% w/w and, more preferably at least 90 or about 95% w/w purity.

As used herein, the term “autologous” is meant to refer to any material derived from the same individual to whom it is later to be re-introduced.

The term “xenogeneic” refers to any material derived from a different animal species than the animal species that becomes the recipient animal host in a transplantation or vaccination procedure.

The term “allogeneic” refers to any material derived from an animal that is of the same animal species but genetically different in one or more genetic loci as the animal that becomes the “recipient host”. This usually applies to cells transplanted from one animal to another non-identical animal of the same species.

The term “syngeneic” refers to any material derived from an animal which is of the same animal species and has the same genetic composition for most genotypic and phenotypic markers as the animal who becomes the recipient host of that cell line in a transplantation or vaccination procedure. This usually applies to cells transplanted from identical twins or may be applied to cells transplanted between highly inbred animals.

NETZEN: is a computational algorithm to predict master regulators of biological processes and cell fate determinants.

Slingshot is an algorithm designed to predict single cell lineage trajectory analysis (Street, K. et al., Cell reports 27. 12 (2019) 3846-3499). PCA100 slingshot is a slingshot analysis in which the input is the single cell dataset expressed with principle component analysis (PCA) 100 (100 dimension).

UMAP 3D slingshot a slingshot analysis in which the input is the single cell dataset expressed with uniform manifold approximation & projection analysis of 3 dimension.

Tradeseq is an R package computational method that allows analysis of gene expression along trajectories (Van den Berge et al. Nature communications, 11(1), 1-13).

Examples of a cardiac disorder are myocardial infarction, coronary artery disease, ischemic cardiomyopathy, cardiac fibrosis, congestive heart failure (CHF), end-stage heart failure, cardiomyopathy, dilated cardiomyopathy, restrictive cardiomyopathy, and hypertrophic cardiomyopathy, viral cardiomyopathy, myocarditis, chemical-induced cardiomyopathy, post-partum cardiomyopathy, cardiomyopathy due to endocrine disorders, high cholesterol diseases, hemochromatosis and sarcoidosis.

The term “patient” includes human and other mammalian subjects that receive either prophylactic or therapeutic treatment.

Use of the singular forms “a,” “an,” and “the” include plural references unless the context clearly dictates otherwise. Thus, for example, reference to “a ribonucleotide” includes a plurality of ribonucleotides, reference to “a deoxyribonucleotide” includes a plurality of deoxyribonucleotides, reference to “a CFD” includes a plurality of CFDs, and the like.

Where a combination is disclosed, each sub combination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Unless defined otherwise herein, all technical and scientific terms used herein have the same meaning as commonly understood by one of ordinary skill in the art to which this invention belongs. Singleton, et. al., Dictionary of Microbiology and Molecular Biology, 2nd Ed., John Wiley and Sons, New York (1994), and Hale & Marham, The Harper Collins Dictionary of Biology, Harper Perennial, N Y, 1991, provide one of skill with a general dictionary of many of the terms used in this invention. Although any methods and materials similar or equivalent to those described herein can be used in the practice or testing of the present invention, the preferred methods and materials are described. Unless otherwise indicated, nucleic acids are written left to right in 5′ to 3′ orientation; amino acid sequences are written left to right in amino to carboxy orientation, respectively. The terms defined immediately below are more fully defined by reference to the specification as a whole.

Compositions or methods “comprising” or “including” one or more recited elements may include other elements not specifically recited. For example, a composition that “comprises” or “includes” a ribonucleotide or ribonucleotides or a deoxyribonucleotide or deoxyribonucleotides may contain the ribonucleotide or ribonucleotides or deoxyribonucleotide or deoxyribonucleotides alone or in combination with other ingredients. When the disclosure refers to a feature comprising specified elements, the disclosure should alternatively be understood as referring to the feature consisting essentially of or consisting of the specified elements.

Designation of a range of values includes all integers within or defining the range, and all subranges defined by integers within the range.

Unless otherwise apparent from the context, the term “about” encompasses insubstantial variations, such as values within a standard margin of error of measurement (e.g., SEM) of a stated value.

Statistical significance means p≤0.05.

### DETAILED DESCRIPTION

### I. General

The invention provides compositions for treating a cardiac disorder. The compositions comprise a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B. The compositions are also useful for reprogramming a mesenchymal stem cell (MSC) to an autologous induced cardiomyocyte (iCM).

The inventors developed NETZEN, a deep learning algorithm, to identify cell fate determinants (CFDs) from public genomics data to direct highly efficient transdifferentiation of mesenchymal stem cells (MSCs), a nearly inexhaustible autologous source, to autologous induced CMs (iCMs).

NETZEN takes RNA sequencing expression datasets in both the origin and destination cells and ranks upstream CFDs that are predicted to fully complete fate transformation between the 2 cell types. In the human MSCs to iCMs conversion, the inventors performed combinatorial perturbation using the top 20 predicted CFDs followed by single cell RNA sequencing analysis and identified a cell cluster with significant overlaps with human primary CMs. Detailed analysis of this cell cluster, especially cells closest to the computationally determined center of the human primary CM cluster revealed several combinations of exogenous CFDs with some previously shown to be critical for cardiac development and functions, including GATA4 and HAND2. Remarkably, novel exogenous CFDs were also identified in this cluster that appear to be critical drivers for the transdifferentiation in cooperation with GATA4 and/or HAND2 but have not been previously demonstrated to regulate cardiac differentiation and functions.

The inventors have identified combinations of each least two CFDs selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B to direct transdifferentiation of mesenchymal stem cells (MSCs) to autologous induced CMs (iCMs). Some combinations comprise each least two to five CFDs selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B.

Preferably the combinations are (a) POU2F1, HAND1, GATA4, NACA2, and TSHZ2; (b) GATA4, IKZF4, NACA2, and TSHZ2; (c) POU2F1, HAND1, GATA4, and HAND2; (d) GATA4, HAND2, and IKZF4; (e) POU2F1, GATA4, and TSHZ2; (f) HAND1, GATA4, IKZF4, and NACA; (g) HAND1, GATA4, and NACA2; (h) POU2F1, HAND1, GATA4, IKZF4, and NACA2; (i) POU2F1, HAND1, GATA4, JUP, and TSHZ2; (j) ACTN2, POU2F1, HAND1, and GATA4; (k) HAND1 and at least one of PBX2, ACTN2, POU2F1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B; (l) HAND2 and at least one of PBX2, ACTN2, POU2F1, HAND1, TRIM24, GATA4, PBX1, ZBTB39, IKZF4, NROB2, NACA2, SMYD1, JUP, NEUROD1, CKMT2, TSHZ2, MITF, MYOCD, and PPARGC1B; (m) HAND1, GATA4, and at least one of PBX2, ACTN2, POU2F1, TRIM24, PBX1, ZBTB39, HAND2, IKZF4, NROB2, NACA2, SMYD1, JUP, NEUROD1, CKMT2, TSHZ2, MITF, MYOCD, and PPARGC1B; (n) HAND2, GATA4, and at least one of PBX2, ACTN2, POU2F1, HAND1, TRIM24, PBX1, ZBTB39, IKZF4, NROB2, NACA2, SMYD1, JUP, NEUROD1, CKMT2, TSHZ2, MITF, MYOCD, and PPARGC1B; (o) HAND1, HAND2, and at least one of PBX2, ACTN2, POU2F1, TRIM24, GATA4, PBX1, ZBTB39, IKZF4, NROB2, NACA2, SMYD1, JUP, NEUROD1, CKMT2, TSHZ2, MITF, MYOCD, and PPARGC1B; (p) HAND1, HAND2, and GATA4; or (q) HAND1, HAND2, GATA4, and at least one of PBX2, ACTN2, POU2F1, TRIM24, PBX1, ZBTB39, IKZF4, NROB2, NACA2, SMYD1, JUP, NEUROD1, CKMT2, TSHZ2, MITF, MYOCD, and PPARGC1B.

The inventors have validated successful conversion of MSCs to iCMs with CFD combinations of the invention. Immunocytochemistry in early transdifferentiated cells demonstrated alpha myosin heavy chain-positive muscle-like fibers and initial sarcomeric formation. The most efficient CFD combination will proceed to planned preclinical testing in a cardiac fibrosis model.

Embodiments of the invention are presented in the drawings and in the Examples.

Exemplary cell fate determinants (CFDs) of the invention are presented in Table 1.

TABLE 1
Cell Fate Determinants

| Symbol and | | | NCBI Transcript | NCBI Protein |
|---|---|---|---|---|
| CFD Number | Gene Name | Protein name | number | number |
| PBX2 (C1) | Homo sapiens | pre-B-cell | NM_002586.5 | NP_002577.2 |
| | PBX | leukemia | (SEQ ID NO: 1) | (SEQ ID NO: 2) |
| | homeobox 2 | transcription | | |
| | | factor 2 | | |
| ACTN2 (C2) | Homo sapiens | alpha-actinin-2 | V1: NM_001103.4 | I1: NP_001094.1 |
| | actinin alpha 2 | | (SEQ ID NO: 3) | (SEQ ID NO: 4) |
| | | | V2: NM_001278343.2 | I2: NP_001265272.1 |
| | | | (SEQ ID NO: 5) | (SEQ ID NO: 6) |
| | | | V3: NM_001278344.2 | I3: NP_001265273.1 |
| | | | (SEQ ID NO: 7) | (SEQ ID NO: 8) |
| POU2F1 (C3) | Homo sapiens | POU domain, | V1: NM_002697.4 | I1: NP_002688.3 |
| | POU class 2 | class 2, | (SEQ ID NO: 9) | (SEQ ID NO: 10) |
| | homeobox 1 | transcription | V2: NM_001198783.2 | I2: NP_001185712.1 |
| | | factor 1 | (SEQ ID NO: 11) | (SEQ ID NO: 12) |
| | | | V3: NM_001198786.2 | I3: NP_001185715.1 |
| | | | (SEQ ID NO: 13) | (SEQ ID NO: 14) |
| | | | V6: NM_001365849.1 | I4: NP_001352778.1 |
| | | | (SEQ ID NO: 15) | (SEQ ID NO: 16) |
| | | | V5: NM_001365848.1 | (Note: V6 and V5 |
| | | | (SEQ ID NO: 15) | encode I4) |
| HAND1 (C4) | Homo sapiens | heart- and neural | NM_004821.3 | NP_004812.1 |
| | heart and | crest derivatives- | (SEQ ID NO: 17) | (SEQ ID NO: 18) |
| | neural crest | expressed protein 1 | XM_005268531.2 | XP_005268588.1 |
| | derivatives | | (SEQ ID NO: 19) | (SEQ ID NO: 20) |
| | expressed 1 | | V2: NM_003852.4 | Ib: NP_003843.3 |
| TRIM24 (C5) | Homo sapiens | transcription | (SEQ ID NO: 21) | (SEQ ID NO: 22) |
| | tripartite | intermediary | V1: NM_015905.3 | Ia: NP_056989.2 |
| | motif | factor 1-alpha | (SEQ ID NO: 23) | (SEQ ID NO: 24) |
| | containing 24 | | | |
| GATA4 (C6) | Homo sapiens | transcription | V2: NM_002052.5 | I2: NP_002043.2 |
| | GATA | factor GATA-4 | (SEQ ID NO: 25) | (SEQ ID NO: 26) |
| | binding protein 4 | | V1: NM_001308093.3 | I1: NP_001295022.1 |
| | | | (SEQ ID NO: 27) | (SEQ ID NO: 28) |
| | | | V3: NM_001308094.2 | I3: NP_001295023.1 |
| | | | (SEQ ID NO: 29) | (SEQ ID NO: 30) |
| | | | V4: NM_001374273.1 | I3: NP_001361202.1 |
| | | | (SEQ ID NO: 29) | (SEQ ID NO: 30) |
| | | | V5: NM_001374274.1 | I4: NP_001361203.1 |
| | | | (SEQ ID NO: 31) | (SEQ ID NO: 32) |
| PBX1 (C7) | Homo sapiens | pre-B-cell | XM_005245229.4 | XP_005245286.1 |
| | PBX | leukemia | (SEQ ID NO: 33) | (SEQ ID NO: 34) |
| | homeobox 1 | transcription | | |
| | | factor 1 | | |
| ZBTB39 (C8) | Homo sapiens | zinc finger and | NM_014830.3 | NP_055645.1 |
| | zinc finger | BTB domain- | (SEQ ID NO: 35) | (SEQ ID NO: 36) |
| | and BTB domain | containing | | |
| | containing 39 | protein 39 | | |
| HAND2 (C9) | Homo sapiens | heart- and neural | NM_021973.3 | NP_068808.1 |
| | heart and | crest derivatives- | (SEQ ID NO: 37) | (SEQ ID NO: 38) |
| | neural crest | expressed protein 2 | | |
| | derivatives | | | |
| | expressed 2 | | | |
| IKZF4 (C10) | Homo sapiens | zinc finger | NM_001351091.2 | NP_001338020.1 |
| | IKAROS | protein Eos | (SEQ ID NO: 39) | (SEQ ID NO: 40) |
| | family zinc | | | |
| | finger 4 | | | |
| NR0B2 (C11) | Homo sapiens | nuclear receptor | NM_021969.3 | NP_068804.1 |
| | nuclear receptor | subfamily 0 | (SEQ ID NO: 41) | (SEQ ID NO: 42) |
| | subfamily 0 | group B member 2 | | |
| | group B | | | |
| | member 2 | | | |
| NACA2 (C12) | Homo sapiens | nascent | NM_199290.4 | NP_954984.1 |
| | nascent | polypeptide- | (SEQ ID NO: 43) | (SEQ ID NO: 44) |
| | polypeptide | associated | | |
| | associated | complex subunit | | |
| | complex subunit | alpha-2 | | |
| | alpha 2 | | | |
| SMYD1 (C13) | Homo sapiens | histone-lysine N- | V1: NM_198274.4 | I1: NP_938015.1 |
| | SET and MYND | methyltransferase | (SEQ ID NO: 45) | (SEQ ID NO: 46) |
| | domain | | V2: NM_001330364.2 | I2: NP_001317293.1 |
| | containing 1 | | (SEQ ID NO: 47) | (SEQ ID NO: 48) |
| JUP (C14) | Homo sapiens | junction | NM_021991.4 | NP_068831.1 |
| | junction | plakoglobin | (SEQ ID NO: 49) | (SEQ ID NO: 50) |
| | plakoglobin | | | |
| NEUROD1 | Homo sapiens | neurogenic | NM_002500.5 | NP_002491.3 |
| (C15) | neuronal | differentiation | (SEQ ID NO: 51) | (SEQ ID NO: 52) |
| | differentiation 1 | factor 1 | | |
| CKMT2 (C16) | Homo sapiens | creatine kinase S- | NM_001099736.2 | NP_001093206.1 |
| | creatine kinase, | type, mitochondrial | (SEQ ID NO: 53) | (SEQ ID NO: 54) |
| | mitochondrial 2 | precursor | | |
| TSHZ2 (C17) | Homo sapiens | teashirt homolog 2 | V1: NM_173485.6 | I1: NP_775756.3 |
| | teashirt zinc | | (SEQ ID NO: 55) | (SEQ ID NO: 56) |
| | finger | | V2: NM_001193421.2 | I2: NP_001180350.1 |
| | homeobox 2 | | (SEQ ID NO: 57) | (SEQ ID NO: 58) |
| MITF (C18) | Homo sapiens | microphthalmia- | NM_198159.3 | NP_937802.1 |
| | melanocyte | associated | (SEQ ID NO: 59) | (SEQ ID NO: 60) |
| | inducing | transcription | | |
| | transcription | factor | | |
| | factor | | | |
| MYOCD (C19) | Homo sapiens | myocardin | V1: NM_001146312.3 | I1: NP_001139784.1 |
| | myocardin | | (SEQ ID NO: 61) | (SEQ ID NO: 62) |
| | | | V2: NM_153604.4 | I2: NP_705832.1 |
| | | | (SEQ ID NO: 63) | (SEQ ID NO: 64) |
| | | | V3: NM_001378306.1 | I3: NP_001365235.1 |
| | | | (SEQ ID NO: 65) | (SEQ ID NO: 66) |
| PPARGC1B | Homo sapiens | peroxisome | NM_133263.4 | NP_573570.3 |
| (C20) | PPARG | proliferator- | (SEQ ID NO: 67) | (SEQ ID NO: 68) |
| | coactivator 1 | activated receptor | | |
| | beta | gamma coactivator | | |
| | | 1-beta | | |

### II. Nucleic Acids and Vectors

The invention further provides nucleic acids encoding any of the CFDs described above (e.g., SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, and 68). Exemplary nucleotide sequences include SEQ ID NOS:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, and 67. Optionally, such nucleic acids further encode a signal peptide and can be expressed with the signal peptide linked to the CFD. Coding sequences of nucleic acids can be operably linked with regulatory sequences to ensure expression of the coding sequences, such as a promoter, enhancer, ribosome binding site, transcription termination signal, and the like. The regulatory sequences can include a promoter, for example, a prokaryotic promoter or a eukaryotic promoter. The nucleic acid encoding a CFD can be codon-optimized for expression in a host cell. The nucleic acid encoding a CFD can encode a selectable gene. The nucleic acid encoding a CFD can occur in isolated form or can be cloned into one or more vectors. The nucleic acid can be synthesized by, for example, solid state synthesis or PCR of overlapping oligonucleotides. Nucleic acids encoding at least two CFDs can be joined as one contiguous nucleic acid, e.g., within an expression vector, or can be separate, e.g., each cloned into its own expression vector.

### III. Pharmaceutical Compositions and Methods of Use

Compositions comprising a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B can be used in the treatment of a cardiac disorder in a patient. Compositions of the invention are useful as therapeutic agents in the treatment of a cardiac disorder in a patient. Examples of such cardiac disorders include myocardial infarction, coronary artery disease, ischemic cardiomyopathy, cardiac fibrosis, congestive heart failure (CHF), end-stage heart failure, cardiomyopathy, dilated cardiomyopathy, restrictive cardiomyopathy, and hypertrophic cardiomyopathy, viral cardiomyopathy, myocarditis, chemical-induced cardiomyopathy, post-partum cardiomyopathy, cardiomyopathy due to endocrine disorders, high cholesterol diseases, hemochromatosis and sarcoidosis. In an example, the compositions are administered to a patient. Expression of at least two CFDs of the invention in the patient is useful in the treatment of a cardiac disorder.

In another example, the compositions can be incorporated in cells ex vivo, for example in cells explanted from an individual patient (e.g., bone marrow aspirates, umbilical cord tissue, molar cells, amniotic fluid, adipose tissue, tissue biopsy) or universal donor mesenchymal stem cells, followed by reimplantation of the cells into a patient, usually after selection for cells which have incorporated the transgenes. (see, e.g., WO 2017/091512). In some embodiments, the compositions reprogram explanted cells to induced cardiomyocytes (iCMs). Some explanted cells are mesenchymal stem cells. For example, mesenchymal stem cells can be reprogrammed to iCMs. iCMs implanted into a patient for treatment of a cardiac disorder can be autologous, syngeneic, allogeneic, xenogeneic or combinations thereof. The administered iCMs populate and repair damaged tissue, for example, cardiac tissue. These cells differentiate into the various lineages resulting in the regeneration and repair of damaged tissue. Examples of such cardiac disorders include myocardial infarction, coronary artery disease, ischemic cardiomyopathy, cardiac fibrosis, congestive heart failure (CHF), end-stage heart failure, cardiomyopathy, dilated cardiomyopathy, restrictive cardiomyopathy, and hypertrophic cardiomyopathy, viral cardiomyopathy, myocarditis, chemical-induced cardiomyopathy, post-partum cardiomyopathy, cardiomyopathy due to endocrine disorders, high cholesterol diseases, hemochromatosis and sarcoidosis.

A vector or segment therefrom encoding a CFD can be introduced into any region of interest in cells ex vivo, such as an albumin gene or other safe harbor gene. Cells incorporating the vector can be implanted with or without prior differentiation. Cells can be implanted into a specific tissue, such as a cardiac tissue or a location of pathology, or systemically, such as by infusion into the blood. For example, cells can be implanted into a cardiac tissue of a patient, such as the heart, optionally with prior differentiation to cells present in that tissue, such as cardiomyocytes in the case of a heart. Implantation of the iCMs in the patient is useful in treatment of a cardiac disorder in the patient.

Nucleic acids encoding at least CFD of the invention can be delivered in naked form (i.e., without colloidal or encapsulating materials). Vector systems can be used to deliver ribonucleotides or deoxyribonucleotides of the invention, including viral vectors such as retroviral systems (see, e.g., Lawrie and Tumin, Cur. Opin. Genet. Develop. 3, 102-109 (1993)) including retrovirus derived vectors such MMLV, HIV-1, and ALV; adenoviral vectors {see, e.g., Bett et al, J. Virol. 67, 591 1 (1993)); adeno-associated virus vectors {see, e.g., Zhou et al., J. Exp. Med. 179, 1867 (1994)), lentiviral vectors such as those based on HIV or FIV gag sequences, viral vectors from the pox family including vaccinia virus and the avian pox viruses, viral vectors from the alpha virus genus such as those derived from Sindbis and Semliki Forest Viruses (see, e.g., Dubensky et al., J. Virol. 70, 508-519 (1996)), Venezuelan equine encephalitis virus (see U.S. Pat. No. 5,643,576), rhabdoviruses, such as vesicular stomatitis virus (see WO 96/34625), papillomaviruses (Ohe et al., Human Gene Therapy 6, 325-333 (1995); Woo et al, WO 94/12629 and Xiao & Brandsma, Nucleic Acids. Res. 24, 2630-2622 (1996)), and baculoviruses (Haines et al, Baculoviruses: Expression Vector, Encyclopedia of Virology (third edition), 237-246 (2008)), and nonviral vectors such as lipid-based vectors, polymeric vectors, dendrimer vectors, polypeptide vectors, and nanoparticles (Mintzer and Simanek, Nonviral Vectors for Gene Delivery, Chem. Rev 109, 259-302 (2009)).

A nucleic acid encoding a CFD, or a vector containing the same, can be packaged into liposomes. Suitable lipids and related analogs are described by U.S. Pat. Nos. 5,208,036, 5,264,618, 5,279,833, and 5,283,185. Vectors and DNA encoding an immunogen or encoding the CFDs can also be adsorbed to or associated with particulate carriers, examples of which include polymethyl methacrylate polymers and polylactides and poly(lactide-co-glycolides), (see, e.g., McGee et al., J. Micro Encap. 1996).

Patients amenable to treatment include individuals at risk of a cardiac disorder, but not showing symptoms, as well as patients presently showing symptoms. Optionally, presence or absence of symptoms, signs or risk factors of a disease is determined before beginning treatment.

In some prophylactic applications, a composition of the invention is administered to a patient susceptible to, or otherwise at risk of a cardiac disorder in regime (dose, frequency and route of administration) effective to reduce the risk, lessen the severity, or delay the onset of at least one sign or symptom of the disease. In some prophylactic applications, a composition of the invention is used to reprogram a stem cell to an iCM, and the iCM is administered to a patient susceptible to, or otherwise at risk of a cardiac disorder in regime (dose, frequency and route of administration) effective to reduce the risk, lessen the severity, or delay the onset of at least one sign or symptom of the disease. In some therapeutic applications, a composition of the invention is administered to a patient suspected of, or already suffering from a cardiac disorder in a regime (dose, frequency and route of administration) effective to ameliorate or at least inhibit further deterioration of at least one sign or symptom of the disease. In some therapeutic applications, a composition of the invention is used to reprogram a stem cell to an iCM, and the iCM administered to a patient suspected of, or already suffering from a cardiac disorder in a regime (dose, frequency and route of administration) effective to ameliorate or at least inhibit further deterioration of at least one sign or symptom of the disease.

A regime is considered therapeutically or prophylactically effective if an individual treated patient achieves an outcome more favorable than the mean outcome in a control population of comparable patients not treated by methods of the invention, or if a more favorable outcome is demonstrated in treated patients versus control patients in a controlled clinical trial (e.g., a phase II, phase II/III or phase III trial) at the p<0.05 or 0.01 or even 0.001 level.

Effective doses of vary depending on many different factors, such as means of administration, target site, physiological state of the patient, whether the patient is human or an animal, other medications administered, and whether treatment is prophylactic or therapeutic.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen.

An effective amount of a composition is sufficient to generate a desired response, such as reduce or eliminate a sign or symptom of a cardiac disorder. In some embodiments, an “effective amount” is one that treats (including prophylaxis) one or more symptoms and/or underlying causes of any of a cardiac disorder. In some embodiments, an effective amount is a therapeutically effective amount. In some embodiments, an effective amount is an amount that prevents one or more signs or symptoms of a particular disease or condition from developing, such as one or more signs or symptoms associated with a cardiac disorder. The invention can be readily employed in a variety of therapeutic or prophylactic applications, e.g., for treating a cardiac disorder in a patient or for reprogramming a stem cell to an iCM useful in treating a cardiac disorder in a patient. Depending on the specific subject and conditions, pharmaceutical compositions of the invention can be administered to subjects by a variety of administration modes known to the person of ordinary skill in the art, for example, topical, intravenous, oral, subcutaneous, intraarterial, intra-articular, intracranial, intrathecal, intraperitoneal, intranasal, intraocular, parenteral, or intramuscular routes. A subcutaneous or intramuscular injection is most typically performed in the arm or leg muscles.

For prophylactic applications, the composition, or an iCM produced by a composition and/or by a method of the invention, is provided in advance of any symptom, for example in advance of a cardiac disorder. The prophylactic administration of the compositions or iCMs produced using a composition and method of the invention, serves to prevent or ameliorate any subsequent cardiac disorder. Thus, in some embodiments, a subject to be treated is one who has, or is at risk for developing, a cardiac disorder. Following administration of a therapeutically effective amount of the disclosed therapeutic compositions or of an iCM produced using a composition and method of the invention, the subject can be monitored for a cardiac disorder, symptoms associated with a cardiac disorder, or both.

For therapeutic applications, the composition or an iCM produced using a composition and method of the invention, is provided at or after the onset of a symptom of a cardiac disorder, for example after development of a symptom of a cardiac disorder, or after diagnosis of the cardiac disorder. The pharmaceutical composition of the invention or an iCM produced with a composition of and/or by a method of the invention, can be combined with other agents known in the art for treating or preventing a cardiac disorder.

### IV. Kits

The invention further provides kits (e.g., containers) comprising compositions disclosed herein and related materials, such as instructions for use (e.g., package insert). The instructions for use may contain, for example, instructions for administration of the compositions or of administration of an iCM produced using a composition of and/or by a method of the invention and optionally one or more additional agents. The containers of the compositions may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products.

Kits can also include a second container comprising a pharmaceutically-acceptable buffer, such as bacteriostatic water for injection (BWFI), phosphate-buffered saline, Ringer's solution and dextrose solution. It can also include other materials desirable from a commercial and user standpoint, including other buffers, diluents, filters, needles, and syringes.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### EXAMPLES

### Example 1: AI-Directed Transdifferentiation of Mesenchymal Stem Cells to Cardiomyocytes

Introduction & Objective

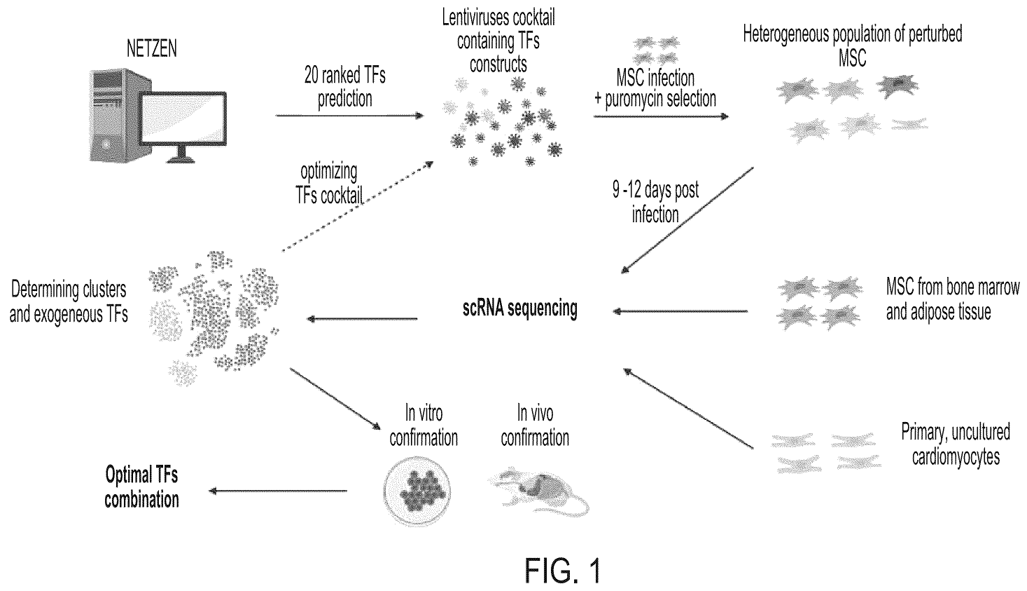

An abundant source of cardiomyocytes (CM) is critical for regenerative applications for cardiac fibrosis. Historically, cellular transdifferentiation relied on the highly inefficient and time consuming induced pluripotent stem cell intermediary. Moreover, mass production of autologous CMs remains the main obstacle to making conversion-sourced autologous cell transplantation a clinical reality. NETZEN, a deep learning algorithm, identifies cell fate determinants (CFDs) from public data to direct highly efficient transdifferentiation of mesenchymal stem cells (MSC), a nearly inexhaustible autologous source, to autologous induced CMs. By combining single cell RNA sequencing (scRNA-seq) and random viral integration, the inventors generated a heterogenous population of perturbed MSCs with different CFDs combinations (Duan, Jialei, et al. Cell reports 27.12 (2019): 3486-3499). Using lentiviral proportional and limited integration of the top 20 predicted CFDs (Table 2) in MSCs, followed by scRNA-seq analysis of reprogrammed cells, the inventors identified the most effective CFDs combination for the direct conversion (FIG. 1).

TABLE 2
MSC - Cardio CFDs

| CFD Number | CFD Name |
|---|---|
| C1 | PBX2 |
| C2 | ACTN2 |
| C3 | POU2F1 |
| C4 | HAND1 |
| C5 | TRIM24 |
| C6 | GATA4 |
| C7 | PBX1 |
| C8 | ZBTB39 |
| C9 | HAND2 |
| C10 | IKZF4 |
| C11 | NR0B2 |
| C12 | NACA2 |
| C13 | SMYD1 |
| C14 | JUP |
| C15 | NEUROD1 |
| C16 | CKMT2 |
| C17 | TSHZ2 |
| C18 | MITF |
| C19 | MYOCD |
| C20 | PPARGC1B |

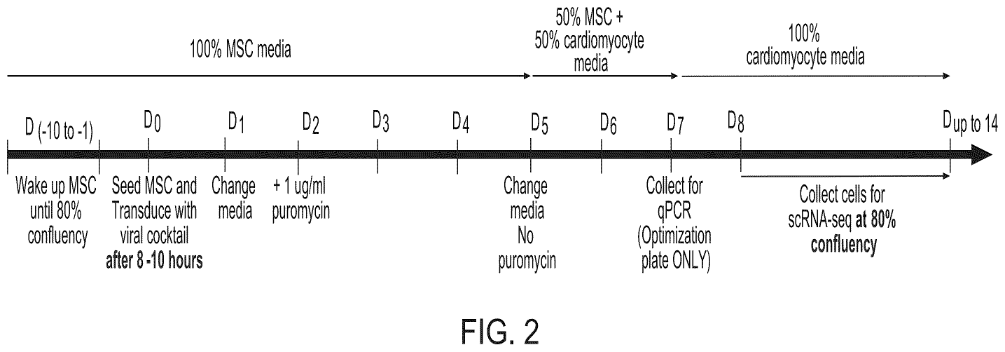

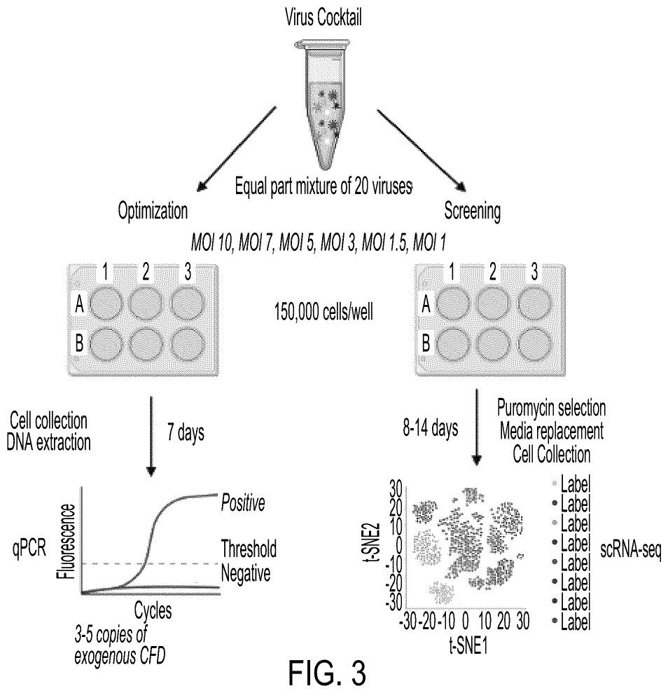

Materials & Methods
NETZEN takes RNA-seq datasets in both the origin and destination cells and ranks upstream CFDs predicted to fully complete fate transformation between the 2 cell types.
Plasmids for the 20 predicted CFDs under a CMV promoter were synthesized by GeneCopeia.
Lentiviral production was performed in Lenti-X 293T cells and viral titers determined by qPCR of transduced Lenti-X 293T cells genomes, using STOX2 as a standard.
The key objective for the optimization experiment was to determine the cocktail MOI that resulted in the integration of 3-5 copies of exogenous CFDs.
To ensure accuracy, the inventors performed concurrent optimization and screening assays of the same virus cocktail in the same individual MSC line (5 independent lines total) (FIGS. 2 and 3).
10× Chromium Single Cell 3′ GEM, Library & Gel Bead Kit v3 was used to create single cell cDNA and construct library for sequencing by Illumina.
Further analysis utilizing slingshot (Street, K., et al. BMC genomics, 19(1), 1-16) downstream of scRNA-seq dataset per MSC line with 100 PCA dimensions and UMAP 3D provided pseudotime trajectories under supervision (input starting and ending clusters).
TradeSeq (Van den Berge, et al. Nature communications, 11(1), 1-13) fitted the expression counts of a subset of 93 genes to a negative binomial generalized additive model (NB-GAM) and graphs the expression profile of cells along each pseudotime lineages computed by slingshot.
Immunocytochemistry was performed for cardiac markers (α-myosin heavy chain, cardiac troponin T and α-actinin).

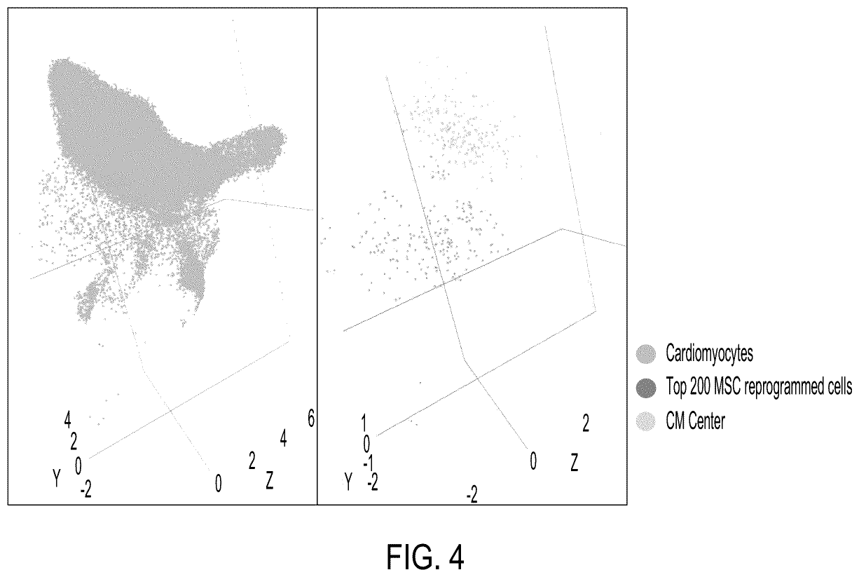

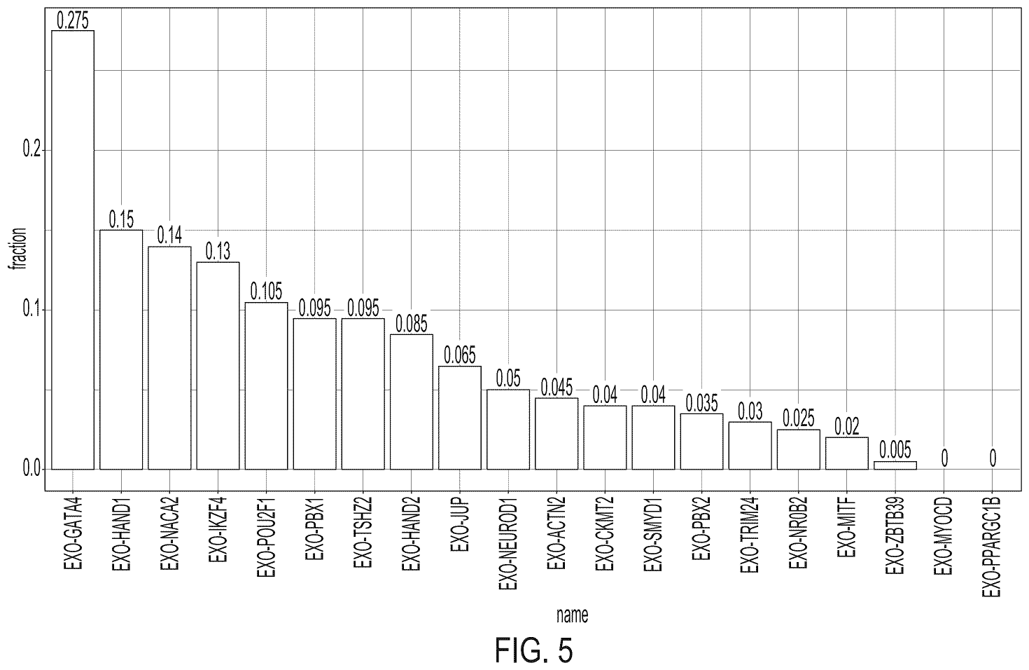

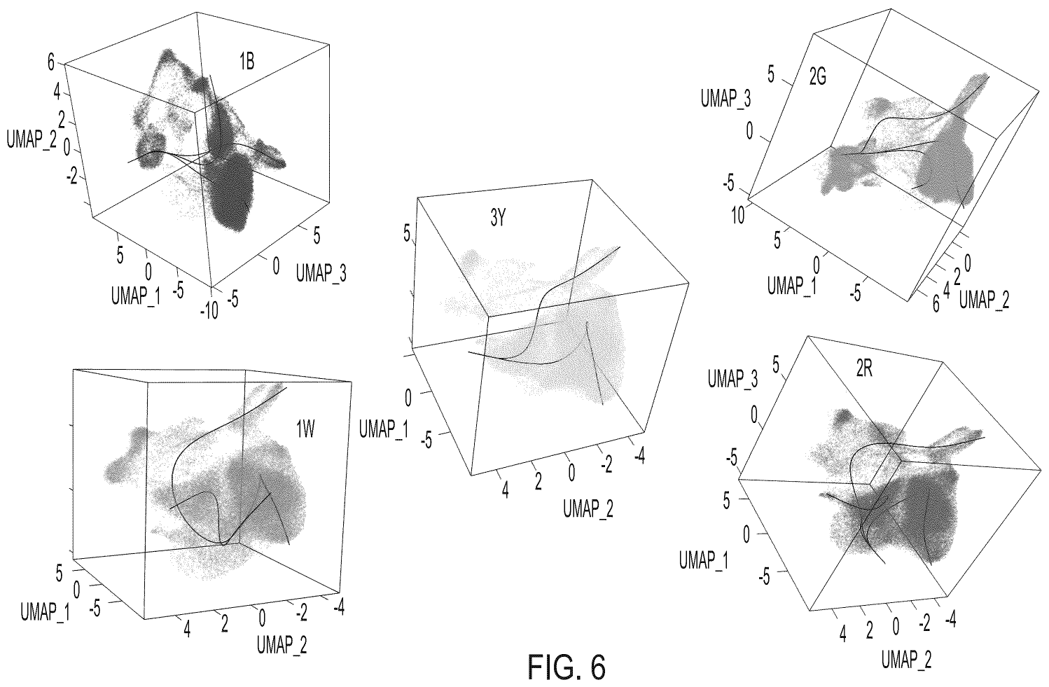

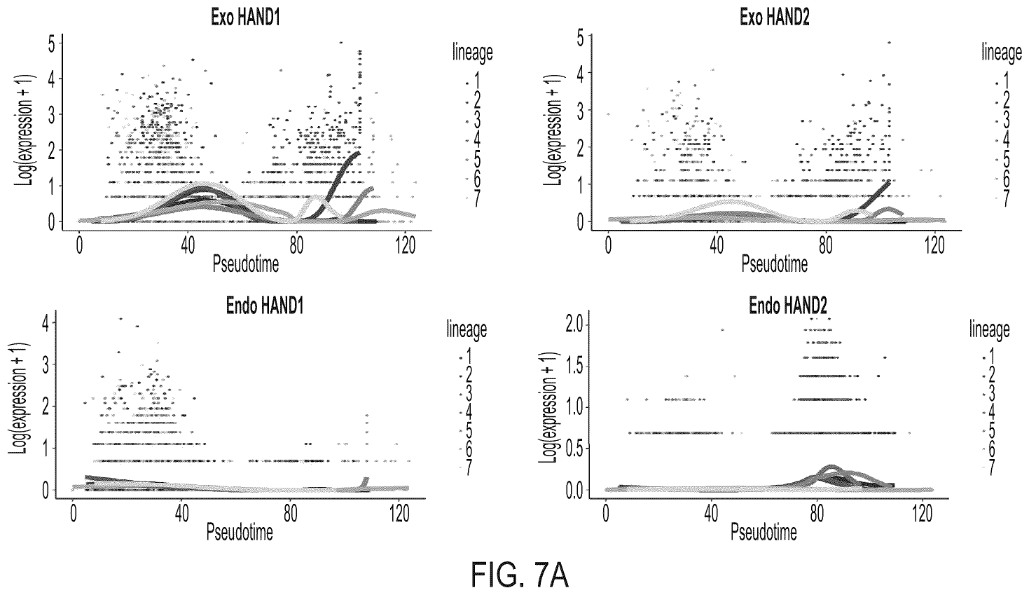

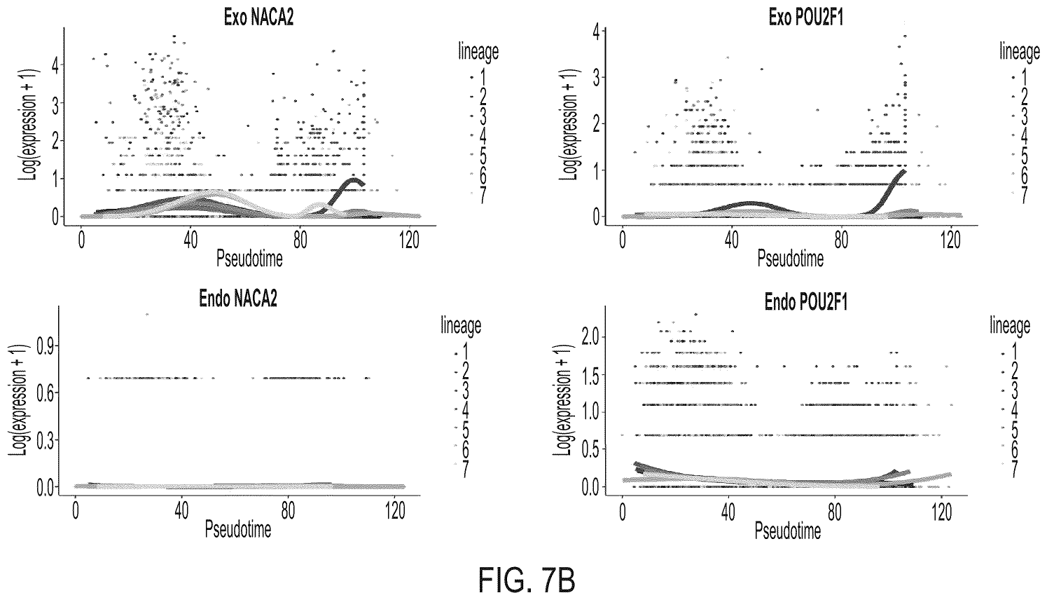

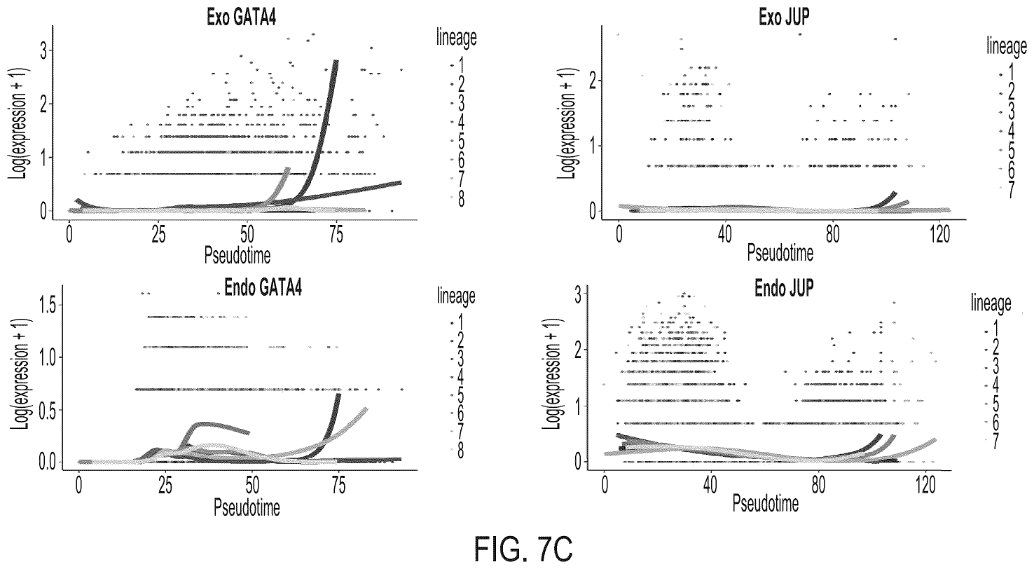

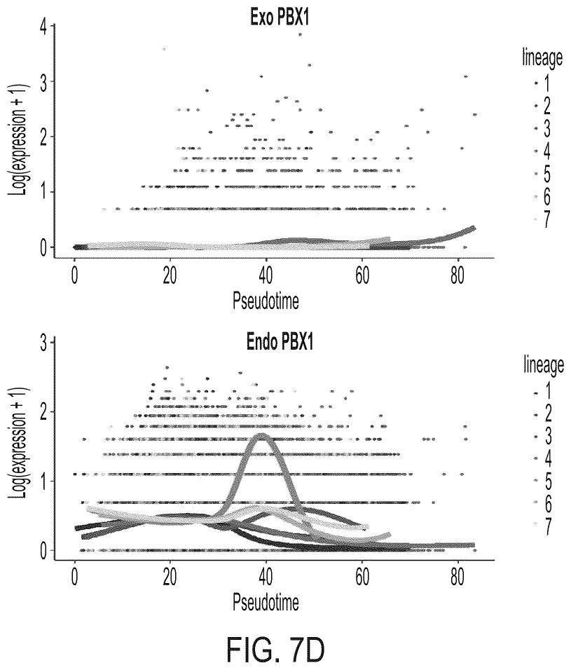

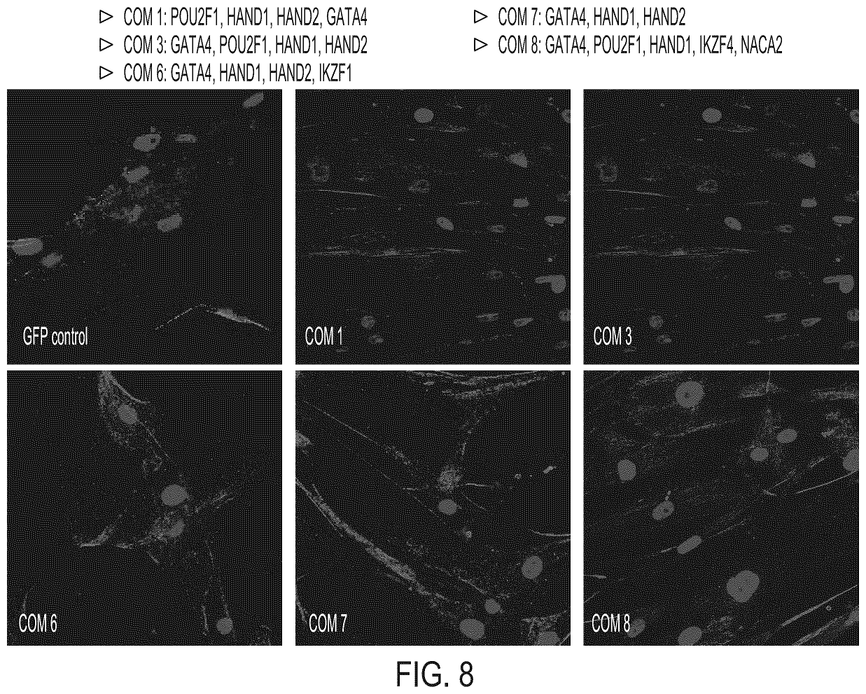

Result
The main operations were performed using the Seurat R package (3.2.2) (Butler, et al. Nature Biotech, 36(5):411-20). Sequencing data was aligned to the reference genome GRCh38 (GENCODE v.24) and gene count performed using the cellranger software (10× Genomics, version 4.0.0).
Dimensions were reduced via PCA and t-SNE and clustering normalized through an internal batch effect control.
The CM Center was determined as the average/central point of 75 dimensions of 30,000 primary CM from 5 donors.
The 200 transduced MSC cluster with the shortest distance to the CM center showed significant overlap with the CM cluster (FIG. 4).
Within this cluster, the 20 exogenous CFDs are compiled as fraction of the 200 cells (FIG. 5). GATA4, a known factor for CM development and functions, were present at the highest frequency in the top 200 transduced MSCs.
The expression profiles of exogenous CFDs and the corresponding endogenous CFDs of cell clusters along different lineages of the 5 MSC lines created by slingshot (FIG. 6) revealed patterns that correlated with the ranking of CFDs in the top 200 reprogrammed cells (HAND1, HAND2, GATA4 and NACA2) (FIGS. 7A-D).
Four combinations of CFDs were deduced and transduced into MSCs. Immunocytochemistry (ICC) for the cardiac marker alpha myosin heavy chain (MYH6) showed MYH6 expression fibrous patterns (red) similar to CMs, when compared to MSCs expressing GFP alone (FIG. 8). Nuclei (blue).

Conclusion
Using combinatorial perturbation, the inventors identified potential CFD combinations for MSCs to CM conversion from thousands of possible combinations.
Pseudotime trajectory and differential expression analyses revealed potential expression patterns of CFDs, which correlated with the high ranking CFDs in the top 200 reprogrammed cells (HAND1, HAND2, NACA2)
Preliminary ICC images provided a general guidelines for in vitro confirmation. Ongoing work is focused on validating the identity and functions of these reprogrammed MSCs both in vitro and in vivo models of cardiac fibrosis.

### Example 2: Combinatorial Perturbation of Mesenchymal Stem Cell (MSC) for Direct Reprogramming to Cardiomyocytes

Introduction

Direct reprogramming via exogeneous transcription factors (TFs) has the potential for multiple applications in medicine and science. As the in silico process of determining the most likely TFs for a direct conversion between two cell types become more intricate and fine-tuned, a new challenge emerges: optimization of the TFs combination experimentally for a specific conversion. The inventors determined the optimal TFs combination in the shortest amount of time and cover most of the possible combinations with combinatorial perturbation.

Application

Cardiomyocytes are vital for normal working of the hearts. Diseases/conditions that cause cardiomyocytes death such as myocardial infarction can lead to abnormal functioning of the heart or death.—MSC-induced Cardiomyocytes stands as a potential treatments to regenerate some functions of the patient's heart.

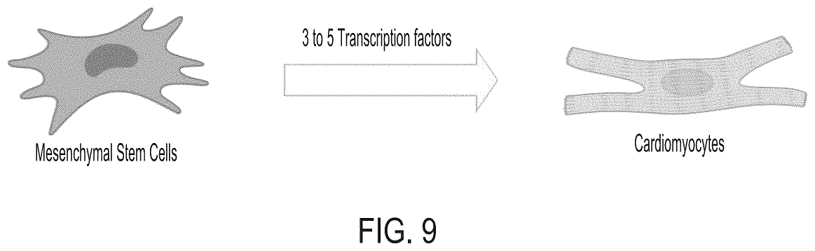

FIG. 9 is a schematic showing reprogramming of mesenchymal stem cells with 3 to 5 transcription factors to cardiomyocytes. Table 3 shows Lentiviruses expressing CFDs for MSCs-Cardiomyocyte Conversion.

TABLE 3
Lentiviruses expressing CFDs for
MSCs-Cardiomyocyte Conversion.

| MSC - Cardio CFDs | IU/ml |
|---|---|
| (C1) PBX2 | 7.809E+08 |
| (C2) ACTN2 | 4.787E+07 |
| (C3) POU2F1 | 1.226E+07 |
| (C4) HAND1 | 3.227E+07 |
| (C5) TRIM24 | 1.076E+08 |
| (C6)GATA4 | 5.212E+07 |
| (C7) PBX1 | 2.062E+08 |
| (C8) ZBTB39 | 1.070E+08 |
| (C9) HAND2 | 1.964E+08 |
| (C10) IKZF4 | 8.227E+07 |
| (C11) NR0B2 | 9.535E+08 |
| (C12) NACA2 | 2.099E+08 |
| (C13) SMYD1 | 5.113E+08 |
| (C14) JUP | 3.638E+08 |
| (C15) NEUROD1 | 4.470E+08 |
| (C16)CKMT2 | 1.397E+08 |
| (C17) TSHZ2 | 3.037E+07 |
| (C18) MITF | 5.164E+08 |
| (C19) MYOCD | 5.331E+08 |
| (C20) PPARGC1B | 1.962E+08 |

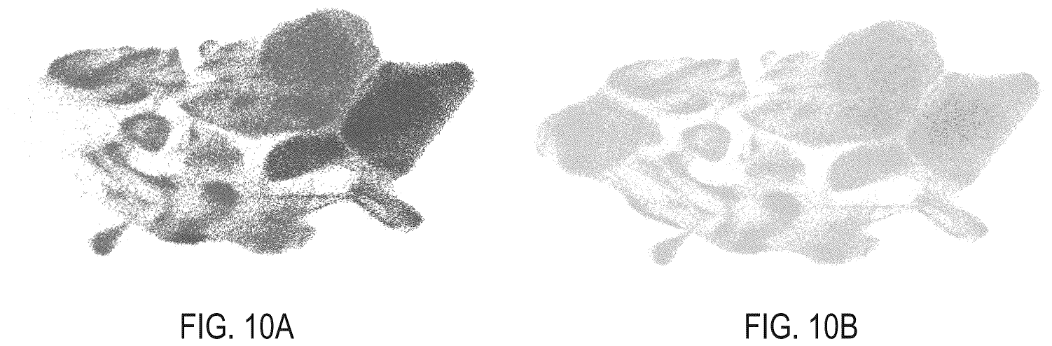

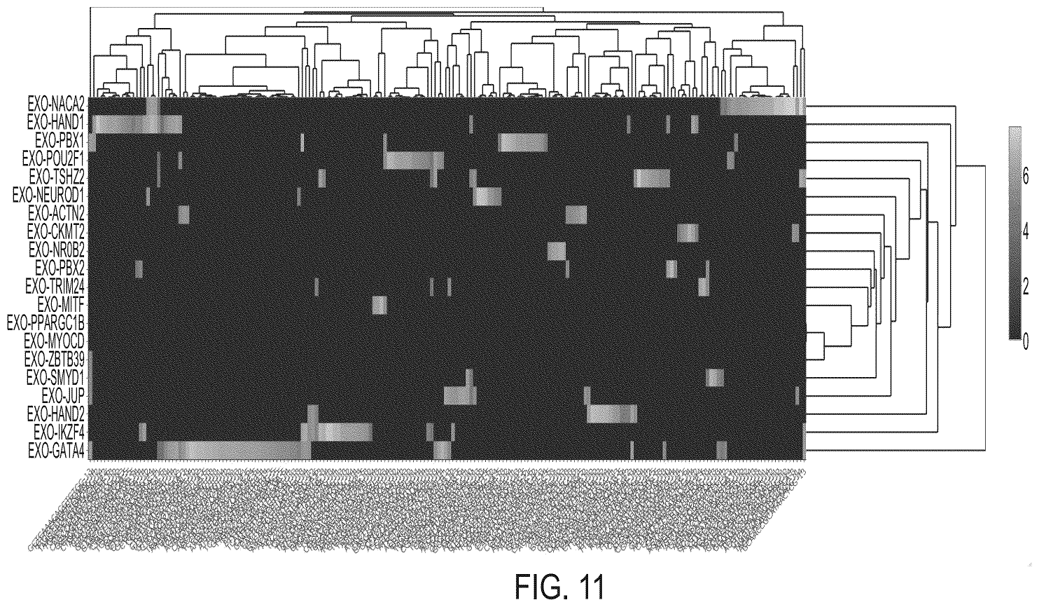

FIG. 2 shows CFD Combination Screen Schema. FIG. 3 shows optimization/screening plan. Table 4 shows example of viral cocktail calculation. Table 5 shows ScRNA-seq samples. FIGS. 10AB shows 3D t-SNE comparison of UMAP of all cells (FIG. 10A) vs. UMAP of top 200 reprogrammed cells and cardiomyocytes closest to the cardio center (FIG. 10B) FIG. 5 shows Fractions of top 200 cells containing an exogenous gene. Names of genes on x-axis and fraction on y-axis. FIG. 11 shows Expression of exogenous genes in top 200 reprogrammed cells. Names of genes on y-axis.

TABLE 4
example of viral cocktail calculation.

| Dilution | MSC - Cardio CFDs | IU/ml | MOI 1 | MOI 1.5 | MOI 3 | MOI 5 | MOI 7 | MOI 10 | Cocktail | Cocktail x2 |
|---|---|---|---|---|---|---|---|---|---|---|
| 1:10 | (C1) PBX2 | 7.809E+07 | 0.096 | 0.144 | 0.288 | 0.480 | 0.576 | 0.960 | 2.545 | 5.600 |
| | (C2) ACTN2 | 4.787E+07 | 0.157 | 0.235 | 0.470 | 0.783 | 0.940 | 1.567 | 4.152 | 9.134 |
| | (C3) POU2F1 | 1.226E+07 | 0.612 | 0.918 | 1.836 | 3.059 | 3.671 | 6.119 | 16.214 | 35.672 |
| | (C4) HAND1 | 3.227E+07 | 0.232 | 0.349 | 0.697 | 1.162 | 1.394 | 2.324 | 6.159 | 13.550 |
| | (C5) TRIM24 | 1.076E+08 | 0.070 | 0.105 | 0.209 | 0.349 | 0.418 | 0.697 | 1.848 | 4.065 |
| | (C6)GATA4 | 5.212E+07 | 0.144 | 0.216 | 0.432 | 0.719 | 0.863 | 1.439 | 3.813 | 8.389 |
| | (C7) PBX1 | 2.062E+08 | 0.036 | 0.055 | 0.109 | 0.182 | 0.218 | 0.364 | 0.964 | 2.121 |
| | (C8) ZBTB39 | 1.070E+08 | 0.070 | 0.105 | 0.210 | 0.350 | 0.420 | 0.701 | 1.857 | 4.085 |
| | (C9) HAND2 | 1.964E+08 | 0.038 | 0.057 | 0.115 | 0.191 | 0.229 | 0.382 | 1.012 | 2.227 |
| | (C10) IKZF4 | 8.227E+07 | 0.091 | 0.137 | 0.273 | 0.456 | 0.547 | 0.912 | 2.416 | 5.315 |
| 1:10 | (C11) NR0B2 | 9.535E+07 | 0.079 | 0.118 | 0.236 | 0.393 | 0.472 | 0.787 | 2.084 | 4.586 |
| | (C12) NACA2 | 2.099E+08 | 0.036 | 0.054 | 0.107 | 0.179 | 0.214 | 0.357 | 0.947 | 2.083 |
| 1:10 | (C13) SMYD1 | 5.113E+07 | 0.147 | 0.220 | 0.440 | 0.733 | 0.880 | 1.467 | 3.887 | 8.551 |
| 1:10 | (C14) JUP | 3.638E+07 | 0.206 | 0.309 | 0.618 | 1.031 | 1.237 | 2.061 | 5.463 | 12.018 |
| 1:10 | (C15) NEUROD1 | 4.470E+07 | 0.168 | 0.252 | 0.503 | 0.839 | 1.007 | 1.678 | 4.447 | 9.783 |
| | (C16)CKMT2 | 1.397E+08 | 0.054 | 0.081 | 0.161 | 0.268 | 0.322 | 0.537 | 1.422 | 3.129 |
| | (C17) TSHZ2 | 3.037E+07 | 0.247 | 0.370 | 0.741 | 1.235 | 1.482 | 2.470 | 6.545 | 14.399 |
| 1:10 | (C18) MITF | 5.164E+07 | 0.145 | 0.218 | 0.436 | 0.726 | 0.871 | 1.452 | 3.849 | 8.468 |
| 1:10 | (C19) MYOCD | 5.331E+07 | 0.141 | 0.211 | 0.422 | 0.703 | 0.844 | 1.407 | 3.728 | 8.201 |
| | (C20) PPARGC1B | 1.962E+08 | 0.038 | 0.057 | 0.115 | 0.191 | 0.229 | 0.382 | 1.013 | 2.229 |
| | | | 2.806 | 4.209 | 8.419 | 14.031 | 16.837 | 28.062 | 74.365 | 163.602 |

TABLE 5
ScRNA-seq samples

| Dilution | MSC - Cardio CFDs | IU/ml | MOI 1 | MOI 1.5 | MOI 3 | MOI 5 | MOI 7 | MOI 10 | Cocktail | Cocktail x2 |
|---|---|---|---|---|---|---|---|---|---|---|
| 1:10 | (C1) PBX2 | 7.809E+07 | 0.096 | 0.144 | 0.288 | 0.480 | 0.576 | 0.960 | 2.545 | 5.600 |
| | (C2) ACTN2 | 4.787E+07 | 0.157 | 0.235 | 0.470 | 0.783 | 0.940 | 1.567 | 4.152 | 9.134 |
| | (C3) POU2F1 | 1.226E+07 | 0.612 | 0.918 | 1.836 | 3.059 | 3.671 | 6.119 | 16.214 | 35.672 |
| | (C4) HAND1 | 3.227E+07 | 0.232 | 0.349 | 0.697 | 1.162 | 1.394 | 2.324 | 6.159 | 13.550 |
| | (C5) TRIM24 | 1.076E+08 | 0.070 | 0.105 | 0.209 | 0.349 | 0.418 | 0.697 | 1.848 | 4.065 |
| | (C6)GATA4 | 5.212E+07 | 0.144 | 0.216 | 0.432 | 0.719 | 0.863 | 1.439 | 3.813 | 8.389 |
| | (C7) PBX1 | 2.062E+08 | 0.036 | 0.055 | 0.109 | 0.182 | 0.218 | 0.364 | 0.964 | 2.121 |
| | (C8) ZBTB39 | 1.070E+08 | 0.070 | 0.105 | 0.210 | 0.350 | 0.420 | 0.701 | 1.857 | 4.085 |
| | (C9) HAND2 | 1.964E+08 | 0.038 | 0.057 | 0.115 | 0.191 | 0.229 | 0.382 | 1.012 | 2.227 |
| | (C10) IKZF4 | 8.227E+07 | 0.091 | 0.137 | 0.273 | 0.456 | 0.547 | 0.912 | 2.416 | 5.315 |
| 1:10 | (C11) NROB2 | 9.535E+07 | 0.079 | 0.118 | 0.236 | 0.393 | 0.472 | 0.787 | 2.084 | 4.586 |
| | (C12) NACA2 | 2.099E+08 | 0.036 | 0.054 | 0.107 | 0.179 | 0.214 | 0.357 | 0.947 | 2.083 |
| 1:10 | (C13) SMYD1 | 5.113E+07 | 0.147 | 0.220 | 0.440 | 0.733 | 0.880 | 1.467 | 3.887 | 8.551 |
| 1:10 | (C14) JUP | 3.638E+07 | 0.206 | 0.309 | 0.618 | 1.031 | 1.237 | 2.061 | 5.463 | 12.018 |
| 1:10 | (C15) NEUROD1 | 4.470E+07 | 0.168 | 0.252 | 0.503 | 0.839 | 1.007 | 1.678 | 4.447 | 9.783 |
| | (C16)CKMT2 | 1.397E+08 | 0.054 | 0.081 | 0.161 | 0.268 | 0.322 | 0.537 | 1.422 | 3.129 |
| | (C17) TSHZ2 | 3.037E+07 | 0.247 | 0.370 | 0.741 | 1.235 | 1.482 | 2.470 | 6.545 | 14.399 |
| 1:10 | (C18) MITF | 5.164E+07 | 0.145 | 0.218 | 0.436 | 0.726 | 0.871 | 1.452 | 3.849 | 8.468 |
| 1:10 | (C19) MYOCD | 5.331E+07 | 0.141 | 0.211 | 0.422 | 0.703 | 0.844 | 1.407 | 3.728 | 8.201 |
| | (C20) PPARGC1B | 1.962E+08 | 0.038 | 0.057 | 0.115 | 0.191 | 0.229 | 0.382 | 1.013 | 2.229 |
| | | | 2.806 | 4.209 | 8.419 | 14.031 | 16.837 | 28.062 | 74.365 | 163.602 |

Bioinformatic Pipeline

Further analysis utilizing slingshot (Street, K., et al. BMC genomics, 19(1), 1-16) downstream of single cell RNA dataset per MSC line with 100 PCA dimensions and UMAP 3D reduced from ˜20,000 genes provide pseudotime trajectories under supervision (input starting and ending cluster) TradeSeq (Van den Berge, et al. Nature communications, 11(1), 1-13) fits the expression counts of a subset of 93 genes to a negative binomial generalized additive model (NB-GAM) and graphs the expression profile of cells along each pseudotime lineages computed by slingshot. By examining the expression profile of overexpressed predicted transcription factors as well as the corresponding endogenous of cells along lineages, the inventors observed expression patterns correlate with the high ranking genes in the top 200 reprogrammed cells (HAND1, HAND2, NACA2)

FIG. 6 shows UMAP 3D slingshot pseudotime lineages in 5 MSC lines with similar end points.

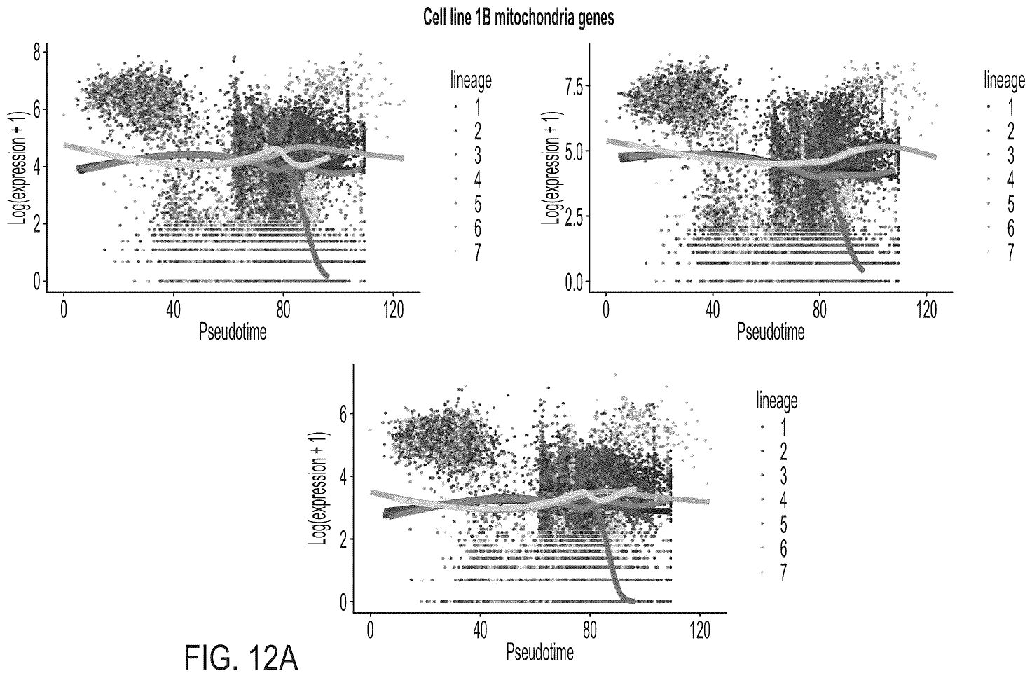

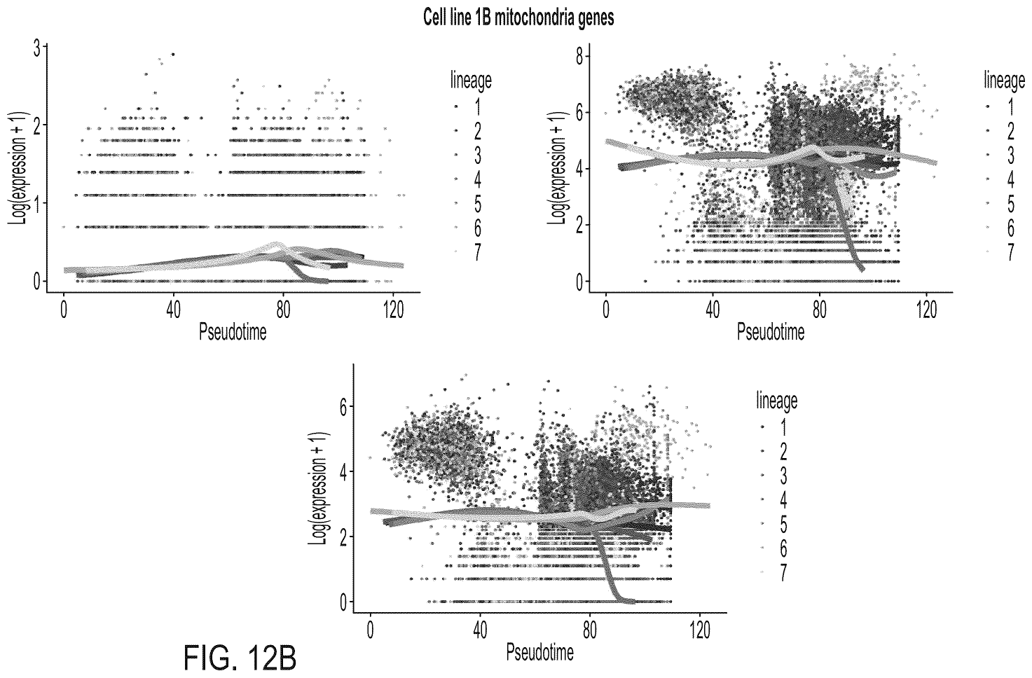

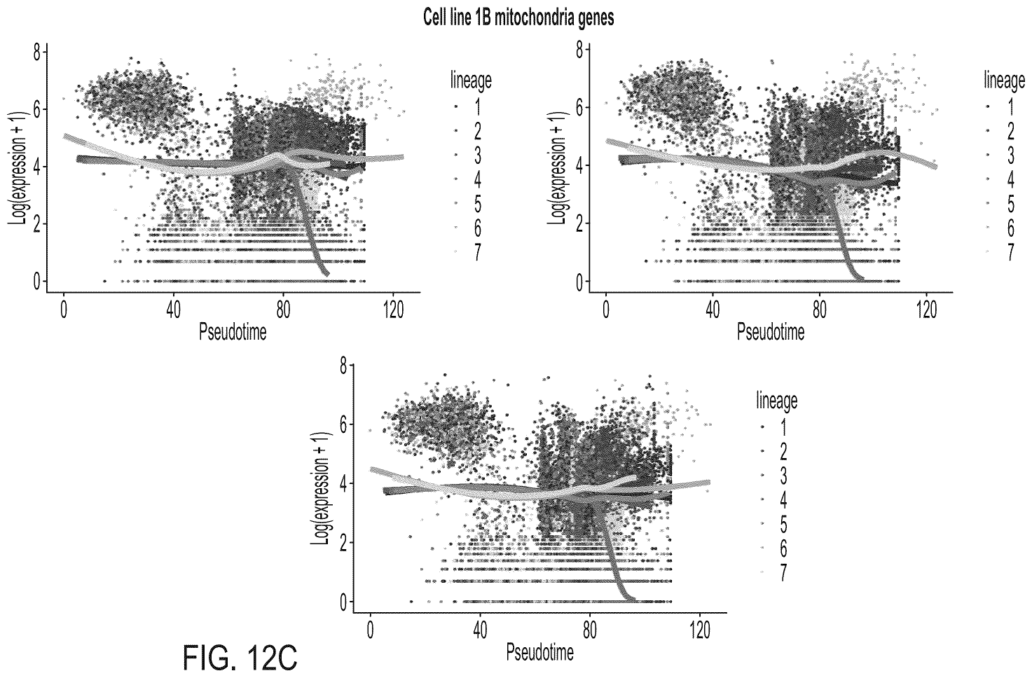

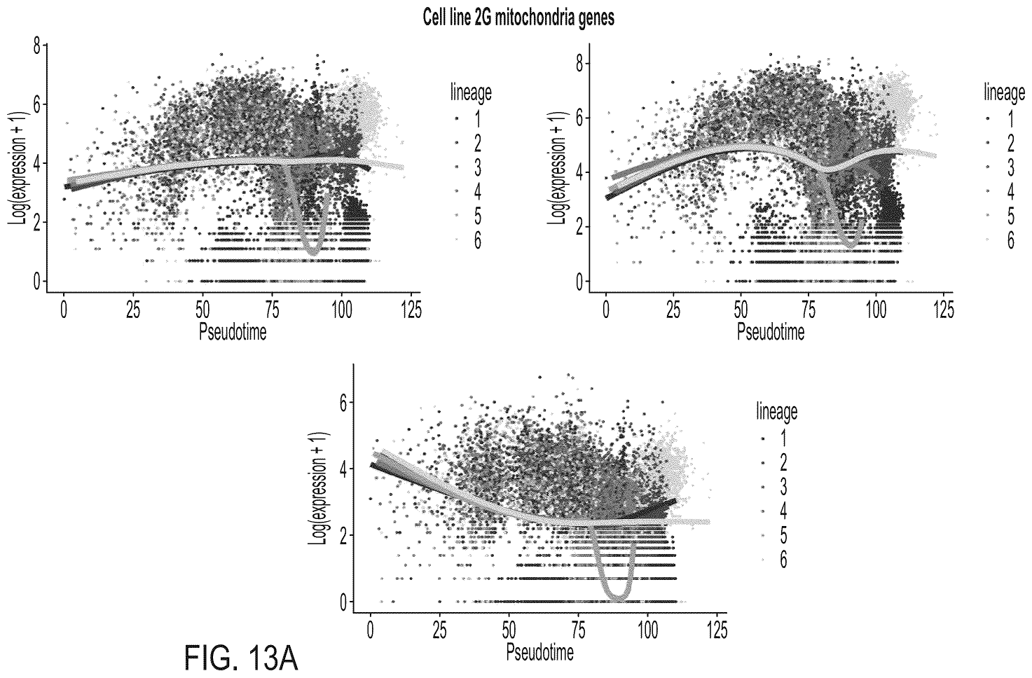

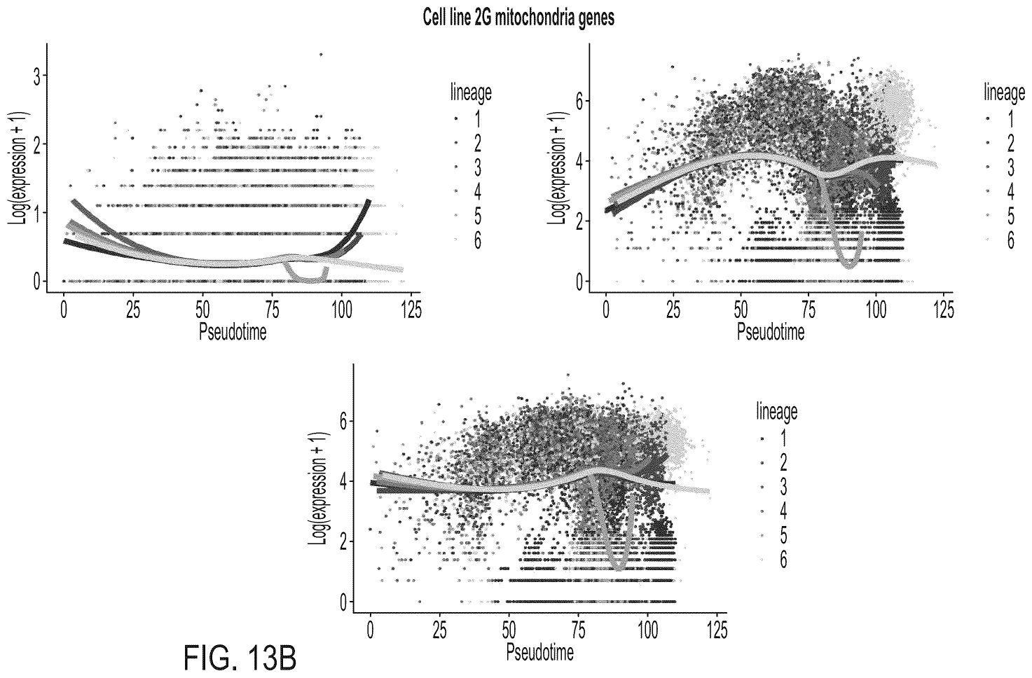

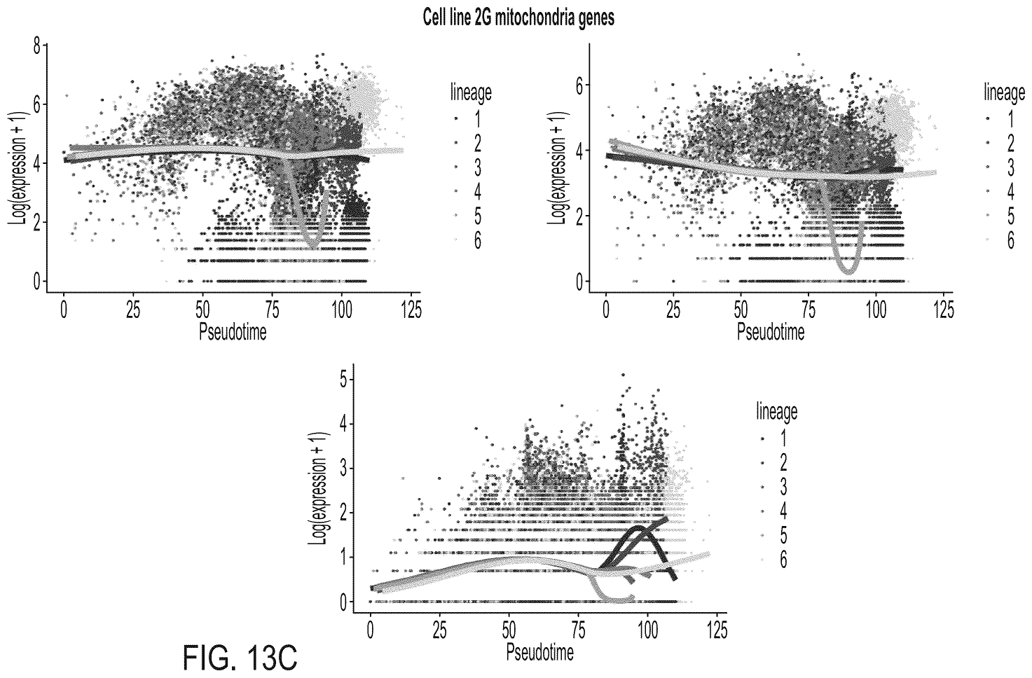

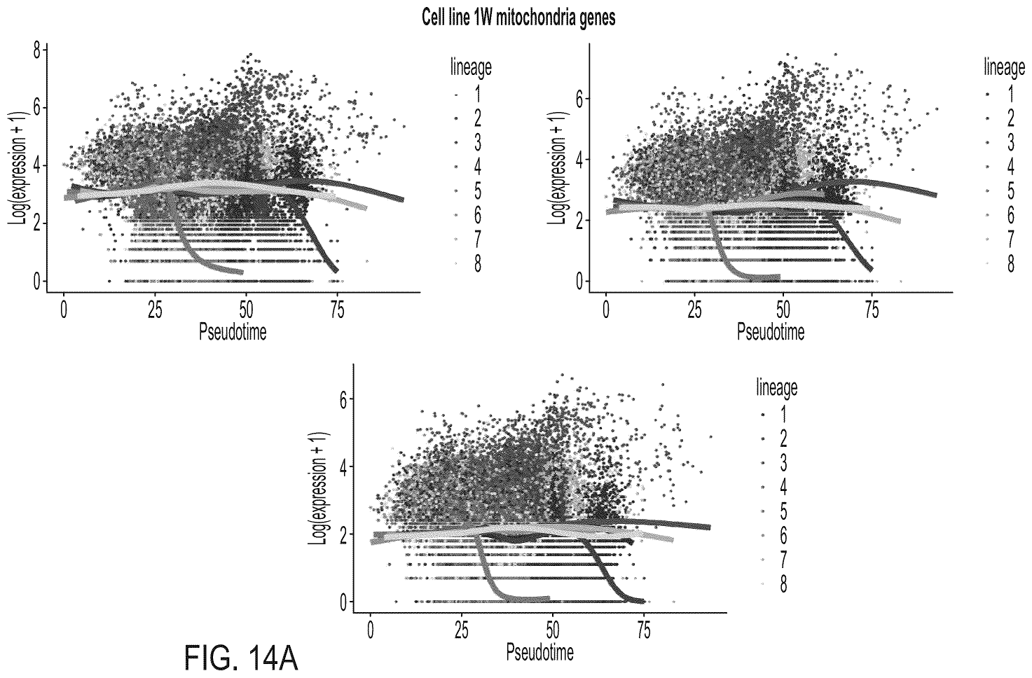

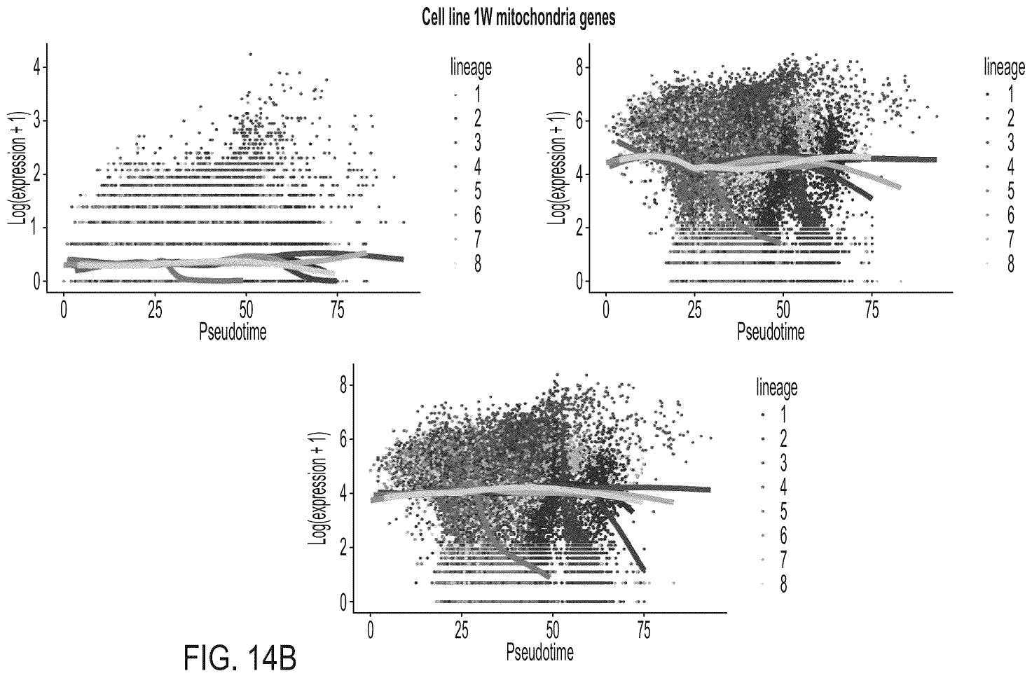

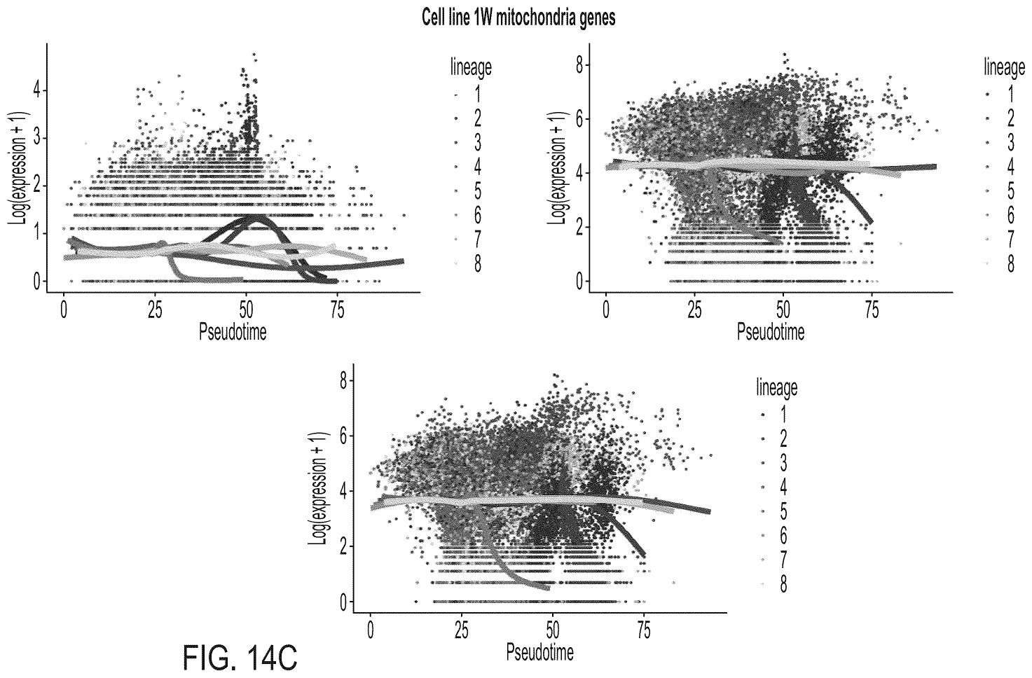

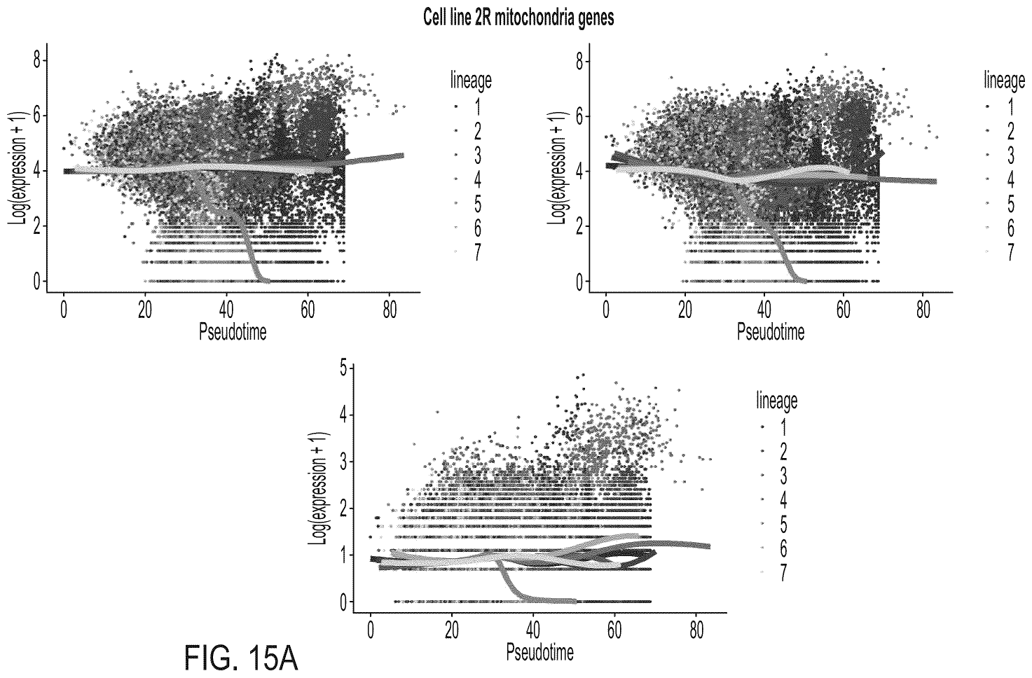

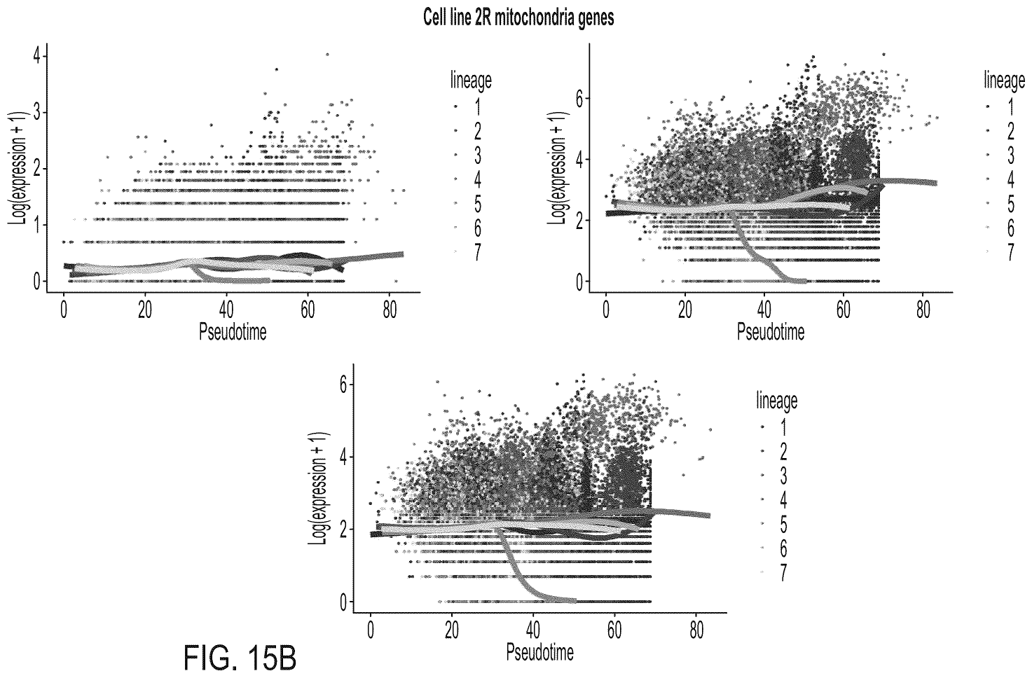

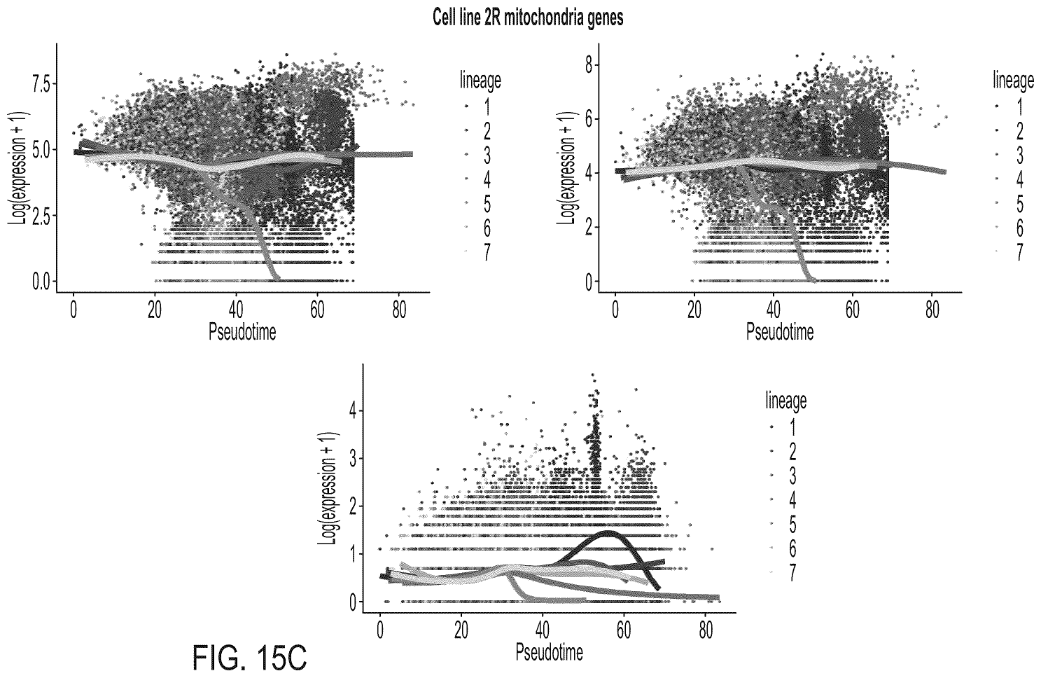

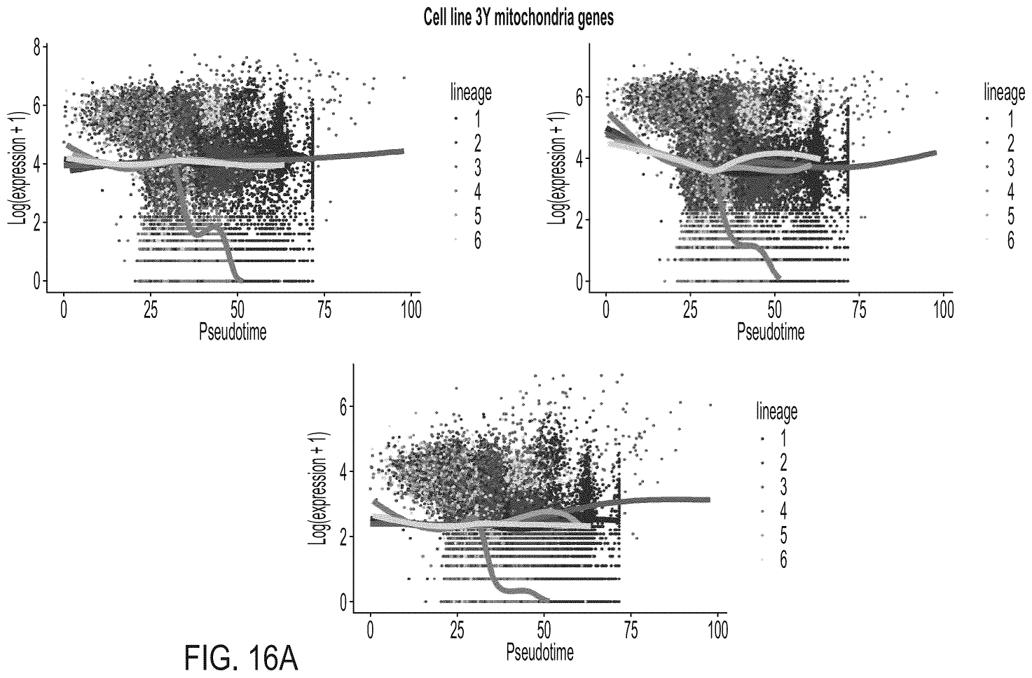

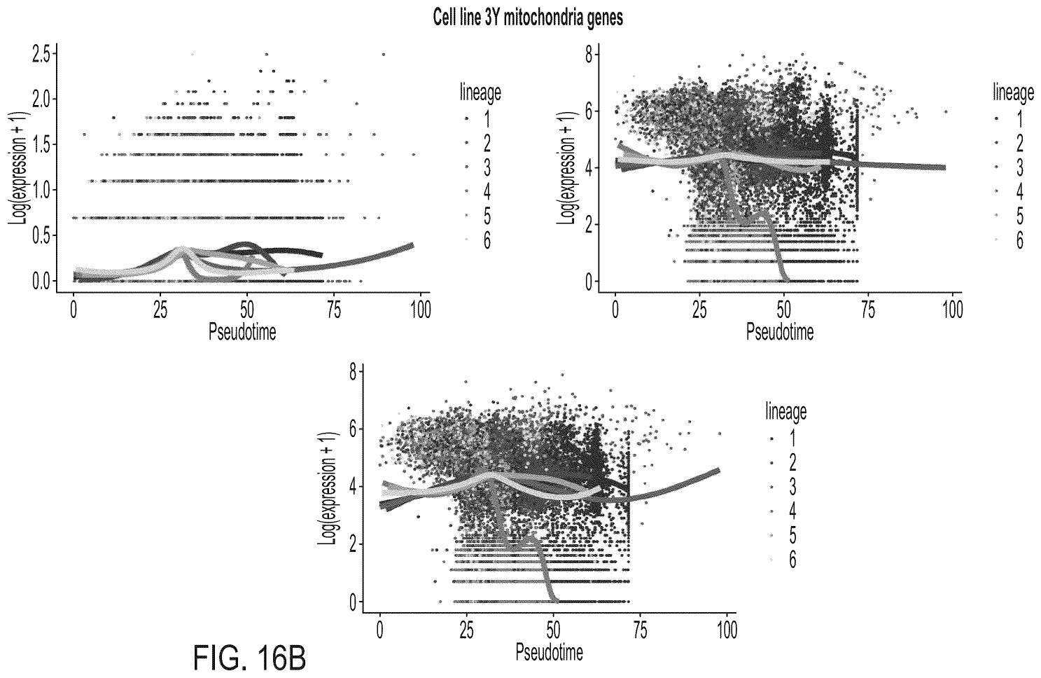

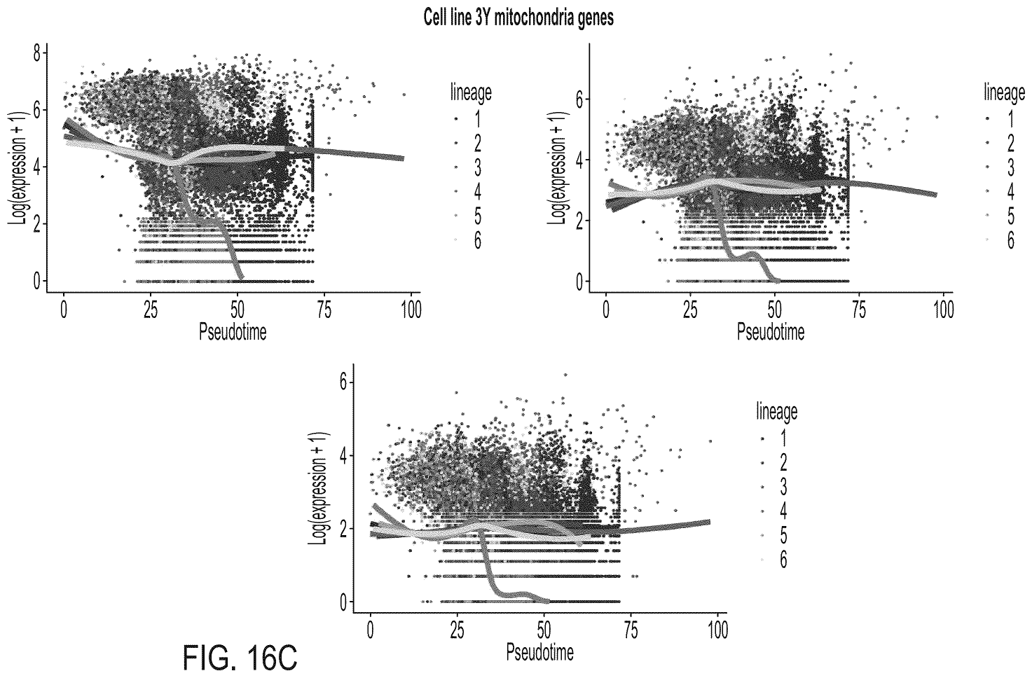

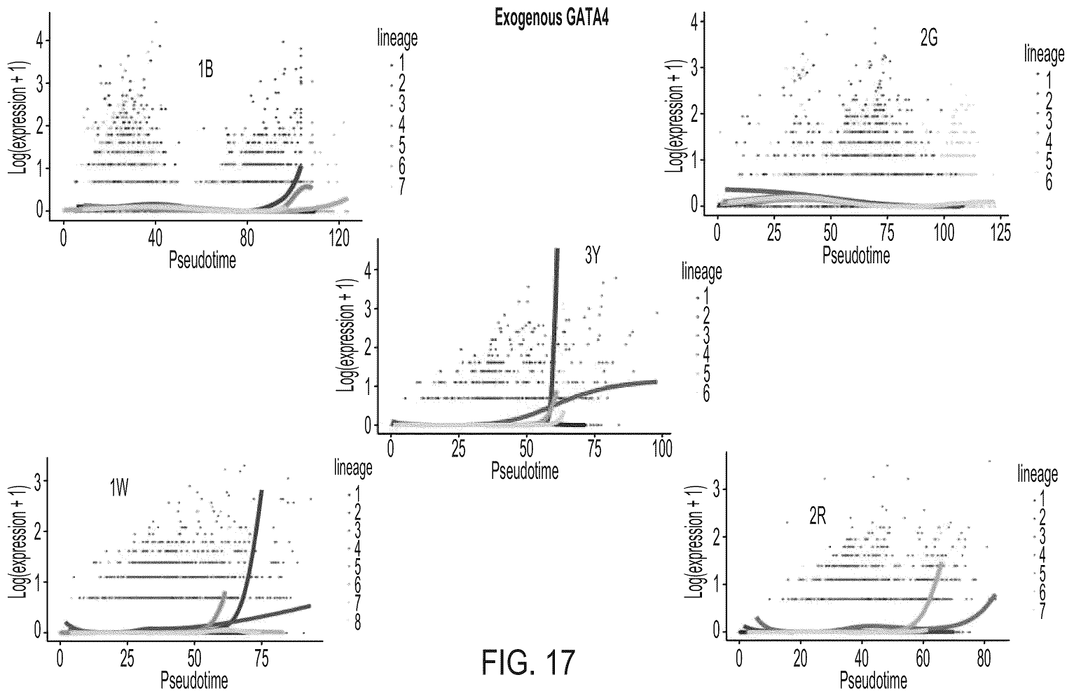

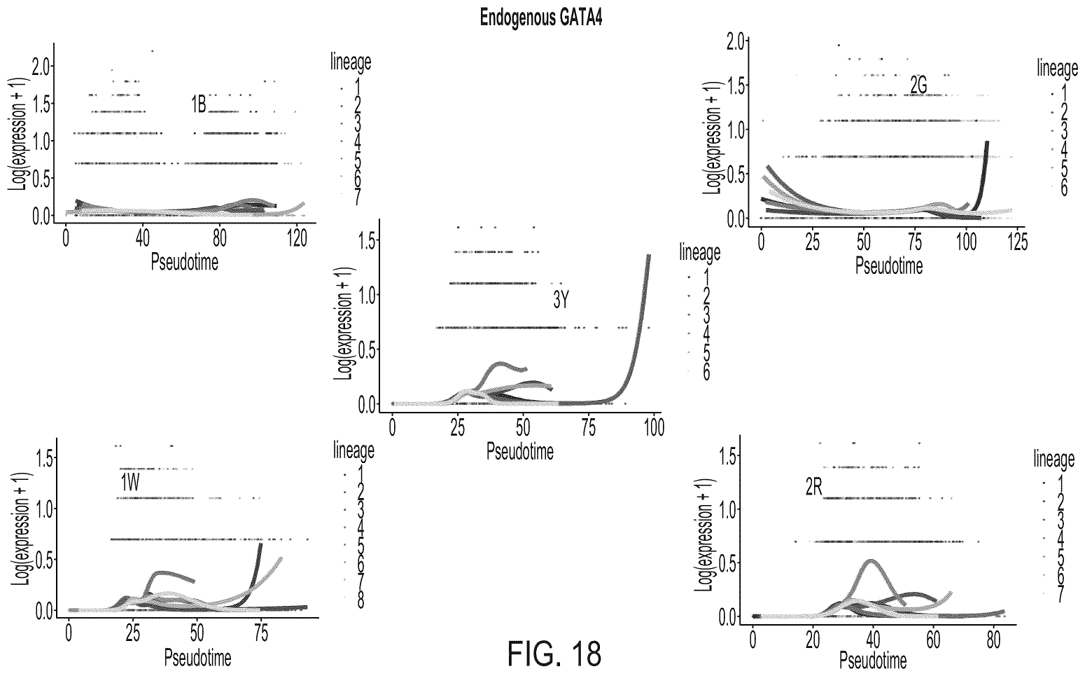

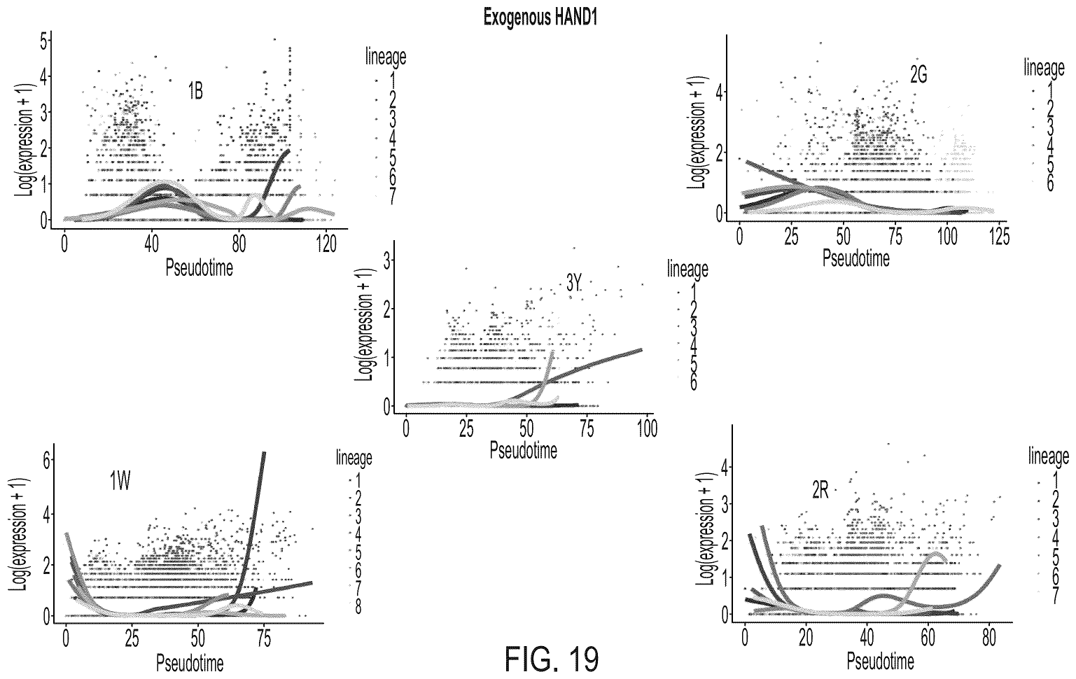

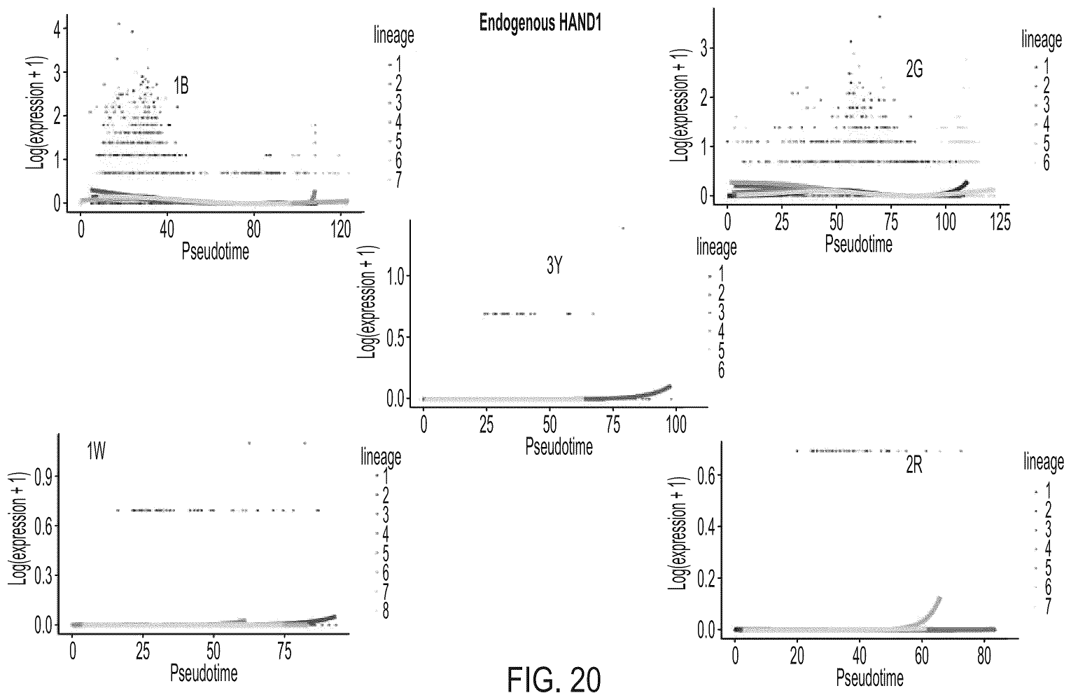

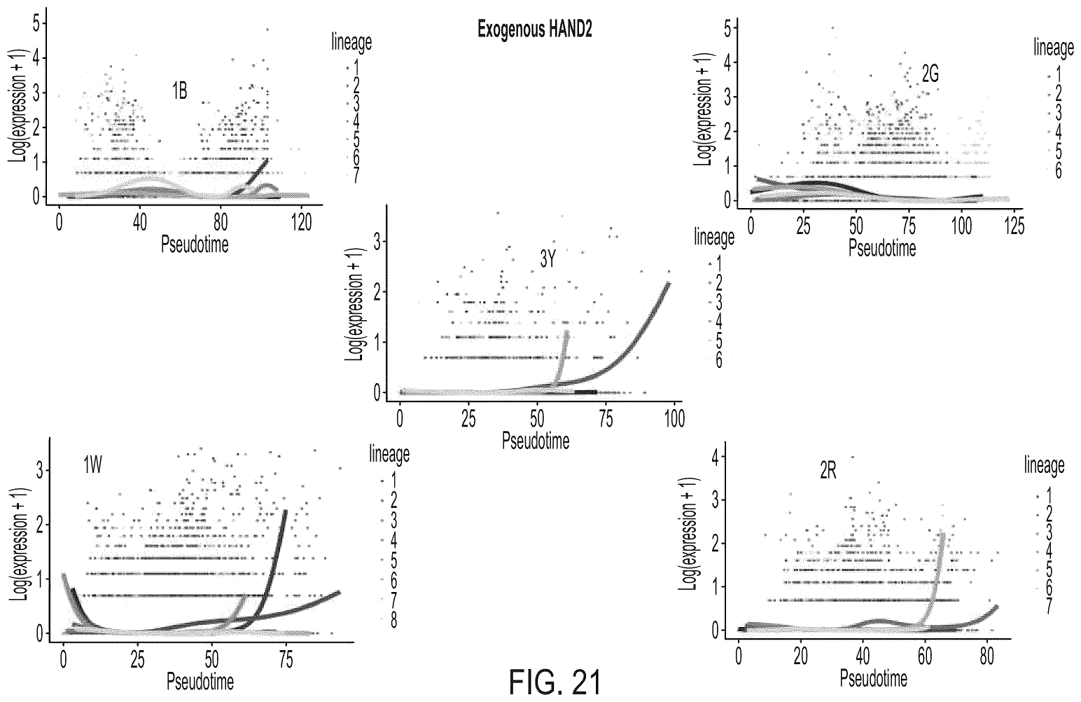

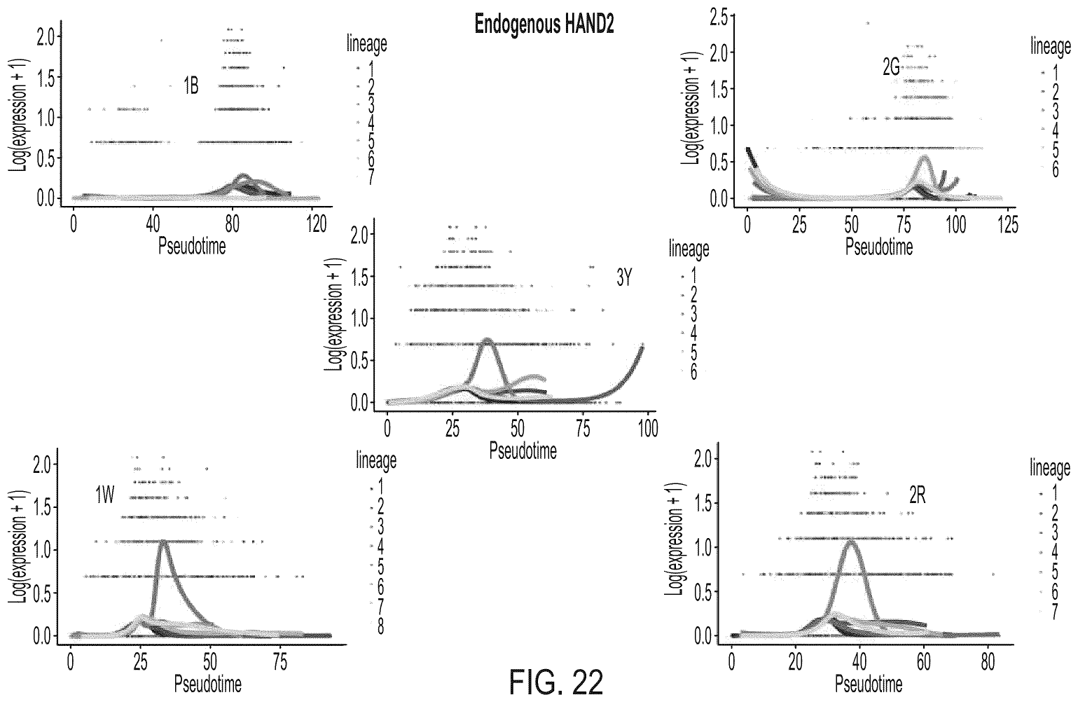

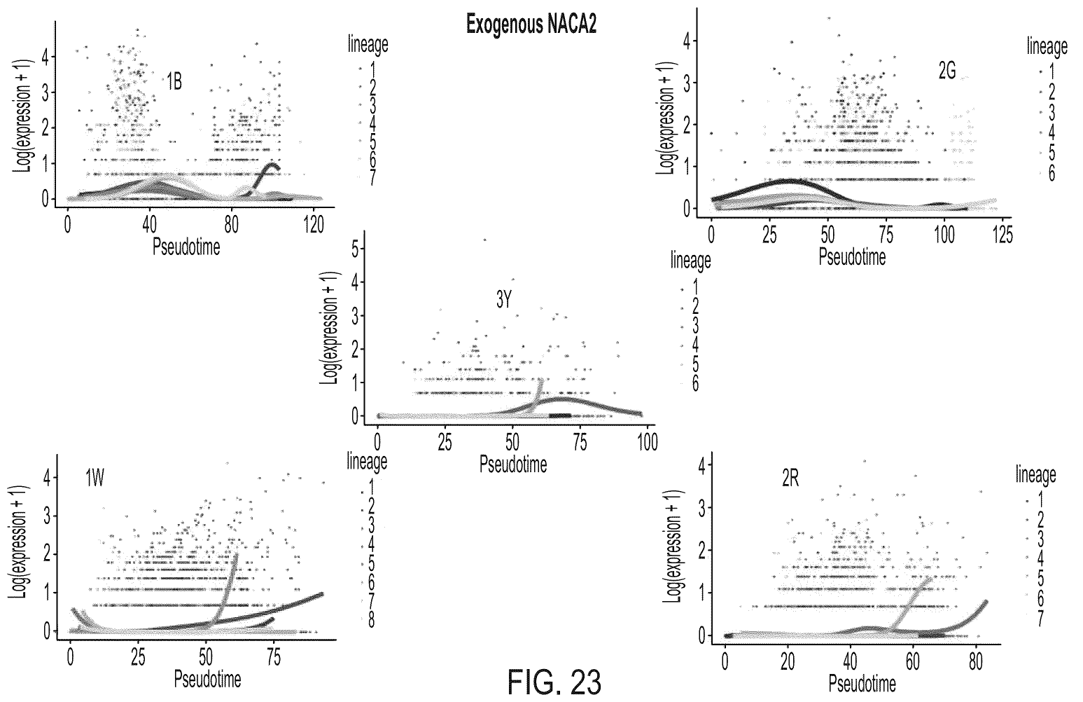

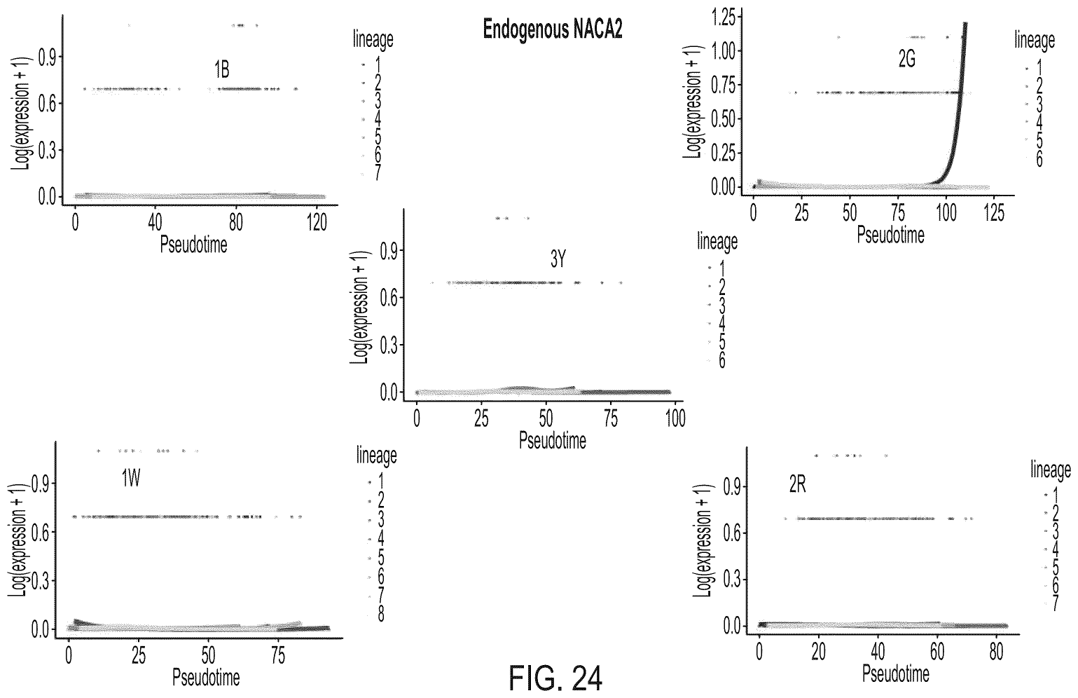

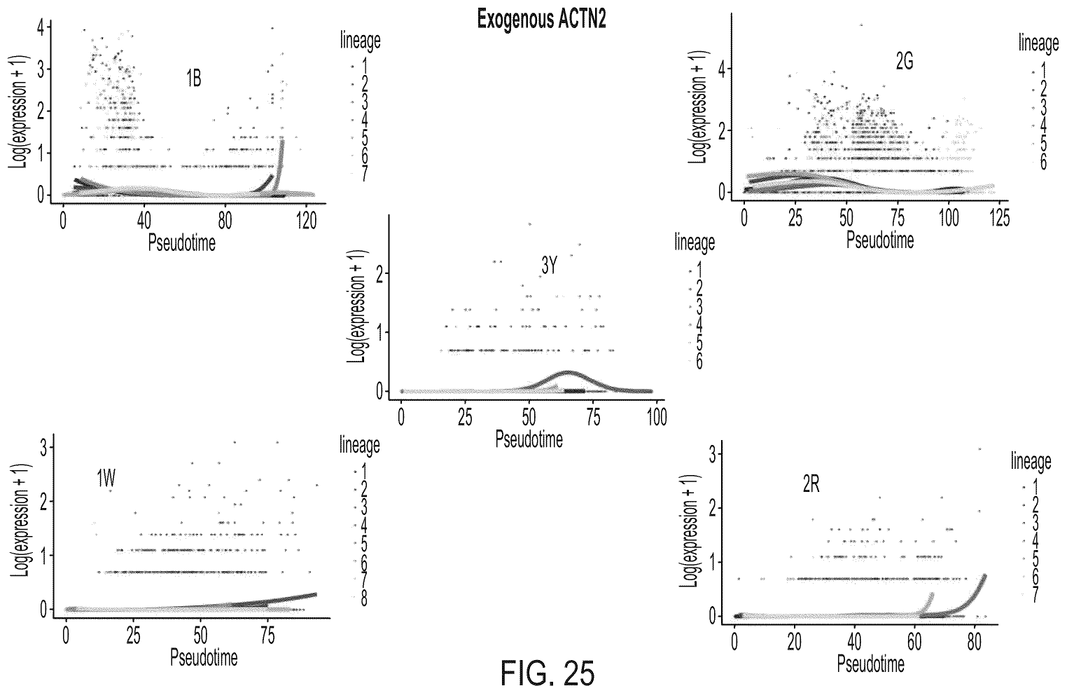

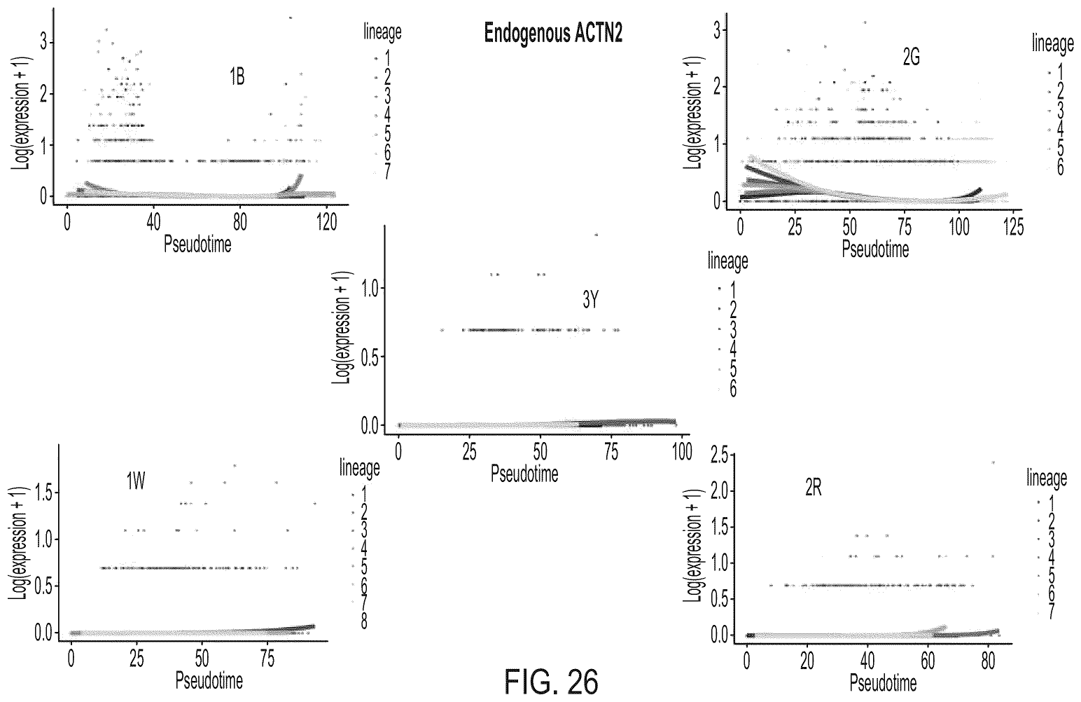

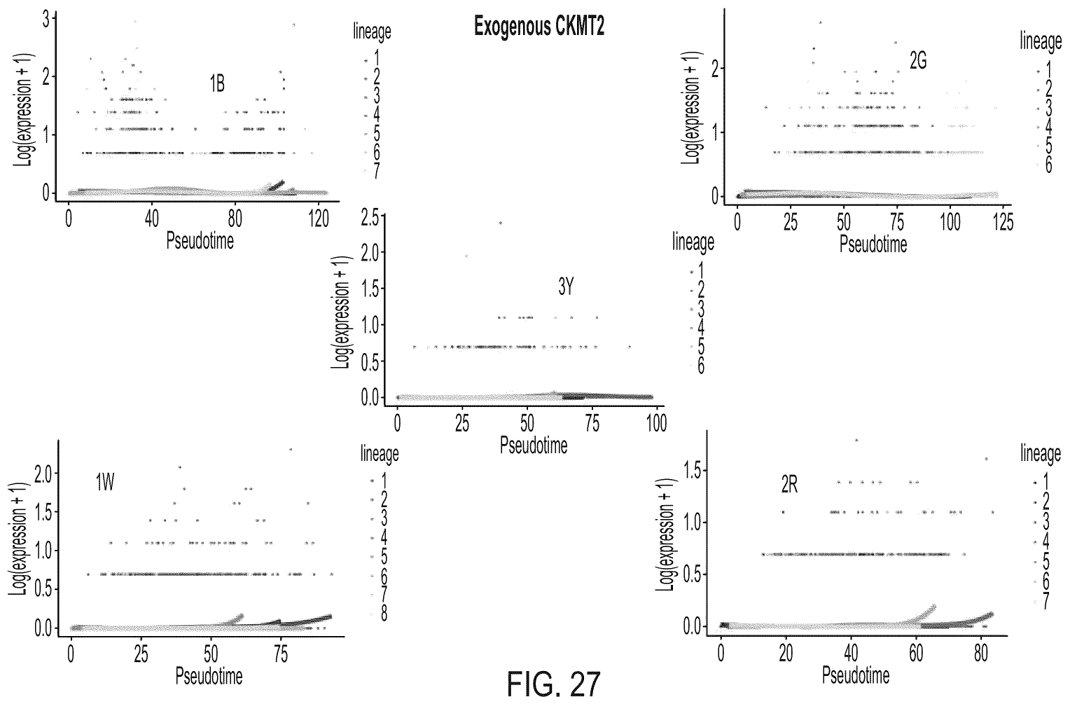

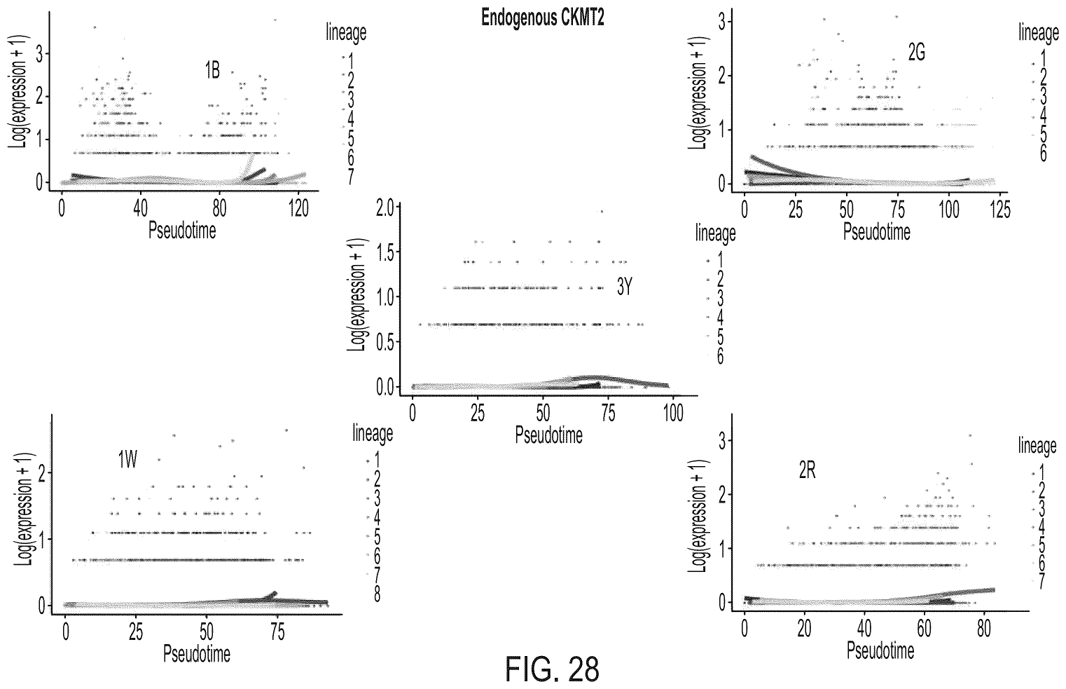

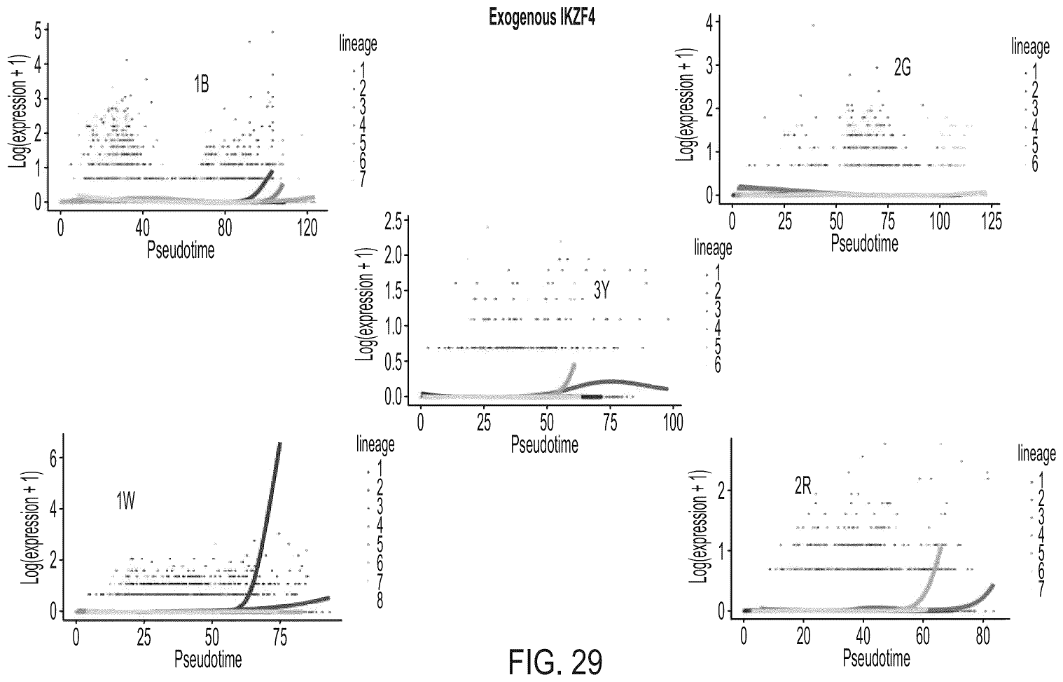

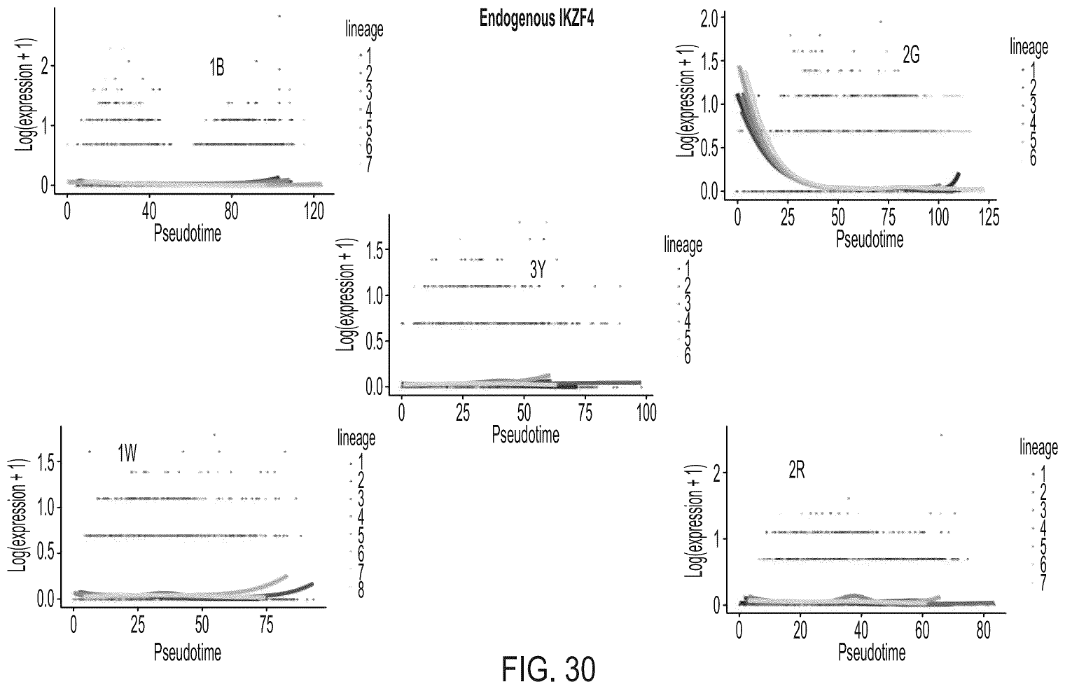

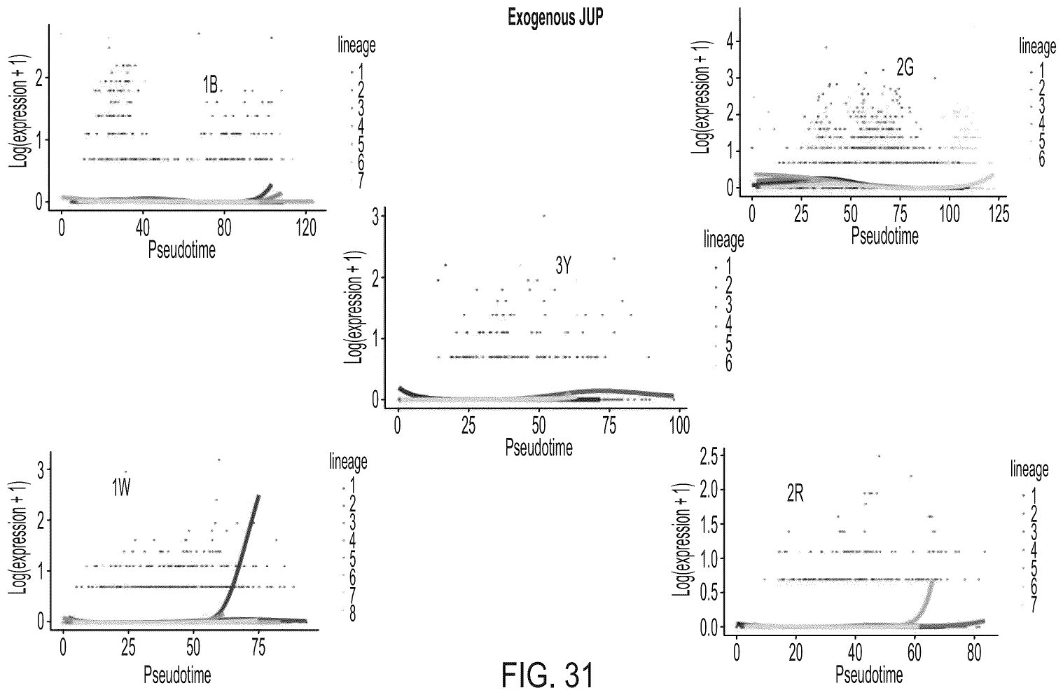

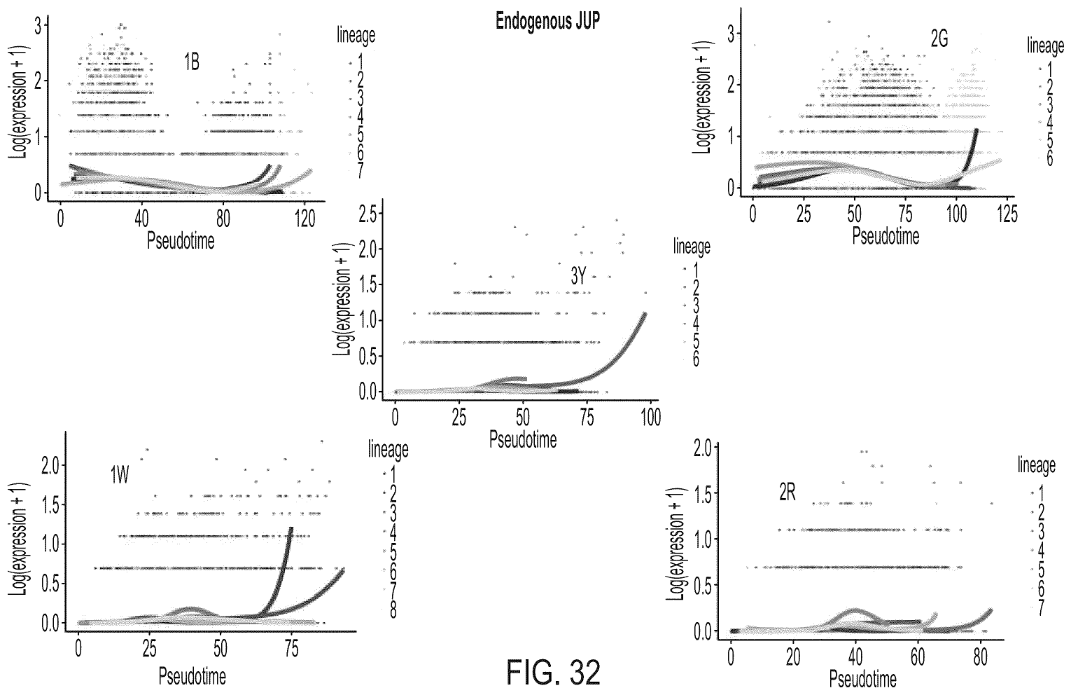

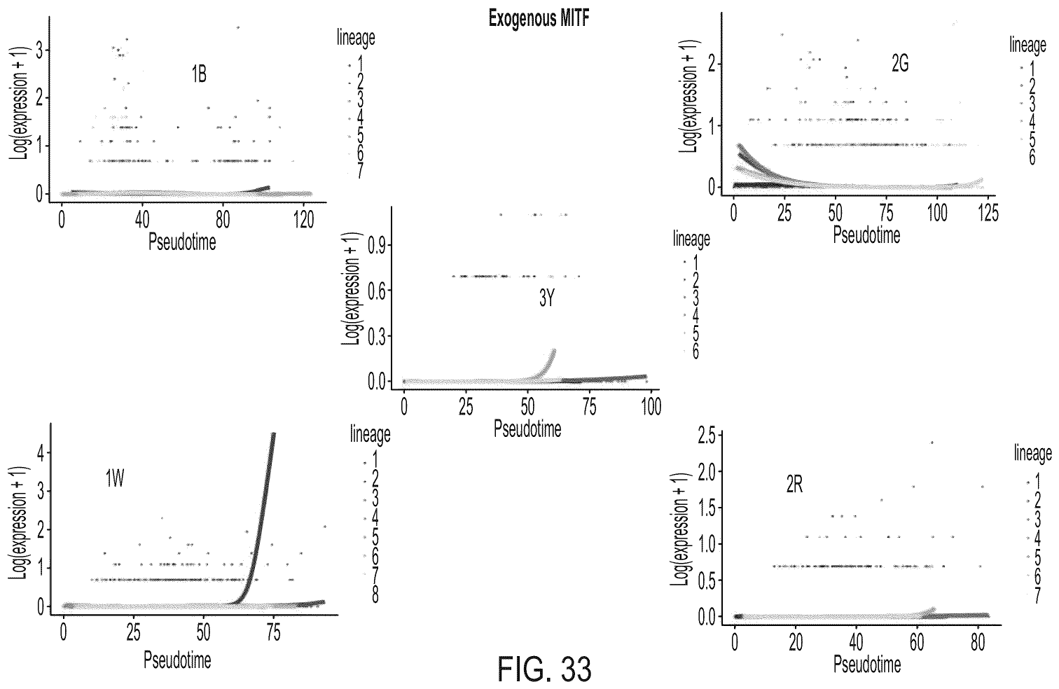

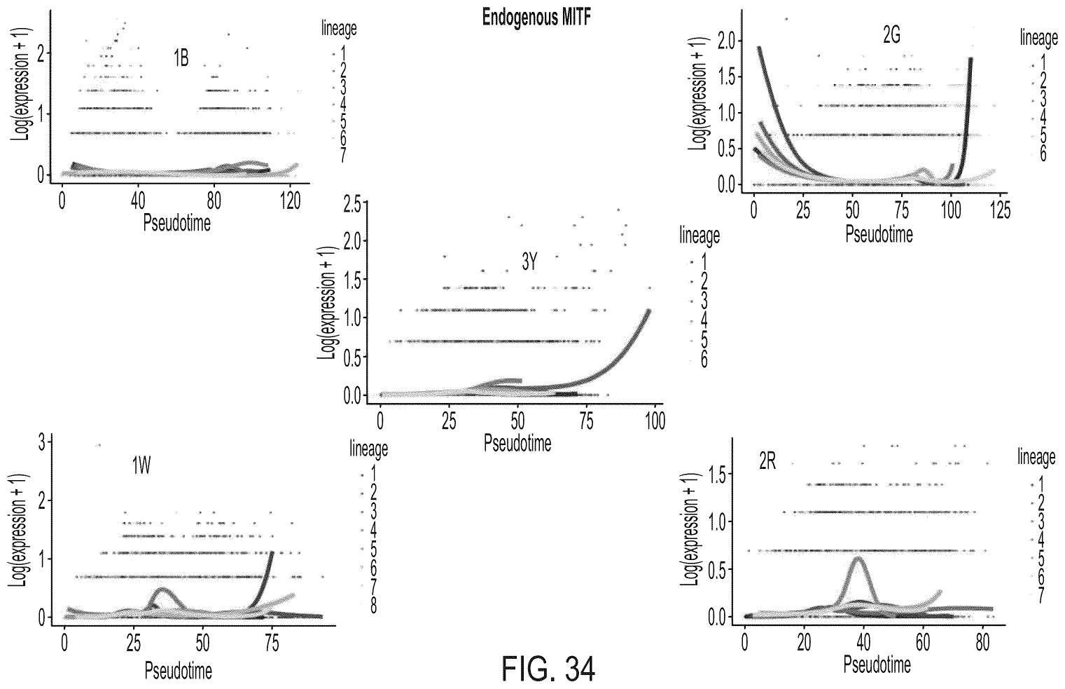

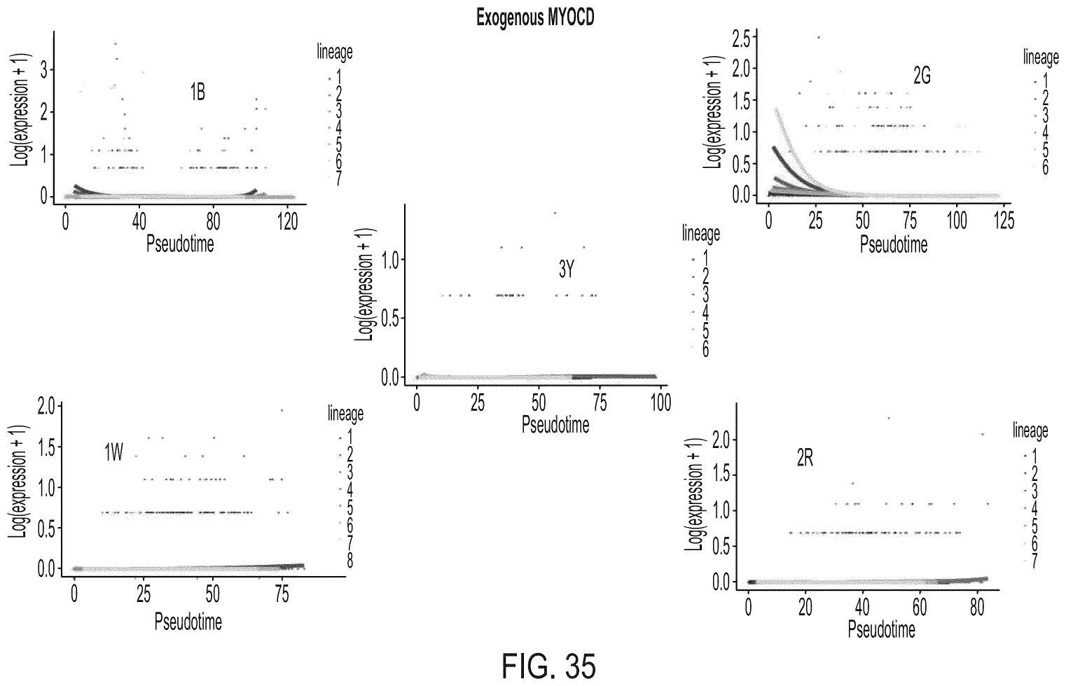

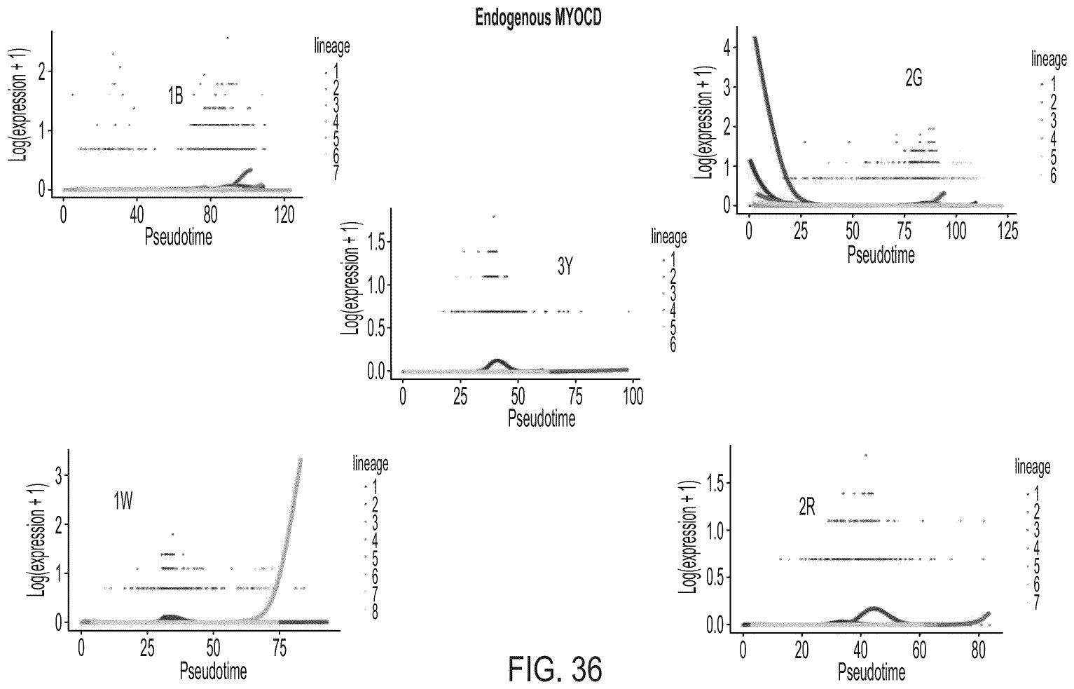

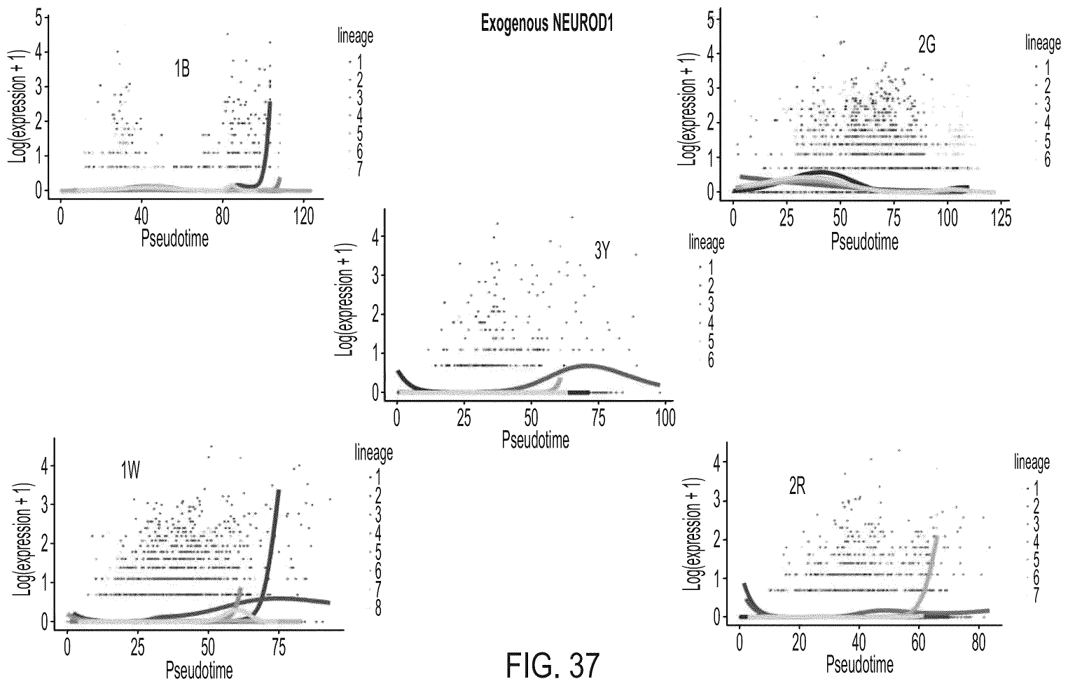

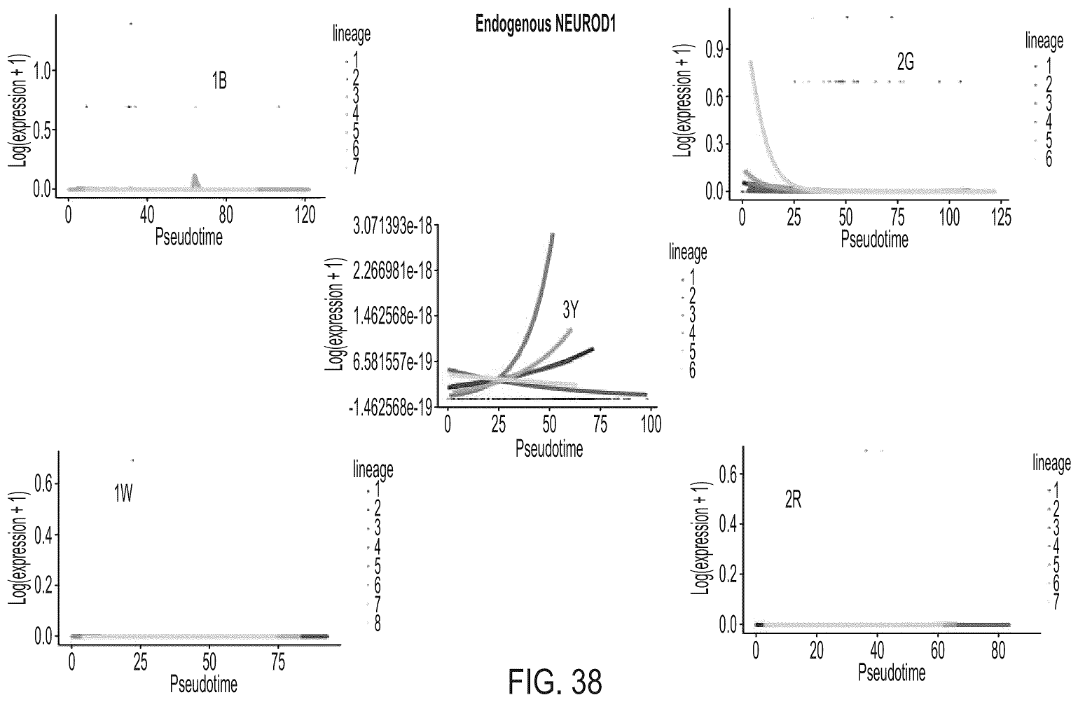

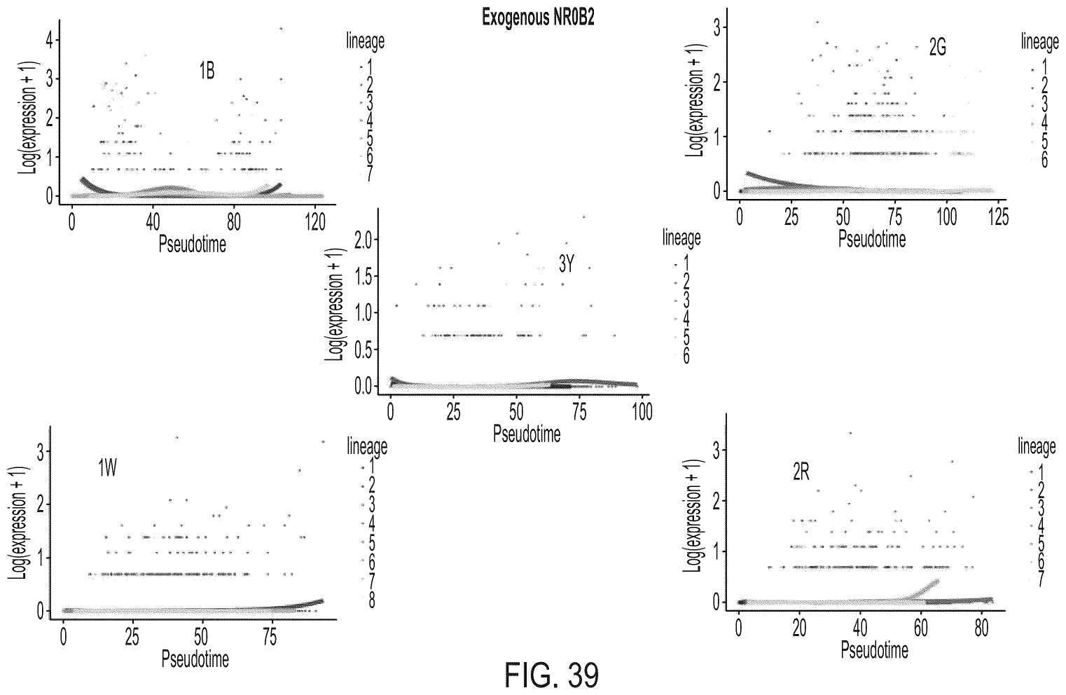

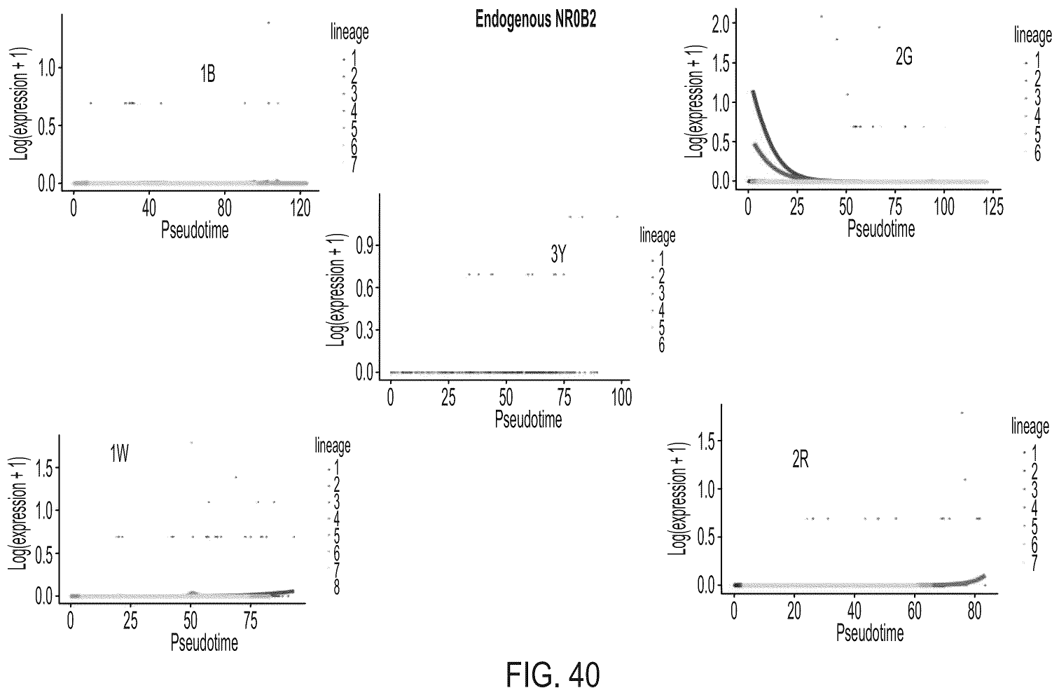

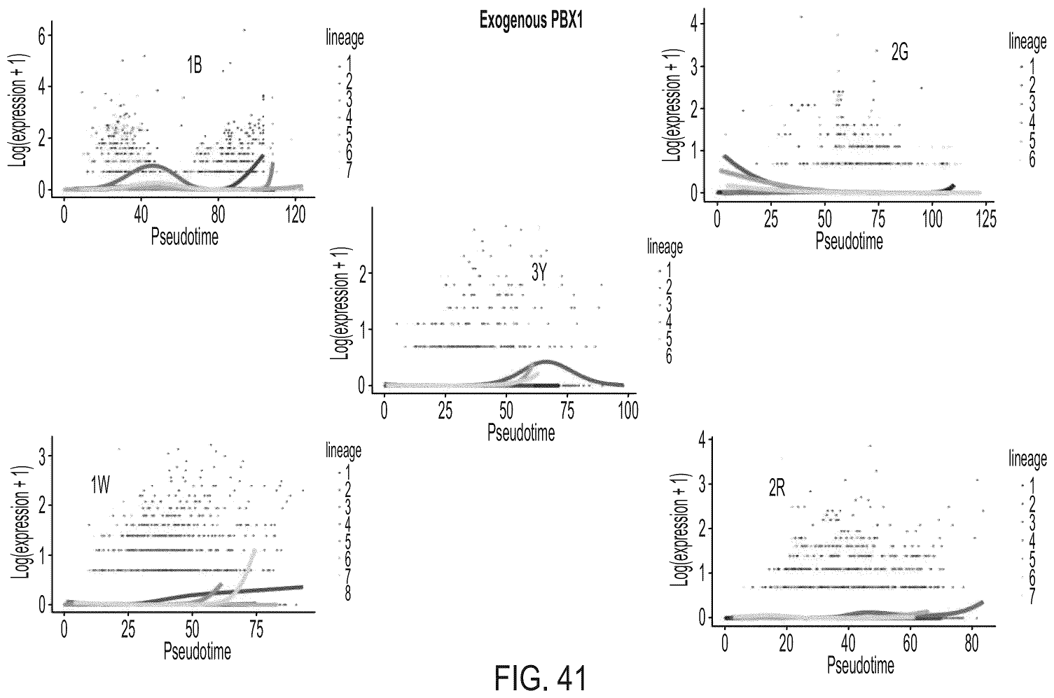

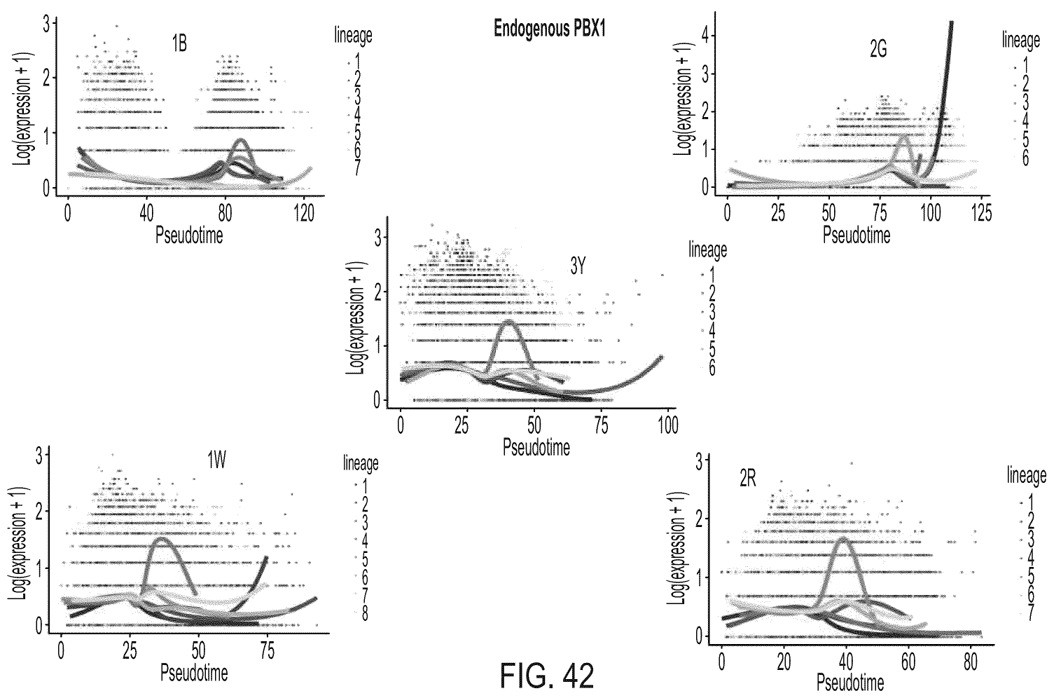

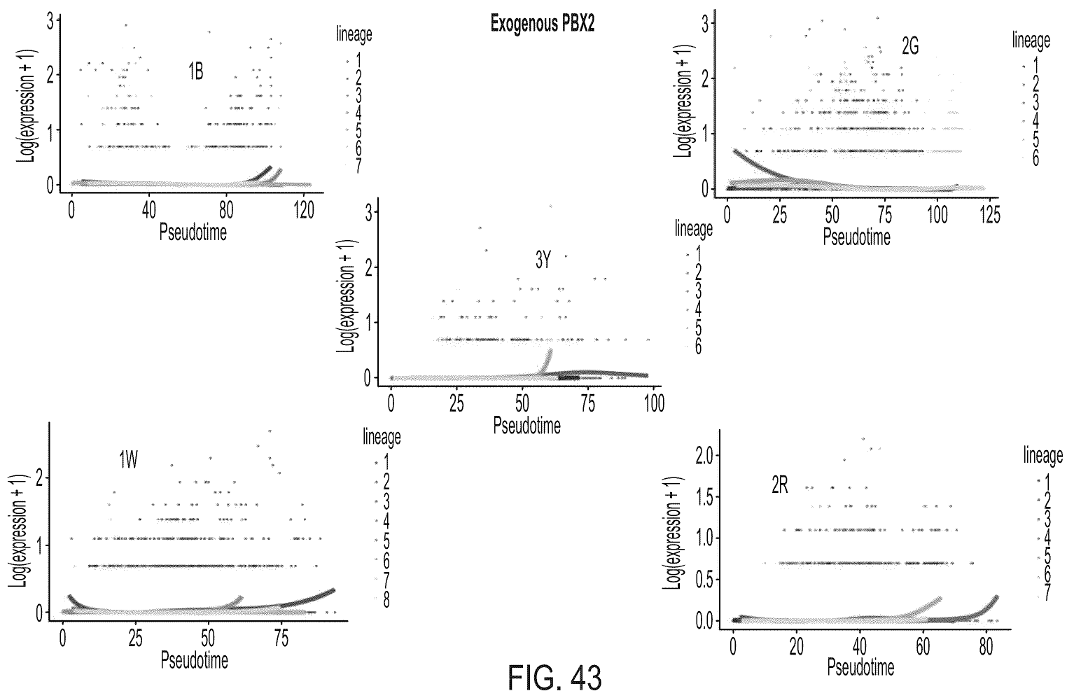

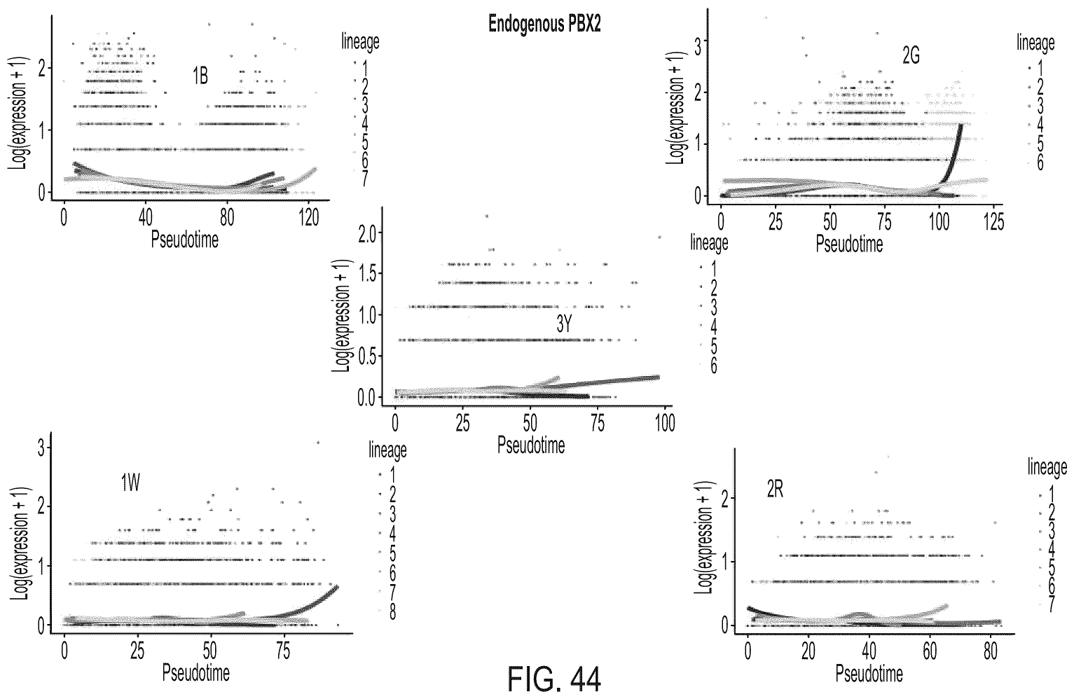

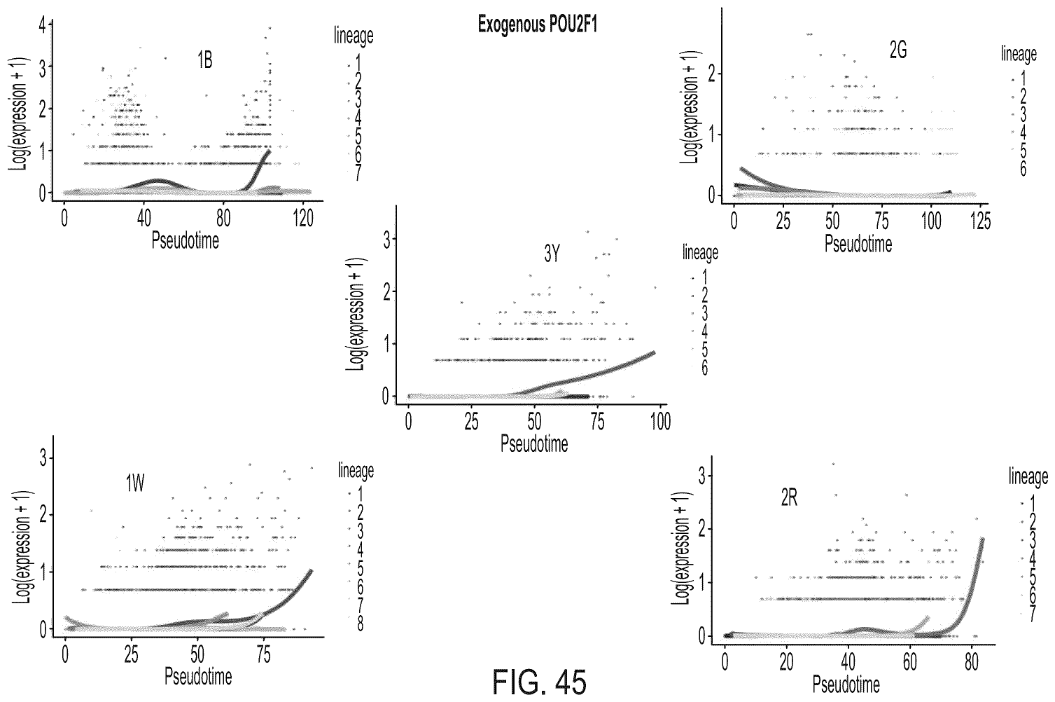

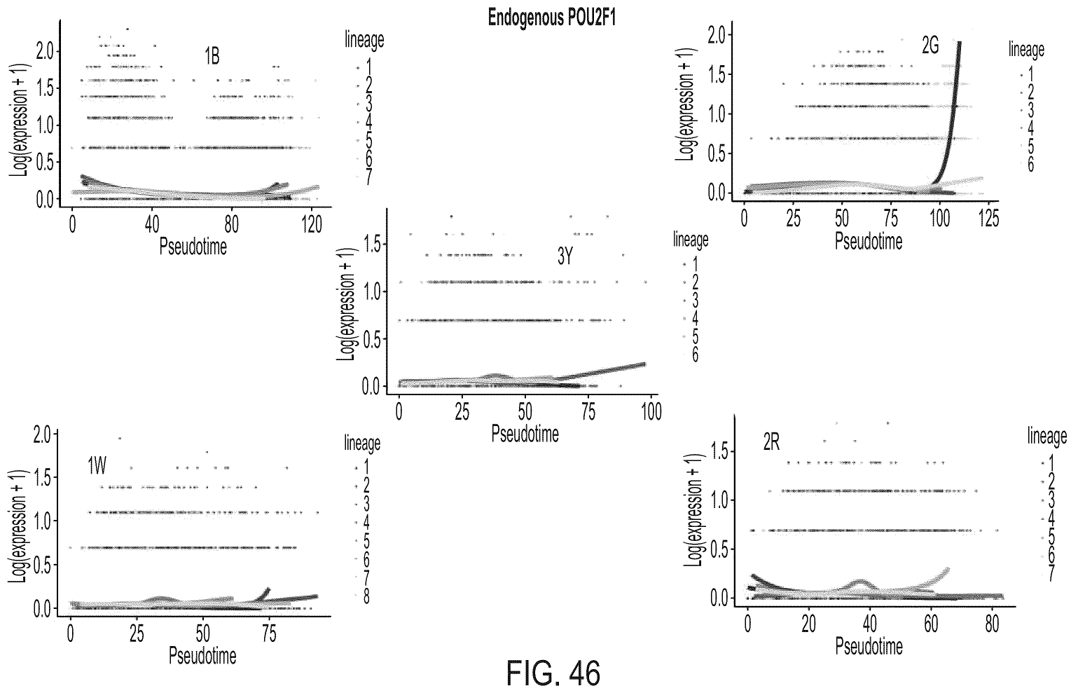

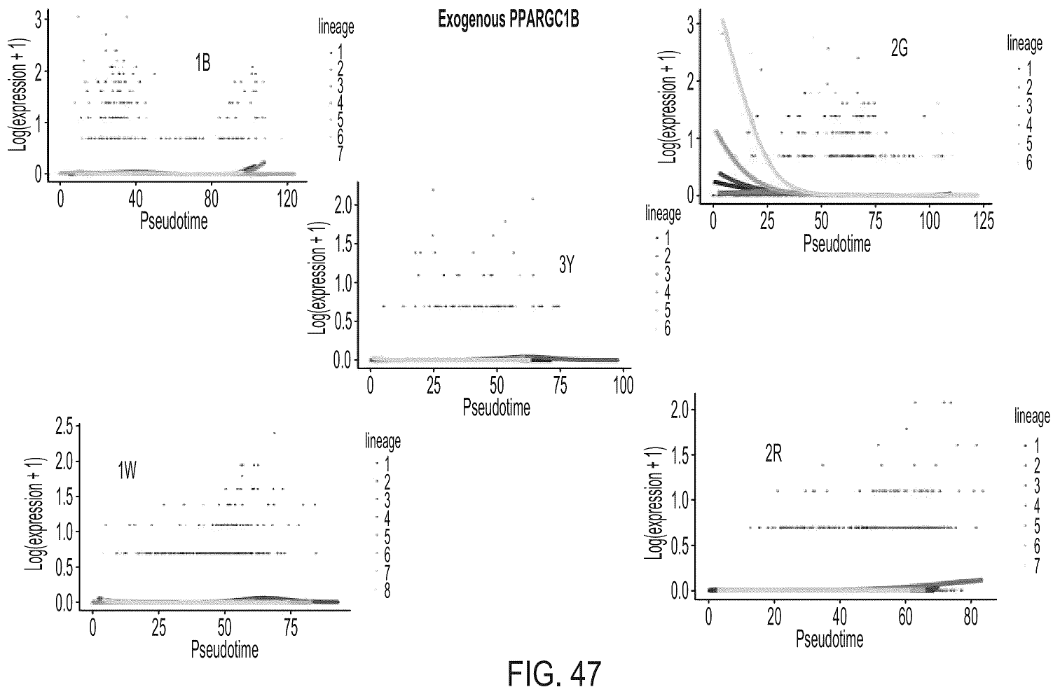

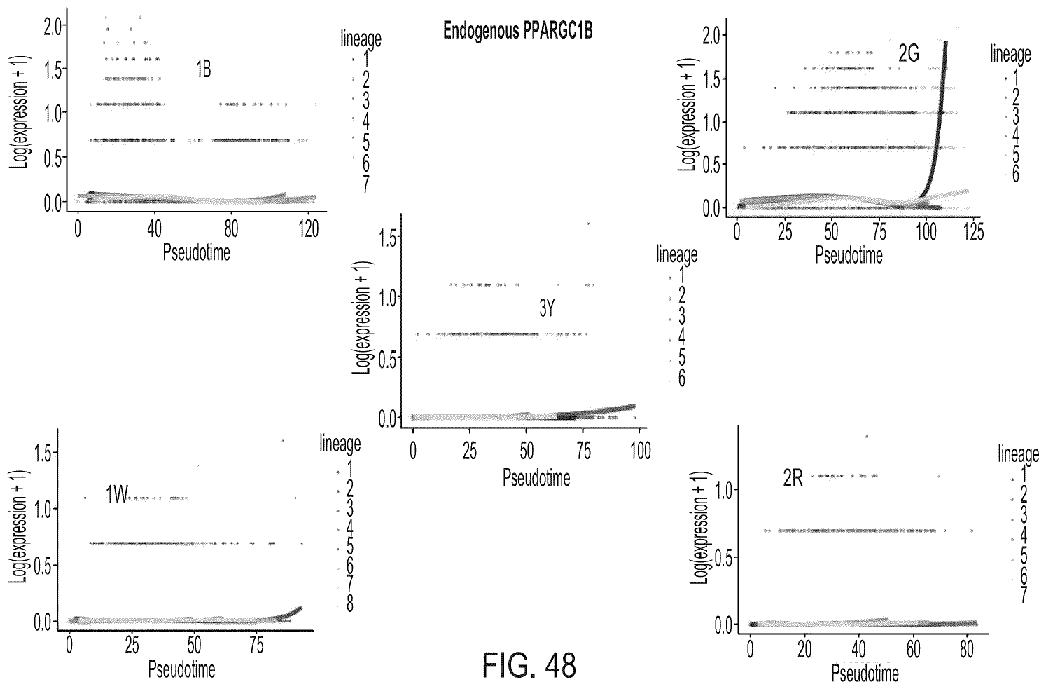

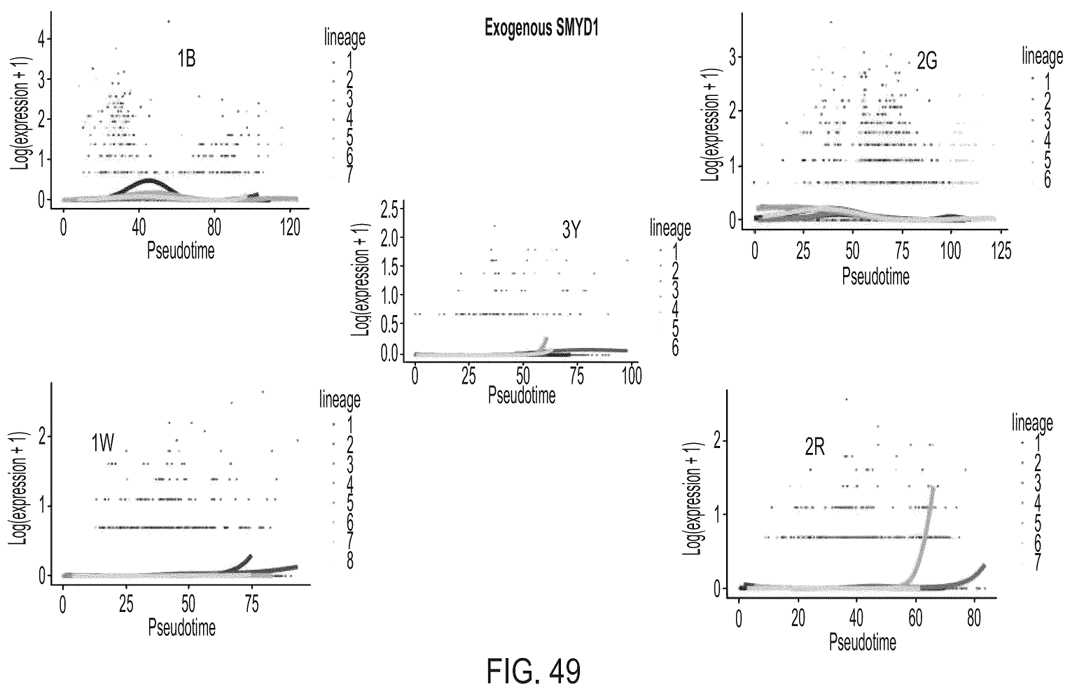

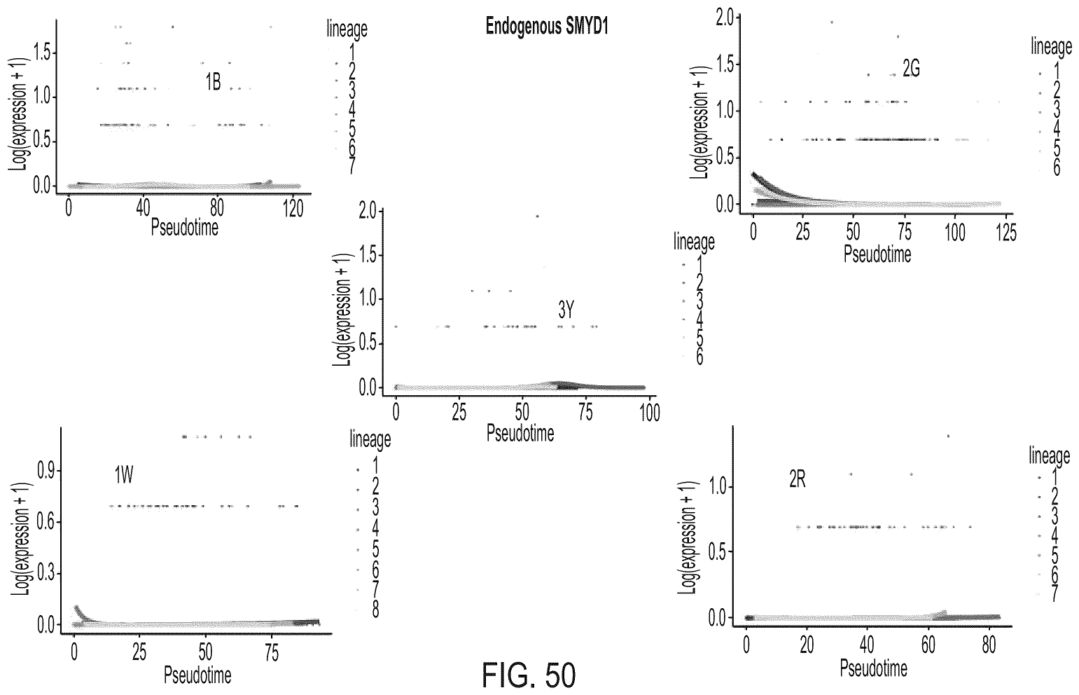

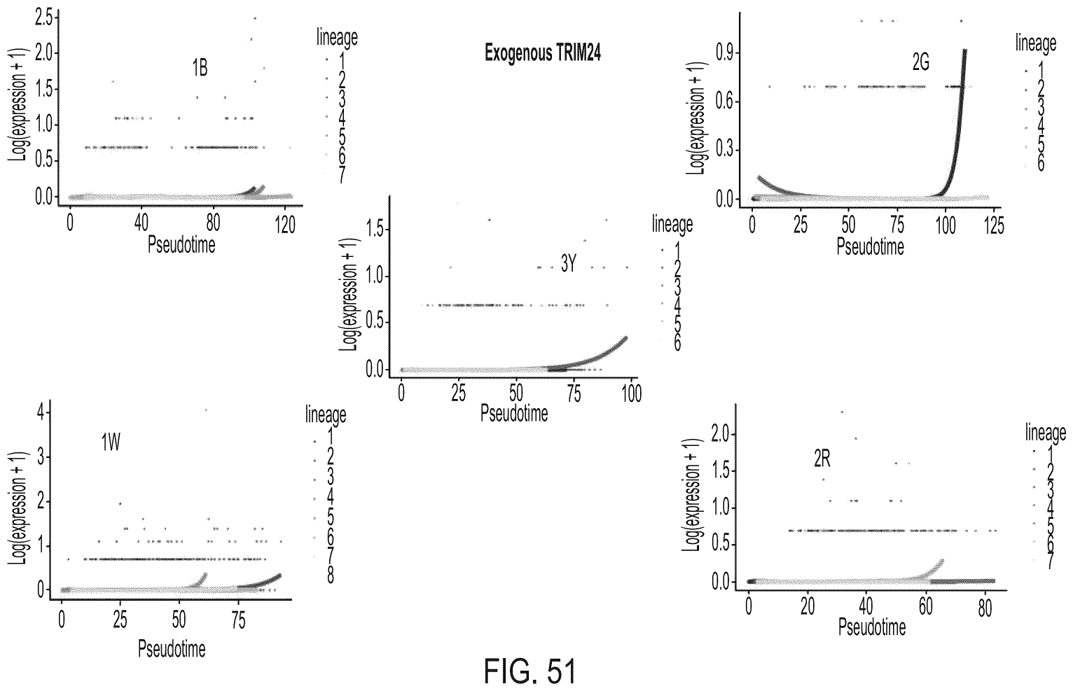

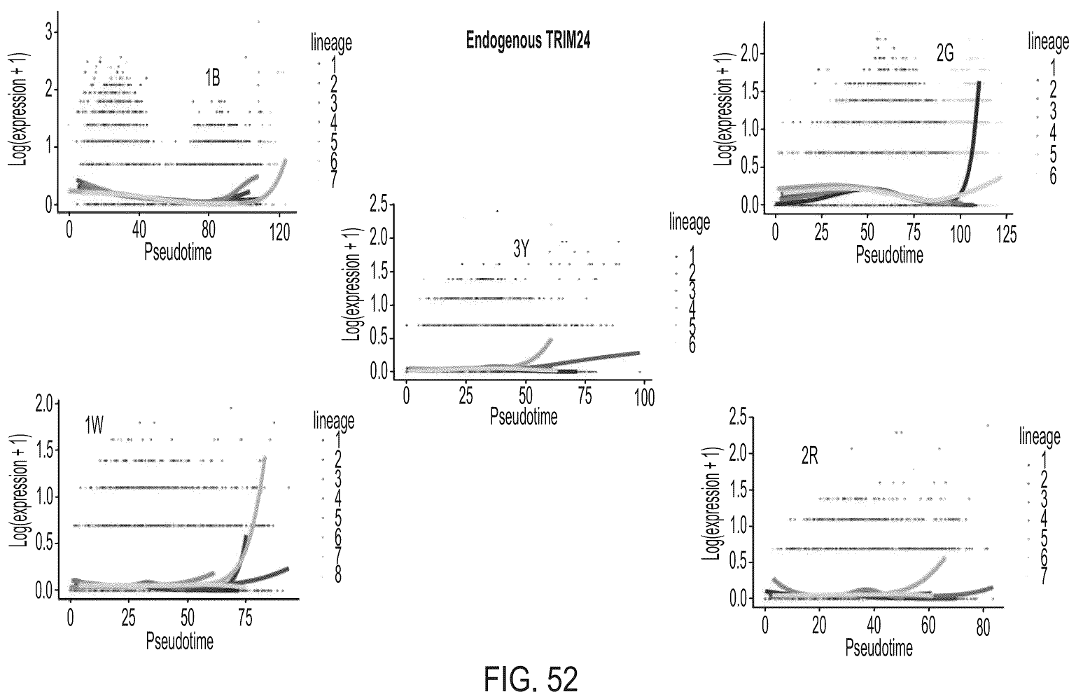

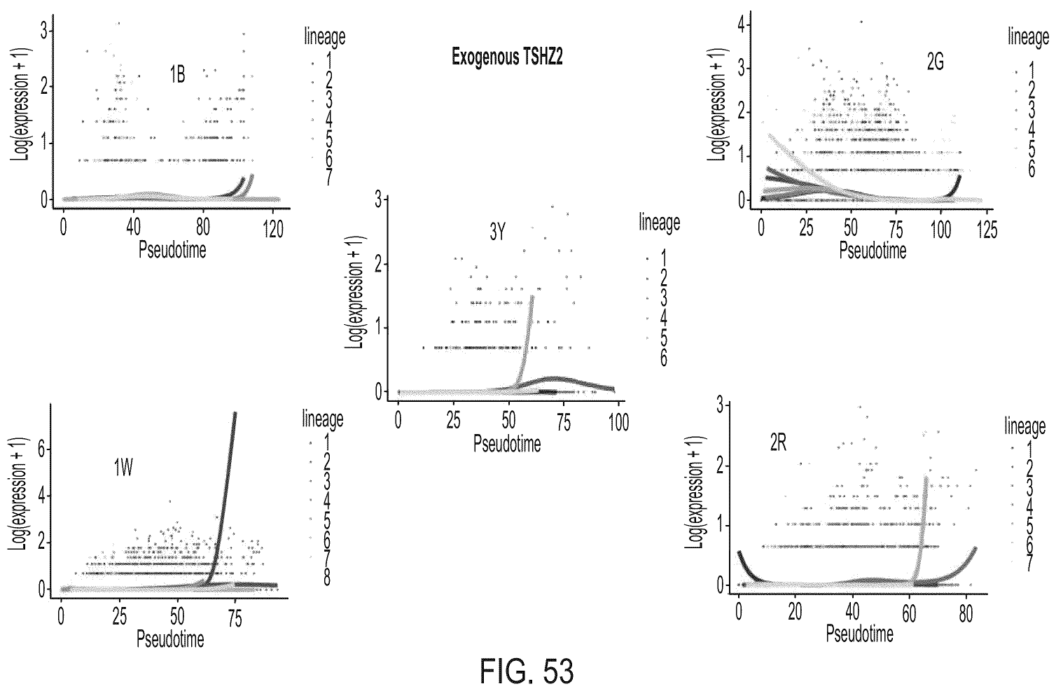

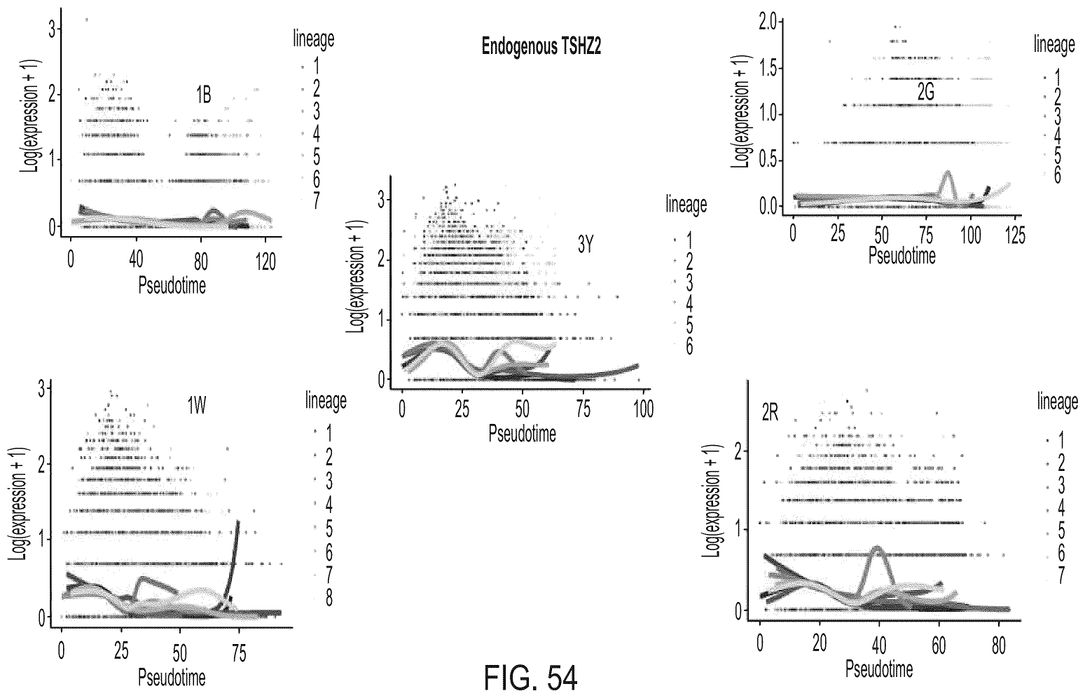

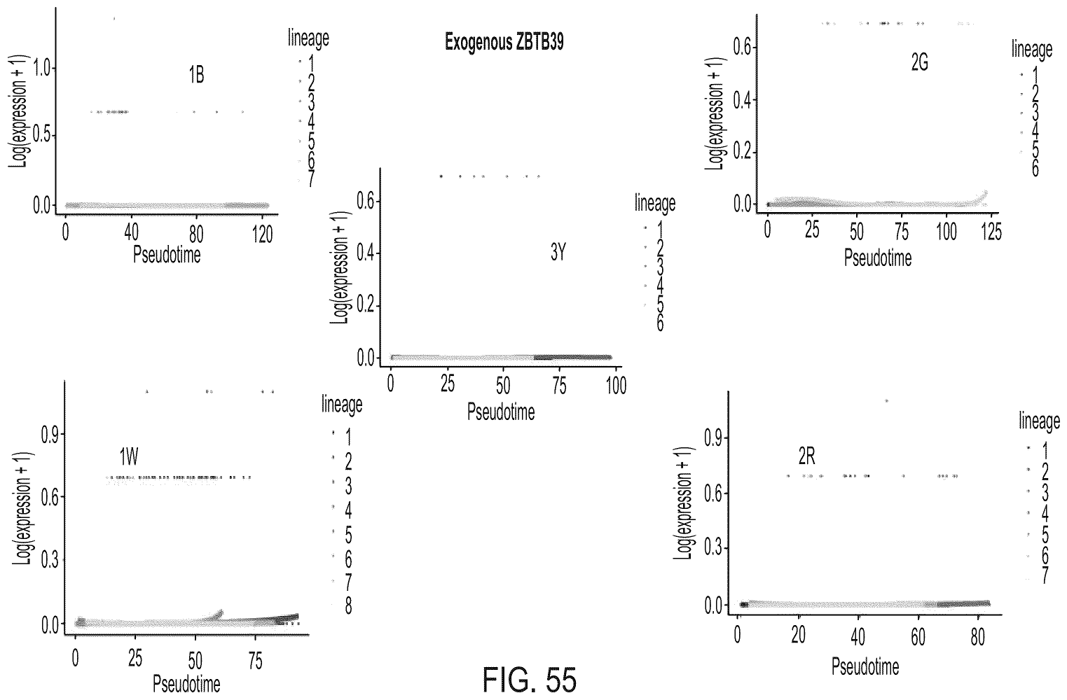

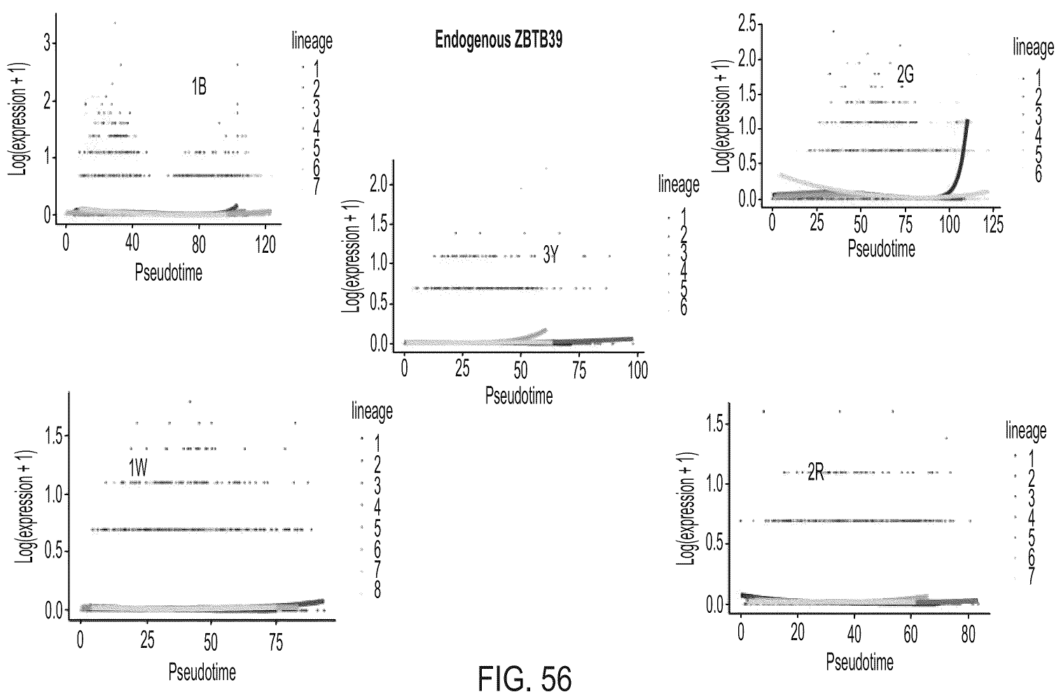

FIGS. 12-56 depict tradeSeq with PCA 100 slingshot. FIGS. 12A-C show Cell line 1B mitochondria genes, FIGS. 13A-C show Cell line 2G mitochondria genes. FIGS. 14A-C show Cell line 1W mitochondria genes. FIGS. 15A-C show Cell line 2R mitochondria genes. FIGS. 16A-C show Cell line 3Y mitochondria genes. Table 6 indicates tradeSeq with PCA 100 slingshot Figure numbers for results for indicated genes.

TABLE 6
Figure numbers for tradeSeq with PCA 100
slingshot results for indicated genes.

| | FIG. Number | FIG. Number |
|---|---|---|
| Gene | for Exogenous | for Endogenous |
| GATA4 | 17 | 18 |
| HAND1 | 19 | 20 |
| HAND2 | 21 | 22 |
| NACA2 | 23 | 24 |
| ACTN2 | 25 | 26 |
| CKMT2 | 27 | 28 |
| IKZF4 | 29 | 30 |
| JUP | 31 | 32 |
| MITF | 33 | 34 |
| MYOCD | 35 | 36 |
| NEUROD1 | 37 | 38 |
| NROB2 | 39 | 40 |
| PBX1 | 41 | 42 |
| PBX2 | 43 | 44 |
| POU2F1 | 45 | 46 |
| PPARGC1B | 47 | 48 |
| SMYD1 | 49 | 50 |
| TRIM24 | 51 | 52 |
| TSHZ2 | 53 | 54 |
| ZBTB39 | 55 | 56 |

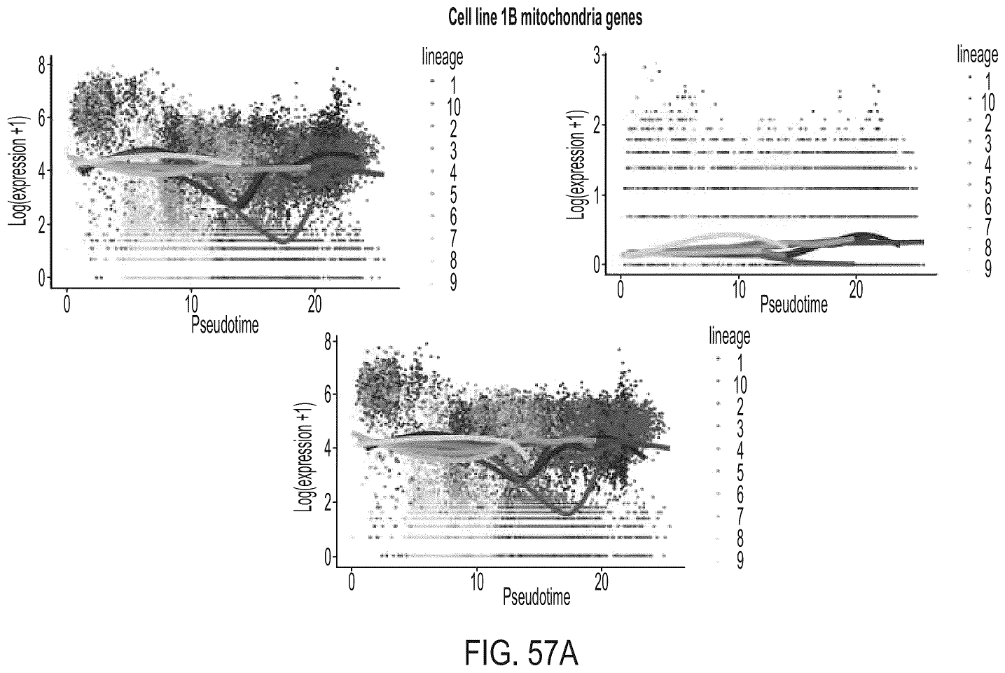

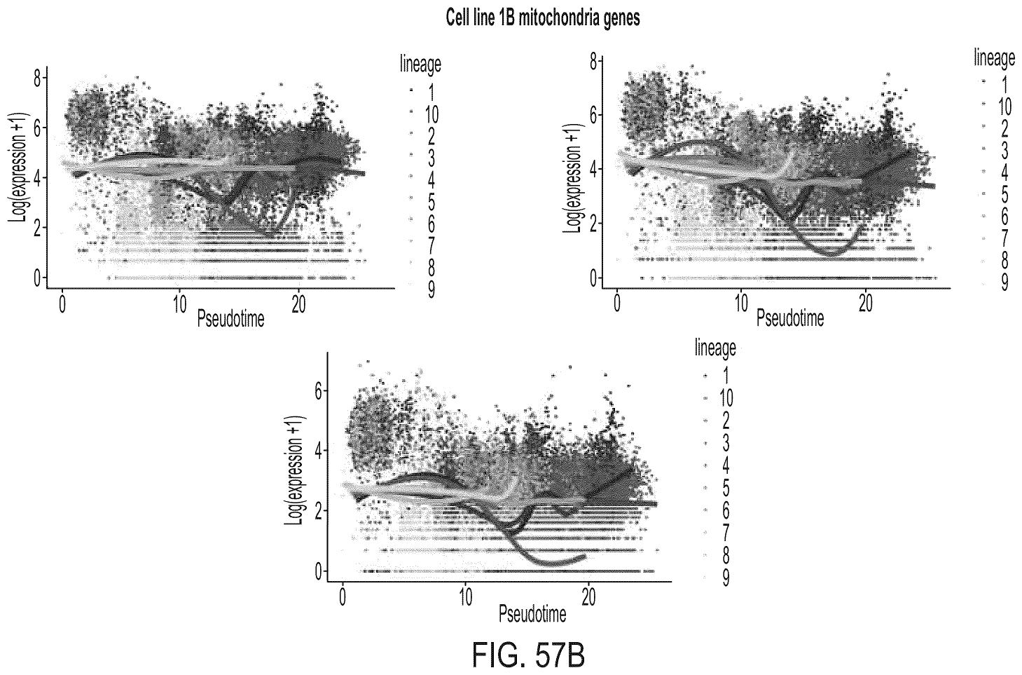

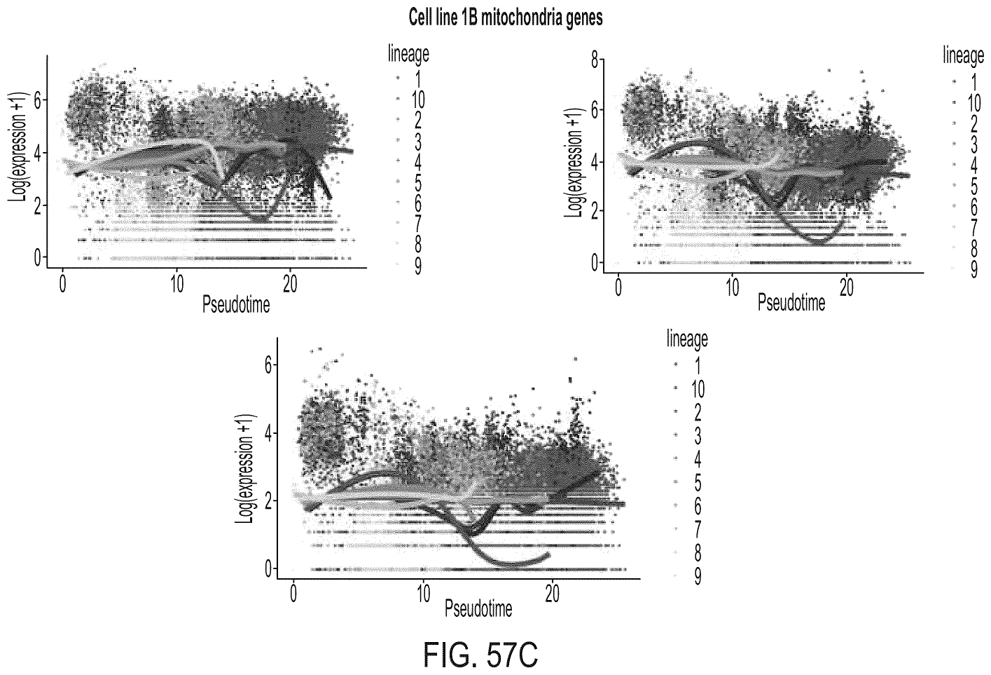

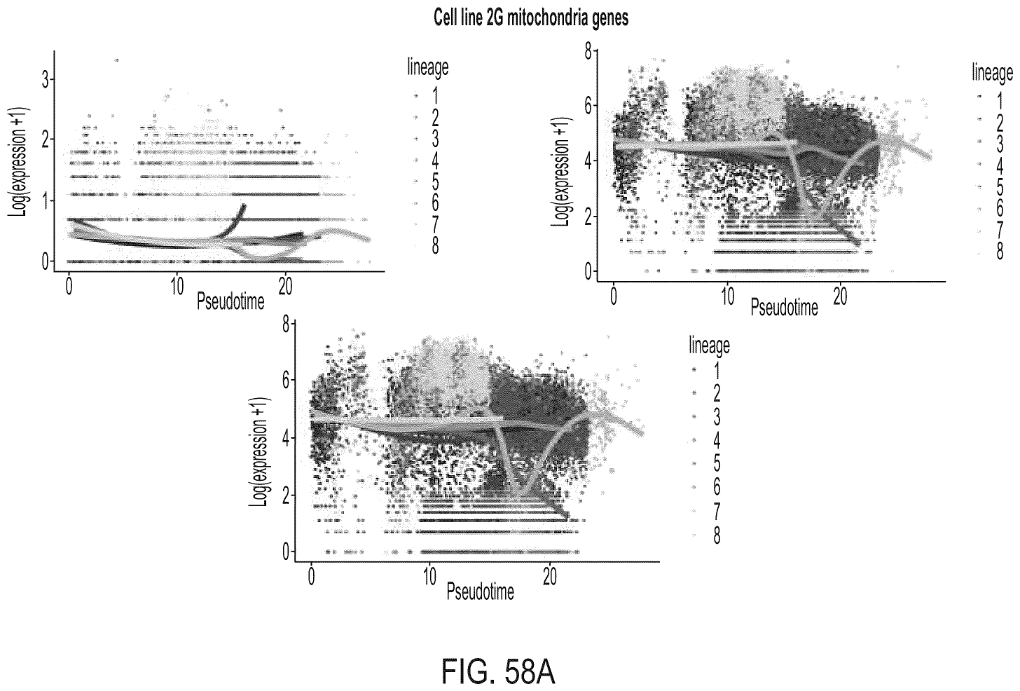

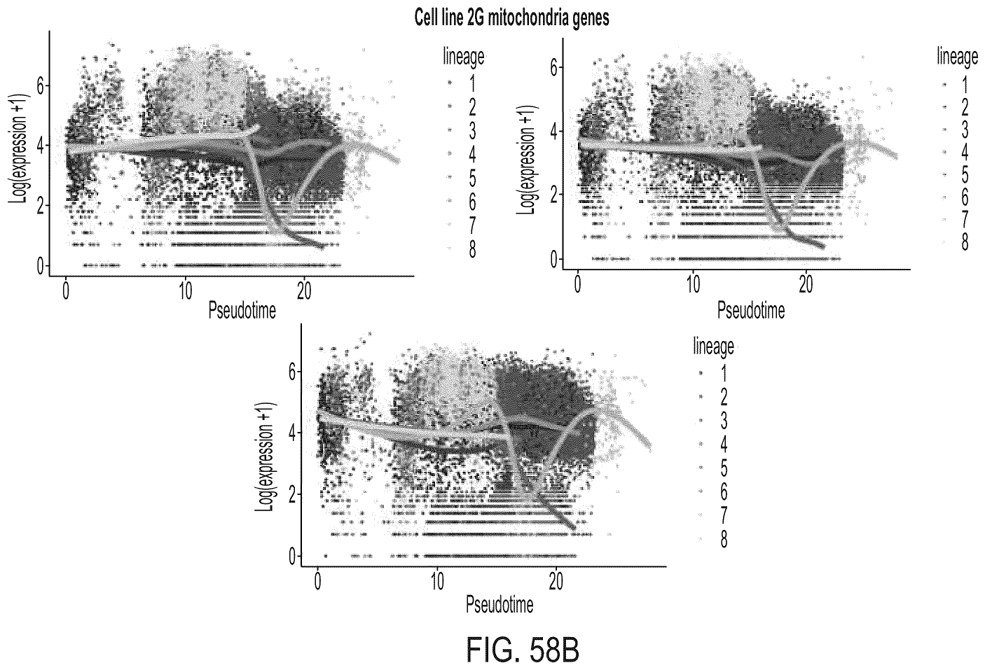

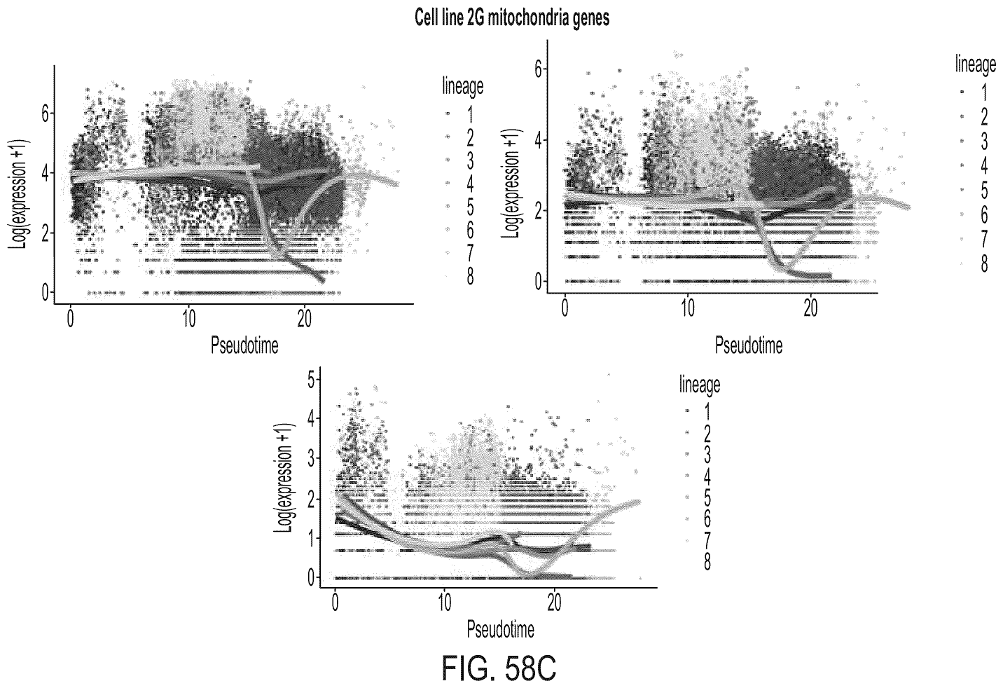

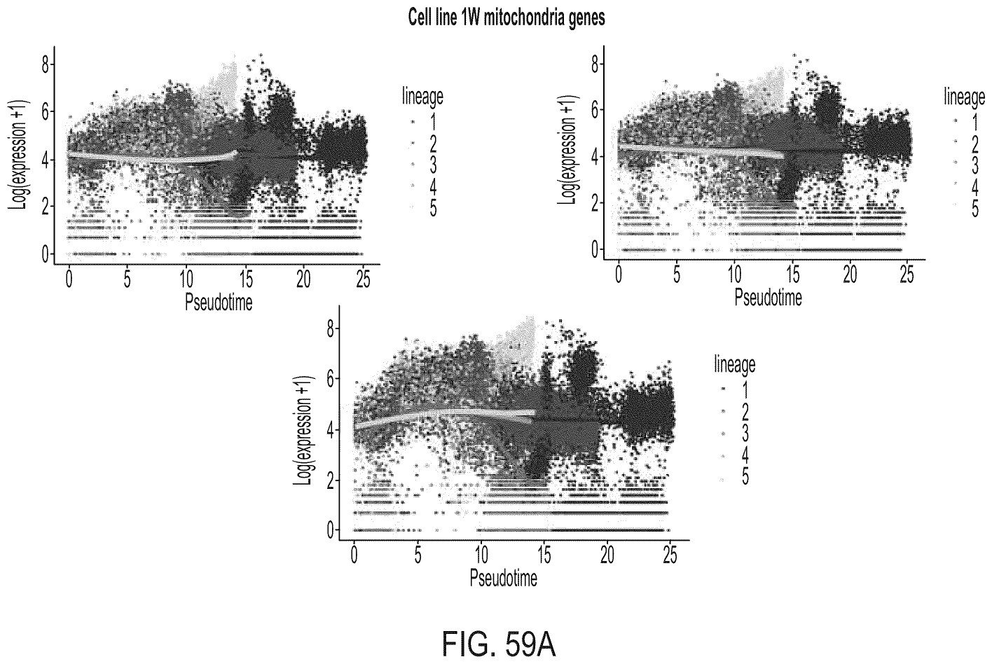

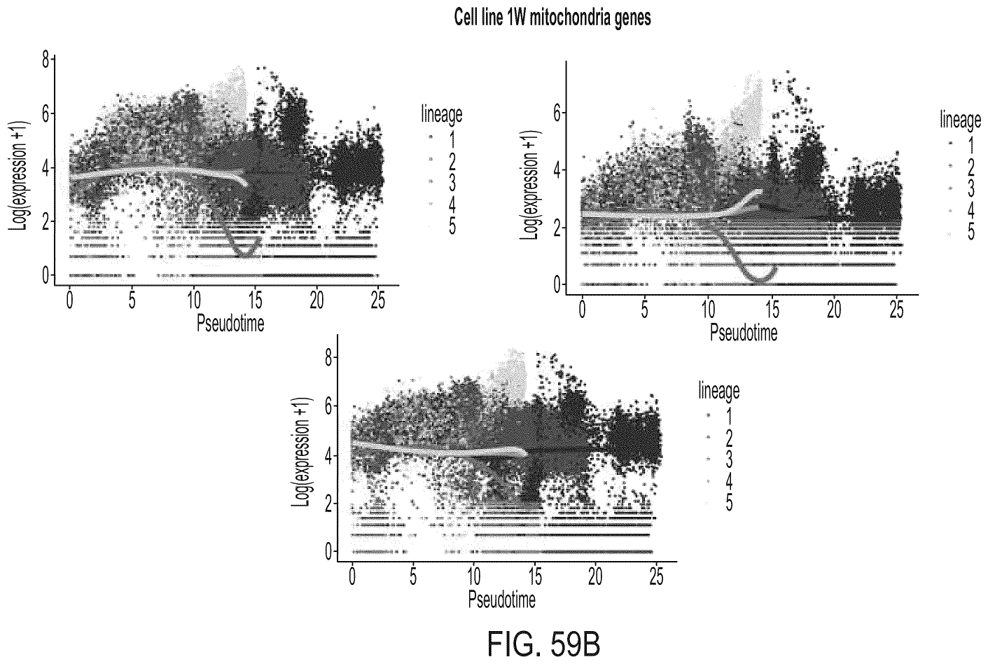

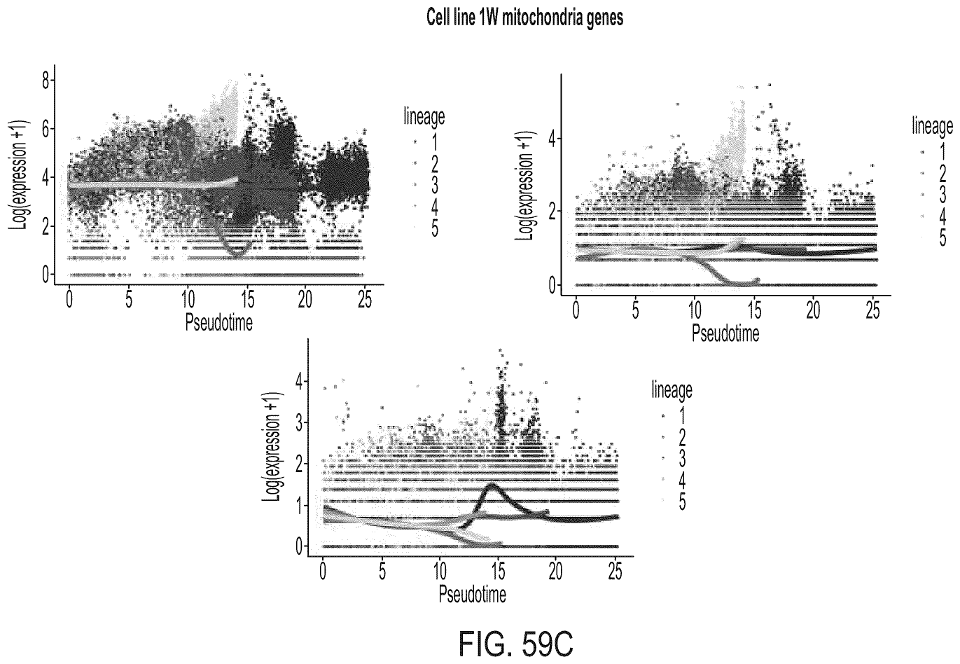

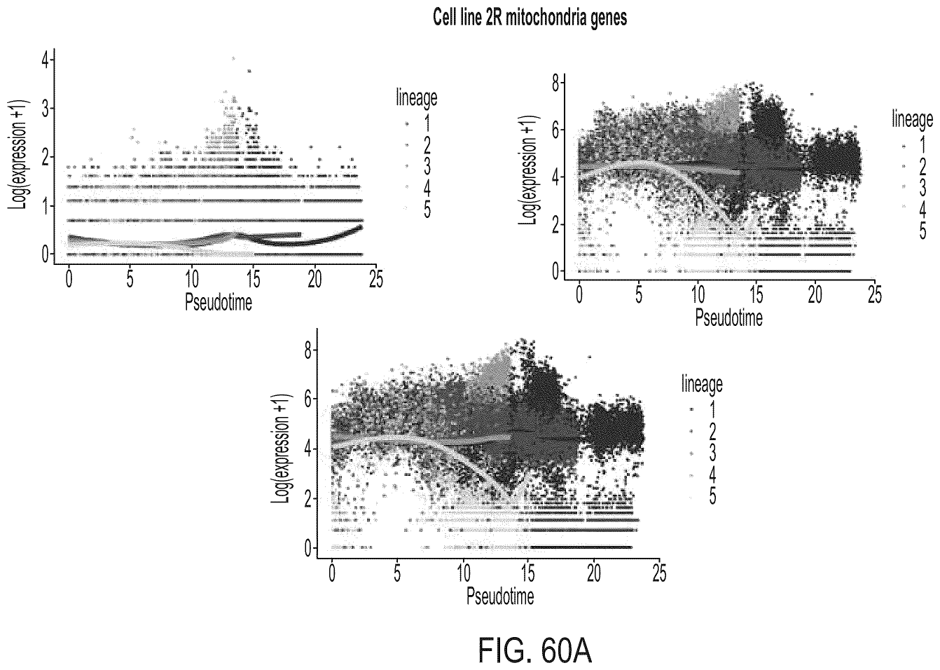

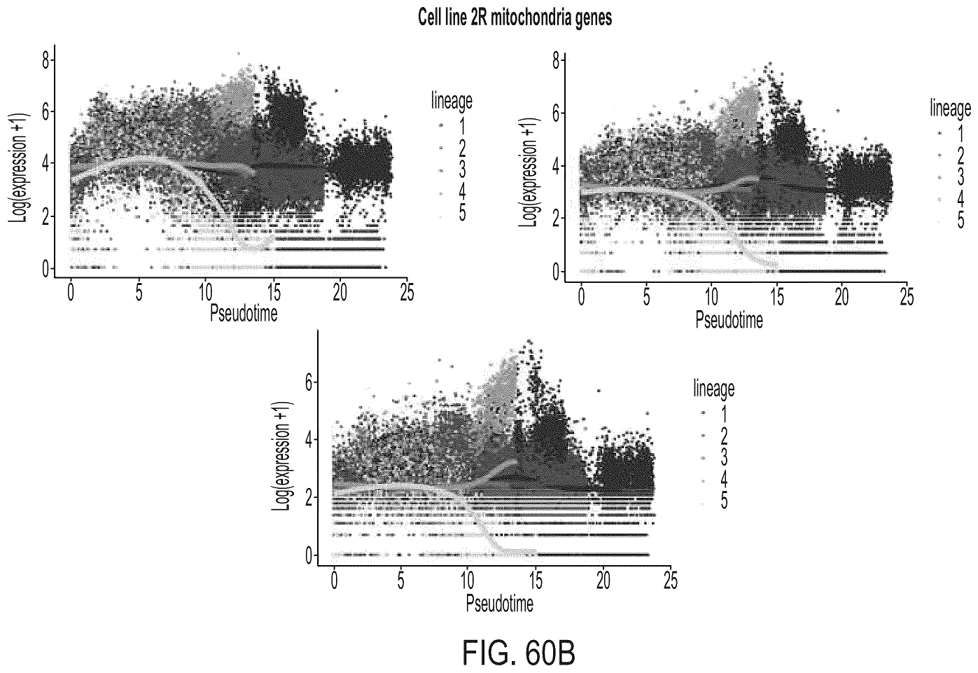

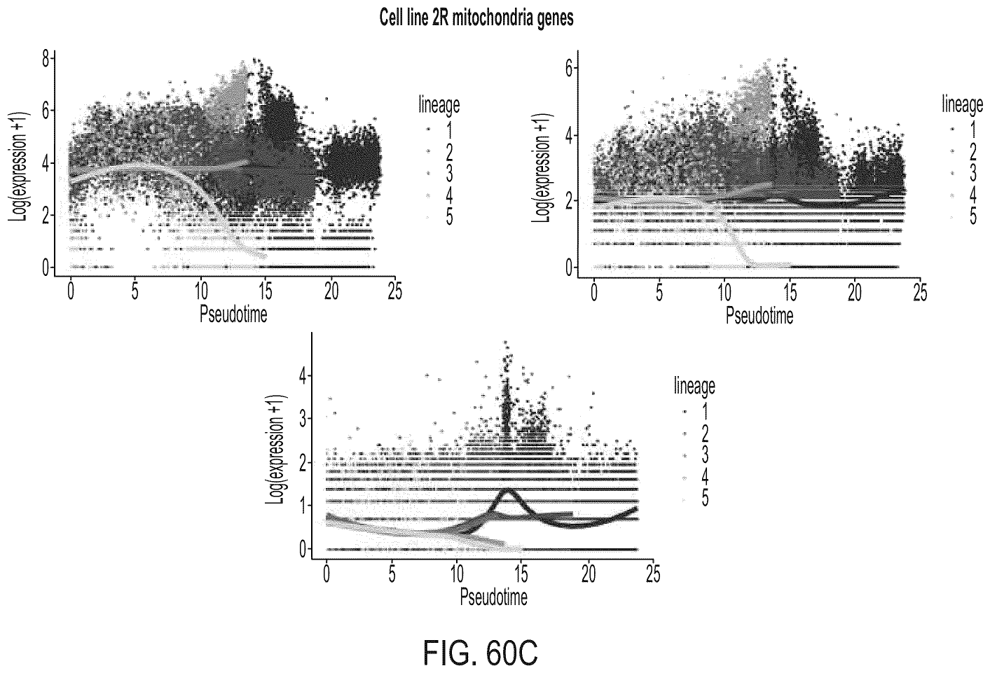

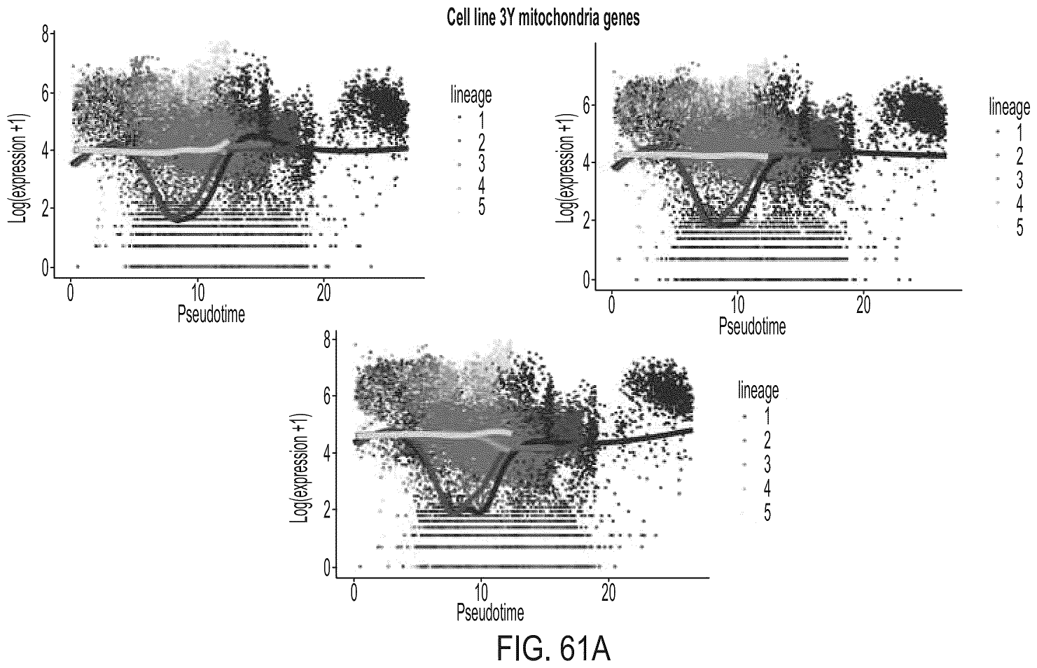

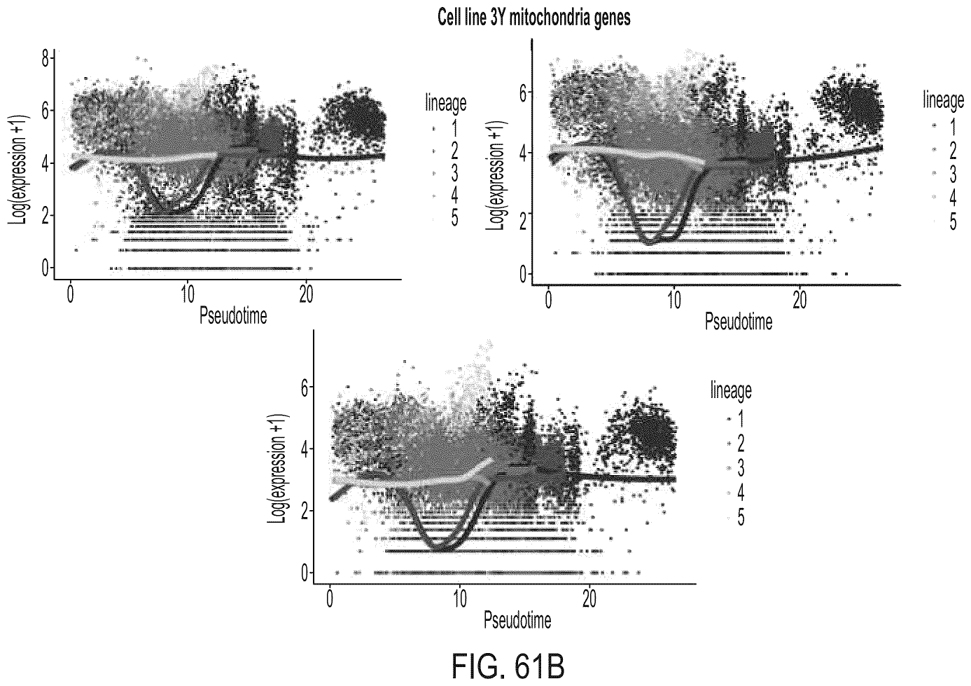

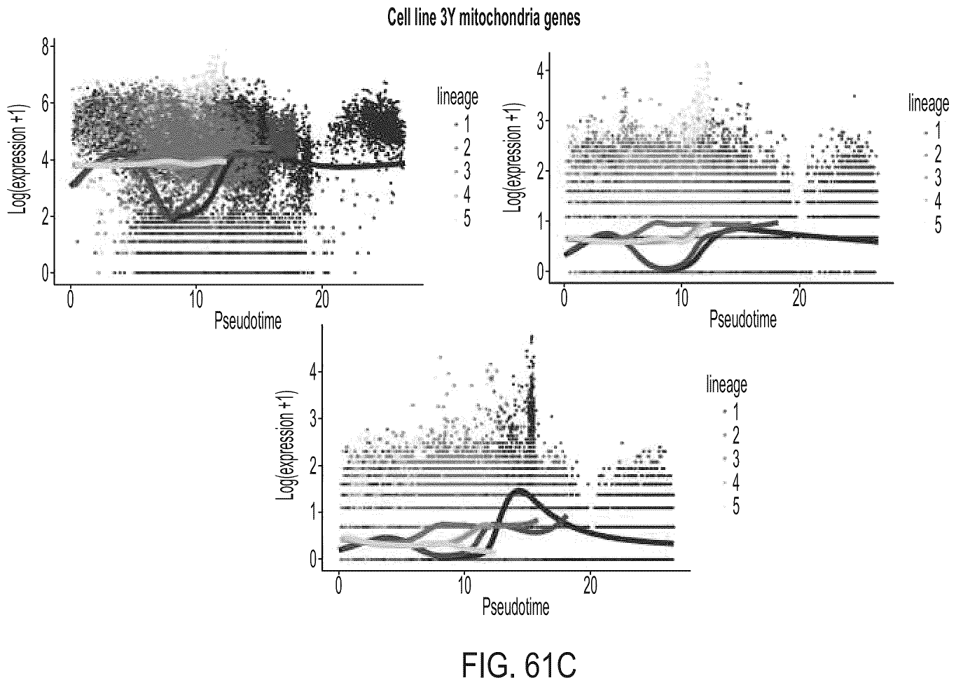

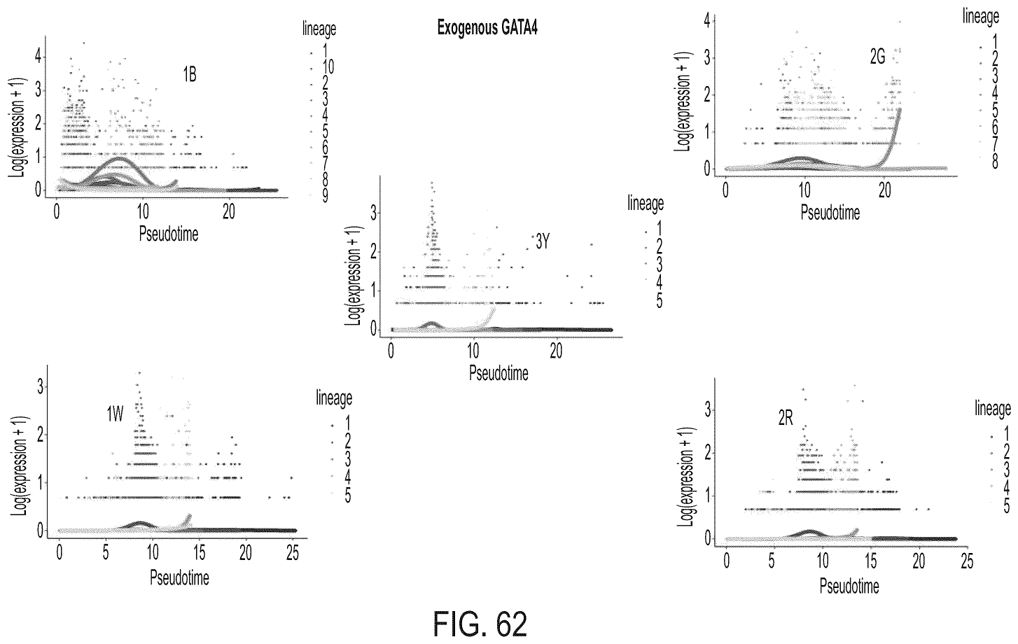

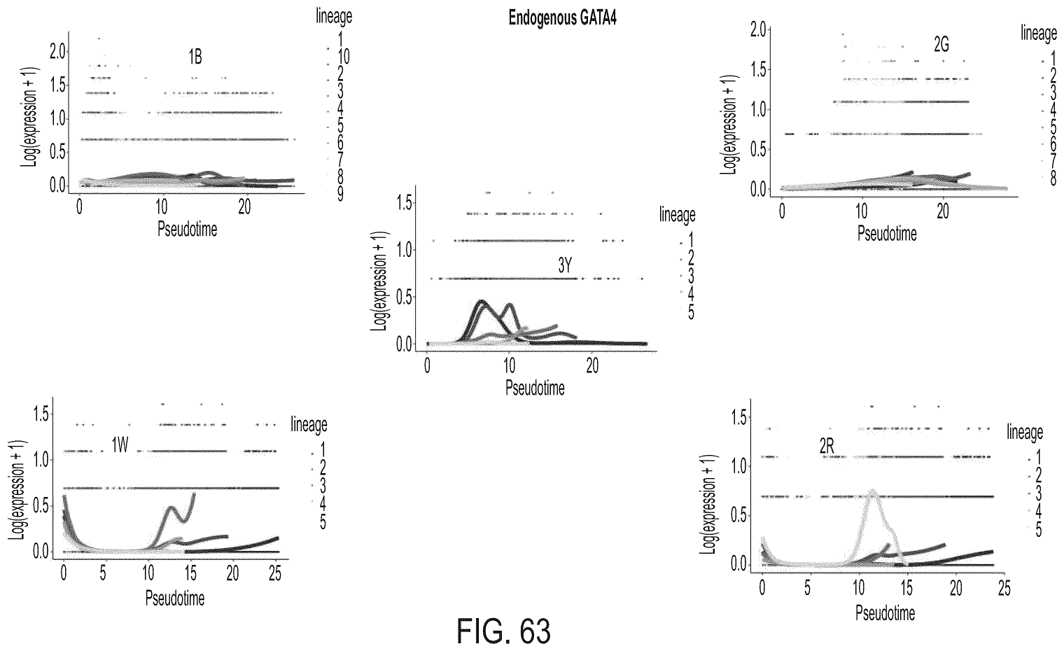

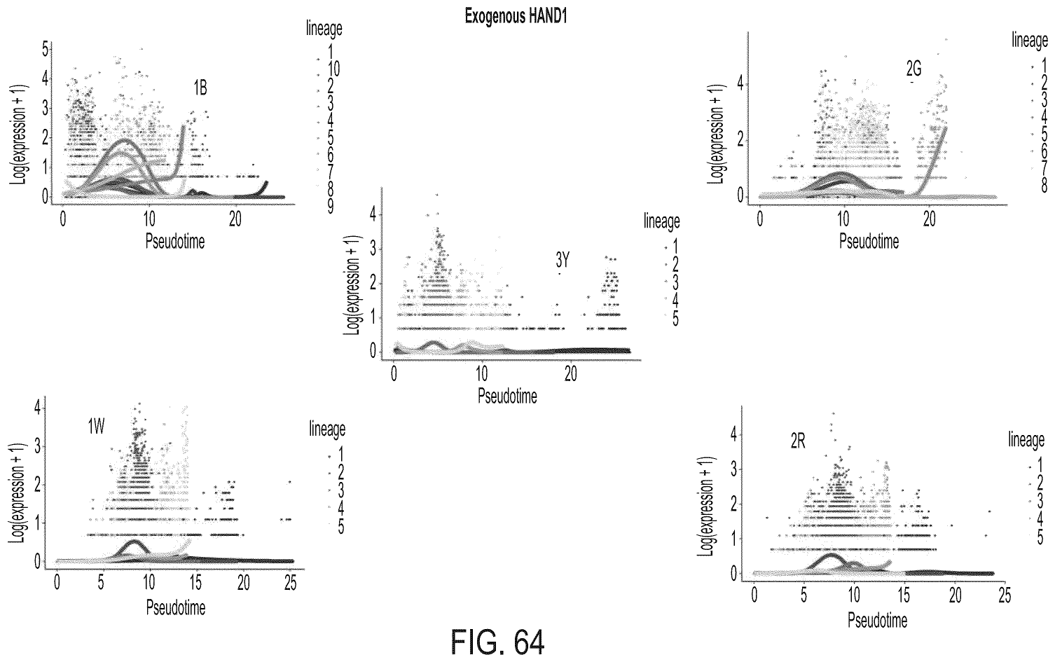

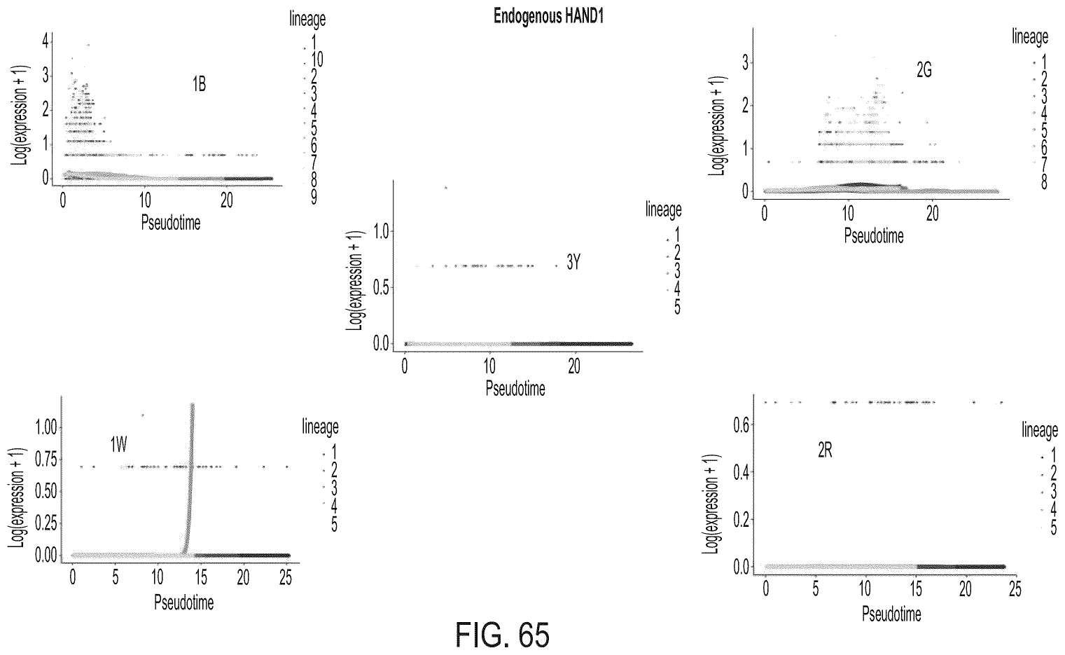

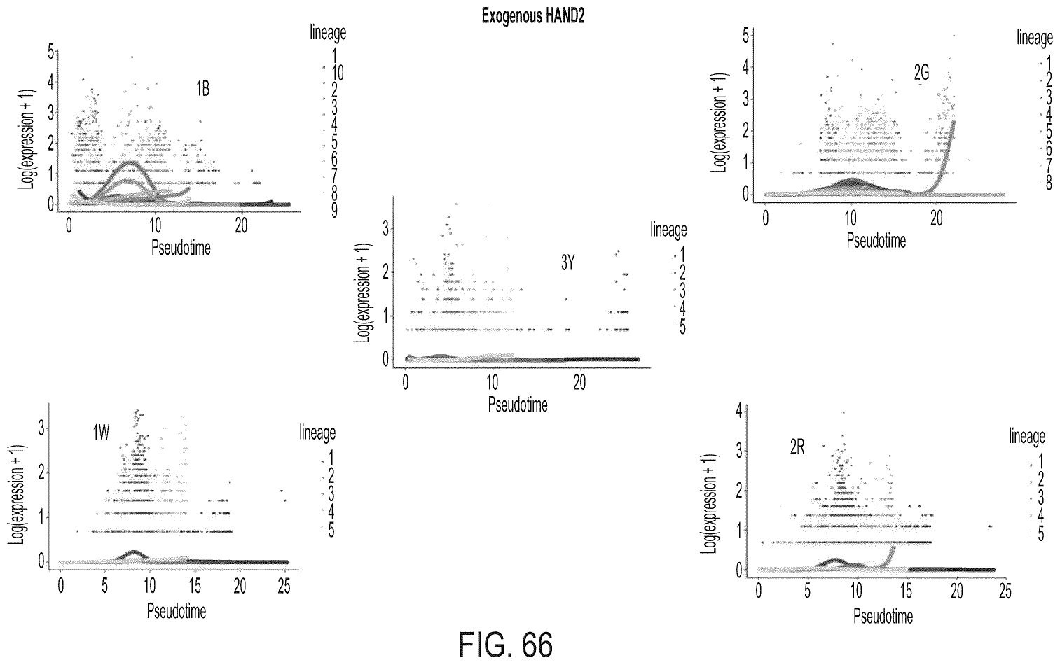

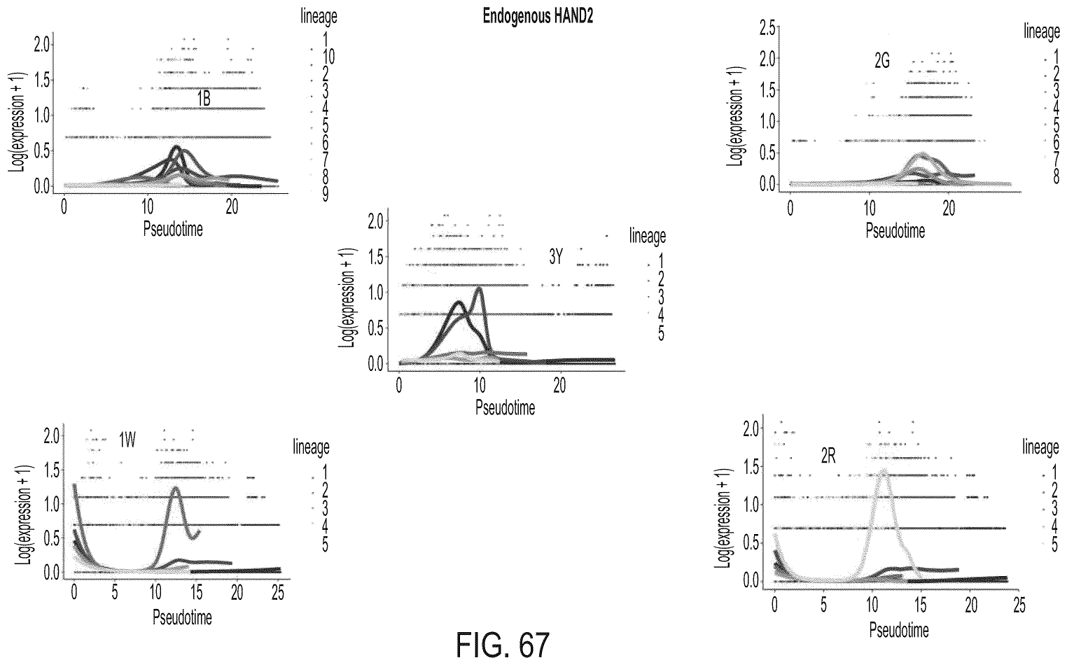

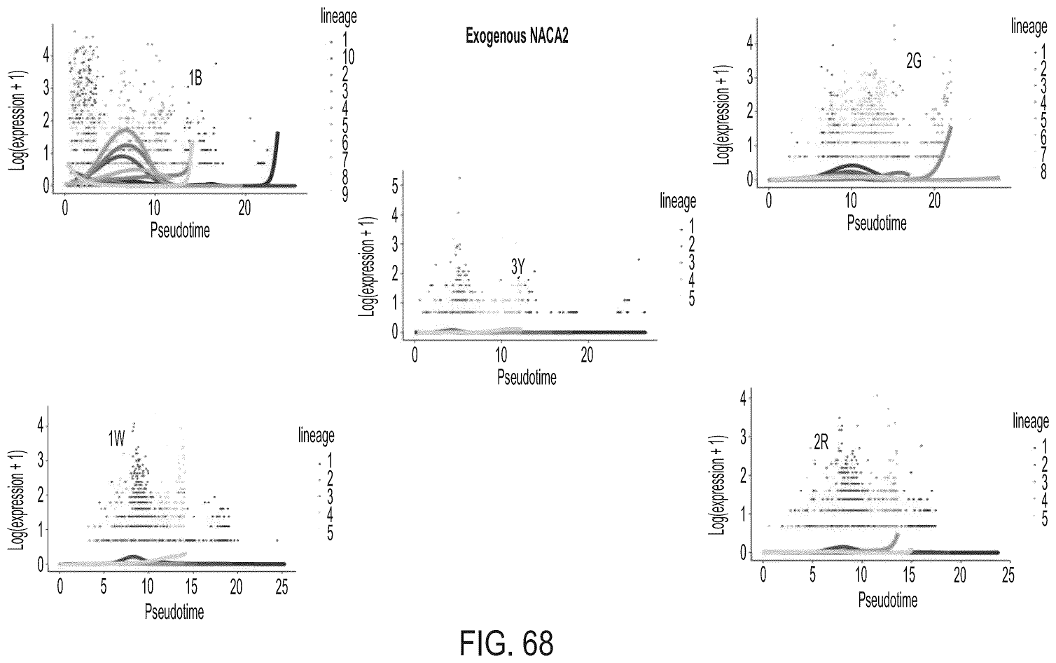

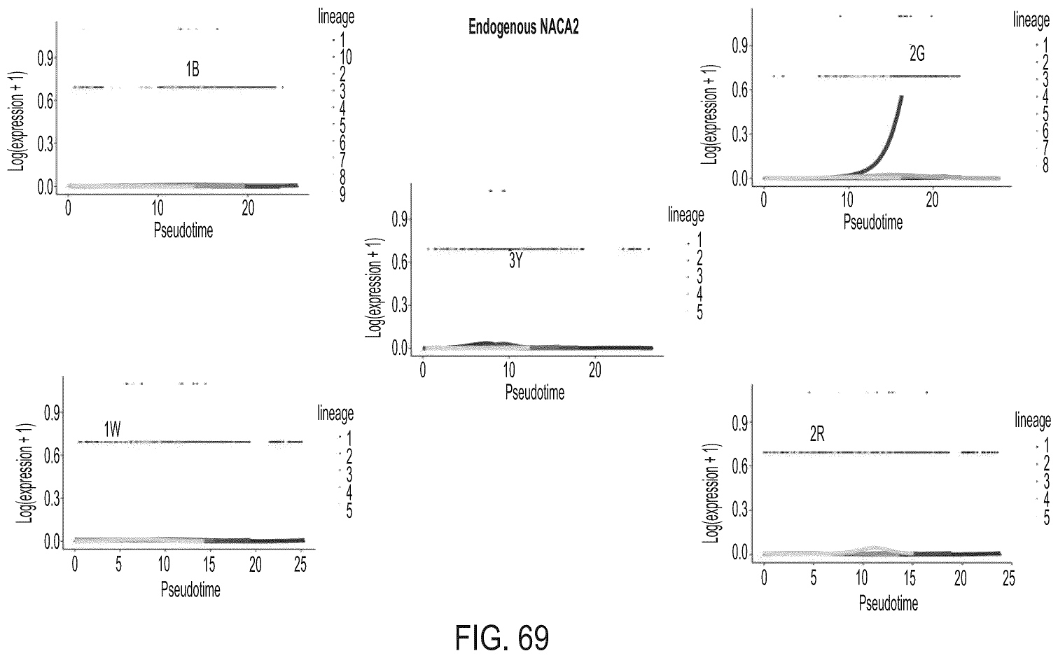

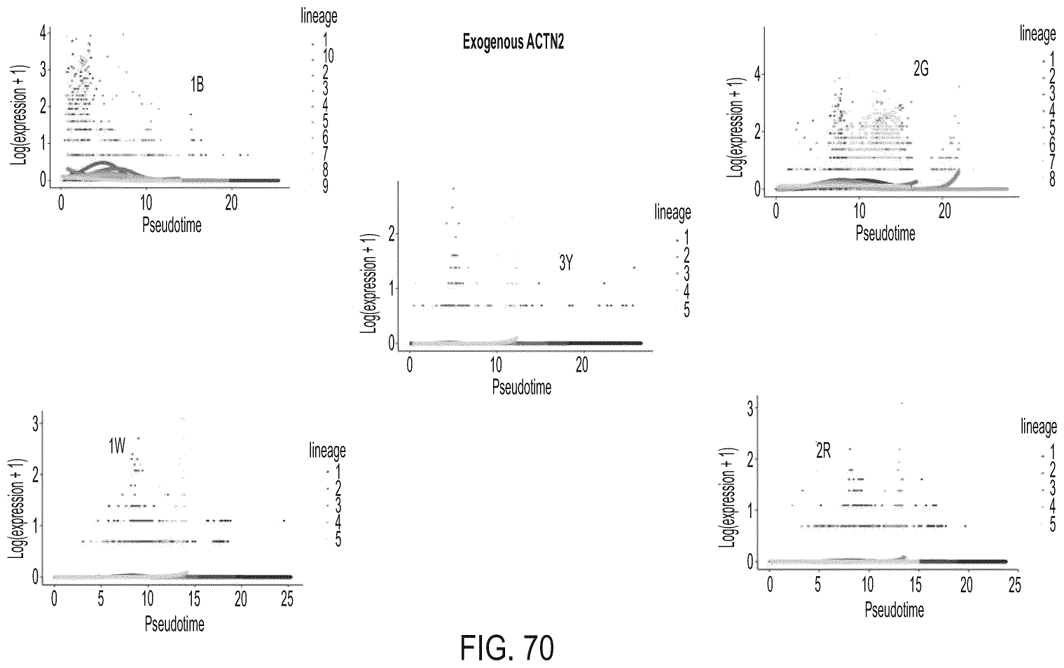

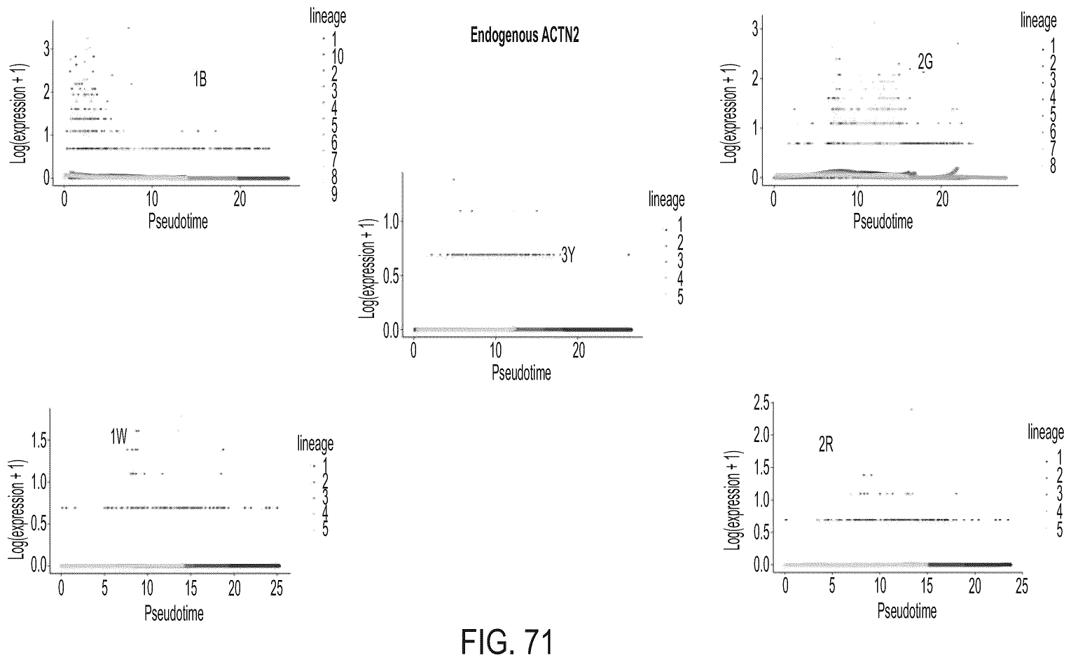

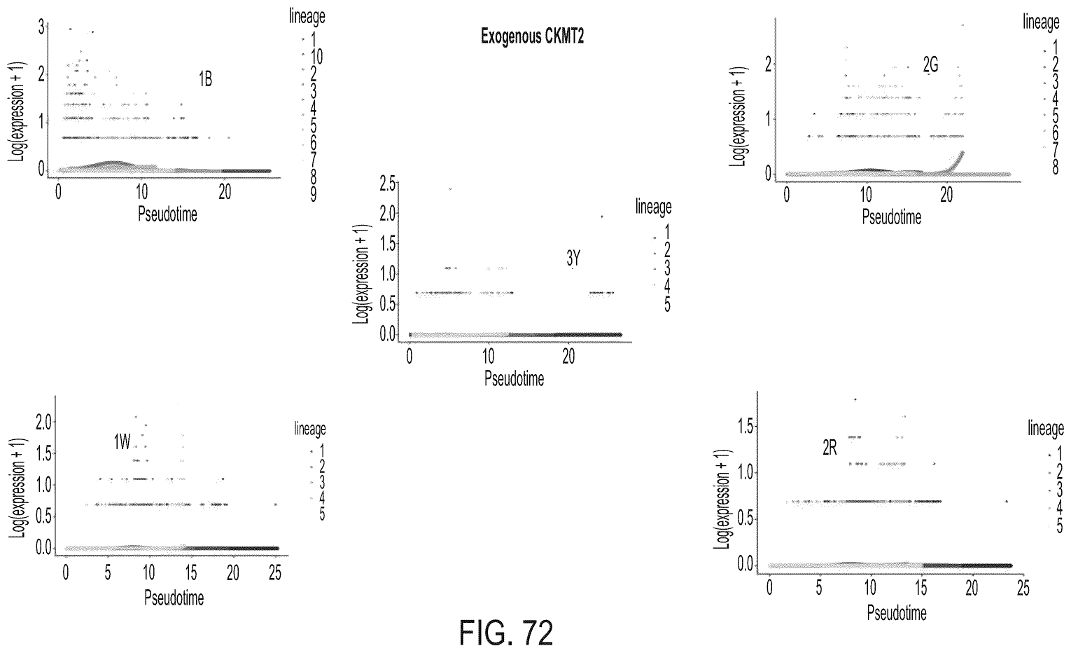

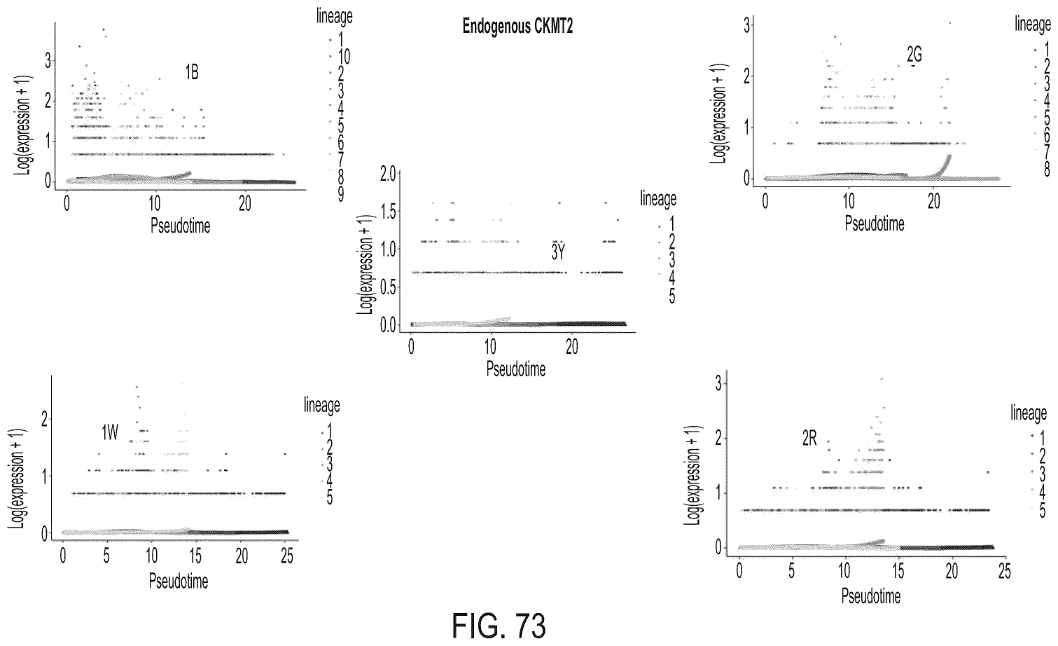

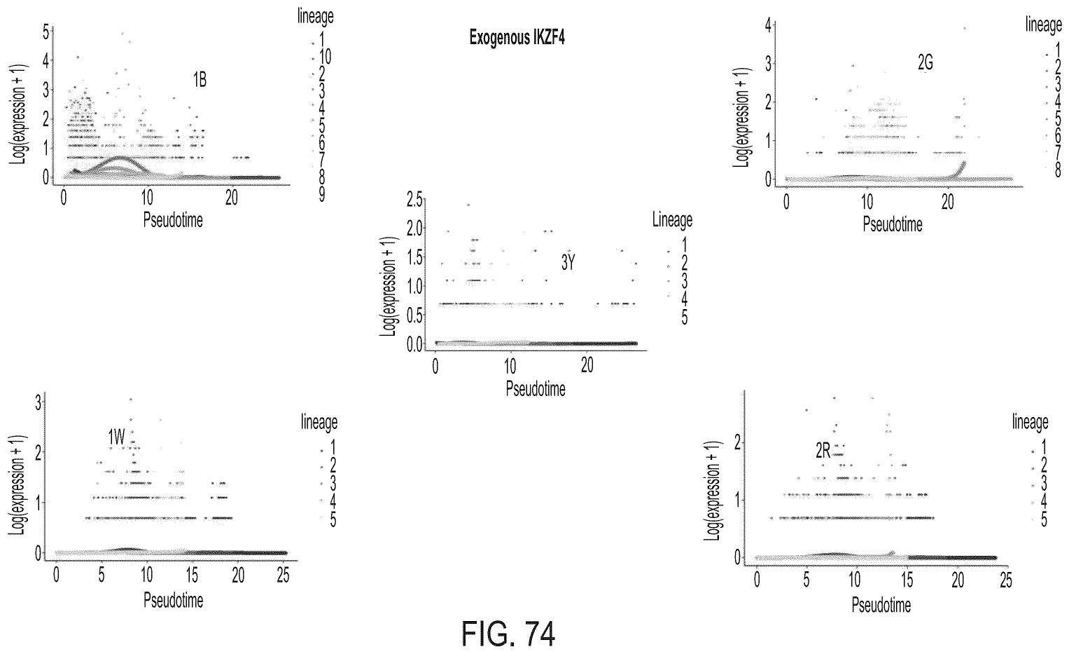

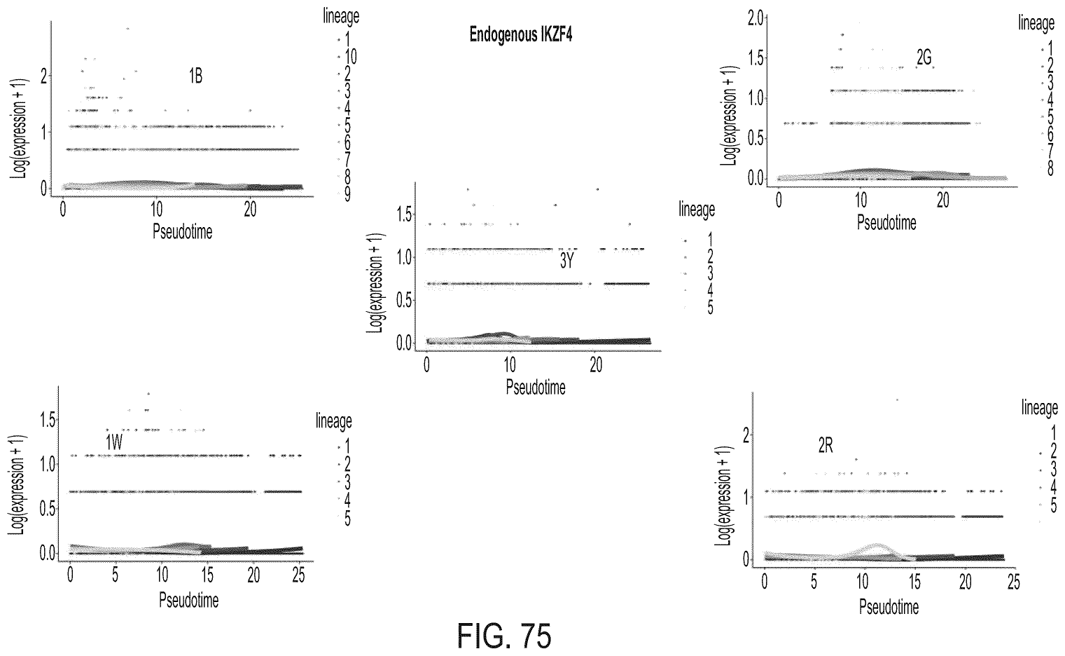

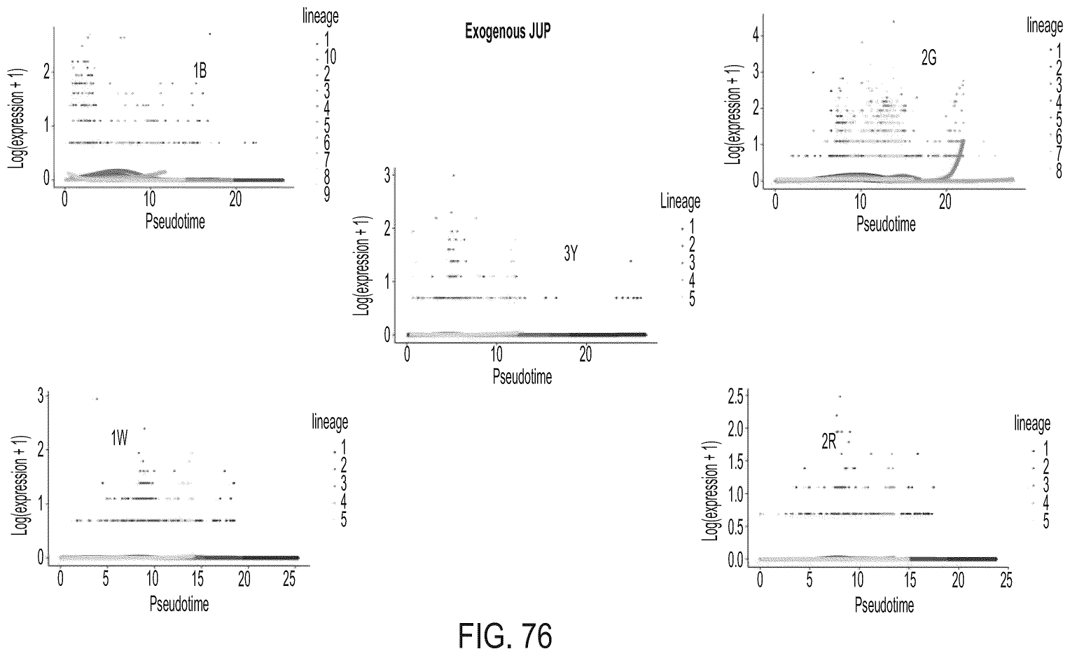

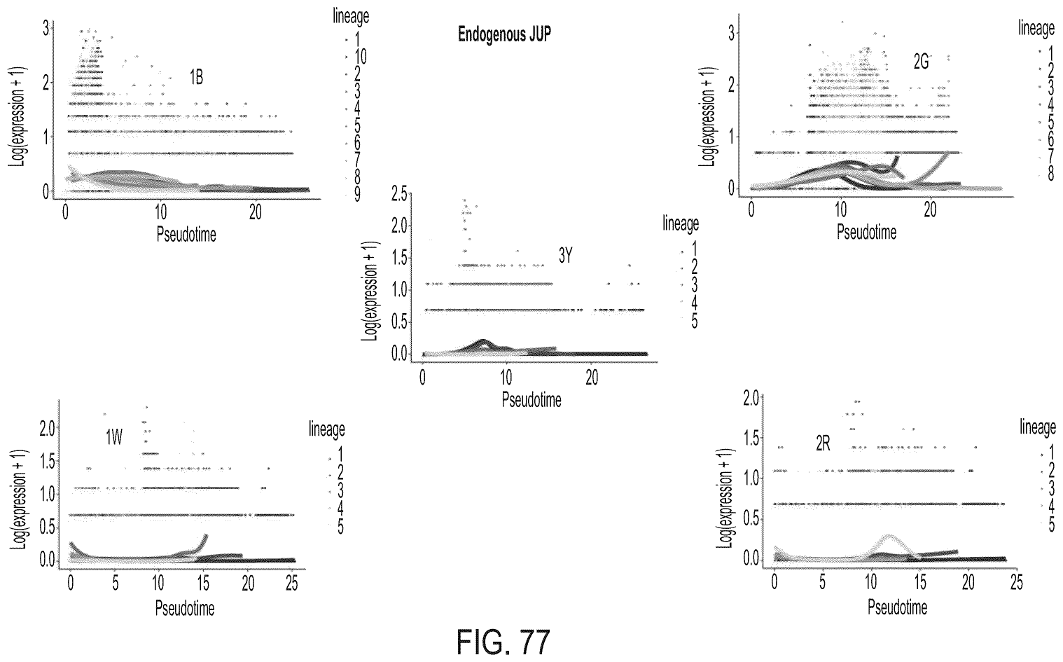

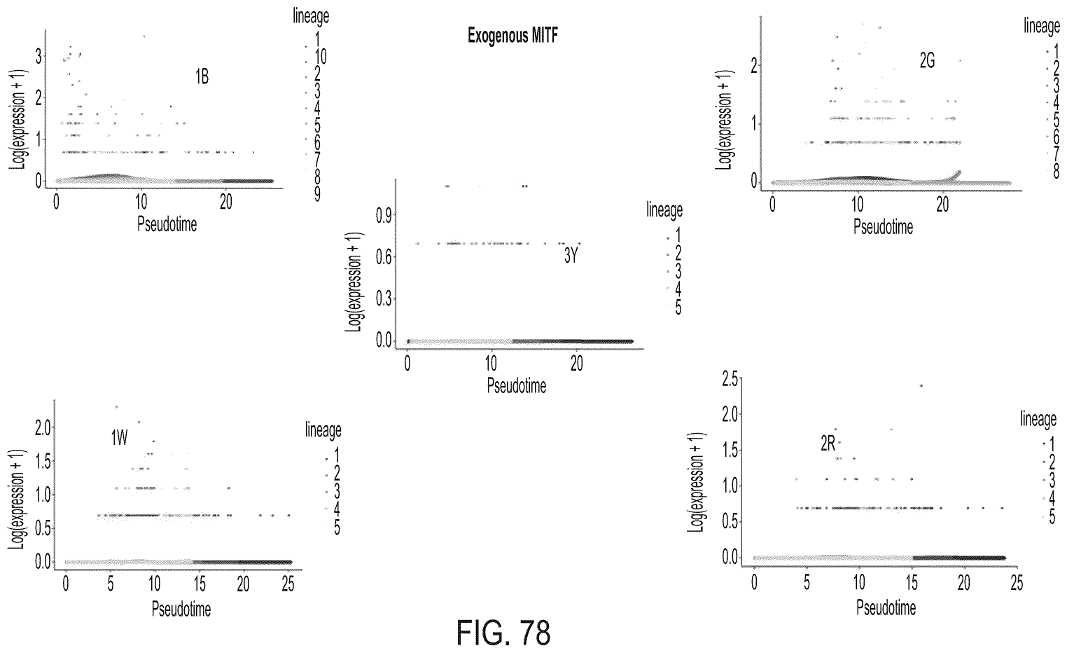

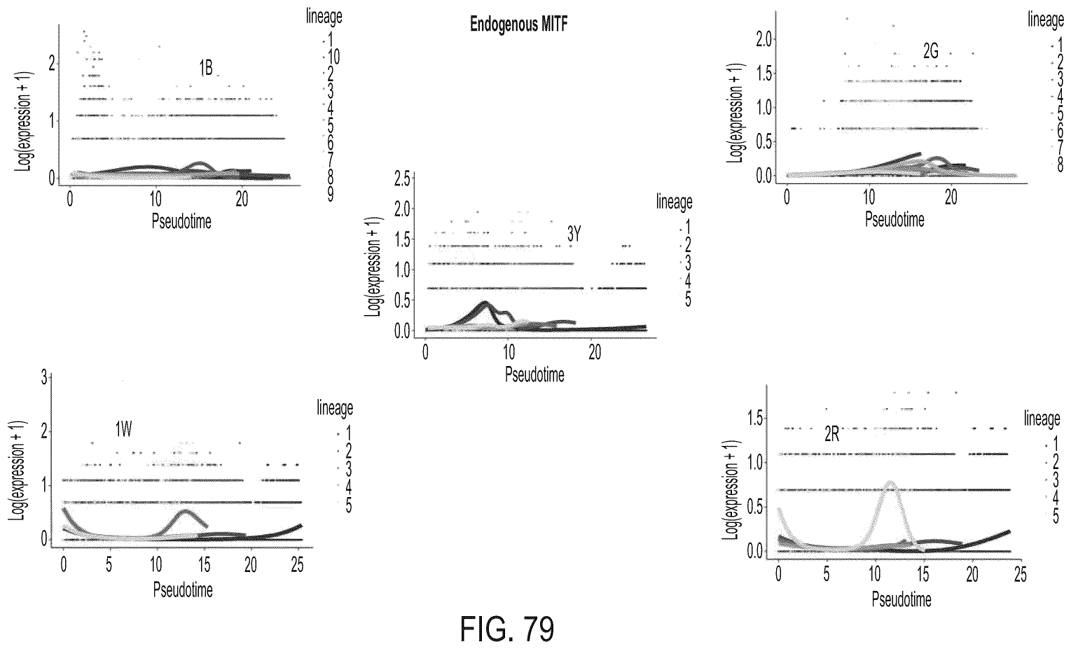

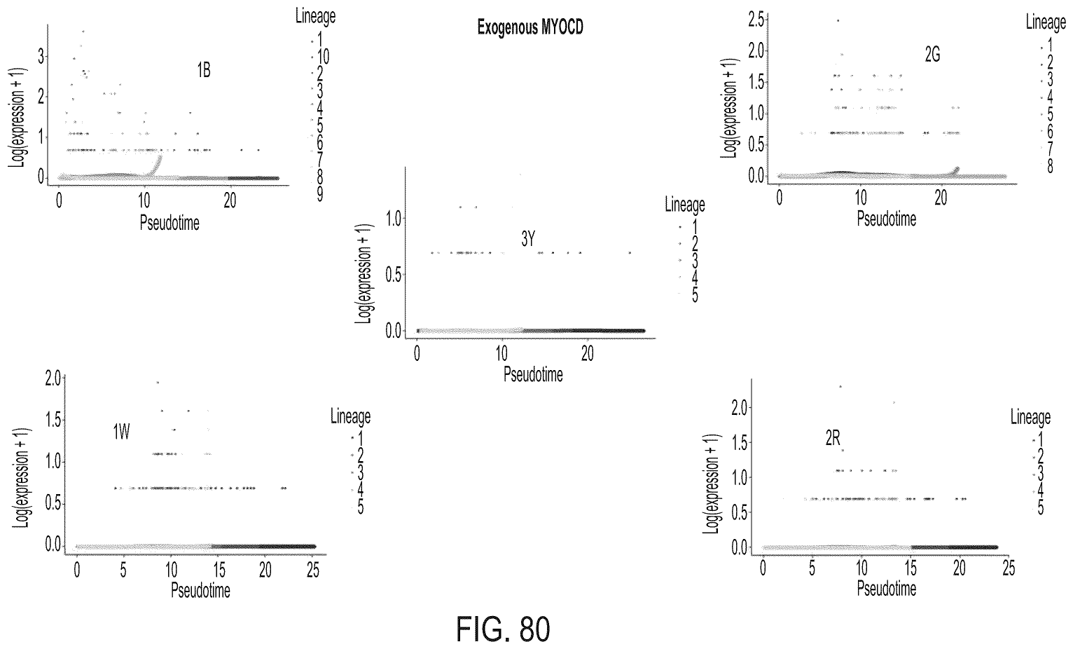

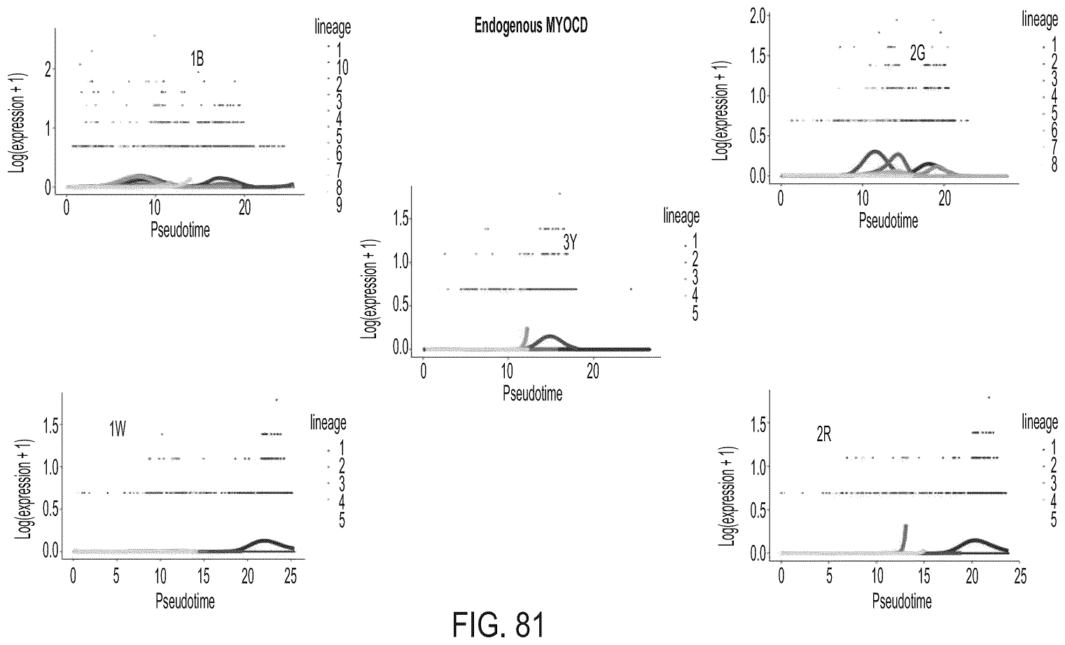

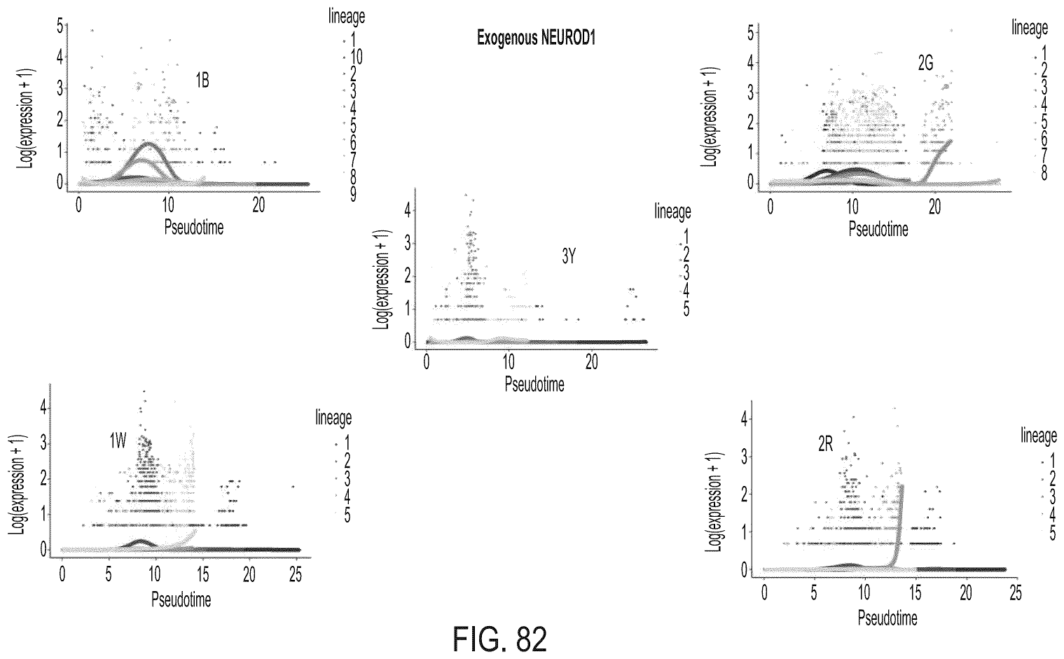

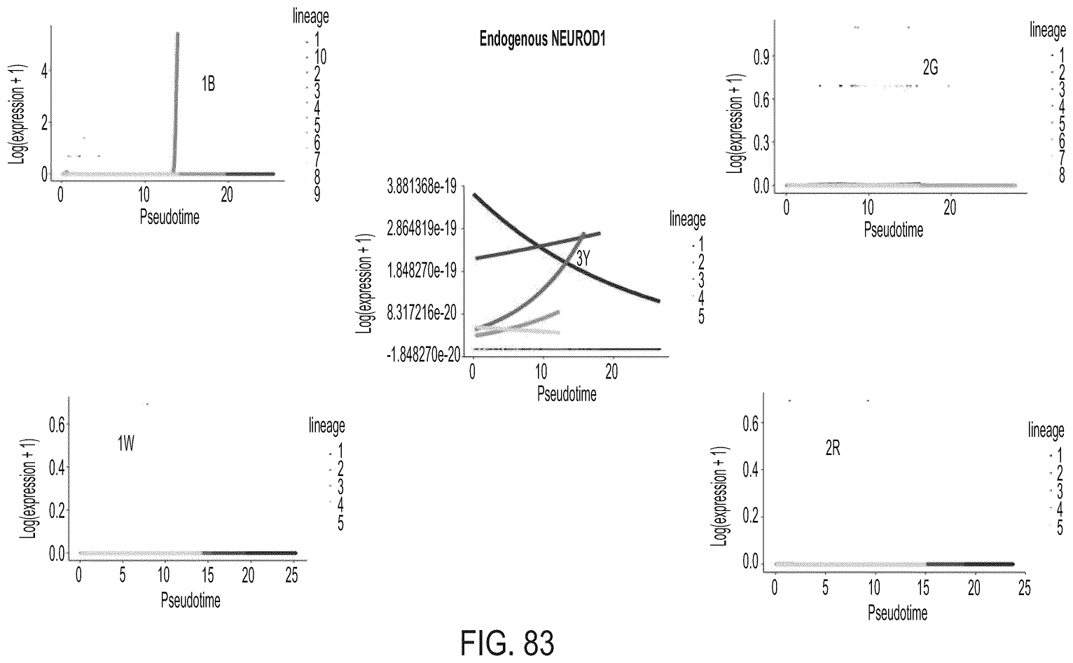

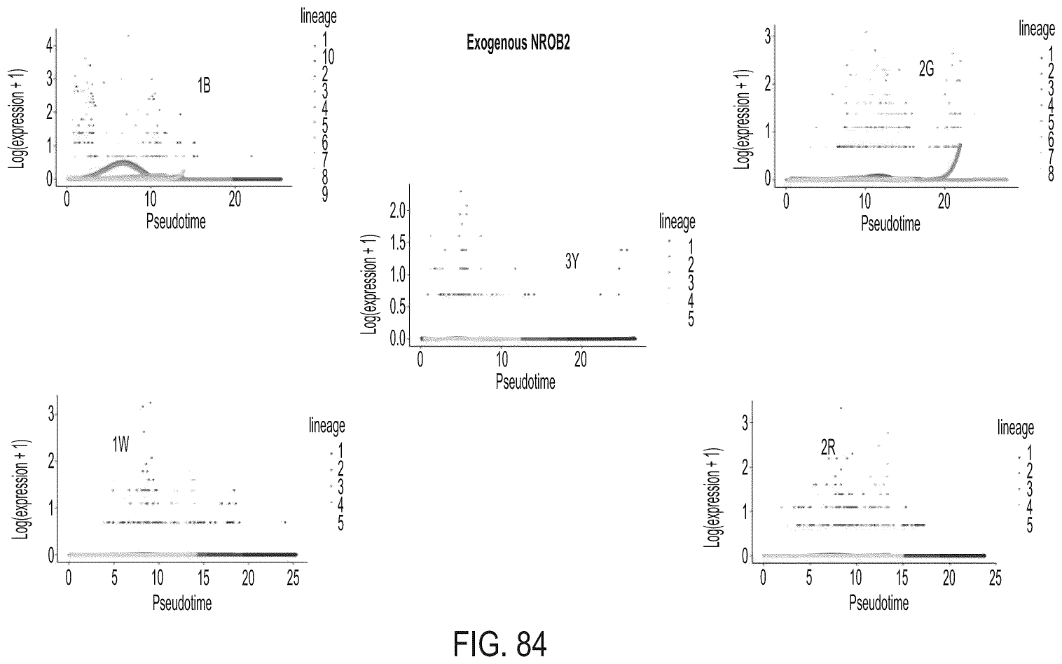

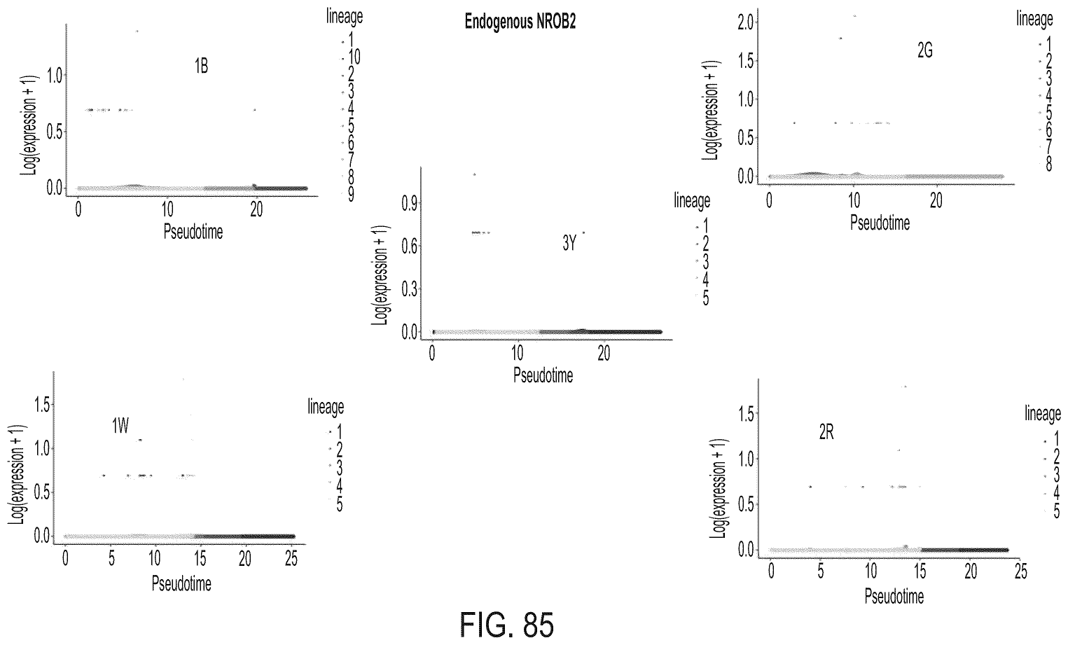

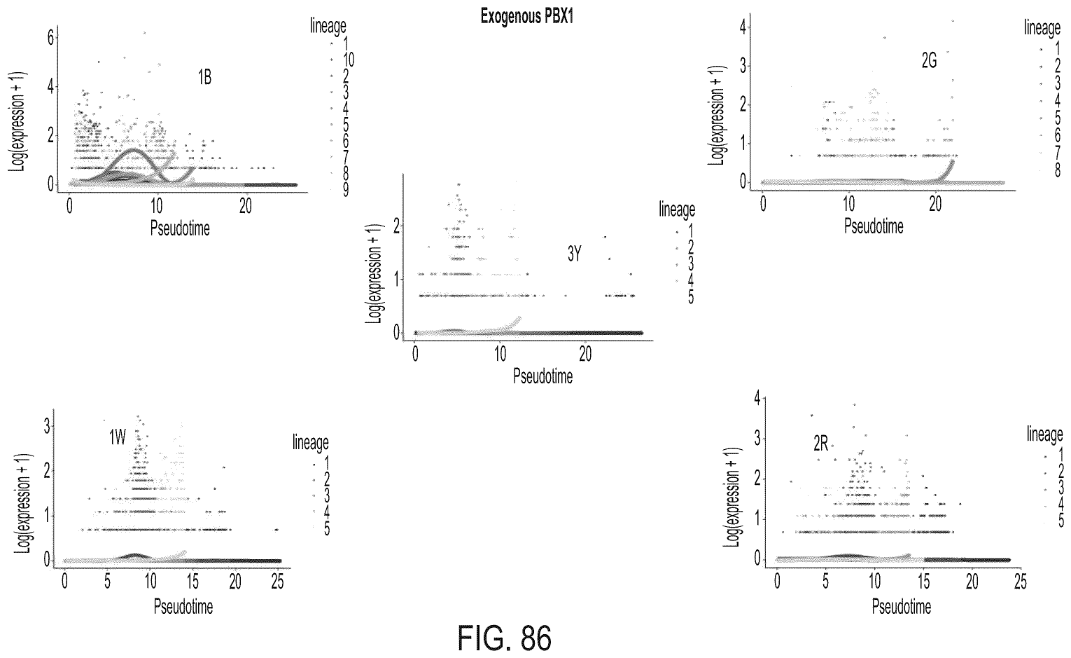

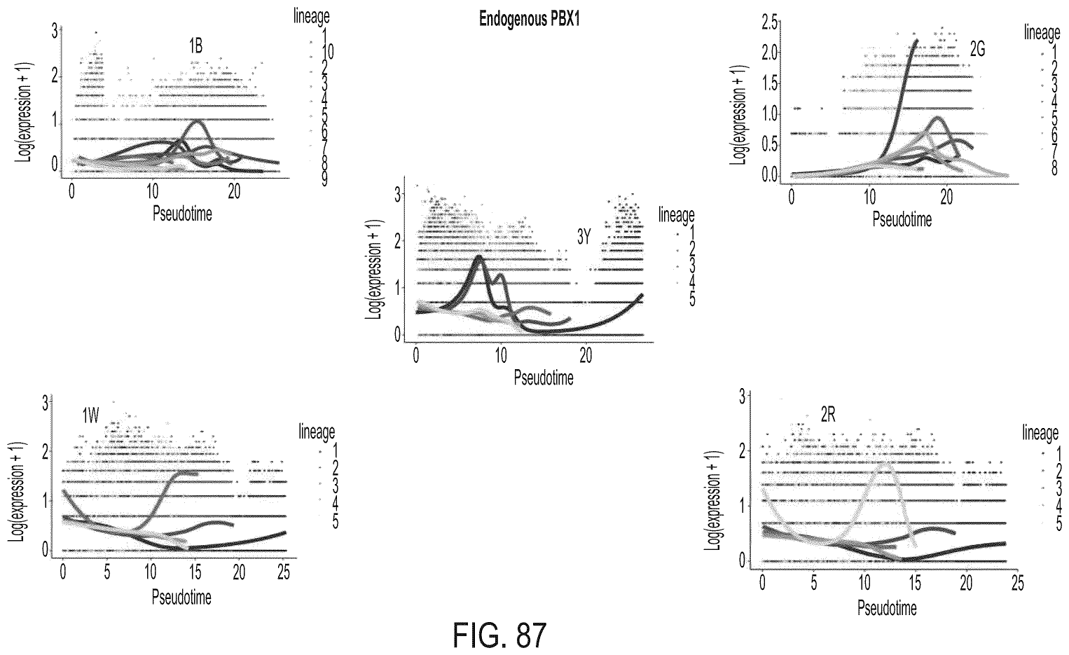

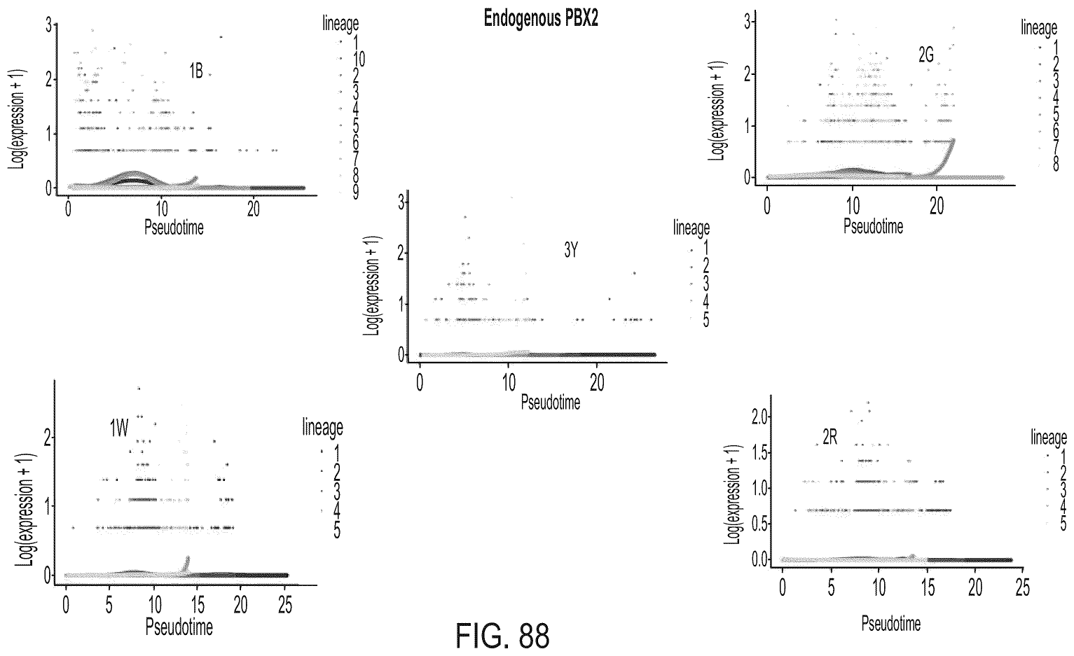

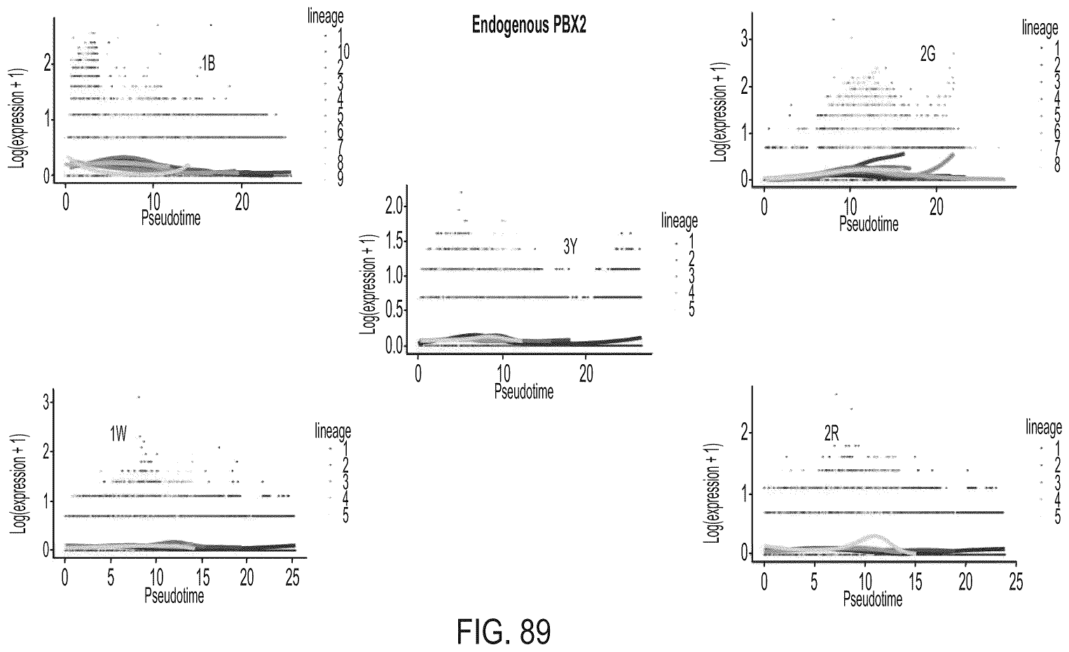

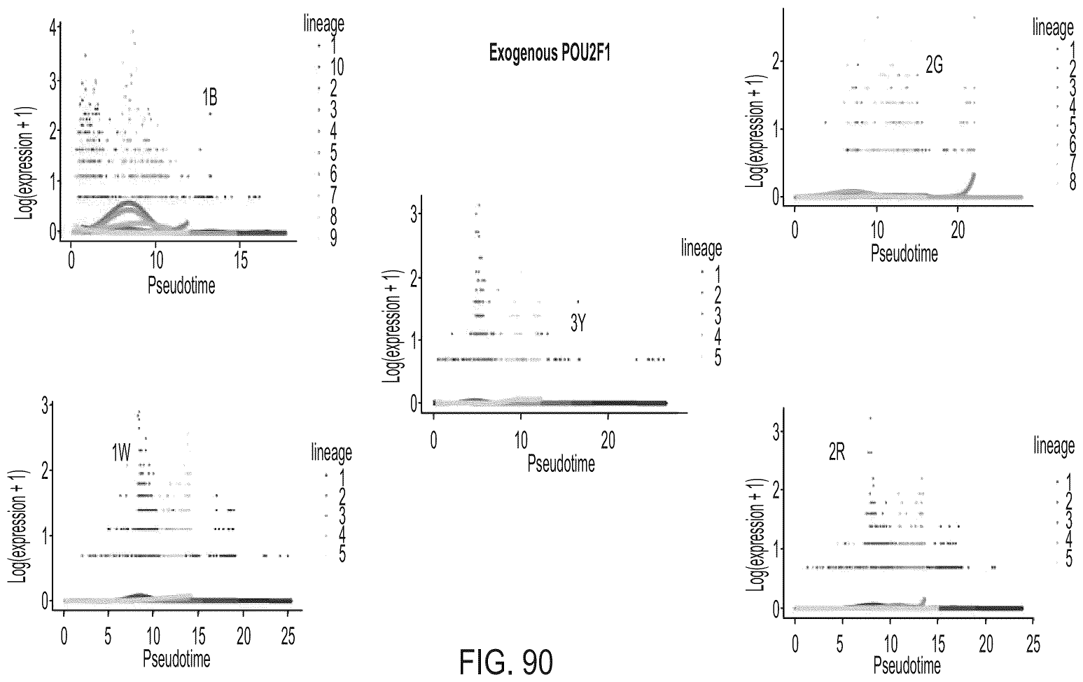

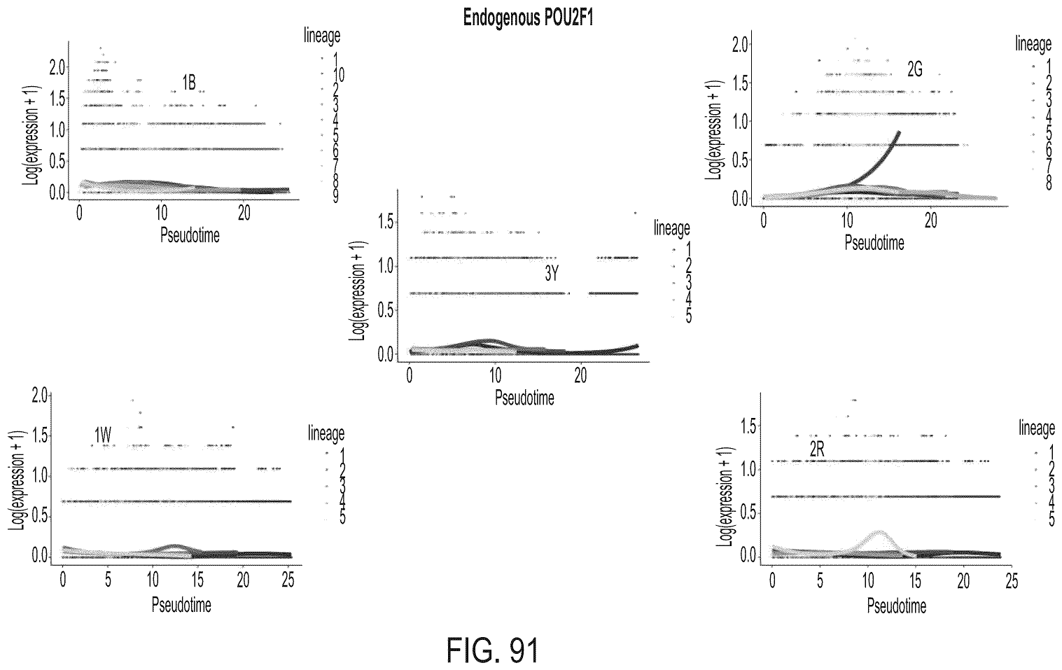

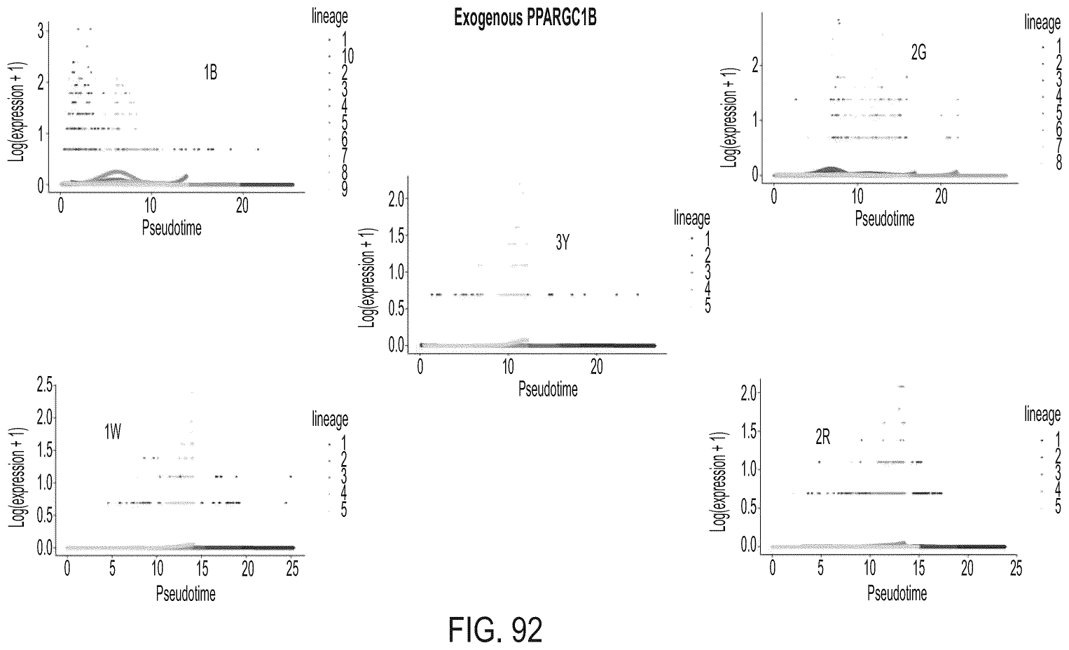

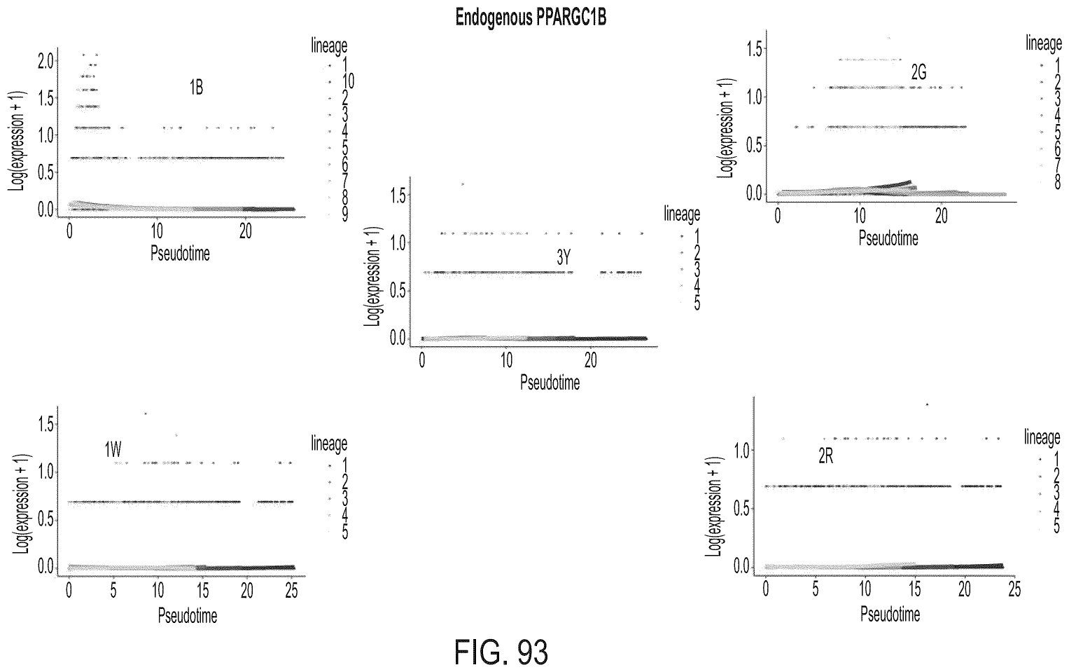

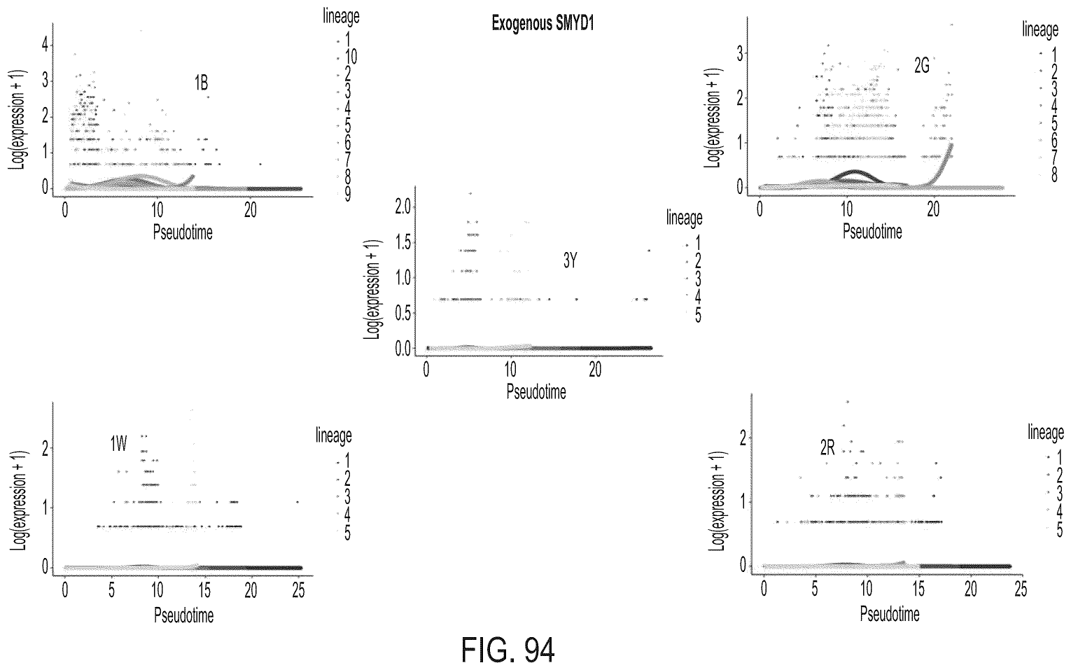

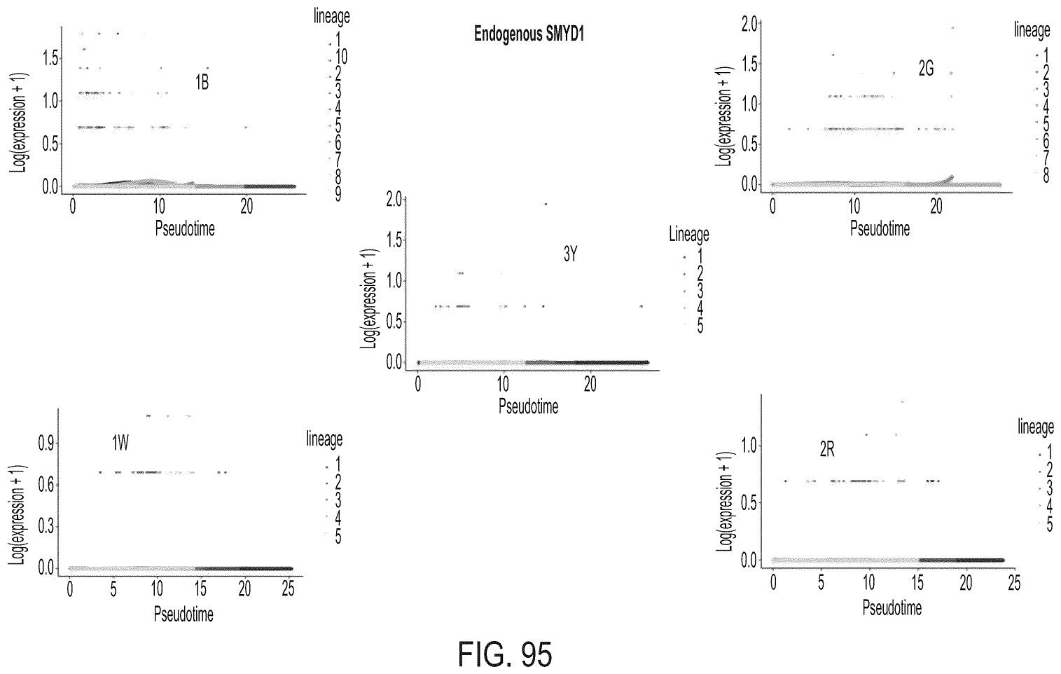

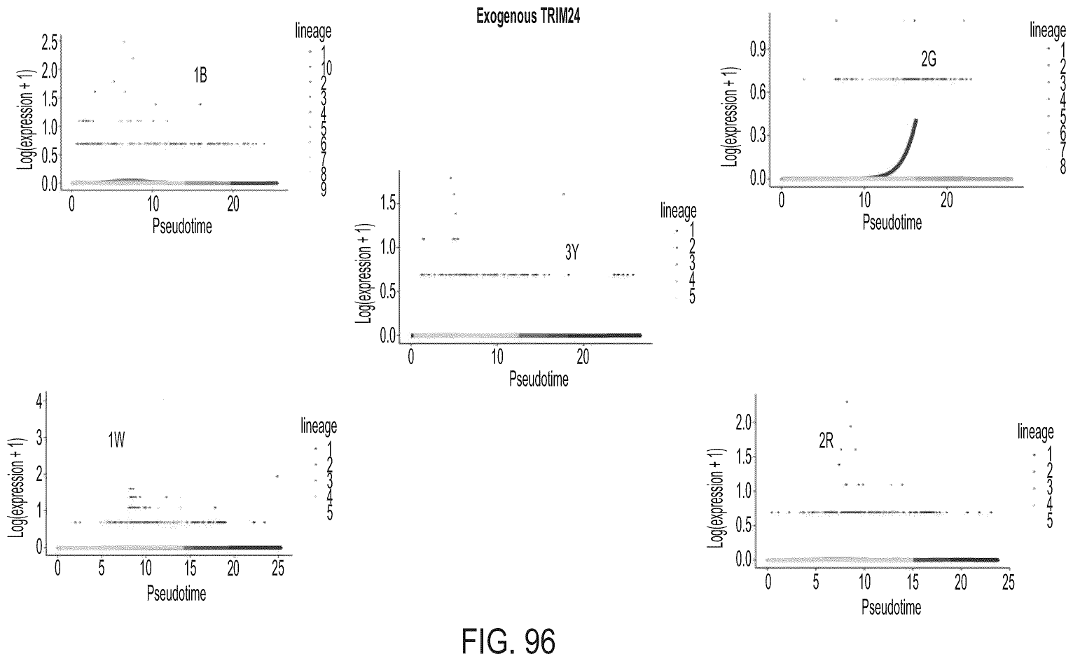

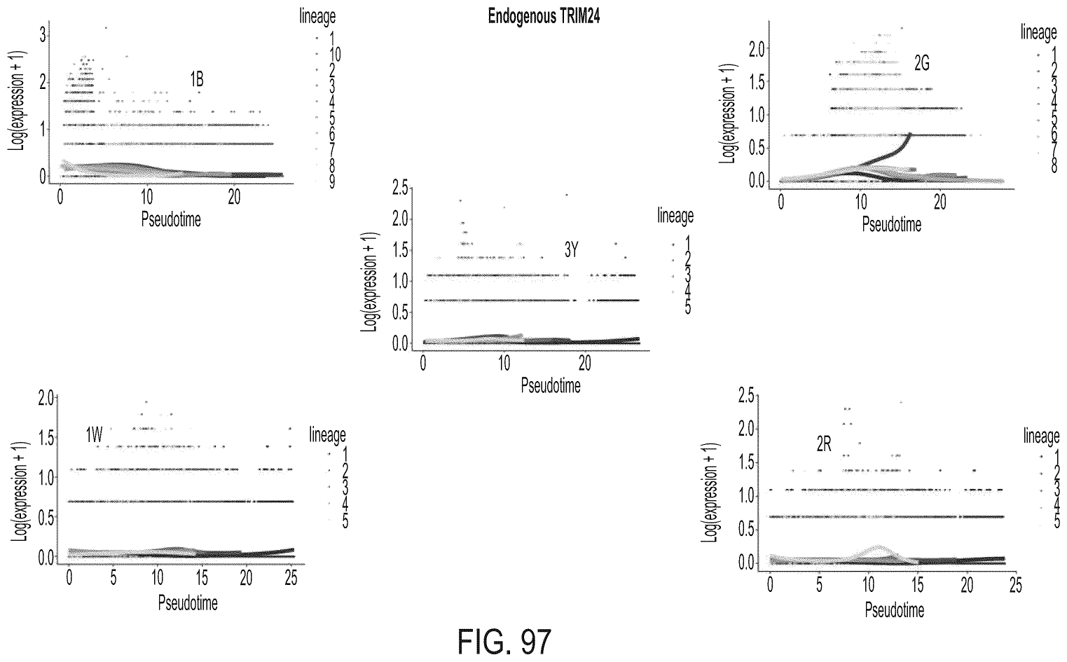

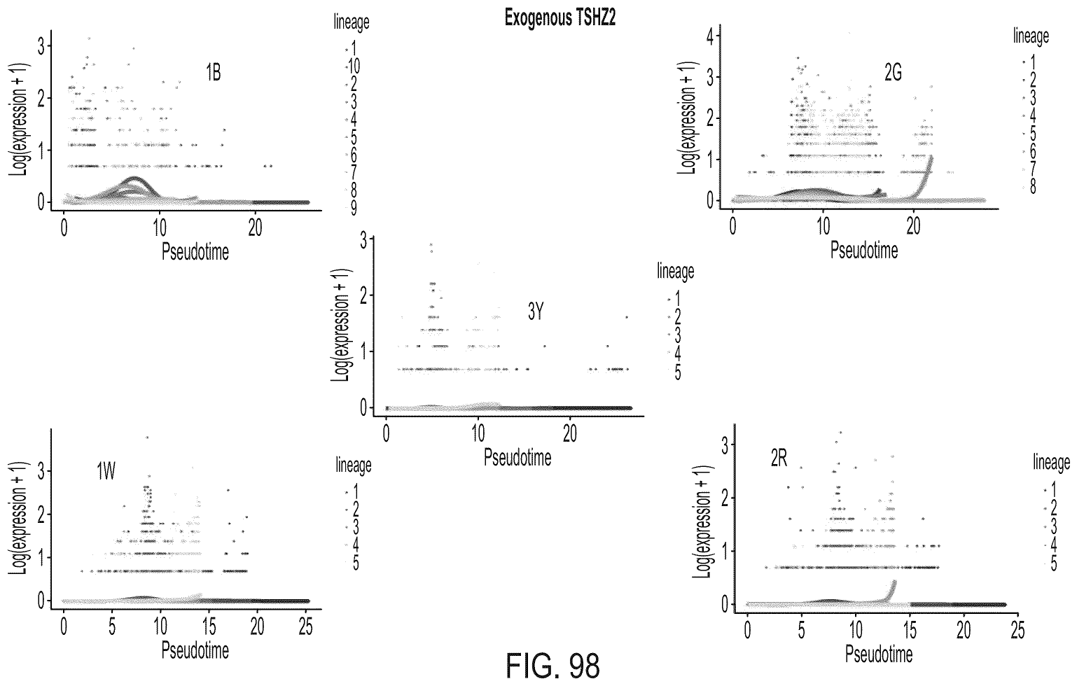

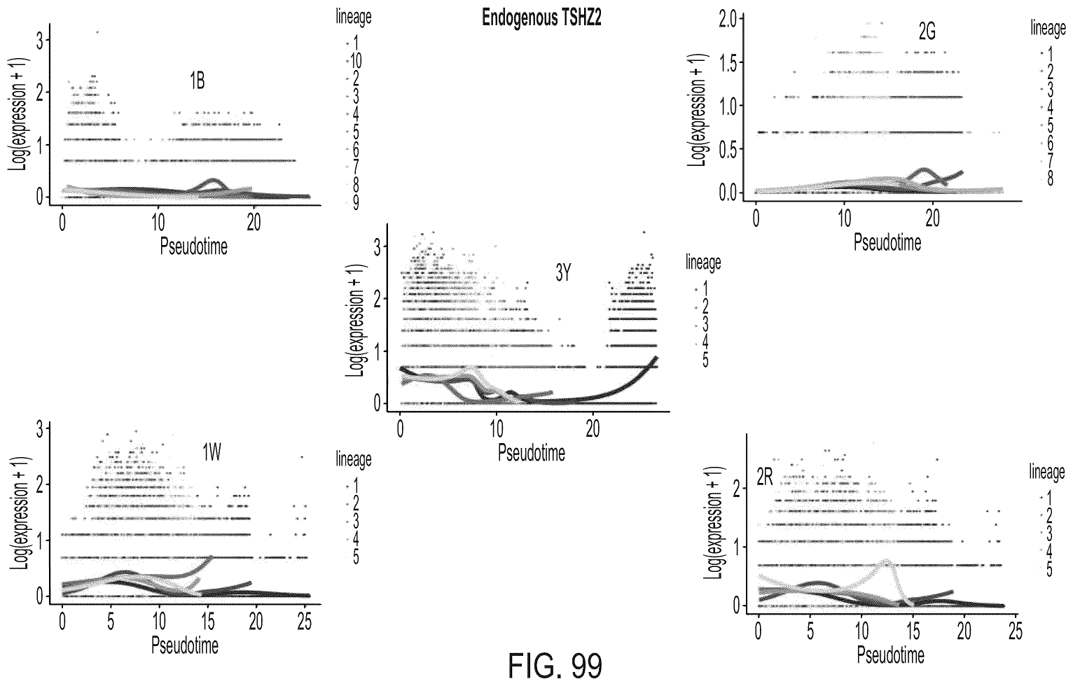

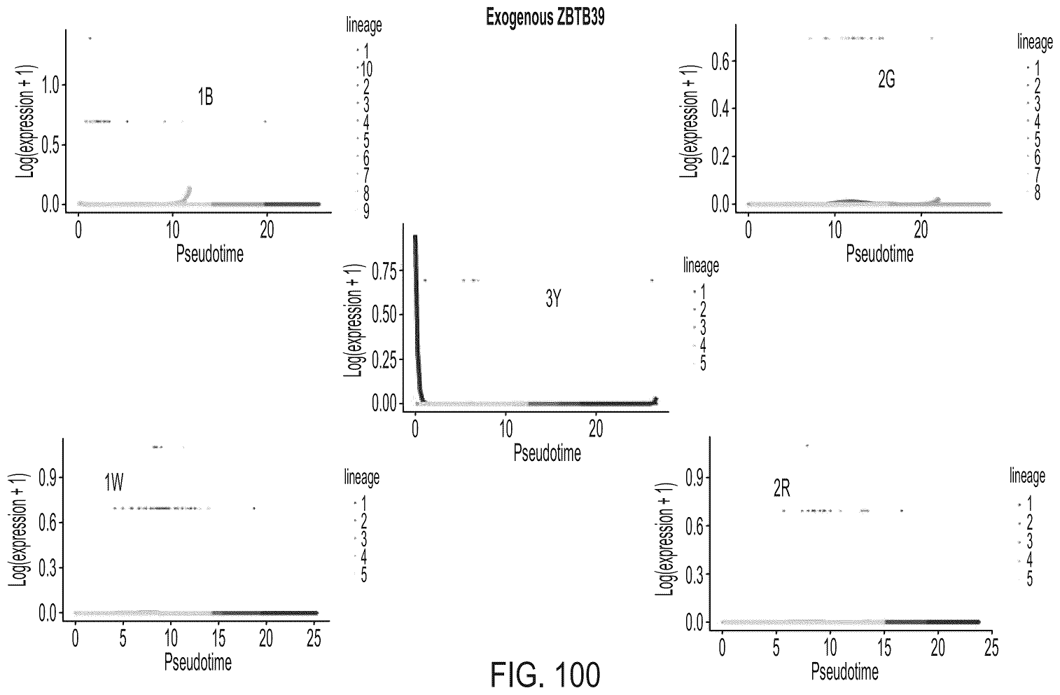

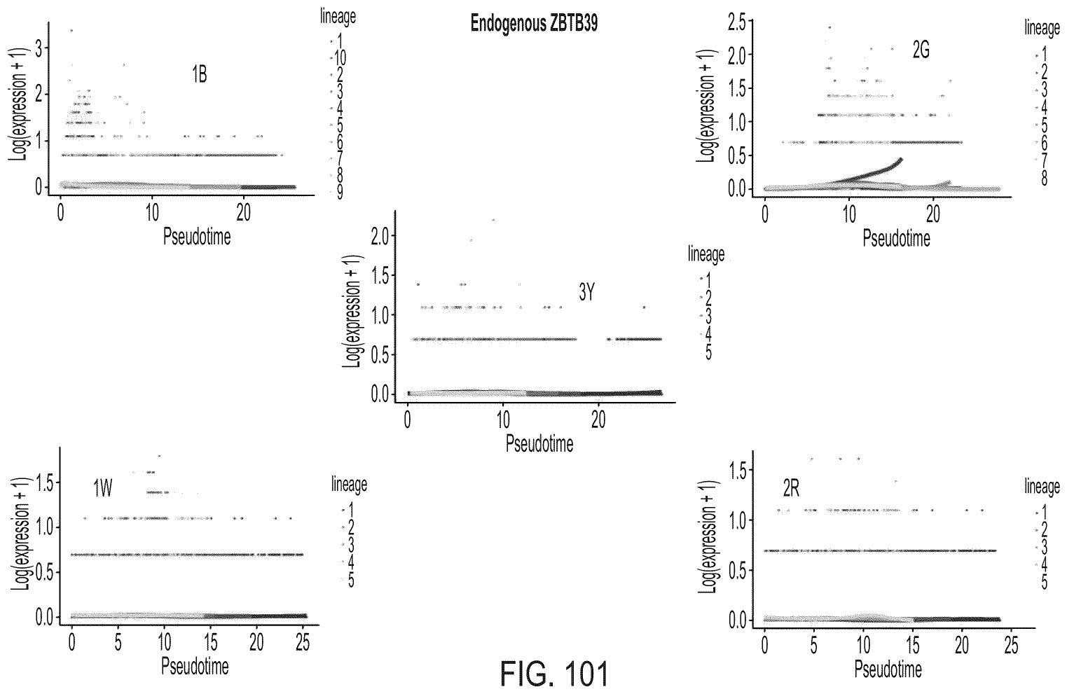

FIGS. 57-101 depict tradeSeq with UMAP 3D slingshot. FIGS. 57A-C show Cell line 1B mitochondria genes, FIGS. 58A-C show Cell line 2G mitochondria genes. FIGS. 59A-C show Cell line 1W mitochondria genes. FIGS. 60A-C show Cell line 2R mitochondria genes. FIGS. 61A-C show Cell line 3Y mitochondria genes. Table 7 indicates Figure numbers for tradeSeq with UMAP 3D slingshot results for indicated genes.

TABLE 7
Figure numbers for tradeSeq with UMAP 3D
slingshot results for indicated genes.

| | FIG. Number | FIG. Number |
|---|---|---|
| Gene | for Exogenous | for Endogenous |
| GATA4 | 62 | 63 |
| HAND1 | 64 | 64 |
| HAND2 | 66 | 67 |
| NACA2 | 68 | 69 |
| ACTN2 | 70 | 71 |
| CKMT2 | 72 | 73 |
| IKZF4 | 74 | 75 |
| JUP | 76 | 77 |
| MITF | 78 | 79 |
| MYOCD | 80 | 81 |
| NEUROD1 | 82 | 83 |
| NROB2 | 84 | 85 |
| PBX1 | 86 | 87 |
| PBX2 | 88 | 89 |
| POU2F1 | 90 | 91 |
| PPARGC1B | 92 | 93 |
| SMYD1 | 94 | 95 |
| TRIM24 | 96 | 97 |
| TSHZ2 | 98 | 99 |
| ZBTB39 | 100 | 101 |

CFD Combinations were identified:
COM 1: C6 (GATA4), C3 (POU2F1), C4 (HAND1), C12 (NACA2), C17 (TSHZ2)
COM 2: C6 (GATA4), C10 (IKZF4), C12 (NACA2), C17 (TSHZ2)
COM 3: C6 (GATA4), C3 (POU2F1), C4 (HAND1), C9 (HAND2)
COM 4: C6 (GATA4), C9 (HAND2), C10 (IKZF4)
COM 5: C6 (GATA4), C3 (POU2F1), C17 (TSHZ2)
COM 6: C6 (GATA4), C4 (HAND1), C12 (NACA2), C10 (IKZF4)
COM 7: C6 (GATA4), C4 (HAND1), C12 (NACA2)
COM 8: C6 (GATA4), C3 (POU2F1), C4 (HAND1), C10 (IKZF4), C12 (NACA2)
COM 9: C6 (GATA4), C3 (POU2F1), C4 (HAND1), C14 (JUP), C17 (TSHZ2)
COM 10: C6 (GATA4), C2 (ACTN2), C3 (POU2F1), C4 (HAND1)

Table 8 presents results for iCM functional analysis for Indicated CFD Combinations

TABLE 8
Results for iCM functional analysis
for Indicated CFD Combinations

| NETZEN ranking | IU/ml | Experimental ranking | Fraction |
|---|---|---|---|
| (C1) PBX2 | 7.809E+08 | (C6)GATA4 | 0.275 |
| (C2) ACTN2 | 4.787E+07 | (C4) HAND1 | 0.15 |
| (C3) POU2F1 | 1.226E+07 | (C12) NACA2 | 0.14 |
| (C4) HAND1 | 3.227E+07 | (C10) IKZF4 | 0.13 |
| (C5) TRIM24 | 1.076E+08 | (C3) POU2F1 | 0.105 |
| (C6)GATA4 | 5.212E+07 | (C7) PBX1 | 0.095 |
| (C7) PBX1 | 2.062E+08 | (C17) TSHZ2 | 0.095 |
| (C8) ZBTB39 | 1.070E+08 | (C9) HAND2 | 0.085 |
| (C9) HAND2 | 1.964E+08 | (C14) JUP | 0.065 |
| (C10) IKZF4 | 8.227E+07 | (C15) NEUROD1 | 0.05 |
| (C11) NR0B2 | 9.535E+08 | (C2) ACTN2 | 0.045 |
| (C12) NACA2 | 2.099E+08 | (C16)CKMT2 | 0.04 |
| (C13) SMYD1 | 5.113E+08 | (C13) SMYD1 | 0.04 |
| (C14) JUP | 3.638E+08 | (C1) PBX2 | 0.035 |
| (C15) NEUROD1 | 4.470E+08 | (C5) TRIM24 | 0.03 |
| (C16)CKMT2 | 1.397E+08 | (C11) NR0B2 | 0.025 |
| (C17) TSHZ2 | 3.037E+07 | (C18) MITF | 0.02 |
| (C18) MITF | 5.164E+08 | (C8) ZBTB39 | 0.005 |
| (C19) MYOCD | 5.331E+08 | (C19) MYOCD | 0 |
| (C20) PPARGC1B | 1.962E+08 | (C20) PPARGC1B | 0 |

Immunocytochemistry (ICC)

3 main cardiac markers with distinct structure that made up the sarcomeres: Alpha Myosin Heavy Chain (MYH6), Cardiac Troponin T (cTnT or TNNT2 gene), Alpha-actinin (ACTN2).
Cells were seeded to poly-D-lysine coated glass-bottomed chamber wells.
4% PFA as fixing agent.
0.1% of Triton X-100 in PBS as permeabilization agent.
10% goat serum as blocking agent.

The following is data for anti-MYH6 ICC

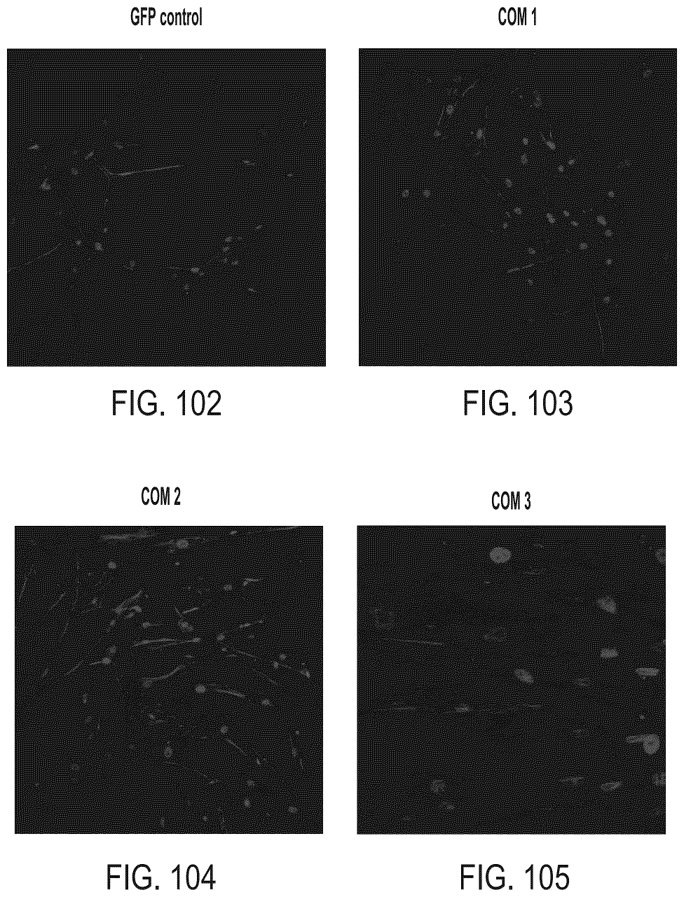

FIGS. 102-109 show results of immunocytochemistry studies of cells treated with indicated CFD combinations or GFP control. Table 9 indicates figure number for indicated treatment of cells.

TABLE 9
figure number for indicated treatment of cells.

| Treatment | FIG. |
|---|---|
| GFP control | 102 |
| COM1 C6 (GATA4), C3 (POU2F1), C4 (HAND1), | 103 |
| C12 (NACA2), C17 (TSHZ2) | |
| COM2 C6 (GATA4), C10 (IKZF4), C12 (NACA2), C17 (TSHZ2) | 104 |
| COM3 C6 (GATA4), C3 (POU2F1), C4 (HAND1), C9 (HAND2) | 105 |
| COM4 C6 (GATA4), C9 (HAND2), C10 (IKZF4) | 106 |
| COM6 C6 (GATA4), C4 (HAND1), C12 (NACA2), C10 (IKZF4) | 107 |
| COM7 C6 (GATA4), C4 (HAND1), C12 (NACA2) | 108 |
| COM8 C6 (GATA4), C3 (POU2F1), C4 (HAND1), C10 (IKZF4), | 109 |
| C12 (NACA2) | |

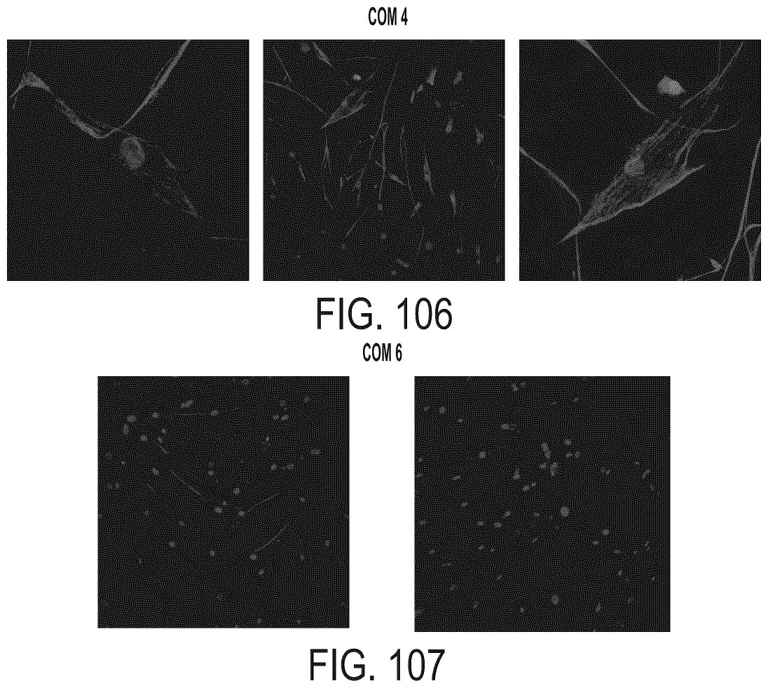

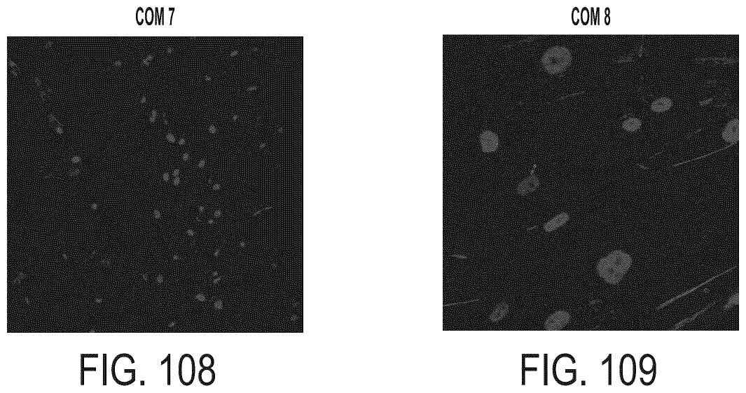

Cells transduced with COM1 (FIG. 103), COM2 (FIG. 104), COM3 (FIG. 105), COM4 (FIG. 106), COM6 (FIG. 107), COM7 (FIG. 108), and COM8 (FIG. 109) showed MYH6 expression fibrous patterns (red) similar to cardiomyocytes, when compared to MSCs expressing GFP alone (FIG. 102). Nuclei (blue).

SEQUENCES OF THE INVENTION

Note: V1 stands for transcript variant 1, I1 stands for isoform 1
PBX2
Transcript variant: NM_002586.5 (SEQ ID NO: 1)
ATGGACGAACGGCTACTGGGGCCGCCCCCTCCAGGCGGGGGCCGGGGGGGCCTGGGATTGGTGAGTGGGGAGC
CTGGGGGCCCTGGCGAGCCTCCCGGTGGCGGAGACCCCGGTGGGGGTAGCGGGGGGGTCCCGGGAGGCCGAG
GGAAGCAAGACATCGGGGACATTCTGCAGCAGATAATGACCATCACCGACCAGAGCCTGGACGAGGCCCAGGCC
AAGAAACACGCCCTAAACTGCCACCGAATGAAGCCTGCTCTCTTTAGCGTCCTGTGTGAAATCAAGGAGAAAACTG
GCCTCAGCATTCGGAGCTCCCAGGAGGAGGAGCCGGTGGACCCACAGCTGATGCGCTTGGACAACATGCTTCTGG
CAGAGGGTGTGGCTGGGCCCGAGAAAGGGGGGGGCTCAGCAGCAGCAGCTGCAGCCGCTGCAGCCTCTGGTGG
TGGTGTGTCCCCTGACAACTCCATCGAACACTCGGACTATCGCAGCAAACTTGCCCAGATCCGTCACATATACCACT
CGGAGCTGGAGAAGTATGAGCAGGCATGTAATGAGTTCACGACCCATGTCATGAACCTGCTGAGGGAGCAGAGC
CGCACCAGGCCCGTGGCCCCCAAAGAGATGGAACGCATGGTGAGCATCATCCATCGAAAGTTCAGCGCCATCCAG
ATGCAGCTGAAGCAGAGCACCTGCGAGGCTGTGATGATCCTGCGCTCCCGTTTCCTGGATGCCAGACGAAAGCGC
CGTAACTTCAGCAAACAGGCCACTGAGGTCCTAAATGAGTATTTCTACTCCCACCTGAGTAACCCATATCCTAGTGA
GGAGGCCAAGGAGGAGCTTGCCAAGAAGTGTGGCATCACCGTGTCTCAGGTCTCCAACTGGTTTGGCAACAAGA
GGATTCGCTATAAGAAAAACATCGGAAAGTTCCAAGAGGAGGCAAACATCTATGCTGTCAAGACCGCCGTGTCAG
TCACCCAGGGGGGCCACAGCCGCACCAGCTCCCCGACACCCCCTTCCTCTGCAGGCTCTGGCGGCTCTTTCAATCT
CTCAGGATCTGGAGACATGTTTCTGGGGATGCCTGGGCTCAACGGAGATTCCTATTCTGCTTCCCAGGTGGAATCA
CTCCGACACTCGATGGGGCCAGGGGGCTATGGGGATAACCTCGGGGGAGGCCAGATGTACAGCCCACGGGAAAT
GAGGGCAAATGGCAGCTGGCAAGAGGCTGTGACCCCCTCTTCAGTGACATCCCCAACGGAGGGACCAGGGAGTG
TTCACTCTGATACCTCCAACTGA
Protein variant: NP_002577.2 (SEQ ID NO: 2)
MDERLLGPPPPGGGRGGLGLVSGEPGGPGEPPGGGDPGGGSGGVPGGRGKQDIGDILQQIMTITDQSLDEAQAKKH
ALNCHRMKPALFSVLCEIKEKTGLSIRSSQEEEPVDPQLMRLDNMLLAEGVAGPEKGGGSAAAAAAAAASGGGVSPD
NSIEHSDYRSKLAQIRHIYHSELEKYEQACNEFTTHVMNLLREQSRTRPVAPKEMERMVSIIHRKFSAIQMQLKQSTCEA
VMILRSRFLDARRKRRNFSKQATEVLNEYFYSHLSNPYPSEEAKEELAKKCGITVSQVSNWFGNKRIRYKKNIGKFQEEA
NIYAVKTAVSVTQGGHSRTSSPTPPSSAGSGGSFNLSGSGDMFLGMPGLNGDSYSASQVESLRHSMGPGGYGDNLGG
GQMYSPREMRANGSWQEAVTPSSVTSPTEGPGSVHSDTSN
ACTN2
V1: NM_001103.4 (SEQ ID NO: 3)
ATGAACCAGATAGAGCCCGGCGTGCAGTACAACTACGTGTACGACGAGGATGAGTACATGATCCAGGAGGAGGA
GTGGGACCGCGACCTGCTCCTGGACCCAGCCTGGGAGAAGCAGCAGAGGAAGACCTTCACTGCCTGGTGTAACTC
CCACCTAAGGAAAGCCGGCACCCAGATTGAGAACATCGAGGAAGACTTCAGGAATGGCCTTAAGCTCATGCTGCT
TTTGGAAGTCATCTCAGGGGAAAGGCTGCCCAAACCTGACCGGGGAAAAATGCGGTTCCACAAAATTGCTAATGT
CAACAAAGCTTTGGATTACATAGCCAGCAAAGGGGTGAAACTGGTGTCCATTGGCGCTGAAGAAATTGTTGATGG
CAACGTGAAAATGACCCTGGGTATGATCTGGACCATCATCCTTCGCTTTGCTATTCAGGATATTTCGGTTGAAGAA
ACATCTGCCAAAGAAGGTCTGCTGCTTTGGTGTCAGAGGAAAACTGCTCCTTATAGAAATGTGAACATTCAGAACT
TCCATACTAGCTGGAAAGATGGCCTTGGACTCTGTGCCCTCATCCACCGACACCGGCCTGACCTCATTGACTACTCA
AAGCTTAACAAGGATGACCCCATAGGAAATATTAACCTGGCCATGGAAATCGCTGAGAAGCACCTGGATATTCCT
AAAATGTTGGATGCTGAAGACATCGTGAACACCCCTAAACCCGATGAAAGAGCCATCATGACGTACGTCTCTTGCT
TCTACCACGCTTTTGCGGGCGCGGAGCAGGCCGAGACAGCGGCTAACAGGATATGTAAGGTTCTTGCTGTGAATC
AAGAGAATGAGAGGCTGATGGAAGAATATGAGAGGCTAGCGAGTGAGCTTTTGGAATGGATTCGTCGCACGATC
CCCTGGCTGGAGAACCGGACTCCCGAGAAGACCATGCAAGCCATGCAGAAGAAGCTGGAGGACTTCCGGGATTA
CCGCCGGAAGCACAAGCCACCCAAGGTGCAGGAGAAATGCCAGCTGGAGATCAACTTCAACACGCTGCAGACCA
AGCTGCGGATCAGCAACCGTCCTGCCTTCATGCCCTCCGAGGGCAAGATGGTGTCGGATATTGCTGGTGCCTGGC
AGAGGCTGGAGCAGGCTGAGAAGGGTTACGAGGAGTGGTTGCTCAATGAGATTCGGAGACTGGAGCGCTTGGA
ACACCTGGCTGAGAAGTTCAGGCAGAAGGCCTCAACGCACGAGACTTGGGCTTATGGCAAAGAGCAGATCTTGCT
GCAGAAGGATTACGAGTCGGCGTCGCTGACAGAGGTGCGGGCTCTGCTGCGGAAGCACGAGGCGTTCGAGAGC
GACCTGGCAGCGCACCAGGACCGCGTGGAGCAGATCGCAGCCATCGCGCAGGAGCTCAATGAACTGGACTATCA
CGACGCTGTGAATGTCAATGATCGGTGCCAGAAAATTTGTGACCAGTGGGACCGACTGGGAACGCTTACTCAGAA
GAGGAGAGAAGCCCTAGAGAGAATGGAGAAATTGCTAGAAACCATTGATCAGCTTCACCTGGAGTTTGCCAAGA
GGGCTGCTCCTTTCAACAATTGGATGGAGGGCGCTATGGAGGATCTGCAAGATATGTTCATTGTCCACAGCATTGA
GGAGATCCAGAGTCTGATCACTGCGCATGAGCAGTTCAAGGCCACGCTGCCCGAGGCGGACGGAGAGCGGCAGT
CCATCATGGCCATCCAGAACGAGGTGGAGAAGGTGATTCAGAGCTACAACATCAGAATCAGCTCAAGCAACCCGT
ACAGCACTGTCACCATGGATGAGCTCCGGACCAAGTGGGACAAGGTGAAGCAACTCGTGCCCATCCGCGATCAAT
CCCTGCAGGAGGAGCTGGCTCGCCAGCATGCTAACGAGCGTCTGAGGCGCCAGTTTGCTGCCCAAGCCAATGCCA
TTGGGCCCTGGATCCAGAACAAGATGGAGGAGATTGCCCGGAGCTCCATCCAGATCACAGGAGCCCTGGAAGAC
CAGATGAACCAGCTGAAGCAGTATGAGCACAACATCATCAACTATAAGAACAACATCGACAAGCTGGAGGGAGA
CCATCAGCTCATCCAGGAGGCCCTTGTCTTTGACAACAAGCACACGAACTACACGATGGAGCACATTCGTGTTGGA
TGGGAGCTGCTGCTGACAACCATCGCCAGAACCATCAATGAGGTGGAGACTCAGATCCTGACGAGAGATGCGAA
GGGCATCACCCAGGAGCAGATGAATGAGTTCAGAGCCTCCTTCAACCACTTTGACAGGAGGAAGAATGGCCTGAT
GGATCATGAGGATTTCAGAGCCTGCCTGATTTCCATGGGTTATGACCTGGGTGAAGCCGAATTTGCCCGCATTATG
ACCCTGGTAGATCCCAACGGGCAAGGCACCGTCACCTTCCAATCCTTCATCGACTTCATGACTAGAGAGACGGCTG
ACACCGACACTGCCGAGCAGGTCATCGCCTCCTTCCGGATCCTGGCTTCTGATAAGCCATACATCCTGGCGGAGGA
GCTGCGTCGGGAGCTGCCCCCGGATCAGGCCCAGTACTGCATCAAGAGGATGCCCGCCTACTCGGGCCCAGGCA
GTGTGCCTGGTGCACTGGATTACGCTGCGTTCTCTTCCGCACTCTACGGGGAGAGCGATCTGTGA
I1: NP_001094.1 (SEQ ID NO: 4)
MNQIEPGVQYNYVYDEDEYMIQEEEWDRDLLLDPAWEKQQRKTFTAWCNSHLRKAGTQIENIEEDFRNGLKLMLLLE
VISGERLPKPDRGKMRFHKIANVNKALDYIASKGVKLVSIGAEEIVDGNVKMTLGMIWTIILRFAIQDISVEETSAKEGLLL
WCQRKTAPYRNVNIQNFHTSWKDGLGLCALIHRHRPDLIDYSKLNKDDPIGNINLAMEIAEKHLDIPKMLDAEDIVNTP
KPDERAIMTYVSCFYHAFAGAEQAETAANRICKVLAVNQENERLMEEYERLASELLEWIRRTIPWLENRTPEKTMQAM
QKKLEDFRDYRRKHKPPKVQEKCQLEINFNTLQTKLRISNRPAFMPSEGKMVSDIAGAWQRLEQAEKGYEEWLLNEIR
RLERLEHLAEKFRQKASTHETWAYGKEQILLQKDYESASLTEVRALLRKHEAFESDLAAHQDRVEQIAAIAQELNELDYH
DAVNVNDRCQKICDQWDRLGTLTQKRREALERMEKLLETIDQLHLEFAKRAAPFNNWMEGAMEDLQDMFIVHSIEEI
QSLITAHEQFKATLPEADGERQSIMAIQNEVEKVIQSYNIRISSSNPYSTVTMDELRTKWDKVKQLVPIRDQSLQEELAR
QHANERLRRQFAAQANAIGPWIQNKMEEIARSSIQITGALEDQMNQLKQYEHNIINYKNNIDKLEGDHQLIQEALVFD
NKHTNYTMEHIRVGWELLLTTIARTINEVETQILTRDAKGITQEQMNEFRASFNHFDRRKNGLMDHEDFRACLISMGY
DLGEAEFARIMTLVDPNGQGTVTFQSFIDFMTRETADTDTAEQVIASFRILASDKPYILAEELRRELPPDQAQYCIKRMP
AYSGPGSVPGALDYAAFSSALYGESDL
V2: NM_001278343.2 (SEQ ID NO: 5)
ATGAACCAGATAGAGCCCGGCGTGCAGTACAACTACGTGTACGACGAGGATGAGTACATGATCCAGGAGGAGGA
GTGGGACCGCGACCTGCTCCTGGACCCAGCCTGGGAGAAGCAGCAGAGGAAGACCTTCACTGCCTGGTGTAACTC
CCACCTAAGGAAAGCCGGCACCCAGATTGAGAACATCGAGGAAGACTTCAGGAATGGCCTTAAGCTCATGCTGCT
TTTGGAAGTCATCTCAGGGGAAAGGCTGCCCAAACCTGACCGGGGAAAAATGCGGTTCCACAAAATTGCTAATGT
CAACAAAGCTTTGGATTACATAGCCAGCAAAGGGGTGAAACTGGTGTCCATTGGCGCTGAAGAAATTGTTGATGG
CAACGTGAAAATGACCCTGGGTATGATCTGGACCATCATCCTTCGCTTTGCTATTCAGGATATTTCGGTTGAAGAA
ACATCTGCCAAAGAAGGTCTGCTGCTTTGGTGTCAGAGGAAAACTGCTCCTTATAGAAATGTGAACATTCAGAACT
TCCATACTAGCTGGAAAGATGGCCTTGGACTCTGTGCCCTCATCCACCGACACCGGCCTGACCTCATTGACTACTCA
AAGCTTAACAAGGATGACCCCATAGGAAATATTAACCTGGCCATGGAAATCGCTGAGAAGCACCTGGATATTCCT
AAAATGTTGGATGCTGAAGATTTAGTATACACTGCCAGACCCGATGAAAGAGCCATAATGACTTATGTTTCCTGTT
ACTATCATGCTTTTGCTGGTGCACAGAAGGCCGAGACAGCGGCTAACAGGATATGTAAGGTTCTTGCTGTGAATC
AAGAGAATGAGAGGCTGATGGAAGAATATGAGAGGCTAGCGAGTGAGCTTTTGGAATGGATTCGTCGCACGATC
CCCTGGCTGGAGAACCGGACTCCCGAGAAGACCATGCAAGCCATGCAGAAGAAGCTGGAGGACTTCCGGGATTA
CCGCCGGAAGCACAAGCCACCCAAGGTGCAGGAGAAATGCCAGCTGGAGATCAACTTCAACACGCTGCAGACCA
AGCTGCGGATCAGCAACCGTCCTGCCTTCATGCCCTCCGAGGGCAAGATGGTGTCGGATATTGCTGGTGCCTGGC
AGAGGCTGGAGCAGGCTGAGAAGGGTTACGAGGAGTGGTTGCTCAATGAGATTCGGAGACTGGAGCGCTTGGA
ACACCTGGCTGAGAAGTTCAGGCAGAAGGCCTCAACGCACGAGACTTGGGCTTATGGCAAAGAGCAGATCTTGCT
GCAGAAGGATTACGAGTCGGCGTCGCTGACAGAGGTGCGGGCTCTGCTGCGGAAGCACGAGGCGTTCGAGAGC
GACCTGGCAGCGCACCAGGACCGCGTGGAGCAGATCGCAGCCATCGCGCAGGAGCTCAATGAACTGGACTATCA
CGACGCTGTGAATGTCAATGATCGGTGCCAGAAAATTTGTGACCAGTGGGACCGACTGGGAACGCTTACTCAGAA
GAGGAGAGAAGCCCTAGAGAGAATGGAGAAATTGCTAGAAACCATTGATCAGCTTCACCTGGAGTTTGCCAAGA
GGGCTGCTCCTTTCAACAATTGGATGGAGGGCGCTATGGAGGATCTGCAAGATATGTTCATTGTCCACAGCATTGA
GGAGATCCAGAGTCTGATCACTGCGCATGAGCAGTTCAAGGCCACGCTGCCCGAGGCGGACGGAGAGCGGCAGT
CCATCATGGCCATCCAGAACGAGGTGGAGAAGGTGATTCAGAGCTACAACATCAGAATCAGCTCAAGCAACCCGT
ACAGCACTGTCACCATGGATGAGCTCCGGACCAAGTGGGACAAGGTGAAGCAACTCGTGCCCATCCGCGATCAAT
CCCTGCAGGAGGAGCTGGCTCGCCAGCATGCTAACGAGCGTCTGAGGCGCCAGTTTGCTGCCCAAGCCAATGCCA
TTGGGCCCTGGATCCAGAACAAGATGGAGGAGATTGCCCGGAGCTCCATCCAGATCACAGGAGCCCTGGAAGAC
CAGATGAACCAGCTGAAGCAGTATGAGCACAACATCATCAACTATAAGAACAACATCGACAAGCTGGAGGGAGA
CCATCAGCTCATCCAGGAGGCCCTTGTCTTTGACAACAAGCACACGAACTACACGATGGAGCACATTCGTGTTGGA
TGGGAGCTGCTGCTGACAACCATCGCCAGAACCATCAATGAGGTGGAGACTCAGATCCTGACGAGAGATGCGAA
GGGCATCACCCAGGAGCAGATGAATGAGTTCAGAGCCTCCTTCAACCACTTTGACAGGAGGAAGAATGGCCTGAT
GGATCATGAGGATTTCAGAGCCTGCCTGATTTCCATGGGTTATGACCTGGGTGAAGCCGAATTTGCCCGCATTATG
ACCCTGGTAGATCCCAACGGGCAAGGCACCGTCACCTTCCAATCCTTCATCGACTTCATGACTAGAGAGACGGCTG
ACACCGACACTGCCGAGCAGGTCATCGCCTCCTTCCGGATCCTGGCTTCTGATAAGCCATACATCCTGGCGGAGGA
GCTGCGTCGGGAGCTGCCCCCGGATCAGGCCCAGTACTGCATCAAGAGGATGCCCGCCTACTCGGGCCCAGGCA
GTGTGCCTGGTGCACTGGATTACGCTGCGTTCTCTTCCGCACTCTACGGGGAGAGCGATCTGTGA
I2: NP_001265272.1 (SEQ ID NO: 6)
MNQIEPGVQYNYVYDEDEYMIQEEEWDRDLLLDPAWEKQQRKTFTAWCNSHLRKAGTQIENIEEDFRNGLKLMLLLE
VISGERLPKPDRGKMRFHKIANVNKALDYIASKGVKLVSIGAEEIVDGNVKMTLGMIWTIILRFAIQDISVEETSAKEGLLL
WCQRKTAPYRNVNIQNFHTSWKDGLGLCALIHRHRPDLIDYSKLNKDDPIGNINLAMEIAEKHLDIPKMLDAEDLVYTA
RPDERAIMTYVSCYYHAFAGAQKAETAANRICKVLAVNQENERLMEEYERLASELLEWIRRTIPWLENRTPEKTMQAM
QKKLEDFRDYRRKHKPPKVQEKCQLEINFNTLQTKLRISNRPAFMPSEGKMVSDIAGAWQRLEQAEKGYEEWLLNEIR
RLERLEHLAEKFRQKASTHETWAYGKEQILLQKDYESASLTEVRALLRKHEAFESDLAAHQDRVEQIAAIAQELNELDYH
DAVNVNDRCQKICDQWDRLGTLTQKRREALERMEKLLETIDQLHLEFAKRAAPFNNWMEGAMEDLQDMFIVHSIEEI
QSLITAHEQFKATLPEADGERQSIMAIQNEVEKVIQSYNIRISSSNPYSTVTMDELRTKWDKVKQLVPIRDQSLQEELAR
QHANERLRRQFAAQANAIGPWIQNKMEEIARSSIQITGALEDQMNQLKQYEHNIINYKNNIDKLEGDHQLIQEALVFD
NKHTNYTMEHIRVGWELLLTTIARTINEVETQILTRDAKGITQEQMNEFRASFNHFDRRKNGLMDHEDFRACLISMGY
DLGEAEFARIMTLVDPNGQGTVTFQSFIDFMTRETADTDTAEQVIASFRILASDKPYILAEELRRELPPDQAQYCIKRMP
AYSGPGSVPGALDYAAFSSALYGESDL
V3: NM_001278344.2 (SEQ ID NO: 7)
ATGACGTACGTCTCTTGCTTCTACCACGCTTTTGCGGGCGCGGAGCAGGTTAGACAAAGTCTTAAAGCACACTCAG
CTCTGTGGAAGGATCCCCCTCCAGAAAGTTCTACATGTTCATATCAGGAGATGAGGAGGTCTTCAGTGAATTCAAG
TGCAATGGCCGAGACAGCGGCTAACAGGATATGTAAGGTTCTTGCTGTGAATCAAGAGAATGAGAGGCTGATGG
AAGAATATGAGAGGCTAGCGAGTGAGCTTTTGGAATGGATTCGTCGCACGATCCCCTGGCTGGAGAACCGGACTC
CCGAGAAGACCATGCAAGCCATGCAGAAGAAGCTGGAGGACTTCCGGGATTACCGCCGGAAGCACAAGCCACCC
AAGGTGCAGGAGAAATGCCAGCTGGAGATCAACTTCAACACGCTGCAGACCAAGCTGCGGATCAGCAACCGTCCT
GCCTTCATGCCCTCCGAGGGCAAGATGGTGTCGGATATTGCTGGTGCCTGGCAGAGGCTGGAGCAGGCTGAGAA
GGGTTACGAGGAGTGGTTGCTCAATGAGATTCGGAGACTGGAGCGCTTGGAACACCTGGCTGAGAAGTTCAGGC
AGAAGGCCTCAACGCACGAGACTTGGGCTTATGGCAAAGAGCAGATCTTGCTGCAGAAGGATTACGAGTCGGCG
TCGCTGACAGAGGTGCGGGCTCTGCTGCGGAAGCACGAGGCGTTCGAGAGCGACCTGGCAGCGCACCAGGACCG
CGTGGAGCAGATCGCAGCCATCGCGCAGGAGCTCAATGAACTGGACTATCACGACGCTGTGAATGTCAATGATCG
GTGCCAGAAAATTTGTGACCAGTGGGACCGACTGGGAACGCTTACTCAGAAGAGGAGAGAAGCCCTAGAGAGAA
TGGAGAAATTGCTAGAAACCATTGATCAGCTTCACCTGGAGTTTGCCAAGAGGGCTGCTCCTTTCAACAATTGGAT
GGAGGGCGCTATGGAGGATCTGCAAGATATGTTCATTGTCCACAGCATTGAGGAGATCCAGAGTCTGATCACTGC
GCATGAGCAGTTCAAGGCCACGCTGCCCGAGGCGGACGGAGAGCGGCAGTCCATCATGGCCATCCAGAACGAGG
TGGAGAAGGTGATTCAGAGCTACAACATCAGAATCAGCTCAAGCAACCCGTACAGCACTGTCACCATGGATGAGC
TCCGGACCAAGTGGGACAAGGTGAAGCAACTCGTGCCCATCCGCGATCAATCCCTGCAGGAGGAGCTGGCTCGCC
AGCATGCTAACGAGCGTCTGAGGCGCCAGTTTGCTGCCCAAGCCAATGCCATTGGGCCCTGGATCCAGAACAAGA
TGGAGGAGATTGCCCGGAGCTCCATCCAGATCACAGGAGCCCTGGAAGACCAGATGAACCAGCTGAAGCAGTAT
GAGCACAACATCATCAACTATAAGAACAACATCGACAAGCTGGAGGGAGACCATCAGCTCATCCAGGAGGCCCTT
GTCTTTGACAACAAGCACACGAACTACACGATGGAGCACATTCGTGTTGGATGGGAGCTGCTGCTGACAACCATC
GCCAGAACCATCAATGAGGTGGAGACTCAGATCCTGACGAGAGATGCGAAGGGCATCACCCAGGAGCAGATGAA
TGAGTTCAGAGCCTCCTTCAACCACTTTGACAGGAGGAAGAATGGCCTGATGGATCATGAGGATTTCAGAGCCTG
CCTGATTTCCATGGGTTATGACCTGGGTGAAGCCGAATTTGCCCGCATTATGACCCTGGTAGATCCCAACGGGCAA
GGCACCGTCACCTTCCAATCCTTCATCGACTTCATGACTAGAGAGACGGCTGACACCGACACTGCCGAGCAGGTCA
TCGCCTCCTTCCGGATCCTGGCTTCTGATAAGCCATACATCCTGGCGGAGGAGCTGCGTCGGGAGCTGCCCCCGGA
TCAGGCCCAGTACTGCATCAAGAGGATGCCCGCCTACTCGGGCCCAGGCAGTGTGCCTGGTGCACTGGATTACGC
TGCGTTCTCTTCCGCACTCTACGGGGAGAGCGATCTGTGA
I3: NP_001265273.1 (SEQ ID NO: 8)
MTYVSCFYHAFAGAEQVRQSLKAHSALWKDPPPESSTCSYQEMRRSSVNSSAMAETAANRICKVLAVNQENERLMEE
YERLASELLEWIRRTIPWLENRTPEKTMQAMQKKLEDFRDYRRKHKPPKVQEKCQLEINFNTLQTKLRISNRPAFMPSE
GKMVSDIAGAWQRLEQAEKGYEEWLLNEIRRLERLEHLAEKFRQKASTHETWAYGKEQILLQKDYESASLTEVRALLRK
HEAFESDLAAHQDRVEQIAAIAQELNELDYHDAVNVNDRCQKICDQWDRLGTLTQKRREALERMEKLLETIDQLHLEF
AKRAAPFNNWMEGAMEDLQDMFIVHSIEEIQSLITAHEQFKATLPEADGERQSIMAIQNEVEKVIQSYNIRISSSNPYST
VTMDELRTKWDKVKQLVPIRDQSLQEELARQHANERLRRQFAAQANAIGPWIQNKMEEIARSSIQITGALEDQMNQL
KQYEHNIINYKNNIDKLEGDHQLIQEALVFDNKHTNYTMEHIRVGWELLLTTIARTINEVETQILTRDAKGITQEQMNEF
RASFNHFDRRKNGLMDHEDFRACLISMGYDLGEAEFARIMTLVDPNGQGTVTFQSFIDFMTRETADTDTAEQVIASFR
ILASDKPYILAEELRRELPPDQAQYCIKRMPAYSGPGSVPGALDYAAFSSALYGESDL
POU2F1
V1: NM_002697.4 (SEQ ID NO: 9)
ATGGCGGACGGAGGAGCAGCGAGTCAAGATGAGAGTTCAGCCGCGGCGGCAGCAGCAGCAGACTCAAGAATGA
ACAATCCGTCAGAAACCAGTAAACCATCTATGGAGAGTGGAGATGGCAACACAGGCACACAAACCAATGGTCTGG
ACTTTCAGAAGCAGCCTGTGCCTGTAGGAGGAGCAATCTCAACAGCCCAGGCGCAGGCTTTCCTTGGACATCTCCA
TCAGGTCCAACTCGCTGGAACAAGTTTACAGGCTGCTGCTCAGTCTTTAAATGTACAGTCTAAATCTAATGAAGAA
TCGGGGGATTCGCAGCAGCCAAGCCAGCCTTCCCAGCAGCCTTCAGTGCAGGCAGCCATTCCCCAGACCCAGCTT
ATGCTAGCTGGAGGACAGATAACTGGGCTTACTTTGACGCCTGCCCAGCAACAGTTACTACTCCAGCAGGCACAG
GCACAGGCACAGCTGCTGGCTGCTGCAGTGCAGCAGCACTCCGCCAGCCAGCAGCACAGTGCTGCTGGAGCCACC
ATCTCCGCCTCTGCTGCCACGCCCATGACGCAGATCCCCCTGTCTCAGCCCATACAGATCGCACAGGATCTTCAACA
ACTGCAACAGCTTCAACAGCAGAATCTCAACCTGCAACAGTTTGTGTTGGTGCATCCAACCACCAATTTGCAGCCA
GCGCAGTTTATCATCTCACAGACGCCCCAGGGCCAGCAGGGTCTCCTGCAAGCGCAAAATCTTCTAACGCAACTAC
CTCAGCAAAGCCAAGCCAACCTCCTACAGTCGCAGCCAAGCATCACCCTCACCTCCCAGCCAGCAACCCCAACACG
CACAATAGCAGCAACCCCAATTCAGACACTTCCACAGAGCCAGTCAACACCAAAGCGAATTGATACTCCCAGCTTG
GAGGAGCCCAGTGACCTTGAGGAGCTTGAGCAGTTTGCCAAGACCTTCAAACAAAGACGAATCAAACTTGGATTC
ACTCAGGGTGATGTTGGGCTCGCTATGGGGAAACTATATGGAAATGACTTCAGCCAAACTACCATCTCTCGATTTG
AAGCCTTGAACCTCAGCTTTAAGAACATGTGCAAGTTGAAGCCACTTTTAGAGAAGTGGCTAAATGATGCAGAGA
ACCTCTCATCTGATTCGTCCCTCTCCAGCCCAAGTGCCCTGAATTCTCCAGGAATTGAGGGCTTGAGCCGTAGGAG
GAAGAAACGCACCAGCATAGAGACCAACATCCGTGTGGCCTTAGAGAAGAGTTTCTTGGAGAATCAAAAGCCTAC
CTCGGAAGAGATCACTATGATTGCTGATCAGCTCAATATGGAAAAAGAGGTGATTCGTGTTTGGTTCTGTAACCGC
CGCCAGAAAGAAAAAAGAATCAACCCACCAAGCAGTGGTGGGACCAGCAGCTCACCTATTAAAGCAATTTTCCCC
AGCCCAACTTCACTGGTGGCGACCACACCAAGCCTTGTGACTAGCAGTGCAGCAACTACCCTCACAGTCAGCCCTG
TCCTCCCTCTGACCAGTGCTGCTGTGACGAATCTTTCAGTTACAGGCACTTCAGACACCACCTCCAACAACACAGCA
ACCGTGATTTCCACAGCGCCTCCAGCTTCCTCAGCAGTCACGTCCCCCTCTCTGAGTCCCTCCCCTTCTGCCTCAGCC
TCCACCTCCGAGGCATCCAGTGCCAGTGAGACCAGCACAACACAGACCACCTCCACTCCTTTGTCCTCCCCTCTTGG
GACCAGCCAGGTGATGGTGACAGCATCAGGTTTGCAAACAGCAGCAGCTGCTGCCCTTCAAGGAGCTGCACAGTT
GCCAGCAAATGCCAGTCTTGCTGCCATGGCAGCTGCTGCAGGACTAAACCCAAGCCTGATGGCACCCTCACAGTTT
GCGGCTGGAGGTGCCTTACTCAGTCTGAATCCAGGGACCCTGAGCGGTGCTCTCAGCCCAGCTCTAATGAGCAAC
AGTACACTGGCAACTATTCAAGCTCTTGCTTCTGGTGGCTCTCTTCCAATAACATCACTTGATGCAACTGGGAACCT
GGTATTTGCCAATGCGGGAGGAGCCCCCAACATCGTGACTGCCCCTCTGTTCCTGAACCCTCAGAACCTCTCTCTGC
TCACCAGCAACCCTGTTAGCTTGGTCTCTGCCGCCGCAGCATCTGCAGGGAACTCTGCACCTGTAGCCAGCCTTCA
CGCCACCTCCACCTCTGCTGAGTCCATCCAGAACTCTCTCTTCACAGTGGCCTCTGCCAGCGGGGCTGCGTCCACCA
CCACCACCGCCTCCAAGGCACAGTGA
I1: NP_002688.3 (SEQ ID NO: 10)
MADGGAASQDESSAAAAAAADSRMNNPSETSKPSMESGDGNTGTQTNGLDFQKQPVPVGGAISTAQAQAFLGHLH
QVQLAGTSLQAAAQSLNVQSKSNEESGDSQQPSQPSQQPSVQAAIPQTQLMLAGGQITGLTLTPAQQQLLLQQAQA
QAQLLAAAVQQHSASQQHSAAGATISASAATPMTQIPLSQPIQIAQDLQQLQQLQQQNLNLQQFVLVHPTTNLQPA
QFIISQTPQGQQGLLQAQNLLTQLPQQSQANLLQSQPSITLTSQPATPTRTIAATPIQTLPQSQSTPKRIDTPSLEEPSDLE
ELEQFAKTFKQRRIKLGFTQGDVGLAMGKLYGNDFSQTTISRFEALNLSFKNMCKLKPLLEKWLNDAENLSSDSSLSSPS
ALNSPGIEGLSRRRKKRTSIETNIRVALEKSFLENQKPTSEEITMIADQLNMEKEVIRVWFCNRRQKEKRINPPSSGGTSSS
PIKAIFPSPTSLVATTPSLVTSSAATTLTVSPVLPLTSAAVTNLSVTGTSDTTSNNTATVISTAPPASSAVTSPSLSPSPSASAS
TSEASSASETSTTQTTSTPLSSPLGTSQVMVTASGLQTAAAAALQGAAQLPANASLAAMAAAAGLNPSLMAPSQFAA
GGALLSLNPGTLSGALSPALMSNSTLATIQALASGGSLPITSLDATGNLVFANAGGAPNIVTAPLFLNPQNLSLLTSNPVS
LVSAAAASAGNSAPVASLHATSTSAESIQNSLFTVASASGAASTTTTASKAQ
V2: NM_001198783.2 (SEQ ID NO: 11)
ATGCTGGACTGCAGTGACTATGTTCTAGACTCAAGAATGAACAATCCGTCAGAAACCAGTAAACCATCTATGGAGA
GTGGAGATGGCAACACAGGCACACAAACCAATGGTCTGGACTTTCAGAAGCAGCCTGTGCCTGTAGGAGGAGCA
ATCTCAACAGCCCAGGCGCAGGCTTTCCTTGGACATCTCCATCAGGTCCAACTCGCTGGAACAAGTTTACAGGCTG
CTGCTCAGTCTTTAAATGTACAGTCTAAATCTAATGAAGAATCGGGGGATTCGCAGCAGCCAAGCCAGCCTTCCCA
GCAGCCTTCAGTGCAGGCAGCCATTCCCCAGACCCAGCTTATGCTAGCTGGAGGACAGATAACTGGGCTTACTTTG
ACGCCTGCCCAGCAACAGTTACTACTCCAGCAGGCACAGGCACAGGCACAGCTGCTGGCTGCTGCAGTGCAGCAG
CACTCCGCCAGCCAGCAGCACAGTGCTGCTGGAGCCACCATCTCCGCCTCTGCTGCCACGCCCATGACGCAGATCC
CCCTGTCTCAGCCCATACAGATCGCACAGGATCTTCAACAACTGCAACAGCTTCAACAGCAGAATCTCAACCTGCA
ACAGTTTGTGTTGGTGCATCCAACCACCAATTTGCAGCCAGCGCAGTTTATCATCTCACAGACGCCCCAGGGCCAG
CAGGGTCTCCTGCAAGCGCAAAATCTTCTAACGCAACTACCTCAGCAAAGCCAAGCCAACCTCCTACAGTCGCAGC
CAAGCATCACCCTCACCTCCCAGCCAGCAACCCCAACACGCACAATAGCAGCAACCCCAATTCAGACACTTCCACA
GAGCCAGTCAACACCAAAGCGAATTGATACTCCCAGCTTGGAGGAGCCCAGTGACCTTGAGGAGCTTGAGCAGTT
TGCCAAGACCTTCAAACAAAGACGAATCAAACTTGGATTCACTCAGGGTGATGTTGGGCTCGCTATGGGGAAACT
ATATGGAAATGACTTCAGCCAAACTACCATCTCTCGATTTGAAGCCTTGAACCTCAGCTTTAAGAACATGTGCAAGT
TGAAGCCACTTTTAGAGAAGTGGCTAAATGATGCAGAGAACCTCTCATCTGATTCGTCCCTCTCCAGCCCAAGTGC
CCTGAATTCTCCAGGAATTGAGGGCTTGAGCCGTAGGAGGAAGAAACGCACCAGCATAGAGACCAACATCCGTGT
GGCCTTAGAGAAGAGTTTCTTGGAGAATCAAAAGCCTACCTCGGAAGAGATCACTATGATTGCTGATCAGCTCAAT
ATGGAAAAAGAGGTGATTCGTGTTTGGTTCTGTAACCGCCGCCAGAAAGAAAAAAGAATCAACCCACCAAGCAGT
GGTGGGACCAGCAGCTCACCTATTAAAGCAATTTTCCCCAGCCCAACTTCACTGGTGGCGACCACACCAAGCCTTG
TGACTAGCAGTGCAGCAACTACCCTCACAGTCAGCCCTGTCCTCCCTCTGACCAGTGCTGCTGTGACGAATCTTTCA
GTTACAGGCACTTCAGACACCACCTCCAACAACACAGCAACCGTGATTTCCACAGCGCCTCCAGCTTCCTCAGCAGT
CACGTCCCCCTCTCTGAGTCCCTCCCCTTCTGCCTCAGCCTCCACCTCCGAGGCATCCAGTGCCAGTGAGACCAGCA
CAACACAGACCACCTCCACTCCTTTGTCCTCCCCTCTTGGGACCAGCCAGGTGATGGTGACAGCATCAGGTTTGCA
AACAGCAGCAGCTGCTGCCCTTCAAGGAGCTGCACAGTTGCCAGCAAATGCCAGTCTTGCTGCCATGGCAGCTGC
TGCAGGACTAAACCCAAGCCTGATGGCACCCTCACAGTTTGCGGCTGGAGGTGCCTTACTCAGTCTGAATCCAGG
GACCCTGAGCGGTGCTCTCAGCCCAGCTCTAATGAGCAACAGTACACTGGCAACTATTCAAGCTCTTGCTTCTGGT
GGCTCTCTTCCAATAACATCACTTGATGCAACTGGGAACCTGGTATTTGCCAATGCGGGAGGAGCCCCCAACATCG
TGACTGCCCCTCTGTTCCTGAACCCTCAGAACCTCTCTCTGCTCACCAGCAACCCTGTTAGCTTGGTCTCTGCCGCCG
CAGCATCTGCAGGGAACTCTGCACCTGTAGCCAGCCTTCACGCCACCTCCACCTCTGCTGAGTCCATCCAGAACTCT
CTCTTCACAGTGGCCTCTGCCAGCGGGGCTGCGTCCACCACCACCACCGCCTCCAAGGCACAGTGA
I2: NP_001185712.1 (SEQ ID NO: 12)
MLDCSDYVLDSRMNNPSETSKPSMESGDGNTGTQTNGLDFQKQPVPVGGAISTAQAQAFLGHLHQVQLAGTSLQA
AAQSLNVQSKSNEESGDSQQPSQPSQQPSVQAAIPQTQLMLAGGQITGLTLTPAQQQLLLQQAQAQAQLLAAAVQQ
HSASQQHSAAGATISASAATPMTQIPLSQPIQIAQDLQQLQQLQQQNLNLQQFVLVHPTTNLQPAQFIISQTPQGQQ
GLLQAQNLLTQLPQQSQANLLQSQPSITLTSQPATPTRTIAATPIQTLPQSQSTPKRIDTPSLEEPSDLEELEQFAKTFKQR
RIKLGFTQGDVGLAMGKLYGNDFSQTTISRFEALNLSFKNMCKLKPLLEKWLNDAENLSSDSSLSSPSALNSPGIEGLSRR
RKKRTSIETNIRVALEKSFLENQKPTSEEITMIADQLNMEKEVIRVWFCNRRQKEKRINPPSSGGTSSSPIKAIFPSPTSLVA
TTPSLVTSSAATTLTVSPVLPLTSAAVTNLSVTGTSDTTSNNTATVISTAPPASSAVTSPSLSPSPSASASTSEASSASETSTT
QTTSTPLSSPLGTSQVMVTASGLQTAAAAALQGAAQLPANASLAAMAAAAGLNPSLMAPSQFAAGGALLSLNPGTLS
GALSPALMSNSTLATIQALASGGSLPITSLDATGNLVFANAGGAPNIVTAPLFLNPQNLSLLTSNPVSLVSAAAASAGNS
APVASLHATSTSAESIQNSLFTVASASGAASTTTTASKAQ
V3: NM_001198786.2 (SEQ ID NO: 13)
ATGGCGGACGGAGGAGCAGCGAGTCAAGATGAGAGTTCAGCCGCGGCGGCAGCAGCAGCAGACTCAAGAATGA
ACAATCCGTCAGAAACCAGTAAACCATCTATGGAGAGTGGAGATGGCAACACAGGCACACAAACCAATGGTCTGG
ACTTTCAGAAGCAGCCTGTGCCTGTAGGAGGAGCAATCTCAACAGCCCAGGCGCAGGCTTTCCTTGGACATCTCCA
TCAGGTCCAACTCGCTGGAACAAGTTTACAGGCTGCTGCTCAGTCTTTAAATGTACAGTCTAAATCTAATGAAGAA
TCGGGGGATTCGCAGCAGCCAAGCCAGCCTTCCCAGCAGCCTTCAGTGCAGGCAGCCATTCCCCAGACCCAGCTT
ATGCTAGCTGGAGGACAGATAACTGGGGATCTTCAACAACTGCAACAGCTTCAACAGCAGAATCTCAACCTGCAA
CAGTTTGTGTTGGTGCATCCAACCACCAATTTGCAGCCAGCGCAGTTTATCATCTCACAGACGCCCCAGGGCCAGC
AGGGTCTCCTGCAAGCGCAAAATCTTCTAACGCAACTACCTCAGCAAAGCCAAGCCAACCTCCTACAGTCGCAGCC
AAGCATCACCCTCACCTCCCAGCCAGCAACCCCAACACGCACAATAGCAGCAACCCCAATTCAGACACTTCCACAG
AGCCAGTCAACACCAAAGCGAATTGATACTCCCAGCTTGGAGGAGCCCAGTGACCTTGAGGAGCTTGAGCAGTTT
GCCAAGACCTTCAAACAAAGACGAATCAAACTTGGATTCACTCAGGGTGATGTTGGGCTCGCTATGGGGAAACTA
TATGGAAATGACTTCAGCCAAACTACCATCTCTCGATTTGAAGCCTTGAACCTCAGCTTTAAGAACATGTGCAAGTT
GAAGCCACTTTTAGAGAAGTGGCTAAATGATGCAGAGAACCTCTCATCTGATTCGTCCCTCTCCAGCCCAAGTGCC
CTGAATTCTCCAGGAATTGAGGGCTTGAGCCGTAGGAGGAAGAAACGCACCAGCATAGAGACCAACATCCGTGT
GGCCTTAGAGAAGAGTTTCTTGGAGAATCAAAAGCCTACCTCGGAAGAGATCACTATGATTGCTGATCAGCTCAAT
ATGGAAAAAGAGGTGATTCGTGTTTGGTTCTGTAACCGCCGCCAGAAAGAAAAAAGAATCAACCCACCAAGCAGT
GGTGGGACCAGCAGCTCACCTATTAAAGCAATTTTCCCCAGCCCAACTTCACTGGTGGCGACCACACCAAGCCTTG
TGACTAGCAGTGCAGCAACTACCCTCACAGTCAGCCCTGTCCTCCCTCTGACCAGTGCTGCTGTGACGAATCTTTCA
GTTACAGGCACTTCAGACACCACCTCCAACAACACAGCAACCGTGATTTCCACAGCGCCTCCAGCTTCCTCAGCAGT
CACGTCCCCCTCTCTGAGTCCCTCCCCTTCTGCCTCAGCCTCCACCTCCGAGGCATCCAGTGCCAGTGAGACCAGCA
CAACACAGACCACCTCCACTCCTTTGTCCTCCCCTCTTGGGACCAGCCAGGTGATGGTGACAGCATCAGGTTTGCA
AACAGCAGCAGCTGCTGCCCTTCAAGGAGCTGCACAGTTGCCAGCAAATGCCAGTCTTGCTGCCATGGCAGCTGC
TGCAGGACTAAACCCAAGCCTGATGGCACCCTCACAGTTTGCGGCTGGAGGTGCCTTACTCAGTCTGAATCCAGG
GACCCTGAGCGGTGCTCTCAGCCCAGCTCTAATGAGCAACAGTACACTGGCAACTATTCAAGCTCTTGCTTCTGGT
GGCTCTCTTCCAATAACATCACTTGATGCAACTGGGAACCTGGTATTTGCCAATGCGGGAGGAGCCCCCAACATCG
TGACTGCCCCTCTGTTCCTGAACCCTCAGAACCTCTCTCTGCTCACCAGCAACCCTGTTAGCTTGGTCTCTGCCGCCG
CAGCATCTGCAGGGAACTCTGCACCTGTAGCCAGCCTTCACGCCACCTCCACCTCTGCTGAGTCCATCCAGAACTCT
CTCTTCACAGTGGCCTCTGCCAGCGGGGCTGCGTCCACCACCACCACCGCCTCCAAGGCACAGTGA
I3: NP_001185715.1 (SEQ ID NO: 14)
MADGGAASQDESSAAAAAAADSRMNNPSETSKPSMESGDGNTGTQTNGLDFQKQPVPVGGAISTAQAQAFLGHLH
QVQLAGTSLQAAAQSLNVQSKSNEESGDSQQPSQPSQQPSVQAAIPQTQLMLAGGQITGDLQQLQQLQQQNLNLQ
QFVLVHPTTNLQPAQFIISQTPQGQQGLLQAQNLLTQLPQQSQANLLQSQPSITLTSQPATPTRTIAATPIQTLPQSQST
PKRIDTPSLEEPSDLEELEQFAKTFKQRRIKLGFTQGDVGLAMGKLYGNDFSQTTISRFEALNLSFKNMCKLKPLLEKWLN
DAENLSSDSSLSSPSALNSPGIEGLSRRRKKRTSIETNIRVALEKSFLENQKPTSEEITMIADQLNMEKEVIRVWFCNRRQK
EKRINPPSSGGTSSSPIKAIFPSPTSLVATTPSLVTSSAATTLTVSPVLPLTSAAVTNLSVTGTSDTTSNNTATVISTAPPASS
AVTSPSLSPSPSASASTSEASSASETSTTQTTSTPLSSPLGTSQVMVTASGLQTAAAAALQGAAQLPANASLAAMAAAA
GLNPSLMAPSQFAAGGALLSLNPGTLSGALSPALMSNSTLATIQALASGGSLPITSLDATGNLVFANAGGAPNIVTAPLF
LNPQNLSLLTSNPVSLVSAAAASAGNSAPVASLHATSTSAESIQNSLFTVASASGAASTTTTASKAQ
V6: NM_001365849.1 and V5: NM_001365848.1 have identical CDS (SEQ ID NO: 15)
ATGAAGACAAGGATGAAGATCTTTGTGATGATCCACTTCCACTTAATGAATAGCACACAAACCAATGGTCTGGACT
TTCAGAAGCAGCCTGTGCCTGTAGGAGGAGCAATCTCAACAGCCCAGGCGCAGGCTTTCCTTGGACATCTCCATCA
GGTCCAACTCGCTGGAACAAGTTTACAGGCTGCTGCTCAGTCTTTAAATGTACAGTCTAAATCTAATGAAGAATCG
GGGGATTCGCAGCAGCCAAGCCAGCCTTCCCAGCAGCCTTCAGTGCAGGCAGCCATTCCCCAGACCCAGCTTATG
CTAGCTGGAGGACAGATAACTGGGCTTACTTTGACGCCTGCCCAGCAACAGTTACTACTCCAGCAGGCACAGGCA
CAGGCACAGCTGCTGGCTGCTGCAGTGCAGCAGCACTCCGCCAGCCAGCAGCACAGTGCTGCTGGAGCCACCATC
TCCGCCTCTGCTGCCACGCCCATGACGCAGATCCCCCTGTCTCAGCCCATACAGATCGCACAGGATCTTCAACAACT
GCAACAGCTTCAACAGCAGAATCTCAACCTGCAACAGTTTGTGTTGGTGCATCCAACCACCAATTTGCAGCCAGCG
CAGTTTATCATCTCACAGACGCCCCAGGGCCAGCAGGGTCTCCTGCAAGCGCAAAATCTTCTAACGCAACTACCTC
AGCAAAGCCAAGCCAACCTCCTACAGTCGCAGCCAAGCATCACCCTCACCTCCCAGCCAGCAACCCCAACACGCAC
AATAGCAGCAACCCCAATTCAGACACTTCCACAGAGCCAGTCAACACCAAAGCGAATTGATACTCCCAGCTTGGAG
GAGCCCAGTGACCTTGAGGAGCTTGAGCAGTTTGCCAAGACCTTCAAACAAAGACGAATCAAACTTGGATTCACTC
AGGGTGATGTTGGGCTCGCTATGGGGAAACTATATGGAAATGACTTCAGCCAAACTACCATCTCTCGATTTGAAGC
CTTGAACCTCAGCTTTAAGAACATGTGCAAGTTGAAGCCACTTTTAGAGAAGTGGCTAAATGATGCAGAGAACCTC
TCATCTGATTCGTCCCTCTCCAGCCCAAGTGCCCTGAATTCTCCAGGAATTGAGGGCTTGAGCCGTAGGAGGAAGA
AACGCACCAGCATAGAGACCAACATCCGTGTGGCCTTAGAGAAGAGTTTCTTGGAGAATCAAAAGCCTACCTCGG
AAGAGATCACTATGATTGCTGATCAGCTCAATATGGAAAAAGAGGTGATTCGTGTTTGGTTCTGTAACCGCCGCCA
GAAAGAAAAAAGAATCAACCCACCAAGCAGTGGTGGGACCAGCAGCTCACCTATTAAAGCAATTTTCCCCAGCCC
AACTTCACTGGTGGCGACCACACCAAGCCTTGTGACTAGCAGTGCAGCAACTACCCTCACAGTCAGCCCTGTCCTC
CCTCTGACCAGTGCTGCTGTGACGAATCTTTCAGTTACAGGCACTTCAGACACCACCTCCAACAACACAGCAACCGT
GATTTCCACAGCGCCTCCAGCTTCCTCAGCAGTCACGTCCCCCTCTCTGAGTCCCTCCCCTTCTGCCTCAGCCTCCAC
CTCCGAGGCATCCAGTGCCAGTGAGACCAGCACAACACAGACCACCTCCACTCCTTTGTCCTCCCCTCTTGGGACC
AGCCAGGTGATGGTGACAGCATCAGGTTTGCAAACAGCAGCAGCTGCTGCCCTTCAAGGAGCTGCACAGTTGCCA
GCAAATGCCAGTCTTGCTGCCATGGCAGCTGCTGCAGGACTAAACCCAAGCCTGATGGCACCCTCACAGTTTGCG
GCTGGAGGTGCCTTACTCAGTCTGAATCCAGGGACCCTGAGCGGTGCTCTCAGCCCAGCTCTAATGAGCAACAGT
ACACTGGCAACTATTCAAGCTCTTGCTTCTGGTGGCTCTCTTCCAATAACATCACTTGATGCAACTGGGAACCTGGT
ATTTGCCAATGCGGGAGGAGCCCCCAACATCGTGACTGCCCCTCTGTTCCTGAACCCTCAGAACCTCTCTCTGCTCA
CCAGCAACCCTGTTAGCTTGGTCTCTGCCGCCGCAGCATCTGCAGGGAACTCTGCACCTGTAGCCAGCCTTCACGC
CACCTCCACCTCTGCTGAGTCCATCCAGAACTCTCTCTTCACAGTGGCCTCTGCCAGCGGGGCTGCGTCCACCACCA
CCACCGCCTCCAAGGCACAGTGA
I4: NP_001352778.1 and NP_001352777.1 (SEQ ID NO: 16) (both V6 and V5 encode I4)
MKTRMKIFVMIHFHLMNSTQTNGLDFQKQPVPVGGAISTAQAQAFLGHLHQVQLAGTSLQAAAQSLNVQSKSNEES
GDSQQPSQPSQQPSVQAAIPQTQLMLAGGQITGLTLTPAQQQLLLQQAQAQAQLLAAAVQQHSAQQHSAAGATIS
ASAATPMTQIPLSQPIQIAQDLQQLQQLQQQNLNLQQFVLVHPTTNLQPAQFIISQTPQGQQGLLQAQNLLTQLPQQ
SQANLLQSQPSITLTSQPATPTRTIAATPIQTLPQSQSTPKRIDTPSLEEPSDLEELEQFAKTFKQRRIKLGFTQGDVGLAM
GKLYGNDFSQTTISRFEALNLSFKNMCKLKPLLEKWLNDAENLSSDSSLSSPSALNSPGIEGLSRRRKKRTSIETNIRVALEK
SFLENQKPTSEEITMIADQLNMEKEVIRVWFCNRRQKEKRINPPSSGGTSSSPIKAIFPSPTSLVATTPSLVTSSAATTLTVS
PVLPLTSAAVTNLSVTGTSDTTSNNTATVISTAPPASSAVTSPSLSPSPSASASTSEASSASETSTTQTTSTPLSSPLGTSQV
MVTASGLQTAAAAALQGAAQLPANASLAAMAAAAGLNPSLMAPSQFAAGGALLSLNPGTLSGALSPALMSNSTLATI
QALASGGSLPITSLDATGNLVFANAGGAPNIVTAPLFLNPQNLSLLTSNPVSLVSAAAASAGNSAPVASLHATSTSAESIQ
NSLFTVASASGAASTTTTASKAQ
HAND1
NM_004821.3 (SEQ ID NO: 17)
ATGAACCTCGTGGGCAGCTACGCACACCATCACCACCATCACCACCCGCACCCTGCGCACCCCATGCTCCACGAAC
CCTTCCTCTTCGGTCCGGCCTCGCGCTGTCATCAGGAAAGGCCCTACTTCCAGAGCTGGCTGCTGAGCCCGGCTGA
CGCTGCCCCGGACTTCCCTGCGGGCGGGCCGCCGCCCGCGGCCGCTGCAGCCGCCACCGCCTATGGTCCTGACGC
CAGGCCTGGGCAGAGCCCCGGGCGGCTGGAGGCGCTTGGCGGCCGTCTTGGCCGGCGGAAAGGCTCAGGACCC
AAGAAGGAGCGGAGACGCACTGAGAGCATTAACAGCGCATTCGCGGAGTTGCGCGAGTGCATCCCCAACGTGCC
GGCCGACACCAAGCTCTCCAAGATCAAGACTCTGCGCCTAGCCACCAGCTACATCGCCTACCTGATGGACGTGCTG
GCCAAGGATGCACAGTCTGGCGATCCCGAGGCCTTCAAGGCTGAACTCAAGAAGGCGGATGGCGGCCGTGAGAG
CAAGCGGAAAAGGGAGCTGCAGCAGCACGAAGGTTTTCCTCCTGCCCTGGGCCCAGTCGAGAAGAGGATTAAAG
GACGCACCGGCTGGCCGCAGCAAGTCTGGGCGCTGGAGTTAAACCAGTGA
NP_004812.1 (SEQ ID NO: 18)
MNLVGSYAHHHHHHHPHPAHPMLHEPFLFGPASRCHQERPYFQSWLLSPADAAPDFPAGGPPPAAAAAATAYGPD
ARPGQSPGRLEALGGRLGRRKGSGPKKERRRTESINSAFAELRECIPNVPADTKLSKIKTLRLATSYIAYLMDVLAKDAQS
GDPEAFKAELKKADGGRESKRKRELQQHEGFPPALGPVEKRIKGRTGWPQQVWALELNQ
XM_005268531.2 (SEQ ID NO: 19)
ATGAACCTCGTGGGCAGCTACGCACACCATCACCACCATCACCACCCGCACCCTGCGCACCCCATGCTCCACGAAC
CCTTCCTCTTCGGTCCGGCCTCGCGCTGTCATCAGGAAAGGCCCTACTTCCAGAGCTGGCTGCTGAGCCCGGCTGA
CGCTGCCCCGGACTTCCCTGCGGGCGGGCCGCCGCCCGCGGCCGCTGCAGCCGCCACCGCCTATGGTCCTGACGC
CAGGCCTGGGCAGAGCCCCGGGCGGCTGGAGGCGCTTGGCGGCCGTCTTGGCCGGCGGAAAGGCTCAGGACCC
AAGAAGGAGCGGAGACGCACTGAGAGCATTAACAGCGCATTCGCGGAGTTGCGCGAGTGCATCCCCAACGTGCC
GGCCGACACCAAGCTCTCCAAGATCAAGACTCTGCGCCTAGCCACCAGCTACATCGCCTACCTGATGGACGTGCTG
GCCAAGGATGCACAGTCTGGCGATCCCGAGGCCTTCAAGGCTGAACTCAAGAAGGCGGATGGCGGCCGTGAGAG
CAAGCGGAAAAGGGAGCTGCAGCACGAAGGTTTTCCTCCTGCCCTGGGCCCAGTCGAGAAGAGGATTAAAGGAC
GCACCGGCTGGCCGCAGCAAGTCTGGGCGCTGGAGTTAAACCAGTGA
XP_005268588.1 (SEQ ID NO: 20)
MNLVGSYAHHHHHHHPHPAHPMLHEPFLFGPASRCHQERPYFQSWLLSPADAAPDFPAGGPPPAAAAAATAYGPD
ARPGQSPGRLEALGGRLGRRKGSGPKKERRRTESINSAFAELRECIPNVPADTKLSKIKTLRLATSYIAYLMDVLAKDAQS
GDPEAFKAELKKADGGRESKRKRELQHEGFPPALGPVEKRIKGRTGWPQQVWALELNQ
TRIM24
V2: NM_003852.4 (SEQ ID NO: 21)
ATGGAGGTGGCGGTGGAGAAGGCGGTGGCGGCGGCGGCAGCGGCCTCGGCTGCGGCCTCCGGGGGGCCCTCG
GCGGCGCCGAGCGGGGAGAACGAGGCCGAGAGTCGGCAGGGCCCGGACTCGGAGCGCGGCGGCGAGGCGGCC
CGGCTCAACCTGTTGGACACTTGCGCCGTGTGCCACCAGAACATCCAGAGCCGGGCGCCCAAGCTGCTGCCCTGC
CTGCACTCTTTCTGCCAGCGCTGCCTGCCCGCGCCCCAGCGCTACCTCATGCTGCCCGCGCCCATGCTGGGCTCGG
CCGAGACCCCGCCACCCGTCCCTGCCCCCGGCTCGCCGGTCAGCGGCTCGTCGCCGTTCGCCACCCAAGTTGGAGT
CATTCGTTGCCCAGTTTGCAGCCAAGAATGTGCAGAGAGACACATCATAGATAACTTTTTTGTGAAGGACACTACT
GAGGTTCCCAGCAGTACAGTAGAAAAGTCAAATCAGGTATGTACAAGCTGTGAGGACAACGCAGAAGCCAATGG
GTTTTGTGTAGAGTGTGTTGAATGGCTCTGCAAGACGTGTATCAGAGCTCATCAGAGGGTAAAGTTCACAAAAGA
CCACACTGTCAGACAGAAAGAGGAAGTATCTCCAGAGGCAGTTGGTGTCACCAGCCAGCGACCAGTGTTTTGTCC
TTTTCATAAAAAGGAGCAGCTGAAGCTGTACTGTGAGACATGTGACAAACTGACATGTCGAGACTGTCAGTTGTTA
GAACATAAAGAGCATAGATACCAATTTATAGAAGAAGCTTTTCAGAATCAGAAAGTGATCATAGATACACTAATCA
CCAAACTGATGGAAAAAACAAAATACATAAAATTCACAGGAAATCAGATCCAAAACAGAATTATTGAAGTAAATC
AAAATCAAAAGCAGGTGGAACAGGATATTAAAGTTGCTATATTTACACTGATGGTAGAAATAAATAAAAAAGGAA
AAGCTCTACTGCATCAGTTAGAGAGCCTTGCAAAGGACCATCGCATGAAACTTATGCAACAACAACAGGAAGTGG
CTGGACTCTCTAAACAATTGGAGCATGTCATGCATTTTTCTAAATGGGCAGTTTCCAGTGGCAGCAGTACAGCATT
ACTTTATAGCAAACGACTGATTACATACCGGTTACGGCACCTCCTTCGTGCAAGGTGTGATGCATCCCCAGTGACC
AACAACACCATCCAATTTCACTGTGATCCTAGTTTCTGGGCTCAAAATATCATCAACTTAGGTTCTTTAGTAATCGA
GGATAAAGAGAGCCAGCCACAAATGCCTAAGCAGAATCCTGTCGTGGAACAGAATTCACAGCCACCAAGTGGTTT
ATCATCAAACCAGTTATCCAAGTTCCCAACACAGATCAGCCTAGCTCAATTACGGCTCCAGCATATGCAGCAACAG
CAACCGCCTCCACGTTTGATAAACTTTCAGAATCACAGCCCCAAACCCAATGGACCAGTTCTTCCTCCTCATCCTCAA
CAACTGAGATATCCACCAAACCAGAACATACCACGACAAGCAATAAAGCCAAACCCCCTACAGATGGCTTTCTTGG
CTCAACAAGCCATAAAACAGTGGCAGATCAGCAGTGGACAGGGAACCCCATCAACTACCAACAGCACATCCTCTA
CTCCTTCCAGCCCCACGATTACTAGTGCAGCAGGATATGATGGAAAGGCTTTTGGTTCACCTATGATCGATTTGAG
CTCACCAGTGGGAGGGTCTTATAATCTTCCCTCTCTTCCGGATATTGACTGTTCAAGTACTATTATGCTGGACAATA
TTGTGAGGAAAGATACTAATATAGATCATGGCCAGCCAAGACCACCCTCAAACAGAACGGTCCAGTCACCAAATTC
ATCAGTGCCATCTCCAGGCCTTGCAGGACCTGTTACTATGACTAGTGTACACCCCCCAATACGTTCACCTAGTGCCT
CCAGCGTTGGAAGCCGAGGAAGCTCTGGCTCTTCCAGCAAACCAGCAGGAGCTGACTCTACACACAAAGTCCCAG
TGGTCATGCTGGAGCCAATTCGAATAAAACAAGAAAACAGTGGACCACCGGAAAATTATGATTTCCCTGTTGTTAT
AGTGAAGCAAGAATCAGATGAAGAATCTAGGCCTCAAAATGCCAATTATCCAAGAAGCATACTCACCTCCCTGCTC
TTAAATAGCAGTCAGAGCTCTACTTCTGAGGAGACTGTGCTAAGATCAGATGCCCCTGATAGTACAGGAGATCAAC
CTGGACTTCACCAGGACAATTCCTCAAATGGAAAGTCTGAATGGTTGGATCCTTCCCAGAAGTCACCTCTTCATGTT
GGAGAGACAAGGAAAGAGGATGACCCCAATGAGGACTGGTGTGCAGTTTGTCAAAACGGAGGGGAACTCCTCTG
CTGTGAAAAGTGCCCCAAAGTATTCCATCTTTCTTGTCATGTGCCCACATTGACAAATTTTCCAAGTGGAGAGTGGA
TTTGCACTTTCTGCCGAGACTTATCTAAACCAGAAGTTGAATATGATTGTGATGCTCCCAGTCACAACTCAGAAAAA
AAGAAAACTGAAGGCCTTGTTAAGTTAACACCTATAGATAAAAGGAAGTGTGAGCGCCTACTTTTATTTCTTTACT
GCCATGAAATGAGCCTGGCTTTTCAAGACCCTGTTCCTCTAACTGTGCCTGATTATTACAAAATAATTAAAAATCCA
ATGGATTTGTCAACCATCAAGAAAAGACTACAAGAAGATTATTCCATGTACTCAAAACCTGAAGATTTTGTAGCTG
ATTTTAGATTGATCTTTCAAAACTGTGCTGAATTCAATGAGCCTGATTCAGAAGTAGCCAATGCTGGTATAAAACTT
GAAAATTATTITGAAGAACTTCTAAAGAACCTCTATCCAGAAAAAAGGTTTCCCAAACCAGAATTCAGGAATGAAT
CAGAAGATAATAAATTTAGTGATGATTCAGATGATGACTTTGTACAGCCCCGGAAGAAACGCCTCAAAAGCATTG
AAGAACGCCAGTTGCTTAAATAA
Ib: NP_003843.3 (SEQ ID NO: 22)
MEVAVEKAVAAAAAASAAASGGPSAAPSGENEAESRQGPDSERGGEAARLNLLDTCAVCHQNIQSRAPKLLPCLHSFC
QRCLPAPQRYLMLPAPMLGSAETPPPVPAPGSPVSGSSPFATQVGVIRCPVCSQECAERHIIDNFFVKDTTEVPSSTVEK
SNQVCTSCEDNAEANGFCVECVEWLCKTCIRAHQRVKFTKDHTVRQKEEVSPEAVGVTSQRPVFCPFHKKEQLKLYCE
TCDKLTCRDCQLLEHKEHRYQFIEEAFQNQKVIIDTLITKLMEKTKYIKFTGNQIQNRIIEVNQNQKQVEQDIKVAIFTLM
VEINKKGKALLHQLESLAKDHRMKLMQQQQEVAGLSKQLEHVMHFSKWAVSSGSSTALLYSKRLITYRLRHLLRARCD
ASPVTNNTIQFHCDPSFWAQNIINLGSLVIEDKESQPQMPKQNPVVEQNSQPPSGLSSNQLSKFPTQISLAQLRLQHM
QQQQPPPRLINFQNHSPKPNGPVLPPHPQQLRYPPNQNIPRQAIKPNPLQMAFLAQQAIKQWQISSGQGTPSTTNST
SSTPSSPTITSAAGYDGKAFGSPMIDLSSPVGGSYNLPSLPDIDCSSTIMLDNIVRKDTNIDHGQPRPPSNRTVQSPNSSV
PSPGLAGPVTMTSVHPPIRSPSASSVGSRGSSGSSSKPAGADSTHKVPVVMLEPIRIKQENSGPPENYDFPVVIVKQESD
EESRPQNANYPRSILTSLLLNSSQSSTSEETVLRSDAPDSTGDQPGLHQDNSSNGKSEWLDPSQKSPLHVGETRKEDDP
NEDWCAVCQNGGELLCCEKCPKVFHLSCHVPTLTNFPSGEWICTFCRDLSKPEVEYDCDAPSHNSEKKKTEGLVKLTPID
KRKCERLLLFLYCHEMSLAFQDPVPLTVPDYYKIIKNPMDLSTIKKRLQEDYSMYSKPEDFVADFRLIFQNCAEFNEPDSE
VANAGIKLENYFEELLKNLYPEKRFPKPEFRNESEDNKFSDDSDDDFVQPRKKRLKSIEERQLLK
V1: NM_015905.3 (SEQ ID NO: 23)
ATGGAGGTGGCGGTGGAGAAGGCGGTGGCGGCGGCGGCAGCGGCCTCGGCTGCGGCCTCCGGGGGGCCCTCG
GCGGCGCCGAGCGGGGAGAACGAGGCCGAGAGTCGGCAGGGCCCGGACTCGGAGCGCGGCGGCGAGGCGGCC
CGGCTCAACCTGTTGGACACTTGCGCCGTGTGCCACCAGAACATCCAGAGCCGGGCGCCCAAGCTGCTGCCCTGC
CTGCACTCTTTCTGCCAGCGCTGCCTGCCCGCGCCCCAGCGCTACCTCATGCTGCCCGCGCCCATGCTGGGCTCGG
CCGAGACCCCGCCACCCGTCCCTGCCCCCGGCTCGCCGGTCAGCGGCTCGTCGCCGTTCGCCACCCAAGTTGGAGT
CATTCGTTGCCCAGTTTGCAGCCAAGAATGTGCAGAGAGACACATCATAGATAACTTTTTTGTGAAGGACACTACT
GAGGTTCCCAGCAGTACAGTAGAAAAGTCAAATCAGGTATGTACAAGCTGTGAGGACAACGCAGAAGCCAATGG
GTTTTGTGTAGAGTGTGTTGAATGGCTCTGCAAGACGTGTATCAGAGCTCATCAGAGGGTAAAGTTCACAAAAGA
CCACACTGTCAGACAGAAAGAGGAAGTATCTCCAGAGGCAGTTGGTGTCACCAGCCAGCGACCAGTGTTTTGTCC
TTTTCATAAAAAGGAGCAGCTGAAGCTGTACTGTGAGACATGTGACAAACTGACATGTCGAGACTGTCAGTTGTTA
GAACATAAAGAGCATAGATACCAATTTATAGAAGAAGCTTTTCAGAATCAGAAAGTGATCATAGATACACTAATCA
CCAAACTGATGGAAAAAACAAAATACATAAAATTCACAGGAAATCAGATCCAAAACAGAATTATTGAAGTAAATC
AAAATCAAAAGCAGGTGGAACAGGATATTAAAGTTGCTATATTTACACTGATGGTAGAAATAAATAAAAAAGGAA
AAGCTCTACTGCATCAGTTAGAGAGCCTTGCAAAGGACCATCGCATGAAACTTATGCAACAACAACAGGAAGTGG
CTGGACTCTCTAAACAATTGGAGCATGTCATGCATTTTTCTAAATGGGCAGTTTCCAGTGGCAGCAGTACAGCATT
ACTTTATAGCAAACGACTGATTACATACCGGTTACGGCACCTCCTTCGTGCAAGGTGTGATGCATCCCCAGTGACC
AACAACACCATCCAATTTCACTGTGATCCTAGTTTCTGGGCTCAAAATATCATCAACTTAGGTTCTTTAGTAATCGA
GGATAAAGAGAGCCAGCCACAAATGCCTAAGCAGAATCCTGTCGTGGAACAGAATTCACAGCCACCAAGTGGTTT
ATCATCAAACCAGTTATCCAAGTTCCCAACACAGATCAGCCTAGCTCAATTACGGCTCCAGCATATGCAGCAACAG
GTAATGGCTCAGAGGCAACAGGTGCAACGGAGGCCAGCACCTGTGGGTTTACCAAACCCTAGAATGCAGGGGCC
CATCCAGCAACCTTCCATCTCTCATCAGCAACCGCCTCCACGTTTGATAAACTTTCAGAATCACAGCCCCAAACCCA
ATGGACCAGTTCTTCCTCCTCATCCTCAACAACTGAGATATCCACCAAACCAGAACATACCACGACAAGCAATAAA
GCCAAACCCCCTACAGATGGCTTTCTTGGCTCAACAAGCCATAAAACAGTGGCAGATCAGCAGTGGACAGGGAAC
CCCATCAACTACCAACAGCACATCCTCTACTCCTTCCAGCCCCACGATTACTAGTGCAGCAGGATATGATGGAAAG
GCTTTTGGTTCACCTATGATCGATTTGAGCTCACCAGTGGGAGGGTCTTATAATCTTCCCTCTCTTCCGGATATTGA
CTGTTCAAGTACTATTATGCTGGACAATATTGTGAGGAAAGATACTAATATAGATCATGGCCAGCCAAGACCACCC
TCAAACAGAACGGTCCAGTCACCAAATTCATCAGTGCCATCTCCAGGCCTTGCAGGACCTGTTACTATGACTAGTG
TACACCCCCCAATACGTTCACCTAGTGCCTCCAGCGTTGGAAGCCGAGGAAGCTCTGGCTCTTCCAGCAAACCAGC
AGGAGCTGACTCTACACACAAAGTCCCAGTGGTCATGCTGGAGCCAATTCGAATAAAACAAGAAAACAGTGGACC
ACCGGAAAATTATGATTTCCCTGTTGTTATAGTGAAGCAAGAATCAGATGAAGAATCTAGGCCTCAAAATGCCAAT
TATCCAAGAAGCATACTCACCTCCCTGCTCTTAAATAGCAGTCAGAGCTCTACTTCTGAGGAGACTGTGCTAAGATC
AGATGCCCCTGATAGTACAGGAGATCAACCTGGACTTCACCAGGACAATTCCTCAAATGGAAAGTCTGAATGGTT
GGATCCTTCCCAGAAGTCACCTCTTCATGTTGGAGAGACAAGGAAAGAGGATGACCCCAATGAGGACTGGTGTGC
AGTTTGTCAAAACGGAGGGGAACTCCTCTGCTGTGAAAAGTGCCCCAAAGTATTCCATCTTTCTTGTCATGTGCCC
ACATTGACAAATTTTCCAAGTGGAGAGTGGATTTGCACTTTCTGCCGAGACTTATCTAAACCAGAAGTTGAATATG
ATTGTGATGCTCCCAGTCACAACTCAGAAAAAAAGAAAACTGAAGGCCTTGTTAAGTTAACACCTATAGATAAAAG
GAAGTGTGAGCGCCTACTTTTATTTCTTTACTGCCATGAAATGAGCCTGGCTTTTCAAGACCCTGTTCCTCTAACTGT
GCCTGATTATTACAAAATAATTAAAAATCCAATGGATTTGTCAACCATCAAGAAAAGACTACAAGAAGATTATTCC
ATGTACTCAAAACCTGAAGATTTTGTAGCTGATTTTAGATTGATCTTTCAAAACTGTGCTGAATTCAATGAGCCTGA
TTCAGAAGTAGCCAATGCTGGTATAAAACTTGAAAATTATTTTGAAGAACTTCTAAAGAACCTCTATCCAGAAAAA
AGGTTTCCCAAACCAGAATTCAGGAATGAATCAGAAGATAATAAATTTAGTGATGATTCAGATGATGACTTTGTAC
AGCCCCGGAAGAAACGCCTCAAAAGCATTGAAGAACGCCAGTTGCTTAAATAA
Ia: NP_056989.2 (SEQ ID NO: 24)
MEVAVEKAVAAAAAASAAASGGPSAAPSGENEAESRQGPDSERGGEAARLNLLDTCAVCHQNIQSRAPKLLPCLHSFC
QRCLPAPQRYLMLPAPMLGSAETPPPVPAPGSPVSGSSPFATQVGVIRCPVCSQECAERHIIDNFFVKDTTEVPSSTVEK
SNQVCTSCEDNAEANGFCVECVEWLCKTCIRAHQRVKFTKDHTVRQKEEVSPEAVGVTSQRPVFCPFHKKEQLKLYCE
TCDKLTCRDCQLLEHKEHRYQFIEEAFQNQKVIIDTLITKLMEKTKYIKFTGNQIQNRIIEVNQNQKQVEQDIKVAIFTLM
VEINKKGKALLHQLESLAKDHRMKLMQQQQEVAGLSKQLEHVMHFSKWAVSSGSSTALLYSKRLITYRLRHLLRARCD
ASPVTNNTIQFHCDPSFWAQNIINLGSLVIEDKESQPQMPKQNPVVEQNSQPPSGLSSNQLSKFPTQISLAQLRLQHM
QQQVMAQRQQVQRRPAPVGLPNPRMQGPIQQPSISHQQPPPRLINFQNHSPKPNGPVLPPHPQQLRYPPNQNIPR
QAIKPNPLQMAFLAQQAIKQWQISSGQGTPSTTNSTSSTPSSPTITSAAGYDGKAFGSPMIDLSSPVGGSYNLPSLPDID
CSSTIMLDNIVRKDTNIDHGQPRPPSNRTVQSPNSSVPSPGLAGPVTMTSVHPPIRSPSASSVGSRGSSGSSSKPAGADS
THKVPVVMLEPIRIKQENSGPPENYDFPVVIVKQESDEESRPQNANYPRSILTSLLLNSSQSSTSEETVLRSDAPDSTGDQ
PGLHQDNSSNGKSEWLDPSQKSPLHVGETRKEDDPNEDWCAVCQNGGELLCCEKCPKVFHLSCHVPTLTNFPSGEWI
CTFCRDLSKPEVEYDCDAPSHNSEKKKTEGLVKLTPIDKRKCERLLLFLYCHEMSLAFQDPVPLTVPDYYKIIKNPMDLSTI
KKRLQEDYSMYSKPEDFVADFRLIFQNCAEFNEPDSEVANAGIKLENYFEELLKNLYPEKRFPKPEFRNESEDNKFSDDSD
DDFVQPRKKRLKSIEERQLLK
GATA4
V2: NM_002052.5 (SEQ ID NO: 25)
ATGTATCAGAGCTTGGCCATGGCCGCCAACCACGGGCCGCCCCCCGGTGCCTACGAGGCGGGCGGCCCCGGCGC
CTTCATGCACGGCGCGGGCGCCGCGTCCTCGCCAGTCTACGTGCCCACACCGCGGGTGCCCTCCTCCGTGCTGGGC
CTGTCCTACCTCCAGGGCGGAGGCGCGGGCTCTGCGTCCGGAGGCGCCTCGGGCGGCAGCTCCGGTGGGGCCGC
GTCTGGTGCGGGGCCCGGGACCCAGCAGGGCAGCCCGGGATGGAGCCAGGCGGGAGCCGACGGAGCCGCTTAC
ACCCCGCCGCCGGTGTCGCCGCGCTTCTCCTTCCCGGGGACCACCGGGTCCCTGGCGGCCGCCGCCGCCGCTGCC
GCGGCCCGGGAAGCTGCGGCCTACAGCAGTGGCGGCGGAGCGGCGGGTGCGGGCCTGGCGGGCCGCGAGCAG
TACGGGCGCGCCGGCTTCGCGGGCTCCTACTCCAGCCCCTACCCGGCTTACATGGCCGACGTGGGCGCGTCCTGG
GCCGCAGCCGCCGCCGCCTCCGCCGGCCCCTTCGACAGCCCGGTCCTGCACAGCCTGCCCGGCCGGGCCAACCCG
GCCGCCCGACACCCCAATCTCGATATGTTTGACGACTTCTCAGAAGGCAGAGAGTGTGTCAACTGTGGGGCTATGT
CCACCCCGCTCTGGAGGCGAGATGGGACGGGTCACTATCTGTGCAACGCCTGCGGCCTCTACCACAAGATGAACG
GCATCAACCGGCCGCTCATCAAGCCTCAGCGCCGGCTGTCCGCCTCCCGCCGAGTGGGCCTCTCCTGTGCCAACTG
CCAGACCACCACCACCACGCTGTGGCGCCGCAATGCGGAGGGCGAGCCTGTGTGCAATGCCTGCGGCCTCTACAT
GAAGCTCCACGGGGTCCCCAGGCCTCTTGCAATGCGGAAAGAGGGGATCCAAACCAGAAAACGGAAGCCCAAGA
ACCTGAATAAATCTAAGACACCAGCAGCTCCTTCAGGCAGTGAGAGCCTTCCTCCCGCCAGCGGTGCTTCCAGCAA
CTCCAGCAACGCCACCACCAGCAGCAGCGAGGAGATGCGTCCCATCAAGACGGAGCCTGGCCTGTCATCTCACTA
CGGGCACAGCAGCTCCGTGTCCCAGACGTTCTCAGTCAGTGCGATGTCTGGCCATGGGCCCTCCATCCACCCTGTC
CTCTCGGCCCTGAAGCTCTCCCCACAAGGCTATGCGTCTCCCGTCAGCCAGTCTCCACAGACCAGCTCCAAGCAGG
ACTCTTGGAACAGCCTGGTCTTGGCCGACAGTCACGGGGACATAATCACTGCGTAA
I2: NP_002043.2 (SEQ ID NO: 26)
MYQSLAMAANHGPPPGAYEAGGPGAFMHGAGAASSPVYVPTPRVPSSVLGLSYLQGGGAGSASGGASGGSSGGAA
SGAGPGTQQGSPGWSQAGADGAAYTPPPVSPRFSFPGTTGSLAAAAAAAAAREAAAYSSGGGAAGAGLAGREQYG
RAGFAGSYSSPYPAYMADVGASWAAAAAASAGPFDSPVLHSLPGRANPAARHPNLDMFDDFSEGRECVNCGAMST
PLWRRDGTGHYLCNACGLYHKMNGINRPLIKPQRRLSASRRVGLSCANCQTTTTTLWRRNAEGEPVCNACGLYMKLH
GVPRPLAMRKEGIQTRKRKPKNLNKSKTPAAPSGSESLPPASGASSNSSNATTSSSEEMRPIKTEPGLSSHYGHSSSVSQ
TFSVSAMSGHGPSIHPVLSALKLSPQGYASPVSQSPQTSSKQDSWNSLVLADSHGDIITA
V1: NM_001308093.3 (SEQ ID NO: 27)
ATGTATCAGAGCTTGGCCATGGCCGCCAACCACGGGCCGCCCCCCGGTGCCTACGAGGCGGGCGGCCCCGGCGC
CTTCATGCACGGCGCGGGCGCCGCGTCCTCGCCAGTCTACGTGCCCACACCGCGGGTGCCCTCCTCCGTGCTGGGC
CTGTCCTACCTCCAGGGCGGAGGCGCGGGCTCTGCGTCCGGAGGCGCCTCGGGCGGCAGCTCCGGTGGGGCCGC
GTCTGGTGCGGGGCCCGGGACCCAGCAGGGCAGCCCGGGATGGAGCCAGGCGGGAGCCGACGGAGCCGCTTAC
ACCCCGCCGCCGGTGTCGCCGCGCTTCTCCTTCCCGGGGACCACCGGGTCCCTGGCGGCCGCCGCCGCCGCTGCC
GCGGCCCGGGAAGCTGCGGCCTACAGCAGTGGCGGCGGAGCGGCGGGTGCGGGCCTGGCGGGCCGCGAGCAG
TACGGGCGCGCCGGCTTCGCGGGCTCCTACTCCAGCCCCTACCCGGCTTACATGGCCGACGTGGGCGCGTCCTGG
GCCGCAGCCGCCGCCGCCTCCGCCGGCCCCTTCGACAGCCCGGTCCTGCACAGCCTGCCCGGCCGGGCCAACCCG
GCCGCCCGACACCCCAATCTCGTAGATATGTTTGACGACTTCTCAGAAGGCAGAGAGTGTGTCAACTGTGGGGCT
ATGTCCACCCCGCTCTGGAGGCGAGATGGGACGGGTCACTATCTGTGCAACGCCTGCGGCCTCTACCACAAGATG
AACGGCATCAACCGGCCGCTCATCAAGCCTCAGCGCCGGCTGTCCGCCTCCCGCCGAGTGGGCCTCTCCTGTGCCA
ACTGCCAGACCACCACCACCACGCTGTGGCGCCGCAATGCGGAGGGCGAGCCTGTGTGCAATGCCTGCGGCCTCT
ACATGAAGCTCCACGGGGTCCCCAGGCCTCTTGCAATGCGGAAAGAGGGGATCCAAACCAGAAAACGGAAGCCC
AAGAACCTGAATAAATCTAAGACACCAGCAGCTCCTTCAGGCAGTGAGAGCCTTCCTCCCGCCAGCGGTGCTTCCA
GCAACTCCAGCAACGCCACCACCAGCAGCAGCGAGGAGATGCGTCCCATCAAGACGGAGCCTGGCCTGTCATCTC
ACTACGGGCACAGCAGCTCCGTGTCCCAGACGTTCTCAGTCAGTGCGATGTCTGGCCATGGGCCCTCCATCCACCC
TGTCCTCTCGGCCCTGAAGCTCTCCCCACAAGGCTATGCGTCTCCCGTCAGCCAGTCTCCACAGACCAGCTCCAAGC
AGGACTCTTGGAACAGCCTGGTCTTGGCCGACAGTCACGGGGACATAATCACTGCGTAA
I1: NP_001295022.1 (SEQ ID NO: 28)
MYQSLAMAANHGPPPGAYEAGGPGAFMHGAGAASSPVYVPTPRVPSSVLGLSYLQGGGAGSASGGASGGSSGGAA
SGAGPGTQQGSPGWSQAGADGAAYTPPPVSPRFSFPGTTGSLAAAAAAAAAREAAAYSSGGGAAGAGLAGREQYG
RAGFAGSYSSPYPAYMADVGASWAAAAAASAGPFDSPVLHSLPGRANPAARHPNLVDMFDDFSEGRECVNCGAMS
TPLWRRDGTGHYLCNACGLYHKMNGINRPLIKPQRRLSASRRVGLSCANCQTTTTTLWRRNAEGEPVCNACGLYMKL
HGVPRPLAMRKEGIQTRKRKPKNLNKSKTPAAPSGSESLPPASGASSNSSNATTSSSEEMRPIKTEPGLSSHYGHSSSVS
QTFSVSAMSGHGPSIHPVLSALKLSPQGYASPVSQSPQTSSKQDSWNSLVLADSHGDIITA
V3: NM_001308094.2 and V4: NM_001374273.1 both have the same CDS (SEQ ID NO: 29)
and code for 13
ATGTTTGACGACTTCTCAGAAGGCAGAGAGTGTGTCAACTGTGGGGCTATGTCCACCCCGCTCTGGAGGCGAGAT
GGGACGGGTCACTATCTGTGCAACGCCTGCGGCCTCTACCACAAGATGAACGGCATCAACCGGCCGCTCATCAAG
CCTCAGCGCCGGCTGTCCGCCTCCCGCCGAGTGGGCCTCTCCTGTGCCAACTGCCAGACCACCACCACCACGCTGT
GGCGCCGCAATGCGGAGGGCGAGCCTGTGTGCAATGCCTGCGGCCTCTACATGAAGCTCCACGGGGTCCCCAGG
CCTCTTGCAATGCGGAAAGAGGGGATCCAAACCAGAAAACGGAAGCCCAAGAACCTGAATAAATCTAAGACACC
AGCAGCTCCTTCAGGCAGTGAGAGCCTTCCTCCCGCCAGCGGTGCTTCCAGCAACTCCAGCAACGCCACCACCAGC
AGCAGCGAGGAGATGCGTCCCATCAAGACGGAGCCTGGCCTGTCATCTCACTACGGGCACAGCAGCTCCGTGTCC
CAGACGTTCTCAGTCAGTGCGATGTCTGGCCATGGGCCCTCCATCCACCCTGTCCTCTCGGCCCTGAAGCTCTCCCC
ACAAGGCTATGCGTCTCCCGTCAGCCAGTCTCCACAGACCAGCTCCAAGCAGGACTCTTGGAACAGCCTGGTCTTG
GCCGACAGTCACGGGGACATAATCACTGCGTAA
I3: NP_001295023.1 and 13: NP_001361202.1 (SEQ ID NO: 30)
MFDDFSEGRECVNCGAMSTPLWRRDGTGHYLCNACGLYHKMNGINRPLIKPQRRLSASRRVGLSCANCQTTTTTLW
RRNAEGEPVCNACGLYMKLHGVPRPLAMRKEGIQTRKRKPKNLNKSKTPAAPSGSESLPPASGASSNSSNATTSSSEE
MRPIKTEPGLSSHYGHSSSVSQTFSVSAMSGHGPSIHPVLSALKLSPQGYASPVSQSPQTSSKQDSWNSLVLADSHGDII
TA
V5: NM_001374274.1 (SEQ ID NO: 31)
ATGTTTGACGACTTCTCAGAAGGCAGAGAGTGTGTCAACTGTGGGGCTATGTCCACCCCGCTCTGGAGGCGAGAT
GGGACGGGTCACTATCTGTGCAACGCCTGCGGCCTCTACCACAAGATGAACGGCATCAACCGGCCGCTCATCAAG
CCTCAGCGCCGGCTGGTCCCCAGGCCTCTTGCAATGCGGAAAGAGGGGATCCAAACCAGAAAACGGAAGCCCAA
GAACCTGAATAAATCTAAGACACCAGCAGCTCCTTCAGGCAGTGAGAGCCTTCCTCCCGCCAGCGGTGCTTCCAGC
AACTCCAGCAACGCCACCACCAGCAGCAGCGAGGAGATGCGTCCCATCAAGACGGAGCCTGGCCTGTCATCTCAC
TACGGGCACAGCAGCTCCGTGTCCCAGACGTTCTCAGTCAGTGCGATGTCTGGCCATGGGCCCTCCATCCACCCTG
TCCTCTCGGCCCTGAAGCTCTCCCCACAAGGCTATGCGTCTCCCGTCAGCCAGTCTCCACAGACCAGCTCCAAGCA
GGACTCTTGGAACAGCCTGGTCTTGGCCGACAGTCACGGGGACATAATCACTGCGTAA
I4: NP_001361203.1 (Variant 5 code for isoform 4) (SEQ ID NO: 32)
MFDDFSEGRECVNCGAMSTPLWRRDGTGHYLCNACGLYHKMNGINRPLIKPQRRLVPRPLAMRKEGIQTRKRKPKNL
NKSKTPAAPSGSESLPPASGASSNSSNATTSSSEEMRPIKTEPGLSSHYGHSSSVSQTFSVSAMSGHGPSIHPVLSALKLS
PQGYASPVSQSPQTSSKQDSWNSLVLADSHGDIITA
PBX1
XM_005245229.4 (SEQ ID NO: 33)
ATGGACGAGCAGCCCAGGCTGATGCATTCCCATGCTGGGGTCGGGATGGCCGGACACCCCGGCCTGTCCCAGCAC
TTGCAGGATGGGGCCGGAGGGACCGAGGGGGAGGGCGGGAGGAAGCAGGACATTGGAGACATTTTACAGCAA
ATTATGACCATCACAGACCAGAGTTTGGATGAGGCGCAGGCCAGAAAACATGCTTTAAACTGCCACAGAATGAAG
CCTGCCTTGTTTAATGTGTTGTGTGAAATCAAAGAAAAAACAGTTTTGAGTATCCGAGGAGCCCAGGAGGAGGAA
CCCACAGACCCCCAGCTGATGCGGCTGGACAACATGCTGTTAGCGGAAGGCGTGGCGGGGCCTGAGAAGGGCG
GAGGGTCGGCGGCAGCGGCGGCAGCGGCGGCGGCTTCTGGAGGGGCAGGTTCAGACAACTCAGTGGAGCATTC
AGATTACAGAGCCAAACTCTCACAGATCAGACAAATCTACCATACGGAGCTGGAGAAATACGAGCAGGCCTGCAA
CGAGTTCACCACCCACGTGATGAATCTCCTGCGAGAGCAAAGCCGGACCAGGCCCATCTCCCCAAAGGAGATTGA
GCGGATGGTCAGCATCATCCACCGCAAGTTCAGCTCCATCCAGATGCAGCTCAAGCAGAGCACGTGCGAGGCGGT
GATGATCCTGCGTTCCCGATTTCTGGATGCGCGGCGGAAGAGACGGAATTTCAACAAGCAAGCGACAGAAATCCT
GAATGAATATTTCTATTCCCATCTCAGCAACCCTTACCCCAGTGAGGAAGCCAAAGAGGAGTTAGCCAAGAAGTGT
GGCATCACAGTCTCCCAGGTATCAAACTGGTTTGGAAATAAGCGAATCCGGTACAAGAAGAACATAGGTAAATTT
CAAGAGGAAGCCAATATTTATGCTGCCAAAACAGCTGTCACTGCTACCAATGTGTCAGCCCATGGAAGCCAAGCTA
ACTCGCCCTCAACTCCCAACTCGGCTGGTTCTTCCAGTTCTTTTAACATGTCAAACTCTGGAGATTTGTTCATGAGCG
TGCAGTCACTCAATGGGGATTCTTACCAAGGGGCCCAGGTTGGAGCCAACGTGCAATCACAGGTGGATACCCTTC
GCCATGTTATCAGCCAGACAGGAGGATACAGTGATGGACTCGCAGCCAGTCAGATGTACAGTCCGCAGGGCATCA
GTGCTAATGGAGGTTGGCAGGATGCTACTACCCCTTCATCAGTGACCTCCCCTACAGAAGGCCCTGGCAGTGTTCA
CTCTGATACCTCCAACTGA
XP_005245286.1 (SEQ ID NO: 34)
MDEQPRLMHSHAGVGMAGHPGLSQHLQDGAGGTEGEGGRKQDIGDILQQIMTITDQSLDEAQARKHALNCHRMK
PALFNVLCEIKEKTVLSIRGAQEEEPTDPQLMRLDNMLLAEGVAGPEKGGGSAAAAAAAAASGGAGSDNSVEHSDYRA
KLSQIRQIYHTELEKYEQACNEFTTHVMNLLREQSRTRPISPKEIERMVSIIHRKFSSIQMQLKQSTCEAVMILRSRFLDAR
RKRRNFNKQATEILNEYFYSHLSNPYPSEEAKEELAKKCGITVSQVSNWFGNKRIRYKKNIGKFQEEANIYAAKTAVTATN
VSAHGSQANSPSTPNSAGSSSSFNMSNSGDLFMSVQSLNGDSYQGAQVGANVQSQVDTLRHVISQTGGYSDGLAAS
QMYSPQGISANGGWQDATTPSSVTSPTEGPGSVHSDTSN
ZBTB39
NM_014830.3 (SEQ ID NO: 35)
ATGGGCATGAGGATCAAACTGCAAAGCACCAACCACCCCAACAACCTGCTGAAGGAACTCAACAAGTGCCGGCTC
TCAGAGACCATGTGCGACGTCACCATTGTGGTGGGGAGCCGCTCCTTCCCGGCCCACAAGGCTGTGCTGGCCTGT
GCAGCTGGCTACTTCCAGAACCTCTTCCTGAATACTGGGCTTGATGCTGCCAGGACCTATGTGGTGGACTTCATCA
CCCCTGCCAACTTTGAGAAGGTTCTGAGCTTTGTCTACACTTCAGAACTCTTCACAGACCTGATCAATGTTGGGGTC
ATCTACGAGGTAGCTGAGCGTCTGGGTATGGAGGACCTCCTCCAGGCCTGTCACTCTACCTTTCCTGATCTGGAGA
GCACTGCCAGGGCCAAGCCCCTGACCAGCACCAGTGAGAGCCACTCTGGTACCCTGAGTTGTCCTTCGGCAGAAC
CTGCCCATCCCCTTGGAGAACTCCGAGGTGGTGGGGCTACCTTGGTGCTGATAGAAACTATGTGTTGCCCAGTGAT
GCTGGAGGGAGCTATAAAGAGGAAGAGAAGAATGTTGCCAGTGACGCTAACCATAGCCTGCATCTGCCGCAACC
GCCCCCACCACCGCCAAAGACAGAAGACCATGACACCCCTGCTCCCTTCACGTCCATTCCTAGCATGATGACCCAG
CCACTCCTAGGCACTGTCAGCACGGGCATCCAGACCAGCACGAGCTCCTGCCAGCCATACAAAGTTCAAAGCAAT
GGAGACTTCAGTAAAAACAGCTTCCTCACCCCTGACAATGCAGTAGACATTACCACTGGGACCAACTCCTGTCTGA
GCAATAGTGAGCACTCCAAAGATCCTGGCTTTGGGCAGATGGATGAGCTCCAGCTCGAGGACCTGGGGGATGAT
GACTTGCAGTTTGAAGACCCTGCTGAGGATATAGGCACAACTGAGGAGGTGATTGAGCTGAGTGATGACAGTGA
GGATGAGTTGGCTTTTGGAGAGAATGACAATCGGGAGAATAAGGCCATGCCCTGCCAGGTGTGCAAGAAAGTTC
TAGAGCCCAACATTCAACTGATCCGGCAGCATGCTCGGGACCATGTGGACCTGCTGACGGGCAACTGCAAGGTCT
GCGAGACCCACTTCCAGGACCGAAACTCCCGGGTAACTCATGTCCTGTCCCACATTGGTATTTTCCTTTTCTCCTGC
GACATGTGTGAAACTAAGTTCTTTACCCAGTGGCAGCTGACCCTTCACCGACGGGATGGAATATTTGAGAACAACA
TCATTGTCCACCCCAACGATCCCCTGCCAGGGAAGCTGGGTCTCTTTTCAGGGGCAGCCTCCCCAGAGCTGAAATG
CGCTGCCTGTGGGAAAGTATTGGCCAAAGATTTCCATGTGGTCCGGGGCCACATCCTTGACCATCTAAACTTGAAG
GGCCAGGCCTGCAGTGTCTGCGACCAGCGTCACCTTAACCTCTGCAGCCTCATGTGGCACACGCTGTCCCATCTCG
GCATCTCAGTCTTCTCCTGTTCTGTCTGTGCGAACAGCTTTGTGGACTGGCATCTTCTAGAGAAGCACATGGCTGTG
CACCAAAGTCTGGAAGACGCCCTCTTCCACTGCCGCTTGTGCAGCCAGAGCTTCAAGTCAGAGGCTGCCTATCGCT
ACCACGTCAGCCAGCACAAATGCAACAGTGGCCTTGATGCACGGCCTGGTTTTGGGCTGCAGCACCCAGCTCTCCA
GAAGCGGAAGCTGCCAGCAGAGGAGTTTCTGGGTGAAGAGCTGGCGCTGCAGGGCCAACCTGGGAACAGCAAG
TATAGCTGCAAGGTCTGTGGCAAAAGATTTGCCCACACAAGCGAATTCAACTACCACCGGCGGATCCACACGGGG
GAGAAGCCATACCAATGTAAGGTGTGCCACAAGTTCTTTCGAGGCCGCTCGACCATCAAGTGCCACCTAAAGACA
CACTCGGGGGCCCTCATGTACCGCTGCACAGTCTGTGGGCACTACAGTTCCACCCTTAACCTCATGAGCAAACATG
TTGGTGTGCACAAAGGCAGCCTCCCCCCTGACTTCACCATCGAGCAGACCTTCATGTACATCATCCATTCCAAAGA
GGCGGATAAGAACCCGGACAGTTGA
NP_055645.1 (SEQ ID NO: 36)
MGMRIKLQSTNHPNNLLKELNKCRLSETMCDVTIVVGSRSFPAHKAVLACAAGYFQNLFLNTGLDAARTYVVDFITPA
NFEKVLSFVYTSELFTDLINVGVIYEVAERLGMEDLLQACHSTFPDLESTARAKPLTSTSESHSGTLSCPSAEPAHPLGELR
GGGDYLGADRNYVLPSDAGGSYKEEEKNVASDANHSLHLPQPPPPPPKTEDHDTPAPFTSIPSMMTQPLLGTVSTGIQ
TSTSSCQPYKVQSNGDFSKNSFLTPDNAVDITTGTNSCLSNSEHSKDPGFGQMDELQLEDLGDDDLQFEDPAEDIGTTE
EVIELSDDSEDELAFGENDNRENKAMPCQVCKKVLEPNIQLIRQHARDHVDLLTGNCKVCETHFQDRNSRVTHVLSHIG
IFLFSCDMCETKFFTQWQLTLHRRDGIFENNIIVHPNDPLPGKLGLFSGAASPELKCAACGKVLAKDFHVVRGHILDHLN
LKGQACSVCDQRHLNLCSLMWHTLSHLGISVFSCSVCANSFVDWHLLEKHMAVHQSLEDALFHCRLCSQSFKSEAAYR
YHVSQHKCNSGLDARPGFGLQHPALQKRKLPAEEFLGEELALQGQPGNSKYSCKVCGKRFAHTSEFNYHRRIHTGEKPY
QCKVCHKFFRGRSTIKCHLKTHSGALMYRCTVCGHYSSTLNLMSKHVGVHKGSLPPDFTIEQTFMYIIHSKEADKNPDS
HAND2
NM_021973.3 (SEQ ID NO: 37)
ATGAGTCTGGTAGGTGGTTTTCCCCACCACCCGGTGGTGCACCACGAGGGCTACCCGTTTGCCGCCGCCGCCGCC
GCAGCTGCCGCCGCCGCCGCCAGCCGCTGCAGCCATGAGGAGAACCCCTACTTCCATGGCTGGCTCATCGGCCAC
CCCGAGATGTCGCCCCCCGACTACAGCATGGCCCTGTCCTACAGCCCCGAGTATGCCAGCGGCGCCGCCGGCCTG
GACCACTCCCATTACGGGGGGGTGCCGCCGGGCGCCGGGCCCCCGGGCCTGGGGGGGCCGCGCCCGGTGAAGC
GCCGAGGCACCGCCAACCGCAAGGAGCGGCGCAGGACTCAGAGCATCAACAGCGCCTTCGCCGAACTGCGCGAG
TGCATCCCCAACGTACCCGCCGACACCAAACTCTCCAAAATCAAGACCCTGCGCCTGGCCACCAGCTACATCGCCT
ACCTCATGGACCTGCTGGCCAAGGACGACCAGAATGGCGAGGCGGAGGCCTTCAAGGCAGAGATCAAGAAGACC
GACGTGAAAGAGGAGAAGAGGAAGAAGGAGCTGAACGAAATCTTGAAAAGCACAGTGAGCAGCAACGACAAGA
AAACCAAAGGCCGGACGGGCTGGCCGCAGCACGTCTGGGCCCTGGAGCTCAAGCAGTGA
NP_068808.1 (SEQ ID NO: 38)
MSLVGGFPHHPVVHHEGYPFAAAAAAAAAAAASRCSHEENPYFHGWLIGHPEMSPPDYSMALSYSPEYASGAAGLD
HSHYGGVPPGAGPPGLGGPRPVKRRGTANRKERRRTQSINSAFAELRECIPNVPADTKLSKIKTLRLATSYIAYLMDLLAK
DDQNGEAEAFKAEIKKTDVKEEKRKKELNEILKSTVSSNDKKTKGRTGWPQHVWALELKQ
IKZF4
NM_001351091.2 (SEQ ID NO: 39)
ATGGACATAGAAGACTGCAATGGCCGCTCCTATGTGTCTGGTAGCGGGGACTCATCTCTGGAGAAGGAGTTCCTC
GGGGCCCCAGTGGGGCCCTCGGTGAGCACCCCCAACAGCCAGCACTCTTCTCCTAGCCGCTCACTCAGTGCCAACT
CCATCAAGGTGGAGATGTACAGCGATGAGGAGTCAAGCAGACTGCTGGGGCCAGATGAGCGGCTCCTGGAAAAG
GACGACAGCGTGATTGTGGAAGATTCATTGTCTGAGCCCCTGGGCTACTGTGATGGGAGTGGGCCAGAGCCTCAC
TCCCCTGGGGGCATCCGGCTGCCCAATGGCAAGCTCAAGTGTGACGTCTGCGGCATGGTCTGTATTGGACCCAAC
GTGCTCATGGTGCACAAGCGCAGTCACACTGGTGAAAGGCCCTTCCATTGCAACCAGTGTGGTGCCTCCTTCACCC
AGAAGGGGAACCTGCTGCGCCACATCAAGCTGCACTCTGGGGAGAAGCCCTTTAAATGTCCCTTCTGCAACTATGC
CTGCCGCCGGCGTGATGCACTCACTGGTCACCTCCGCACACACTCAGTCTCCTCTCCCACAGTGGGCAAGCCCTAC
AAGTGTAACTACTGTGGCCGGAGCTACAAACAGCAGAGTACCCTGGAGGAGCACAAGGAGCGGTGCCATAACTA
CCTACAGAGTCTCAGCACTGAAGCCCAAGCTTTGGCTGGCCAACCAGGTGACGAAATACGTGACCTGGAGATGGT
GCCAGACTCCATGCTGCACTCATCCTCTGAGCGGCCAACTTTCATCGATCGTCTGGCCAATAGCCTCACCAAACGCA
AGCGTTCCACACCCCAGAAGTTTGTAGGCGAAAAGCAGATGCGCTTCAGCCTCTCAGACCTCCCCTATGATGTGAA
CTCGGGTGGCTATGAAAAGGATGTGGAGTTGGTGGCACACCACAGCCTAGAGCCTGGCTTTGGAAGTTCCCTGGC
CTTTGTGGGTGCAGAGCATCTGCGTCCCCTCCGCCTTCCACCCACCAATTGCATCTCAGAACTCACGCCTGTCATCA
GCTCTGTCTACACCCAGATGCAGCCCCTCCCTGGTCGACTGGAGCTTCCAGGATCCCGAGAAGCAGGTGAGGGAC
CTGAGGACCTGGCTGATGGAGGTCCCCTCCTCTACCGGCCCCGAGGCCCCCTGACTGACCCTGGGGCATCCCCCA
GCAATGGCTGCCAGGACTCCACAGACACAGAAAGCAACCACGAAGATCGGGTTGCGGGGGTGGTATCCCTCCCTC
AGGGTCCCCCACCCCAGCCACCTCCCACCATTGTGGTGGGCCGGCACAGTCCTGCCTACGCCAAAGAGGACCCCA
AGCCACAGGAGGGGTTATTGCGGGGCACCCCAGGCCCCTCCAAGGAAGTGCTTCGGGTGGTGGGCGAGAGTGGT
GAGCCTGTGAAGGCCTTCAAGTGTGAGCACTGCCGTATCCTCTTCCTGGACCACGTCATGTTCACTATCCACATGG
GCTGCCATGGCTTCAGAGACCCTTTTGAGTGCAACATCTGTGGTTATCACAGCCAGGACCGGTACGAATTCTCTTC
CCACATTGTCCGGGGGGAGCATAAGGTGGGCTAG
NP_001338020.1 (SEQ ID NO: 40)
MDIEDCNGRSYVSGSGDSSLEKEFLGAPVGPSVSTPNSQHSSPSRSLSANSIKVEMYSDEESSRLLGPDERLLEKDDSVIV
EDSLSEPLGYCDGSGPEPHSPGGIRLPNGKLKCDVCGMVCIGPNVLMVHKRSHTGERPFHCNQCGASFTQKGNLLRHI
KLHSGEKPFKCPFCNYACRRRDALTGHLRTHSVSSPTVGKPYKCNYCGRSYKQQSTLEEHKERCHNYLQSLSTEAQALA
GQPGDEIRDLEMVPDSMLHSSSERPTFIDRLANSLTKRKRSTPQKFVGEKQMRFSLSDLPYDVNSGGYEKDVELVAHHS
LEPGFGSSLAFVGAEHLRPLRLPPTNCISELTPVISSVYTQMQPLPGRLELPGSREAGEGPEDLADGGPLLYRPRGPLTDP
GASPSNGCQDSTDTESNHEDRVAGVVSLPQGPPPQPPPTIVVGRHSPAYAKEDPKPQEGLLRGTPGPSKEVLRVVGES
GEPVKAFKCEHCRILFLDHVMFTIHMGCHGFRDPFECNICGYHSQDRYEFSSHIVRGEHKVG
NROB2
NM_021969.3 (SEQ ID NO: 41)
ATGAGCACCAGCCAACCAGGGGCCTGCCCATGCCAGGGAGCTGCAAGCCGCCCCGCCATTCTCTACGCACTTCTG
AGCTCCAGCCTCAAGGCTGTCCCCCGACCCCGTAGCCGCTGCCTATGTAGGCAGCACCGGCCCGTCCAGCTATGTG
CACCTCATCGCACCTGCCGGGAGGCCTTGGATGTTCTGGCCAAGACAGTGGCCTTCCTCAGGAACCTGCCATCCTT
CTGGCAGCTGCCTCCCCAGGACCAGCGGCGGCTGCTGCAGGGTTGCTGGGGCCCCCTCTTCCTGCTTGGGTTGGC
CCAAGATGCTGTGACCTTTGAGGTGGCTGAGGCCCCGGTGCCCAGCATACTCAAGAAGATTCTGCTGGAGGAGCC
CAGCAGCAGTGGAGGCAGTGGCCAACTGCCAGACAGACCCCAGCCCTCCCTGGCTGCGGTGCAGTGGCTTCAATG
CTGTCTGGAGTCCTTCTGGAGCCTGGAGCTTAGCCCCAAGGAATATGCCTGCCTGAAAGGGACCATCCTCTTCAAC
CCCGATGTGCCAGGCCTCCAAGCCGCCTCCCACATTGGGCACCTGCAGCAGGAGGCTCACTGGGTGCTGTGTGAA
GTCCTGGAACCCTGGTGCCCAGCAGCCCAAGGCCGCCTGACCCGTGTCCTCCTCACGGCCTCCACCCTCAAGTCCA
TTCCGACCAGCCTGCTTGGGGACCTCTTCTTTCGCCCTATCATTGGAGATGTTGACATCGCTGGCCTTCTTGGGGAC
ATGCTTTTGCTCAGGTGA
NP_068804.1 (SEQ ID NO: 42)
MSTSQPGACPCQGAASRPAILYALLSSSLKAVPRPRSRCLCRQHRPVQLCAPHRTCREALDVLAKTVAFLRNLPSFWQL
PPQDQRRLLQGCWGPLFLLGLAQDAVTFEVAEAPVPSILKKILLEEPSSSGGSGQLPDRPQPSLAAVQWLQCCLESFWS
LELSPKEYACLKGTILFNPDVPGLQAASHIGHLQQEAHWVLCEVLEPWCPAAQGRLTRVLLTASTLKSIPTSLLGDLFFRPI
IGDVDIAGLLGDMLLLR
NACA2
NM_199290.4 (SEQ ID NO: 43)
ATGCCGGGCGAAGCCACAGAAACCGTCCCTGCTACAGAGCAGGAGTTGCCGCAGTCCCAGGCTGAGACAGGGTC
TGGAACAGCATCTGATAGTGGTGAATCAGTACCAGGGATTGAAGAACAGGATTCCACCCAGACCACCACACAAAA
AGCCTGGCTGGTGGCAGCAGCTGAAATTGATGAAGAACCAGTCGGTAAAGCAAAACAGAGTCGGAGTGAAAAGA
GGGCACGGAAGGCTATGTCCAAACTGGGTCTTCTACAGGTTACAGGAGTTACTAGAGTCACTATCTGGAAATCTA
AGAATATCCTCTTTGTCATCACAAAACTGGACGTCTACAAGAGCCCTGCTTCGGATGCCTACATAGTTTTTGGGGA
AGCCAAGATCCAAGATTTATCTCAGCAAGCACAACTAGCAGCTGCGGAGAAATTCAGAGTTCAAGGTGAAGCTGT
CGGAAACATTCAAGAAAACACACAGACTCCAACTGTACAAGAGGAGAGTGAAGAGGAAGAGGTCGATGAAACAG
GTGTAGAAGTTAAAGACGTGAAATTGGTCATGTCACAAGCAAATGTGTCGAGAGCAAAGGCAGTCCGAGCTCTGA
AGAACAACAGTAATGATATTGTAAATGCGATTATGGAATTAACAGTGTAA
NP_954984.1 (SEQ ID NO: 44)
MPGEATETVPATEQELPQSQAETGSGTASDSGESVPGIEEQDSTQTTTQKAWLVAAAEIDEEPVGKAKQSRSEKRARK
AMSKLGLLQVTGVTRVTIWKSKNILFVITKLDVYKSPASDAYIVFGEAKIQDLSQQAQLAAAEKFRVQGEAVGNIQENTQ
TPTVQEESEEEEVDETGVEVKDVKLVMSQANVSRAKAVRALKNNSNDIVNAIMELTV
SMYD1
NM_198274.4 (SEQ ID NO: 45)
ATGACAATAGGGAGAATGGAGAACGTGGAGGTCTTCACCGCTGAGGGCAAAGGAAGGGGTCTGAAGGCCACCA
AGGAGTTCTGGGCTGCAGATATCATCTTTGCTGAGCGGGCTTATTCCGCAGTGGTTTTTGACAGCCTTGTTAATTTT
GTGTGCCACACCTGCTTCAAGAGGCAGGAGAAGCTCCATCGCTGTGGGCAGTGCAAGTTTGCCCATTACTGCGAC
CGCACCTGCCAGAAGGATGCTTGGCTGAACCACAAGAATGAATGTTCGGCCATCAAGAGATATGGGAAGGTGCCC
AATGAGAACATCAGGCTGGCGGCGCGCATCATGTGGGGGTGGAGAGAGAAGGCACCGGGCTCACGGAGGGCT
GCCTGGTGTCCGTGGACGACTTGCAGAACCACGTGGAGCACTTTGGGGAGGAGGAGCAGAAGGACCTGCGGGT
GGACGTGGACACATTCTTGCAGTACTGGCCGCCGCAGAGCCAGCAGTTCAGCATGCAGTACATCTCGCACATCTTC
GGAGTGATTAACTGCAACGGTTTTACTCTCAGTGATCAGAGAGGCCTGCAGGCCGTGGGCGTAGGCATCTTCCCC
AACCTGGGCCTGGTGAACCATGACTGTTGGCCCAACTGTACTGTCATATTTAACAATGGCAATCATGAGGCAGTGA
AATCCATGTTTCATACCCAGATGAGAATTGAGCTCCGGGCCCTAGGCAAGATCTCAGAAGGAGAGGAGCTGACTG
TGTCCTATATTGACTTCCTCAACGTTAGTGAAGAACGCAAGAGGCAGCTGAAGAAGCAGTACTACTTTGACTGCAC
ATGTGAACACTGCCAGAAAAAACTGAAGGATGACCTCTTCCTGGGGGTGAAAGACAACCCCAAGCCCTCTCAGGA
AGTGGTGAAGGAGATGATACAATTCTCCAAGGATACATTGGAAAAGATAGACAAGGCTCGTTCCGAGGGTTTGTA
TCATGAGGTTGTGAAATTATGCCGGGAGTGCCTGGAGAAGCAGGAGCCAGTGTTTGCTGACACCAACATCTACAT
GCTGCGGATGCTGAGCATTGTTTCGGAGGTCCTTTCCTACCTCCAGGCCTTTGAGGAGGCCTCGTTCTATGCCAGG
AGGATGGTGGACGGCTATATGAAGCTCTACCACCCCAACAATGCCCAACTGGGCATGGCCGTGATGCGGGCAGG
GCTGACCAACTGGCATGCTGGTAACATTGAGGTGGGGCACGGGATGATCTGCAAAGCCTATGCCATTCTCCTGGT
GACACACGGACCCTCCCACCCCATCACTAAGGACTTAGAGGCCATGCGGGTGCAGACGGAGATGGAGCTACGCAT
GTTCCGCCAGAACGAATTCATGTACTACAAGATGCGCGAGGCTGCCCTGAACAACCAGCCCATGCAGGTCATGGC
CGAGCCCAGCAATGAGCCATCCCCAGCTCTGTTCCACAAGAAGCAATGA
NP_938015.1 (SEQ ID NO: 46)
MTIGRMENVEVFTAEGKGRGLKATKEFWAADIIFAERAYSAVVFDSLVNFVCHTCFKRQEKLHRCGQCKFAHYCDRTC
QKDAWLNHKNECSAIKRYGKVPNENIRLAARIMWRVEREGTGLTEGCLVSVDDLQNHVEHFGEEEQKDLRVDVDTFL
QYWPPQSQQFSMQYISHIFGVINCNGFTLSDQRGLQAVGVGIFPNLGLVNHDCWPNCTVIFNNGNHEAVKSMFHTQ
MRIELRALGKISEGEELTVSYIDFLNVSEERKRQLKKQYYFDCTCEHCQKKLKDDLFLGVKDNPKPSQEVVKEMIQFSKDT
LEKIDKARSEGLYHEVVKLCRECLEKQEPVFADTNIYMLRMLSIVSEVLSYLQAFEEASFYARRMVDGYMKLYHPNNAQL
GMAVMRAGLTNWHAGNIEVGHGMICKAYAILLVTHGPSHPITKDLEAMRVQTEMELRMFRQNEFMYYKMREAALN
NQPMQVMAEPSNEPSPALFHKKQ
NM_001330364.2 (SEQ ID NO: 47)
ATGACAATAGGGAGAATGGAGAACGTGGAGGTCTTCACCGCTGAGGGCAAAGGAAGGGGTCTGAAGGCCACCA
AGGAGTTCTGGGCTGCAGATATCATCTTTGCTGAGCGGGCTTATTCCGCAGTGGTTTTTGACAGCCTTGTTAATTTT
GTGTGCCACACCTGCTTCAAGAGGCAGGAGAAGCTCCATCGCTGTGGGCAGTGCAAGTTTGCCCATTACTGCGAC
CGCACCTGCCAGAAGGATGCTTGGCTGAACCACAAGAATGAATGTTCGGCCATCAAGAGATATGGGAAGGTGCCC
AATGAGAACATCAGGCTGGCGGCGCGCATCATGTGGCGGGTGGAGAGAGAAGGCACCGGGCTCACGGAGGGCT
GCCTGGTGTCCGTGGACGACTTGCAGAACCACGTGGAGCACTTTGGGGAGGAGGAGCAGAAGGACCTGCGGGT
GGACGTGGACACATTCTTGCAGTACTGGCCGCCGCAGAGCCAGCAGTTCAGCATGCAGTACATCTCGCACATCTTC
GGAGTGATTAACTGCAACGGTTTTACTCTCAGTGATCAGAGAGGCCTGCAGGCCGTGGGCGTAGGCATCTTCCCC
AACCTGGGCCTGGTGAACCATGACTGTTGGCCCAACTGTACTGTCATATTTAACAATGGCAAAATTGAGCTCCGGG
CCCTAGGCAAGATCTCAGAAGGAGAGGAGCTGACTGTGTCCTATATTGACTTCCTCAACGTTAGTGAAGAACGCA
AGAGGCAGCTGAAGAAGCAGTACTACTTTGACTGCACATGTGAACACTGCCAGAAAAAACTGAAGGATGACCTCT
TCCTGGGGGTGAAAGACAACCCCAAGCCCTCTCAGGAAGTGGTGAAGGAGATGATACAATTCTCCAAGGATACAT
TGGAAAAGATAGACAAGGCTCGTTCCGAGGGTTTGTATCATGAGGTTGTGAAATTATGCCGGGAGTGCCTGGAG
AAGCAGGAGCCAGTGTTTGCTGACACCAACATCTACATGCTGCGGATGCTGAGCATTGTTTCGGAGGTCCTTTCCT
ACCTCCAGGCCTTTGAGGAGGCCTCGTTCTATGCCAGGAGGATGGTGGACGGCTATATGAAGCTCTACCACCCCA
ACAATGCCCAACTGGGCATGGCCGTGATGCGGGCAGGGCTGACCAACTGGCATGCTGGTAACATTGAGGTGGGG
CACGGGATGATCTGCAAAGCCTATGCCATTCTCCTGGTGACACACGGACCCTCCCACCCCATCACTAAGGACTTAG
AGGCCATGCGGGTGCAGACGGAGATGGAGCTACGCATGTTCCGCCAGAACGAATTCATGTACTACAAGATGCGC
GAGGCTGCCCTGAACAACCAGCCCATGCAGGTCATGGCCGAGCCCAGCAATGAGCCATCCCCAGCTCTGTTCCAC
AAGAAGCAATGA
NP_001317293.1 (SEQ ID NO: 48)
MTIGRMENVEVFTAEGKGRGLKATKEFWAADIIFAERAYSAVVFDSLVNFVCHTCFKRQEKLHRCGQCKFAHYCDRTC
QKDAWLNHKNECSAIKRYGKVPNENIRLAARIMWRVEREGTGLTEGCLVSVDDLQNHVEHFGEEEQKDLRVDVDTFL
QYWPPQSQQFSMQYISHIFGVINCNGFTLSDQRGLQAVGVGIFPNLGLVNHDCWPNCTVIFNNGKIELRALGKISEGE
ELTVSYIDFLNVSEERKRQLKKQYYFDCTCEHCQKKLKDDLFLGVKDNPKPSQEVVKEMIQFSKDTLEKIDKARSEGLYHE
VVKLCRECLEKQEPVFADTNIYMLRMLSIVSEVLSYLQAFEEASFYARRMVDGYMKLYHPNNAQLGMAVMRAGLTN
WHAGNIEVGHGMICKAYAILLVTHGPSHPITKDLEAMRVQTEMELRMFRQNEFMYYKMREAALNNQPMQVMAEP
SNEPSPALFHKKQ
JUP
NM_021991.4 (SEQ ID NO: 49)
ATGGAGGTGATGAACCTGATGGAGCAGCCTATCAAGGTGACTGAGTGGCAGCAGACATACACCTACGACTCGGG
TATCCACTCGGGCGCCAACACCTGCGTGCCCTCCGTCAGCAGCAAGGGCATCATGGAGGAGGATGAGGCCTGCG
GGCGCCAGTACACGCTCAAGAAAACCACCACTTACACCCAGGGGGTGCCCCCCAGCCAAGGTGATCTGGAGTACC
AGATGTCCACAACAGCCAGGGCCAAACGGGTGCGGGAGGCCATGTGCCCTGGTGTGTCAGGCGAGGACAGCTCG
CTTCTGCTGGCCACCCAGGTGGAGGGGCAGGCCACCAACCTGCAGCGACTGGCCGAGCCGTCCCAGCTGCTCAAG
TCGGCCATTGTGCATCTCATCAACTACCAGGACGATGCCGAGCTGGCCACTCGCGCCCTGCCCGAGCTCACCAAAC
TGCTCAACGACGAGGACCCGGTGGTGGTGACCAAGGCGGCCATGATTGTGAACCAGCTGTCGAAGAAGGAGGCG
TCGCGGCGGGCCCTGATGGGCTCGCCCCAGCTGGTGGCCGCTGTCGTGCGTACCATGCAGAATACCAGCGACCTG
GACACAGCCCGCTGCACCACCAGCATCCTGCACAACCTCTCCCACCACCGGGAGGGGCTGCTCGCCATCTTCAAGT
CGGGTGGCATCCCTGCTCTGGTCCGCATGCTCAGCTCCCCTGTGGAGTCGGTCCTGTTCTATGCCATCACCACGCT
GCACAACCTGCTCCTGTACCAGGAGGGCGCCAAGATGGCCGTGCGCCTGGCCGACGGGCTGCAAAAGATGGTGC
CCCTGCTCAACAAGAACAACCCCAAGTTCCTGGCCATCACCACCGACTGCCTGCAGCTCCTGGCCTACGGCAACCA
GGAGAGCAAGCTGATCATCCTGGCCAATGGTGGGCCCCAGGCCCTCGTGCAGATCATGCGTAACTACAGTTATGA
AAAGCTGCTCTGGACCACCAGTCGTGTGCTCAAGGTGCTATCCGTGTGTCCCAGCAATAAGCCTGCCATTGTGGAG
GCTGGTGGGATGCAGGCCCTGGGCAAGCACCTGACCAGCAACAGCCCCCGCCTGGTGCAGAACTGCCTGTGGAC
CCTGCGCAACCTCTCAGATGTGGCCACCAAGCAGGAGGGCCTGGAGAGTGTGCTGAAGATTCTGGTGAATCAGCT
GAGTGTGGATGACGTCAACGTCCTCACCTGTGCCACGGGCACACTCTCCAACCTGACATGCAACAACAGCAAGAA
CAAGACGCTGGTGACACAGAACAGCGGTGTGGAGGCTCTCATCCATGCCATCCTGCGTGCTGGTGACAAGGACG
ACATCACGGAGCCTGCCGTCTGCGCTCTGCGCCACCTCACTAGCCGCCACCCTGAGGCCGAGATGGCCCAGAACTC
TGTGCGTCTCAACTATGGCATCCCAGCCATCGTGAAGCTGCTCAACCAGCCCAACCAGTGGCCACTGGTCAAGGCA
ACCATCGGCTTGATCAGGAATCTGGCCCTGTGCCCAGCCAACCATGCCCCGCTGCAGGAGGCAGCGGTCATCCCC
CGCCTCGTCCAACTGCTGGTGAAGGCCCACCAGGATGCCCAGCGCCACGTAGCTGCAGGCACACAGCAGCCCTAC
ACGGATGGTGTGAGGATGGAGGAGATTGTGGAGGGCTGCACCGGAGCACTGCACATCCTCGCCCGGGACCCCAT
GAACCGCATGGAGATCTTCCGGCTCAACACCATTCCCCTGTTTGTGCAGCTCCTGTACTCGTCGGTGGAGAACATC
CAGCGCGTGGCTGCCGGGGTGCTGTGTGAGCTGGCCCAGGACAAGGAGGCGGCCGACGCCATTGATGCAGAGG
GGGCCTCGGCCCCACTCATGGAGTTGCTGCACTCCCGCAACGAGGGCACTGCCACCTACGCTGCTGCCGTCCTGTT
CCGCATCTCCGAGGACAAGAACCCAGACTACCGGAAGCGCGTGTCCGTGGAGCTCACCAACTCCCTCTTCAAGCAT
GACCCGGCTGCCTGGGAGGCTGCCCAGAGCATGATTCCCATCAATGAGCCCTATGGAGATGACATGGATGCCACC
TACCGCCCCATGTACTCCAGCGATGTGCCCCTTGACCCGCTGGAGATGCACATGGACATGGATGGAGACTACCCCA
TCGACACCTACAGCGACGGCCTCAGGCCCCCGTACCCCACTGCAGACCACATGCTGGCCTAG
NP_068831.1 (SEQ ID NO: 50)
MEVMNLMEQPIKVTEWQQTYTYDSGIHSGANTCVPSVSSKGIMEEDEACGRQYTLKKTTTYTQGVPPSQGDLEYQM
STTARAKRVREAMCPGVSGEDSSLLLATQVEGQATNLQRLAEPSQLLKSAIVHLINYQDDAELATRALPELTKLLNDEDP
VVVTKAAMIVNQLSKKEASRRALMGSPQLVAAVVRTMQNTSDLDTARCTTSILHNLSHHREGLLAIFKSGGIPALVRML
SSPVESVLFYAITTLHNLLLYQEGAKMAVRLADGLQKMVPLLNKNNPKFLAITTDCLQLLAYGNQESKLIILANGGPQALV
QIMRNYSYEKLLWTTSRVLKVLSVCPSNKPAIVEAGGMQALGKHLTSNSPRLVQNCLWTLRNLSDVATKQEGLESVLKI
LVNQLSVDDVNVLTCATGTLSNLTCNNSKNKTLVTQNSGVEALIHAILRAGDKDDITEPAVCALRHLTSRHPEAEMAQN
SVRLNYGIPAIVKLLNQPNQWPLVKATIGLIRNLALCPANHAPLQEAAVIPRLVQLLVKAHQDAQRHVAAGTQQPYTD
GVRMEEIVEGCTGALHILARDPMNRMEIFRLNTIPLFVQLLYSSVENIQRVAAGVLCELAQDKEAADAIDAEGASAPLM
ELLHSRNEGTATYAAAVLFRISEDKNPDYRKRVSVELTNSLFKHDPAAWEAAQSMIPINEPYGDDMDATYRPMYSSDV
PLDPLEMHMDMDGDYPIDTYSDGLRPPYPTADHMLA
NEUROD1
NM_002500.5 (SEQ ID NO: 51)
ATGACCAAATCGTACAGCGAGAGTGGGCTGATGGGCGAGCCTCAGCCCCAAGGTCCTCCAAGCTGGACAGACGA
GTGTCTCAGTTCTCAGGACGAGGAGCACGAGGCAGACAAGAAGGAGGACGACCTCGAAACCATGAACGCAGAG
GAGGACTCACTGAGGAACGGGGGAGAGGAGGAGGACGAAGATGAGGACCTGGAAGAGGAGGAAGAAGAGGA
AGAGGAGGATGACGATCAAAAGCCCAAGAGACGCGGCCCCAAAAAGAAGAAGATGACTAAGGCTCGCCTGGAG
CGTTTTAAATTGAGACGCATGAAGGCTAACGCCCGGGAGCGGAACCGCATGCACGGACTGAACGCGGCGCTAGA
CAACCTGCGCAAGGTGGTGCCTTGCTATTCTAAGACGCAGAAGCTGTCCAAAATCGAGACTCTGCGCTTGGCCAA
GAACTACATCTGGGCTCTGTCGGAGATCCTGCGCTCAGGCAAAAGCCCAGACCTGGTCTCCTTCGTTCAGACGCTT
TGCAAGGGCTTATCCCAACCCACCACCAACCTGGTTGCGGGCTGCCTGCAACTCAATCCTCGGACTTTTCTGCCTGA
GCAGAACCAGGACATGCCCCCCCACCTGCCGACGGCCAGCGCTTCCTTCCCTGTACACCCCTACTCCTACCAGTCGC
CTGGGCTGCCCAGTCCGCCTTACGGTACCATGGACAGCTCCCATGTCTTCCACGTTAAGCCTCCGCCGCACGCCTAC
AGCGCAGCGCTGGAGCCCTTCTTTGAAAGCCCTCTGACTGATTGCACCAGCCCTTCCTTTGATGGACCCCTCAGCCC
GCCGCTCAGCATCAATGGCAACTTCTCTTTCAAACACGAACCGTCCGCCGAGTTTGAGAAAAATTATGCCTTTACCA
TGCACTATCCTGCAGCGACACTGGCAGGGGCCCAAAGCCACGGATCAATCTTCTCAGGCACCGCTGCCCCTCGCTG
CGAGATCCCCATAGACAATATTATGTCCTTCGATAGCCATTCACATCATGAGCGAGTCATGAGTGCCCAGCTCAAT
GCCATATTTCATGATTAG
NP_002491.3 (SEQ ID NO: 52)
MTKSYSESGLMGEPQPQGPPSWTDECLSSQDEEHEADKKEDDLETMNAEEDSLRNGGEEEDEDEDLEEEEEEEEEDD
DQKPKRRGPKKKKMTKARLERFKLRRMKANARERNRMHGLNAALDNLRKVVPCYSKTQKLSKIETLRLAKNYIWALSEI
LRSGKSPDLVSFVQTLCKGLSQPTTNLVAGCLQLNPRTFLPEQNQDMPPHLPTASASFPVHPYSYQSPGLPSPPYGTMD
SSHVFHVKPPPHAYSAALEPFFESPLTDCTSPSFDGPLSPPLSINGNFSFKHEPSAEFEKNYAFTMHYPAATLAGAQSHGS
IFSGTAAPRCEIPIDNIMSFDSHSHHERVMSAQLNAIFHD
CKMT2
NM_001099736.2 (SEQ ID NO: 53)
ATGGCCAGTATCTTTTCTAAGTTGCTAACTGGCCGCAATGCTTCTCTGCTGTTTGCTACCATGGGCACCAGTGTCCT
GACCACCGGGTACCTGCTGAACCGGCAGAAAGTGTGTGCCGAGGTCCGGGAGCAGCCTAGGCTATTTCCTCCAAG
CGCAGACTACCCAGACCTGCGCAAGCACAACAACTGCATGGCCGAGTGCCTCACCCCCGCCATTTATGCCAAGCTT
CGCAACAAGGTGACACCCAACGGCTACACGCTGGACCAGTGCATCCAGACTGGAGTGGACAACCCTGGCCACCCC
TTCATAAAGACTGTGGGCATGGTGGCTGGTGACGAGGAGTCCTATGAGGTGTTTGCTGACCTTTTTGACCCCGTCA
TCAAACTAAGACACAACGGCTATGACCCCAGGGTGATGAAGCACACAACGGATCTGGATGCATCAAAGATCACCC
AAGGGCAGTTCGACGAGCATTACGTGCTGTCTTCTCGGGTGCGCACTGGCCGCAGCATCCGTGGGCTGAGCCTGC
CTCCAGCCTGCACCCGGGCCGAGCGAAGGGAGGTAGAGAACGTGGCCATCACTGCCCTGGAGGGCCTCAAGGGG
GACCTGGCTGGCCGCTACTACAAGCTGTCCGAGATGACGGAGCAGGACCAGCAGCGGCTCATCGATGACCACTTT
CTGTTTGATAAGCCAGTGTCCCCTTTATTAACATGTGCTGGGATGGCCCGTGACTGGCCAGATGCCAGGGGAATCT
GGCATAATTATGATAAGACATTTCTCATCTGGATAAATGAGGAGGATCACACCAGGGTAATCTCAATGGAAAAAG
GAGGCAATATGAAACGAGTATTTGAGCGATTCTGTCGTGGACTAAAAGAAGTAGAACGGTTAATCCAAGAACGA
GGCTGGGAGTTCATGTGGAATGAGCGCCTAGGATACATTTTGACCTGTCCTTCGAACCTTGGAACAGGACTACGA
GCTGGTGTCCACGTTAGGATCCCAAAGCTCAGCAAGGACCCACGCTTTTCTAAGATCCTGGAAAACCTAAGACTCC
AGAAGCGTGGCACAGGTGGTGTGGACACTGCCGCGGTCGCAGATGTGTACGACATTTCCAACATAGATAGAATTG
GTCGATCAGAGGTTGAGCTTGTTCAGATAGTCATCGATGGAGTCAATTACCTGGTGGATTGTGAAAAGAAGTTGG
AGAGAGGCCAAGATATTAAGGTGCCACCCCCTCTGCCTCAGTTTGGCAAAAAGTAA
NP_001093206.1 (SEQ ID NO: 54)
MASIFSKLLTGRNASLLFATMGTSVLTTGYLLNRQKVCAEVREQPRLFPPSADYPDLRKHNNCMAECLTPAIYAKLRNKV
TPNGYTLDQCIQTGVDNPGHPFIKTVGMVAGDEESYEVFADLFDPVIKLRHNGYDPRVMKHTTDLDASKITQGQFDEH
YVLSSRVRTGRSIRGLSLPPACTRAERREVENVAITALEGLKGDLAGRYYKLSEMTEQDQQRLIDDHFLFDKPVSPLLTCA
GMARDWPDARGIWHNYDKTFLIWINEEDHTRVISMEKGGNMKRVFERFCRGLKEVERLIQERGWEFMWNERLGYIL
TCPSNLGTGLRAGVHVRIPKLSKDPRFSKILENLRLQKRGTGGVDTAAVADVYDISNIDRIGRSEVELVQIVIDGVNYLVD
CEKKLERGQDIKVPPPLPQFGKK
TSHZ2
V1: NM_173485.6 (SEQ ID NO: 55)
ATGCCGAGGAGAAAACAGCAGGCACCCAAGCGGGCGGCAGGCTACGCCCAGGAGGAACAGCTGAAAGAAGAG
GAGGAAATAAAAGAAGAGGAGGAGGAGGAGGACAGCGGTTCAGTAGCTCAACTGCAGGGTGGCAATGACACAG
GGACGGACGAGGAGCTAGAAACGGGCCCAGAGCAAAAAGGCTGCTTCAGCTACCAGAACTCTCCAGGAAGTCAT
TTGTCCAATCAGGATGCCGAGAACGAGTCTCTGCTGAGTGACGCCAGTGATCAGGTGTCGGACATCAAGAGTGTC
TGCGGCAGAGATGCCTCAGACAAGAAAGCACACACTCACGTCAGGCTTCCAAACGAAGCACACAATTGCATGGAT
AAAATGACCGCTGTCTACGCCAACATCCTGTCGGATTCCTACTGGTCAGGCCTGGGCCTTGGCTTCAAGCTGTCCA
ATAGTGAGAGGAGGAACTGTGACACCCGAAACGGCAGCAACAAGAGTGATTTTGATTGGCACCAAGACGCTCTG
TCCAAAAGCCTGCAGCAGAACTTGCCTTCTCGGTCCGTCTCGAAACCCAGCCTGTTCAGCTCGGTGCAGTTGTACC
GACAGAGCAGCAAGATGTGCGGGACTGTGTTCACAGGGGCCAGCAGATTCCGATGCCGACAGTGCAGCGCGGCC
TATGACACCCTAGTCGAGCTGACTGTGCACATGAATGAAACGGGCCACTATCAAGATGACAACCGCAAAAAGGAC
AAGCTCAGACCCACGAGCTATTCAAAGCCCAGGAAAAGGGCTTTCCAGGATATGGACAAAGAGGATGCTCAAAA
GGTTCTGAAATGTATGTTTTGTGGCGACTCCTTTGATTCCCTCCAAGATTTGAGCGTCCACATGATTAAAACAAAAC
ATTACCAAAAAGTGCCTTTGAAGGAGCCAGTCCCAACCATTTCCTCGAAAATGGTCACCCCGGCTAAGAAACGCGT
TTTTGATGTCAATCGGCCGTGTTCCCCCGATTCAACCACAGGATCTTTTGCAGATTCTTTTTCTTCTCAGAAGAACGC
CAACTTGCAGTTGTCCTCCAACAACCGCTATGGCTACCAAAATGGAGCCAGCTACACCTGGCAGTTTGAGGCCTGC
AAGTCCCAGATCTTAAAGTGCATGGAGTGTGGGAGCTCCCATGACACCTTGCAGCAGCTCACCACCCACATGATG
GTCACAGGTCACTTTCTCAAGGTCACCAGCTCTGCCTCCAAGAAAGGGAAGCAGCTGGTATTAGACCCGTTAGCA
GTGGAGAAAATGCAGTCGTTGTCTGAGGCCCCAAACAGTGATTCTCTGGCTCCCAAGCCATCCAGTAACTCAGCAT
CAGATTGTACAGCCTCTACAACTGAGTTAAAGAAAGAGAGTAAAAAAGAAAGGCCAGAGGAAACCAGCAAGGAT
GAGAAAGTCGTGAAAAGCGAGGACTATGAAGATCCTCTACAAAAACCTTTAGACCCTACAATCAAATATCAATACC
TAAGGGAGGAAGACTTGGAAGATGGCTCAAAGGGTGGAGGGGACATTTTGAAATCTTTGGAAAATACTGTCACC
ACAGCCATCAACAAAGCCCAAAACGGGGCCCCCAGCTGGAGTGCCTACCCCAGCATCCACGCAGCCTACCAGCTG
TCTGAGGGCACCAAGCCGCCTTTGCCTATGGGATCCCAGGTACTGCAGATCCGGCCTAATCTCACCAACAAGCTGA
GGCCCATTGCACCAAAGTGGAAAGTGATGCCACTGGTTTCTATGCCCACACACCTGGCCCCTTACACTCAAGTCAA
GAAAGAGTCAGAAGACAAAGATGAAGCGGTGAAGGAGTGTGGGAAAGAAAGTCCCCACGAAGAGGCCTCATCT
TTCAGCCACAGTGAGGGCGATTCTTTCCGCAAAAGTGAAACACCTCCAGAAGCCAAAAAGACCGAGCTGGGTCCC
CTGAAGGAGGAGGAGAAGCTGATGAAAGAGGGCAGCGAGAAGGAGAAACCCCAGCCCCTGGAGCCCACATCTG
CTCTGAGCAATGGGTGCGCCCTCGCCAACCACGCCCCGGCCCTGCCATGCATCAACCCACTCAGCGCCCTGCAGTC
CGTCCTGAACAATCACTTGGGCAAAGCCACGGAGCCCTTGCGCTCACCTTCCTGCTCCAGCCCAAGTTCAAGCACA
ATTTCCATGTTCCACAAGTCGAATCTCAATGTCATGGACAAGCCGGTCTTGAGTCCTGCCTCCACAAGGTCAGCCA
GCGTGTCCAGGCGCTACCTGTTTGAGAACAGCGATCAGCCCATTGACCTGACCAAGTCCAAAAGCAAGAAAGCCG
AGTCCTCGCAAGCACAATCTTGTATGTCCCCACCTCAGAAGCACGCTCTGTCTGACATCGCCGACATGGTCAAAGT
CCTCCCCAAAGCCACCACCCCAAAGCCAGCCTCCTCCTCCAGGGTCCCCCCCATGAAGCTGGAAATGGATGTCAGG
CGCTTTGAGGATGTCTCCAGTGAAGTCTCAACTTTGCATAAAAGAAAAGGCCGGCAGTCCAACTGGAATCCTCAGC
ATCTTCTGATTCTACAAGCCCAGTTTGCCTCGAGCCTCTTCCAGACATCAGAGGGCAAATACCTGCTGTCTGATCTG
GGCCCACAAGAGCGTATGCAAATCTCTAAGTTTACGGGACTCTCAATGACCACTATCAGTCACTGGCTGGCCAACG
TCAAGTACCAGCTTAGGAAAACGGGCGGGACAAAATTTCTGAAAAACATGGACAAAGGCCACCCCATCTTTTATT
GCAGTGACTGTGCCTCCCAGTTCAGAACCCCTTCTACCTACATCAGTCACTTAGAATCTCACCTGGGTTTCCAAATG
AAGGACATGACCCGCTTGTCAGTGGACCAGCAAAGCAAGGTGGAGCAAGAGATCTCCCGGGTATCGTCGGCTCA
GAGGTCTCCAGAAACAATAGCTGCCGAAGAGGACACAGACTCTAAATTCAAGTGTAAGTTGTGCTGTCGGACATT
TGTGAGCAAACATGCGGTAAAACTCCACCTAAGCAAAACGCACAGCAAGTCACCCGAACACCATTCACAGTTTGTA
ACAGACGTGGATGAAGAATAG
I1: NP_775756.3 (SEQ ID NO: 56)
MPRRKQQAPKRAAGYAQEEQLKEEEEIKEEEEEEDSGSVAQLQGGNDTGTDEELETGPEQKGCFSYQNSPGSHLSNQ
DAENESLLSDASDQVSDIKSVCGRDASDKKAHTHVRLPNEAHNCMDKMTAVYANILSDSYWSGLGLGFKLSNSERRNC
DTRNGSNKSDFDWHQDALSKSLQQNLPSRSVSKPSLFSSVQLYRQSSKMCGTVFTGASRFRCRQCSAAYDTLVELTVH
MNETGHYQDDNRKKDKLRPTSYSKPRKRAFQDMDKEDAQKVLKCMFCGDSFDSLQDLSVHMIKTKHYQKVPLKEPV
PTISSKMVTPAKKRVFDVNRPCSPDSTTGSFADSFSSQKNANLQLSSNNRYGYQNGASYTWQFEACKSQILKCMECGS
SHDTLQQLTTHMMVTGHFLKVTSSASKKGKQLVLDPLAVEKMQSLSEAPNSDSLAPKPSSNSASDCTASTTELKKESKK
ERPEETSKDEKVVKSEDYEDPLQKPLDPTIKYQYLREEDLEDGSKGGGDILKSLENTVTTAINKAQNGAPSWSAYPSIHAA
YQLSEGTKPPLPMGSQVLQIRPNLTNKLRPIAPKWKVMPLVSMPTHLAPYTQVKKESEDKDEAVKECGKESPHEEASSF
SHSEGDSFRKSETPPEAKKTELGPLKEEEKLMKEGSEKEKPQPLEPTSALSNGCALANHAPALPCINPLSALQSVLNNHLG
KATEPLRSPSCSSPSSSTISMFHKSNLNVMDKPVLSPASTRSASVSRRYLFENSDQPIDLTKSKSKKAESSQAQSCMSPPQ
KHALSDIADMVKVLPKATTPKPASSSRVPPMKLEMDVRRFEDVSSEVSTLHKRKGRQSNWNPQHLLILQAQFASSLFQ
TSEGKYLLSDLGPQERMQISKFTGLSMTTISHWLANVKYQLRKTGGTKFLKNMDKGHPIFYCSDCASQFRTPSTYISHLE
SHLGFQMKDMTRLSVDQQSKVEQEISRVSSAQRSPETIAAEEDTDSKFKCKLCCRTFVSKHAVKLHLSKTHSKSPEHHSQ
FVTDVDEE
V2: NM_001193421.2 (SEQ ID NO: 57)
ATGATGGCTGCTGCGTTGCTCCATTATACAGGCTACGCCCAGGAGGAACAGCTGAAAGAAGAGGAGGAAATAAA
AGAAGAGGAGGAGGAGGAGGACAGCGGTTCAGTAGCTCAACTGCAGGGTGGCAATGACACAGGGACGGACGA
GGAGCTAGAAACGGGCCCAGAGCAAAAAGGCTGCTTCAGCTACCAGAACTCTCCAGGAAGTCATTTGTCCAATCA
GGATGCCGAGAACGAGTCTCTGCTGAGTGACGCCAGTGATCAGGTGTCGGACATCAAGAGTGTCTGCGGCAGAG
ATGCCTCAGACAAGAAAGCACACACTCACGTCAGGCTTCCAAACGAAGCACACAATTGCATGGATAAAATGACCG
CTGTCTACGCCAACATCCTGTCGGATTCCTACTGGTCAGGCCTGGGCCTTGGCTTCAAGCTGTCCAATAGTGAGAG
GAGGAACTGTGACACCCGAAACGGCAGCAACAAGAGTGATTTTGATTGGCACCAAGACGCTCTGTCCAAAAGCCT
GCAGCAGAACTTGCCTTCTCGGTCCGTCTCGAAACCCAGCCTGTTCAGCTCGGTGCAGTTGTACCGACAGAGCAGC
AAGATGTGCGGGACTGTGTTCACAGGGGCCAGCAGATTCCGATGCCGACAGTGCAGCGCGGCCTATGACACCCTA
GTCGAGCTGACTGTGCACATGAATGAAACGGGCCACTATCAAGATGACAACCGCAAAAAGGACAAGCTCAGACCC
ACGAGCTATTCAAAGCCCAGGAAAAGGGCTTTCCAGGATATGGACAAAGAGGATGCTCAAAAGGTTCTGAAATGT
ATGTTTTGTGGCGACTCCTTTGATTCCCTCCAAGATTTGAGCGTCCACATGATTAAAACAAAACATTACCAAAAAGT
GCCTTTGAAGGAGCCAGTCCCAACCATTTCCTCGAAAATGGTCACCCCGGCTAAGAAACGCGTTTTTGATGTCAAT
CGGCCGTGTTCCCCCGATTCAACCACAGGATCTTTTGCAGATTCTTTTTCTTCTCAGAAGAACGCCAACTTGCAGTT
GTCCTCCAACAACCGCTATGGCTACCAAAATGGAGCCAGCTACACCTGGCAGTTTGAGGCCTGCAAGTCCCAGATC
TTAAAGTGCATGGAGTGTGGGAGCTCCCATGACACCTTGCAGCAGCTCACCACCCACATGATGGTCACAGGTCACT
TTCTCAAGGTCACCAGCTCTGCCTCCAAGAAAGGGAAGCAGCTGGTATTAGACCCGTTAGCAGTGGAGAAAATGC
AGTCGTTGTCTGAGGCCCCAAACAGTGATTCTCTGGCTCCCAAGCCATCCAGTAACTCAGCATCAGATTGTACAGC
CTCTACAACTGAGTTAAAGAAAGAGAGTAAAAAAGAAAGGCCAGAGGAAACCAGCAAGGATGAGAAAGTCGTG
AAAAGCGAGGACTATGAAGATCCTCTACAAAAACCTTTAGACCCTACAATCAAATATCAATACCTAAGGGAGGAA
GACTTGGAAGATGGCTCAAAGGGTGGAGGGGACATTTTGAAATCTTTGGAAAATACTGTCACCACAGCCATCAAC
AAAGCCCAAAACGGGGCCCCCAGCTGGAGTGCCTACCCCAGCATCCACGCAGCCTACCAGCTGTCTGAGGGCACC
AAGCCGCCTTTGCCTATGGGATCCCAGGTACTGCAGATCCGGCCTAATCTCACCAACAAGCTGAGGCCCATTGCAC
CAAAGTGGAAAGTGATGCCACTGGTTTCTATGCCCACACACCTGGCCCCTTACACTCAAGTCAAGAAAGAGTCAGA
AGACAAAGATGAAGCGGTGAAGGAGTGTGGGAAAGAAAGTCCCCACGAAGAGGCCTCATCTTTCAGCCACAGTG
AGGGCGATTCTTTCCGCAAAAGTGAAACACCTCCAGAAGCCAAAAAGACCGAGCTGGGTCCCCTGAAGGAGGAG
GAGAAGCTGATGAAAGAGGGCAGCGAGAAGGAGAAACCCCAGCCCCTGGAGCCCACATCTGCTCTGAGCAATGG
GTGCGCCCTCGCCAACCACGCCCCGGCCCTGCCATGCATCAACCCACTCAGCGCCCTGCAGTCCGTCCTGAACAAT
CACTTGGGCAAAGCCACGGAGCCCTTGCGCTCACCTTCCTGCTCCAGCCCAAGTTCAAGCACAATTTCCATGTTCCA
CAAGTCGAATCTCAATGTCATGGACAAGCCGGTCTTGAGTCCTGCCTCCACAAGGTCAGCCAGCGTGTCCAGGCG
CTACCTGTTTGAGAACAGCGATCAGCCCATTGACCTGACCAAGTCCAAAAGCAAGAAAGCCGAGTCCTCGCAAGC
ACAATCTTGTATGTCCCCACCTCAGAAGCACGCTCTGTCTGACATCGCCGACATGGTCAAAGTCCTCCCCAAAGCCA
CCACCCCAAAGCCAGCCTCCTCCTCCAGGGTCCCCCCCATGAAGCTGGAAATGGATGTCAGGCGCTTTGAGGATGT
CTCCAGTGAAGTCTCAACTTTGCATAAAAGAAAAGGCCGGCAGTCCAACTGGAATCCTCAGCATCTTCTGATTCTA
CAAGCCCAGTTTGCCTCGAGCCTCTTCCAGACATCAGAGGGCAAATACCTGCTGTCTGATCTGGGCCCACAAGAGC
GTATGCAAATCTCTAAGTTTACGGGACTCTCAATGACCACTATCAGTCACTGGCTGGCCAACGTCAAGTACCAGCT
TAGGAAAACGGGCGGGACAAAATTTCTGAAAAACATGGACAAAGGCCACCCCATCTTTTATTGCAGTGACTGTGC
CTCCCAGTTCAGAACCCCTTCTACCTACATCAGTCACTTAGAATCTCACCTGGGTTTCCAAATGAAGGACATGACCC
GCTTGTCAGTGGACCAGCAAAGCAAGGTGGAGCAAGAGATCTCCCGGGTATCGTCGGCTCAGAGGTCTCCAGAA
ACAATAGCTGCCGAAGAGGACACAGACTCTAAATTCAAGTGTAAGTTGTGCTGTCGGACATTTGTGAGCAAACAT
GCGGTAAAACTCCACCTAAGCAAAACGCACAGCAAGTCACCCGAACACCATTCACAGTTTGTAACAGACGTGGAT
GAAGAATAG
I2: NP_001180350.1 (SEQ ID NO: 58)
MMAAALLHYTGYAQEEQLKEEEEIKEEEEEEDSGSVAQLQGGNDTGTDEELETGPEQKGCFSYQNSPGSHLSNQDAE
NESLLSDASDQVSDIKSVCGRDASDKKAHTHVRLPNEAHNCMDKMTAVYANILSDSYWSGLGLGFKLSNSERRNCDTR
NGSNKSDFDWHQDALSKSLQQNLPSRSVSKPSLFSSVQLYRQSSKMCGTVFTGASRFRCRQCSAAYDTLVELTVHMNE
TGHYQDDNRKKDKLRPTSYSKPRKRAFQDMDKEDAQKVLKCMFCGDSFDSLQDLSVHMIKTKHYQKVPLKEPVPTISS
KMVTPAKKRVFDVNRPCSPDSTTGSFADSFSSQKNANLQLSSNNRYGYQNGASYTWQFEACKSQILKCMECGSSHDT
LQQLTTHMMVTGHFLKVTSSASKKGKQLVLDPLAVEKMQSLSEAPNSDSLAPKPSSNSASDCTASTTELKKESKKERPEE
TSKDEKVVKSEDYEDPLQKPLDPTIKYQYLREEDLEDGSKGGGDILKSLENTVTTAINKAQNGAPSWSAYPSIHAAYQLSE
GTKPPLPMGSQVLQIRPNLTNKLRPIAPKWKVMPLVSMPTHLAPYTQVKKESEDKDEAVKECGKESPHEEASSFSHSEG
DSFRKSETPPEAKKTELGPLKEEEKLMKEGSEKEKPQPLEPTSALSNGCALANHAPALPCINPLSALQSVLNNHLGKATEP
LRSPSCSSPSSSTISMFHKSNLNVMDKPVLSPASTRSASVSRRYLFENSDQPIDLTKSKSKKAESSQAQSCMSPPQKHALS
DIADMVKVLPKATTPKPASSSRVPPMKLEMDVRRFEDVSSEVSTLHKRKGRQSNWNPQHLLILQAQFASSLFQTSEGK
YLLSDLGPQERMQISKFTGLSMTTISHWLANVKYQLRKTGGTKFLKNMDKGHPIFYCSDCASQFRTPSTYISHLESHLGF
QMKDMTRLSVDQQSKVEQEISRVSSAQRSPETIAAEEDTDSKFKCKLCCRTFVSKHAVKLHLSKTHSKSPEHHSQFVTD
VDEE
MITF
NM_198159.3 (SEQ ID NO: 59)
ATGCAGTCCGAATCGGGGATCGTGCCGGATTTCGAAGTCGGGGAGGAGTTTCATGAAGAGCCCAAAACCTATTAC
GAACTCAAAAGTCAACCGCTGAAGAGCAGCAGTTCCGCCGAGCATCCTGGGGCCTCCAAGCCTCCGATAAGCTCC
TCCAGTATGACATCACGCATCTTGCTACGCCAGCAACTCATGCGTGAGCAGATGCAGGAGCAGGAGCGCAGGGA
GCAGCAGCAGAAGCTGCAGGCGGCCCAGTTCATGCAACAGAGAGTGCCCGTGAGTCAGACACCAGCCATAAACG
TCAGTGTGCCCACCACCCTTCCCTCTGCCACGCAGGTGCCGATGGAAGTCCTTAAGGTGCAGACCCACCTCGAAAA
CCCCACCAAGTACCACATACAGCAAGCCCAACGGCAGCAGGTAAAGCAGTACCTTTCTACCACTTTAGCAAATAAA
CATGCCAACCAAGTCCTGAGCTTGCCATGTCCAAACCAGCCTGGCGATCATGTCATGCCACCGGTGCCGGGGAGC
AGCGCACCCAACAGCCCCATGGCTATGCTTACGCTTAACTCCAACTGTGAAAAAGAGGGATTTTATAAGTTTGAAG
AGCAAAACAGGGCAGAGAGCGAGTGCCCAGGCATGAACACACATTCACGAGCGTCCTGTATGCAGATGGATGAT
GTAATCGATGACATCATTAGCCTAGAATCAAGTTATAATGAGGAAATCTTGGGCTTGATGGATCCTGCTTTGCAAA
TGGCAAATACGTTGCCTGTCTCGGGAAACTTGATTGATCTTTATGGAAACCAAGGTCTGCCCCCACCAGGCCTCAC
CATCAGCAACTCCTGTCCAGCCAACCTTCCCAACATAAAAAGGGAGCTCACAGAGTCTGAAGCAAGAGCACTGGC
CAAAGAGAGGCAGAAAAAGGACAATCACAACCTGATTGAACGAAGAAGAAGATTTAACATAAATGACCGCATTA
AAGAACTAGGTACTTTGATTCCCAAGTCAAATGATCCAGACATGCGCTGGAACAAGGGAACCATCTTAAAAGCATC
CGTGGACTATATCCGAAAGTTGCAACGAGAACAGCAACGCGCAAAAGAACTTGAAAACCGACAGAAGAAACTGG
AGCACGCCAACCGGCATTTGTTGCTCAGAATACAGGAACTTGAAATGCAGGCTCGAGCTCATGGACTTTCCCTTAT
TCCATCCACGGGTCTCTGCTCTCCAGATTTGGTGAATCGGATCATCAAGCAAGAACCCGTTCTTGAGAACTGCAGC
CAAGACCTCCTTCAGCATCATGCAGACCTAACCTGTACAACAACTCTCGATCTCACGGATGGCACCATCACCTTCAA
CAACAACCTCGGAACTGGGACTGAGGCCAACCAAGCCTATAGTGTCCCCACAAAAATGGGATCCAAACTGGAAGA
CATCCTGATGGACGACACCCTTTCTCCCGTCGGTGTCACTGATCCACTCCTTTCCTCAGTGTCCCCCGGAGCTTCCAA
AACAAGCAGCCGGAGGAGCAGTATGAGCATGGAAGAGACGGAGCACACTTGTTAG
NP_937802.1 (SEQ ID NO: 60)
MQSESGIVPDFEVGEEFHEEPKTYYELKSQPLKSSSSAEHPGASKPPISSSSMTSRILLRQQLMREQMQEQERREQQQK
LQAAQFMQQRVPVSQTPAINVSVPTTLPSATQVPMEVLKVQTHLENPTKYHIQQAQRQQVKQYLSTTLANKHANQV
LSLPCPNQPGDHVMPPVPGSSAPNSPMAMLTLNSNCEKEGFYKFEEQNRAESECPGMNTHSRASCMQMDDVIDDII
SLESSYNEEILGLMDPALQMANTLPVSGNLIDLYGNQGLPPPGLTISNSCPANLPNIKRELTESEARALAKERQKKDNHN
LIERRRRFNINDRIKELGTLIPKSNDPDMRWNKGTILKASVDYIRKLQREQQRAKELENRQKKLEHANRHLLLRIQELEM
QARAHGLSLIPSTGLCSPDLVNRIIKQEPVLENCSQDLLQHHADLTCTTTLDLTDGTITFNNNLGTGTEANQAYSVPTKM
GSKLEDILMDDTLSPVGVTDPLLSSVSPGASKTSSRRSSMSMEETEHTC
MYOCD
V1: NM_001146312.3 (SEQ ID NO: 61)
ATGACACTCCTGGGGTCTGAGCATTCCTTGCTGATTAGGAGCAAGTTCAGATCAGTTTTACAGTTAAGACTTCAAC
AAAGAAGGACCCAGGAACAACTGGCTAACCAAGGCATAATACCACCACTGAAACGTCCAGCTGAATTCCATGAGC
AAAGAAAACATTTGGATAGTGACAAGGCTAAAAATTCCCTGAAGCGCAAAGCCAGAAACAGGTGCAACAGTGCC
GACTTGGTTAATATGCACATACTCCAAGCTTCCACTGCAGAGAGGTCCATTCCAACTGCTCAGATGAAGCTGAAAA
GAGCCCGACTCGCCGATGATCTCAATGAAAAAATTGCTCTACGACCAGGGCCACTGGAGCTGGTGGAAAAAAACA
TTCTTCCTGTGGATTCTGCTGTGAAAGAGGCCATAAAAGGTAACCAGGTGAGTTTCTCCAAATCCACGGATGCTTT
TGCCTTTGAAGAGGACAGCAGCAGCGATGGGCTTTCTCCGGATCAGACTCGAAGTGAAGACCCCCAAAACTCAGC
GGGATCCCCGCCAGACGCTAAAGCCTCAGATACCCCTTCGACAGGTTCTCTGGGGACAAACCAGGATCTTGCTTCT
GGCTCAGAAAATGACAGAAATGACTCAGCCTCACAGCCCAGCCACCAGTCAGATGCGGGGAAGCAGGGGCTTGG
CCCCCCCAGCACCCCCATAGCCGTGCATGCTGCTGTAAAGTCCAAATCCTTGGGTGACAGTAAGAACCGCCACAAA
AAGCCCAAGGACCCCAAGCCAAAGGTGAAGAAGCTTAAATATCACCAGTACATTCCCCCAGACCAGAAGGCAGAG
AAGTCCCCTCCACCTATGGACTCAGCCTACGCTCGGCTGCTCCAGCAACAGCAGCTGTTCCTGCAGCTCCAAATCCT
CAGCCAGCAGCAGCAGCAGCAGCAACACCGATTCAGCTACCTAGGGATGCACCAAGCTCAGCTTAAGGAACCAAA
TGAACAGATGGTCAGAAATCCAAACTCTTCTTCAACGCCACTGAGCAATACCCCCTTGTCTCCTGTCAAAAACAGTT
TTTCTGGACAAACTGGTGTCTCTTCTTTCAAACCAGGCCCACTCCCACCTAACCTGGATGATCTGAAGGTCTCTGAA
TTAAGACAACAGCTTCGAATTCGGGGCTTGCCTGTGTCAGGCACCAAAACGGCTCTCATGGACCGGCTTCGACCCT
TCCAGGACTGCTCTGGCAACCCAGTGCCGAACTTTGGGGATATAACGACTGTCACTTTTCCTGTCACACCCAACAC
GCTGCCCAATTACCAGTCTTCCTCTTCTACCAGTGCCCTGTCCAACGGCTTCTACCACTTTGGCAGCACCAGCTCCA
GCCCCCCGATCTCCCCAGCCTCCTCTGACCTGTCAGTCGCTGGGTCCCTGCCGGACACCTTCAATGATGCCTCCCCC
TCCTTCGGCCTGCACCCGTCCCCAGTCCACGTGTGCACGGAGGAAAGTCTCATGAGCAGCCTGAATGGGGGCTCT
GTTCCTTCTGAGCTGGATGGGCTGGACTCCGAGAAGGACAAGATGCTGGTGGAGAAGCAGAAGGTGATCAATGA
ACTCACCTGGAAACTCCAGCAAGAGCAGAGGCAGGTGGAGGAGCTGAGGATGCAGCTTCAGAAGCAGAAAAGG
AATAACTGTTCAGAGAAGAAGCCGCTGCCTTTCCTGGCTGCCTCCATCAAGCAGGAAGAGGCTGTCTCCAGCTGTC
CTTTTGCATCCCAAGTACCTGTGAAAAGACAAAGCAGCAGCTCAGAGTGTCACCCACCGGCTTGTGAAGCTGCTCA
ACTCCAGCCTCTTGGAAATGCTCATTGTGTGGAGTCCTCAGATCAAACCAATGTACTTTCTTCCACATTTCTCAGCCC
CCAGTGTTCCCCTCAGCATTCACCGCTGGGGGCTGTGAAAAGCCCACAGCACATCAGTTTGCCCCCATCACCCAAC
AACCCTCACTTTCTGCCCTCATCCTCCGGGGCCCAGGGAGAAGGGCACAGGGTCTCCTCGCCCATCAGCAGCCAG
GTGTGCACTGCACAGAACTCAGGAGCACACGATGGCCATCCTCCAAGCTTCTCTCCCCATTCTTCCAGCCTCCACCC
GCCCTTCTCTGGAGCCCAAGCAGACAGCAGTCATGGTGCCGGGGGAAACCCTTGTCCCAAAAGCCCATGTGTACA
GCAAAAGATGGCTGGTTTACACTCTTCTGATAAGGTGGGGCCAAAGTTTTCAATTCCATCCCCAACTTTTTCTAAGT
CAAGTTCAGCAATTTCAGAGGTAACACAGCCTCCATCCTATGAAGATGCCGTAAAGCAGCAAATGACCCGGAGTC
AGCAGATGGATGAACTCCTGGACGTGCTTATTGAAAGCGGAGAAATGCCAGCAGACGCTAGAGAGGATCACTCA
TGTCTTCAAAAAGTCCCAAAGATACCCAGATCTTCCCGAAGTCCAACTGCTGTCCTCACCAAGCCCTCGGCTTCCTT
TGAACAAGCCTCTTCAGGCAGCCAGATCCCCTTTGATCCCTATGCCACCGACAGTGATGAGCATCTTGAAGTCTTAT
TAAATTCCCAGAGCCCCCTAGGAAAGATGAGTGATGTCACCCTTCTAAAAATTGGGAGCGAAGAGCCTCACTTTGA
TGGGATAATGGATGGATTCTCTGGGAAGGCTGCAGAAGACCTCTTCAATGCACATGAGATCTTGCCAGGCCCCCT
CTCTCCAATGCAGACACAGTTTTCACCCTCTTCTGTGGACAGCAATGGGCTGCAGTTAAGCTTCACTGAATCTCCCT
GGGAAACCATGGAGTGGCTGGACCTCACTCCGCCAAATTCCACACCAGGCTTTAGCGCCCTCACCACCAGCAGCCC
CAGCATCTTCAACATCGATTTCCTGGATGTCACTGATCTCAATTTGAATTCTTCCATGGACCTTCACTTGCAGCAGTG
GTAG
I1: NP_001139784.1 (SEQ ID NO: 62)
MTLLGSEHSLLIRSKFRSVLQLRLQQRRTQEQLANQGIIPPLKRPAEFHEQRKHLDSDKAKNSLKRKARNRCNSADLVN
MHILQASTAERSIPTAQMKLKRARLADDLNEKIALRPGPLELVEKNILPVDSAVKEAIKGNQVSFSKSTDAFAFEEDSSSD
GLSPDQTRSEDPQNSAGSPPDAKASDTPSTGSLGTNQDLASGSENDRNDSASQPSHQSDAGKQGLGPPSTPIAVHAA
VKSKSLGDSKNRHKKPKDPKPKVKKLKYHQYIPPDQKAEKSPPPMDSAYARLLQQQQLFLQLQILSQQQQQQQHRFSY
LGMHQAQLKEPNEQMVRNPNSSSTPLSNTPLSPVKNSFSGQTGVSSFKPGPLPPNLDDLKVSELRQQLRIRGLPVSGTK
TALMDRLRPFQDCSGNPVPNFGDITTVTFPVTPNTLPNYQSSSSTSALSNGFYHFGSTSSSPPISPASSDLSVAGSLPDTF
NDASPSFGLHPSPVHVCTEESLMSSLNGGSVPSELDGLDSEKDKMLVEKQKVINELTWKLQQEQRQVEELRMQLQKQ
KRNNCSEKKPLPFLAASIKQEEAVSSCPFASQVPVKRQSSSSECHPPACEAAQLQPLGNAHCVESSDQTNVLSSTFLSPQ
CSPQHSPLGAVKSPQHISLPPSPNNPHFLPSSSGAQGEGHRVSSPISSQVCTAQNSGAHDGHPPSFSPHSSSLHPPFSGA
QADSSHGAGGNPCPKSPCVQQKMAGLHSSDKVGPKFSIPSPTFSKSSSAISEVTQPPSYEDAVKQQMTRSQQMDELL
DVLIESGEMPADAREDHSCLQKVPKIPRSSRSPTAVLTKPSASFEQASSGSQIPFDPYATDSDEHLEVLLNSQSPLGKMSD
VTLLKIGSEEPHFDGIMDGFSGKAAEDLFNAHEILPGPLSPMQTQFSPSSVDSNGLQLSFTESPWETMEWLDLTPPNST
PGFSALTTSSPSIFNIDFLDVTDLNLNSSMDLHLQQW
V2: NM_153604.4 (SEQ ID NO: 63)
ATGACACTCCTGGGGTCTGAGCATTCCTTGCTGATTAGGAGCAAGTTCAGATCAGTTTTACAGTTAAGACTTCAAC
AAAGAAGGACCCAGGAACAACTGGCTAACCAAGGCATAATACCACCACTGAAACGTCCAGCTGAATTCCATGAGC
AAAGAAAACATTTGGATAGTGACAAGGCTAAAAATTCCCTGAAGCGCAAAGCCAGAAACAGGTGCAACAGTGCC
GACTTGGTTAATATGCACATACTCCAAGCTTCCACTGCAGAGAGGTCCATTCCAACTGCTCAGATGAAGCTGAAAA
GAGCCCGACTCGCCGATGATCTCAATGAAAAAATTGCTCTACGACCAGGGCCACTGGAGCTGGTGGAAAAAAACA
TTCTTCCTGTGGATTCTGCTGTGAAAGAGGCCATAAAAGGTAACCAGGTGAGTTTCTCCAAATCCACGGATGCTTT
TGCCTTTGAAGAGGACAGCAGCAGCGATGGGCTTTCTCCGGATCAGACTCGAAGTGAAGACCCCCAAAACTCAGC
GGGATCCCCGCCAGACGCTAAAGCCTCAGATACCCCTTCGACAGGTTCTCTGGGGACAAACCAGGATCTTGCTTCT
GGCTCAGAAAATGACAGAAATGACTCAGCCTCACAGCCCAGCCACCAGTCAGATGCGGGGAAGCAGGGGCTTGG
CCCCCCCAGCACCCCCATAGCCGTGCATGCTGCTGTAAAGTCCAAATCCTTGGGTGACAGTAAGAACCGCCACAAA
AAGCCCAAGGACCCCAAGCCAAAGGTGAAGAAGCTTAAATATCACCAGTACATTCCCCCAGACCAGAAGGCAGAG
AAGTCCCCTCCACCTATGGACTCAGCCTACGCTCGGCTGCTCCAGCAACAGCAGCTGTTCCTGCAGCTCCAAATCCT
CAGCCAGCAGCAGCAGCAGCAGCAACACCGATTCAGCTACCTAGGGATGCACCAAGCTCAGCTTAAGGAACCAAA
TGAACAGATGGTCAGAAATCCAAACTCTTCTTCAACGCCACTGAGCAATACCCCCTTGTCTCCTGTCAAAAACAGTT
TTTCTGGACAAACTGGTGTCTCTTCTTTCAAACCAGGCCCACTCCCACCTAACCTGGATGATCTGAAGGTCTCTGAA
TTAAGACAACAGCTTCGAATTCGGGGCTTGCCTGTGTCAGGCACCAAAACGGCTCTCATGGACCGGCTTCGACCCT
TCCAGGACTGCTCTGGCAACCCAGTGCCGAACTTTGGGGATATAACGACTGTCACTTTTCCTGTCACACCCAACAC
GCTGCCCAATTACCAGTCTTCCTCTTCTACCAGTGCCCTGTCCAACGGCTTCTACCACTTTGGCAGCACCAGCTCCA
GCCCCCCGATCTCCCCAGCCTCCTCTGACCTGTCAGTCGCTGGGTCCCTGCCGGACACCTTCAATGATGCCTCCCCC
TCCTTCGGCCTGCACCCGTCCCCAGTCCACGTGTGCACGGAGGAAAGTCTCATGAGCAGCCTGAATGGGGGCTCT
GTTCCTTCTGAGCTGGATGGGCTGGACTCCGAGAAGGACAAGATGCTGGTGGAGAAGCAGAAGGTGATCAATGA
ACTCACCTGGAAACTCCAGCAAGAGCAGAGGCAGGTGGAGGAGCTGAGGATGCAGCTTCAGAAGCAGAAAAGG
AATAACTGTTCAGAGAAGAAGCCGCTGCCTTTCCTGGCTGCCTCCATCAAGCAGGAAGAGGCTGTCTCCAGCTGTC
CTTTTGCATCCCAAGTACCTGTGAAAAGACAAAGCAGCAGCTCAGAGTGTCACCCACCGGCTTGTGAAGCTGCTCA
ACTCCAGCCTCTTGGAAATGCTCATTGTGTGGAGTCCTCAGATCAAACCAATGTACTTTCTTCCACATTTCTCAGCCC
CCAGTGTTCCCCTCAGCATTCACCGCTGGGGGCTGTGAAAAGCCCACAGCACATCAGTTTGCCCCCATCACCCAAC
AACCCTCACTTTCTGCCCTCATCCTCCGGGGCCCAGGGAGAAGGGCACAGGGTCTCCTCGCCCATCAGCAGCCAG
GTGTGCACTGCACAGATGGCTGGTTTACACTCTTCTGATAAGGTGGGGCCAAAGTTTTCAATTCCATCCCCAACTTT
TTCTAAGTCAAGTTCAGCAATTTCAGAGGTAACACAGCCTCCATCCTATGAAGATGCCGTAAAGCAGCAAATGACC
CGGAGTCAGCAGATGGATGAACTCCTGGACGTGCTTATTGAAAGCGGAGAAATGCCAGCAGACGCTAGAGAGGA
TCACTCATGTCTTCAAAAAGTCCCAAAGATACCCAGATCTTCCCGAAGTCCAACTGCTGTCCTCACCAAGCCCTCGG
CTTCCTTTGAACAAGCCTCTTCAGGCAGCCAGATCCCCTTTGATCCCTATGCCACCGACAGTGATGAGCATCTTGAA
GTCTTATTAAATTCCCAGAGCCCCCTAGGAAAGATGAGTGATGTCACCCTTCTAAAAATTGGGAGCGAAGAGCCTC
ACTTTGATGGGATAATGGATGGATTCTCTGGGAAGGCTGCAGAAGACCTCTTCAATGCACATGAGATCTTGCCAG
GCCCCCTCTCTCCAATGCAGACACAGTTTTCACCCTCTTCTGTGGACAGCAATGGGCTGCAGTTAAGCTTCACTGAA
TCTCCCTGGGAAACCATGGAGTGGCTGGACCTCACTCCGCCAAATTCCACACCAGGCTTTAGCGCCCTCACCACCA
GCAGCCCCAGCATCTTCAACATCGATTTCCTGGATGTCACTGATCTCAATTTGAATTCTTCCATGGACCTTCACTTGC
AGCAGTGGTAG
I2: NP_705832.1 (SEQ ID NO: 64)
MTLLGSEHSLLIRSKFRSVLQLRLQQRRTQEQLANQGIIPPLKRPAEFHEQRKHLDSDKAKNSLKRKARNRCNSADLVN
MHILQASTAERSIPTAQMKLKRARLADDLNEKIALRPGPLELVEKNILPVDSAVKEAIKGNQVSFSKSTDAFAFEEDSSSD
GLSPDQTRSEDPQNSAGSPPDAKASDTPSTGSLGTNQDLASGSENDRNDSASQPSHQSDAGKQGLGPPSTPIAVHAA
VKSKSLGDSKNRHKKPKDPKPKVKKLKYHQYIPPDQKAEKSPPPMDSAYARLLQQQQLFLQLQILSQQQQQQQHRFSY
LGMHQAQLKEPNEQMVRNPNSSSTPLSNTPLSPVKNSFSGQTGVSSFKPGPLPPNLDDLKVSELRQQLRIRGLPVSGTK
TALMDRLRPFQDCSGNPVPNFGDITTVTFPVTPNTLPNYQSSSSTSALSNGFYHFGSTSSSPPISPASSDLSVAGSLPDTF
NDASPSFGLHPSPVHVCTEESLMSSLNGGSVPSELDGLDSEKDKMLVEKQKVINELTWKLQQEQRQVEELRMQLQKQ
KRNNCSEKKPLPFLAASIKQEEAVSSCPFASQVPVKRQSSSSECHPPACEAAQLQPLGNAHCVESSDQTNVLSSTFLSPQ
CSPQHSPLGAVKSPQHISLPPSPNNPHFLPSSSGAQGEGHRVSSPISSQVCTAQMAGLHSSDKVGPKFSIPSPTFSKSSSA
ISEVTQPPSYEDAVKQQMTRSQQMDELLDVLIESGEMPADAREDHSCLQKVPKIPRSSRSPTAVLTKPSASFEQASSGS
QIPFDPYATDSDEHLEVLLNSQSPLGKMSDVTLLKIGSEEPHFDGIMDGFSGKAAEDLFNAHEILPGPLSPMQTQFSPSS
VDSNGLQLSFTESPWETMEWLDLTPPNSTPGFSALTTSSPSIFNIDFLDVTDLNLNSSMDLHLQQW
V3: NM_001378306.1 (SEQ ID NO: 65)
ATGCACATACTCCAAGCTTCCACTGCAGAGAGGTCCATTCCAACTGCTCAGATGAAGCTGAAAAGAGCCCGACTCG
CCGATGATCTCAATGAAAAAATTGCTCTACGACCAGGGCCACTGGAGCTGGTGGAAAAAAACATTCTTCCTGTGG
ATTCTGCTGTGAAAGAGGCCATAAAAGGTAACCAGGTGAGTTTCTCCAAATCCACGGATGCTTTTGCCTTTGAAGA
GGACAGCAGCAGCGATGGGCTTTCTCCGGATCAGACTCGAAGTGAAGACCCCCAAAACTCAGCGGGATCCCCGCC
AGACGCTAAAGCCTCAGATACCCCTTCGACAGGTTCTCTGGGGACAAACCAGGATCTTGCTTCTGGCTCAGAAAAT
GACAGAAATGACTCAGCCTCACAGCCCAGCCACCAGTCAGATGCGGGGAAGCAGGGGCTTGGCCCCCCCAGCAC
CCCCATAGCCGTGCATGCTGCTGTAAAGTCCAAATCCTTGGGTGACAGTAAGAACCGCCACAAAAAGCCCAAGGA
CCCCAAGCCAAAGGTGAAGAAGCTTAAATATCACCAGTACATTCCCCCAGACCAGAAGGCAGAGAAGTCCCCTCC
ACCTATGGACTCAGCCTACGCTCGGCTGCTCCAGCAACAGCAGCTGTTCCTGCAGCTCCAAATCCTCAGCCAGCAG
CAGCAGCAGCAGCAACACCGATTCAGCTACCTAGGGATGCACCAAGCTCAGCTTAAGGAACCAAATGAACAGATG
GTCAGAAATCCAAACTCTTCTTCAACGCCACTGAGCAATACCCCCTTGTCTCCTGTCAAAAACAGTTTTTCTGGACA
AACTGGTGTCTCTTCTTTCAAACCAGGCCCACTCCCACCTAACCTGGATGATCTGAAGGTCTCTGAATTAAGACAAC
AGCTTCGAATTCGGGGCTTGCCTGTGTCAGGCACCAAAACGGCTCTCATGGACCGGCTTCGACCCTTCCAGGACTG
CTCTGGCAACCCAGTGCCGAACTTTGGGGATATAACGACTGTCACTTTTCCTGTCACACCCAACACGCTGCCCAATT
ACCAGTCTTCCTCTTCTACCAGTGCCCTGTCCAACGGCTTCTACCACTTTGGCAGCACCAGCTCCAGCCCCCCGATCT
CCCCAGCCTCCTCTGACCTGTCAGTCGCTGGGTCCCTGCCGGACACCTTCAATGATGCCTCCCCCTCCTTCGGCCTG
CACCCGTCCCCAGTCCACGTGTGCACGGAGGAAAGTCTCATGAGCAGCCTGAATGGGGGCTCTGTTCCTTCTGAG
CTGGATGGGCTGGACTCCGAGAAGGACAAGATGCTGGTGGAGAAGCAGAAGGTGATCAATGAACTCACCTGGAA
ACTCCAGCAAGAGCAGAGGCAGGTGGAGGAGCTGAGGATGCAGCTTCAGAAGCAGAAAAGGAATAACTGTTCA
GAGAAGAAGCCGCTGCCTTTCCTGGCTGCCTCCATCAAGCAGGAAGAGGCTGTCTCCAGCTGTCCTTTTGCATCCC
AAGTACCTGTGAAAAGACAAAGCAGCAGCTCAGAGTGTCACCCACCGGCTTGTGAAGCTGCTCAACTCCAGCCTCT
TGGAAATGCTCATTGTGTGGAGTCCTCAGATCAAACCAATGTACTTTCTTCCACATTTCTCAGCCCCCAGTGTTCCCC
TCAGCATTCACCGCTGGGGGCTGTGAAAAGCCCACAGCACATCAGTTTGCCCCCATCACCCAACAACCCTCACTTTC
TGCCCTCATCCTCCGGGGCCCAGGGAGAAGGGCACAGGGTCTCCTCGCCCATCAGCAGCCAGGTGTGCACTGCAC
AGAACTCAGGAGCACACGATGGCCATCCTCCAAGCTTCTCTCCCCATTCTTCCAGCCTCCACCCGCCCTTCTCTGGA
GCCCAAGCAGACAGCAGTCATGGTGCCGGGGGAAACCCTTGTCCCAAAAGCCCATGTGTACAGCAAAAGATGGCT
GGTTTACACTCTTCTGATAAGGTGGGGCCAAAGTTTTCAATTCCATCCCCAACTTTTTCTAAGTCAAGTTCAGCAATT
TCAGAGGTAACACAGCCTCCATCCTATGAAGATGCCGTAAAGCAGCAAATGACCCGGAGTCAGCAGATGGATGAA
CTCCTGGACGTGCTTATTGAAAGCGGAGAAATGCCAGCAGACGCTAGAGAGGATCACTCATGTCTTCAAAAAGTC
CCAAAGATACCCAGATCTTCCCGAAGTCCAACTGCTGTCCTCACCAAGCCCTCGGCTTCCTTTGAACAAGCCTCTTC
AGGCAGCCAGATCCCCTTTGATCCCTATGCCACCGACAGTGATGAGCATCTTGAAGTCTTATTAAATTCCCAGAGC
CCCCTAGGAAAGATGAGTGATGTCACCCTTCTAAAAATTGGGAGCGAAGAGCCTCACTTTGATGGGATAATGGAT
GGATTCTCTGGGAAGGCTGCAGAAGACCTCTTCAATGCACATGAGATCTTGCCAGGCCCCCTCTCTCCAATGCAGA
CACAGTTTTCACCCTCTTCTGTGGACAGCAATGGGCTGCAGTTAAGCTTCACTGAATCTCCCTGGGAAACCATGGA
GTGGCTGGACCTCACTCCGCCAAATTCCACACCAGGCTTTAGCGCCCTCACCACCAGCAGCCCCAGCATCTTCAAC
ATCGATTTCCTGGATGTCACTGATCTCAATTTGAATTCTTCCATGGACCTTCACTTGCAGCAGTGGTAG
I3: NP_001365235.1 (SEQ ID NO: 66)
MHILQASTAERSIPTAQMKLKRARLADDLNEKIALRPGPLELVEKNILPVDSAVKEAIKGNQVSFSKSTDAFAFEEDSSSD
GLSPDQTRSEDPQNSAGSPPDAKASDTPSTGSLGTNQDLASGSENDRNDSASQPSHQSDAGKQGLGPPSTPIAVHAA
VKSKSLGDSKNRHKKPKDPKPKVKKLKYHQYIPPDQKAEKSPPPMDSAYARLLQQQQLFLQLQILSQQQQQQQHRFSY
LGMHQAQLKEPNEQMVRNPNSSSTPLSNTPLSPVKNSFSGQTGVSSFKPGPLPPNLDDLKVSELRQQLRIRGLPVSGTK
TALMDRLRPFQDCSGNPVPNFGDITTVTFPVTPNTLPNYQSSSSTSALSNGFYHFGSTSSSPPISPASSDLSVAGSLPDTF
NDASPSFGLHPSPVHVCTEESLMSSLNGGSVPSELDGLDSEKDKMLVEKQKVINELTWKLQQEQRQVEELRMQLQKQ
KRNNCSEKKPLPFLAASIKQEEAVSSCPFASQVPVKRQSSSSECHPPACEAAQLQPLGNAHCVESSDQTNVLSSTFLSPQ
CSPQHSPLGAVKSPQHISLPPSPNNPHFLPSSSGAQGEGHRVSSPISSQVCTAQNSGAHDGHPPSFSPHSSSLHPPFSGA
QADSSHGAGGNPCPKSPCVQQKMAGLHSSDKVGPKFSIPSPTFSKSSSAISEVTQPPSYEDAVKQQMTRSQQMDELL
DVLIESGEMPADAREDHSCLQKVPKIPRSSRSPTAVLTKPSASFEQASSGSQIPFDPYATDSDEHLEVLLNSQSPLGKMSD
VTLLKIGSEEPHFDGIMDGFSGKAAEDLFNAHEILPGPLSPMQTQFSPSSVDSNGLQLSFTESPWETMEWLDLTPPNST
PGFSALTTSSPSIFNIDFLDVTDLNLNSSMDLHLQQW
PPARGC1B
_133263.4 (SEQ ID NO: 67)
ATGGCGGGGAACGACTGCGGCGCGCTGCTGGACGAAGAGCTCTCCTCCTTCTTCCTCAACTATCTCGCTGACACGC
AGGGTGGAGGGTCCGGGGAGGAGCAACTCTATGCTGACTTTCCAGAACTTGACCTCTCCCAGCTGGATGCCAGCG
ACTTTGACTCGGCCACCTGCTTTGGGGAGCTGCAGTGGTGCCCAGAGAACTCAGAGACTGAACCCAACCAGTACA
GCCCCGATGACTCCGAGCTCTTCCAGATTGACAGTGAGAATGAGGCCCTCCTGGCAGAGCTCACCAAGACCCTGG
ATGACATCCCTGAAGATGACGTGGGTCTGGCTGCCTTCCCAGCCCTGGATGGTGGAGACGCTCTATCATGCACCTC
AGCTTCGCCTGCCCCCTCATCTGCACCCCCCAGCCCTGCCCCGGAGAAGCCCTCGGCCCCAGCCCCTGAGGTGGAC
GAGCTCTCACTGCTGCAGAAGCTCCTCCTGGCCACATCCTACCCAACATCAAGCTCTGACACCCAGAAGGAAGGGA
CCGCCTGGCGCCAGGCAGGCCTCAGATCTAAAAGTCAACGGCCTTGTGTTAAGGCGGACAGCACCCAAGACAAGA
AGGCTCCCATGATGCAGTCTCAGAGCCGAAGTTGTACAGAACTACATAAGCACCTCACCTCGGCACAGTGCTGCCT
GCAGGATCGGGGTCTGCAGCCACCATGCCTCCAGAGTCCCCGGCTCCCTGCCAAGGAGGACAAGGAGCCGGGTG
AGGACTGCCCGAGCCCCCAGCCAGCTCCAGCCTCTCCCCGGGACTCCCTAGCTCTGGGCAGGGCAGACCCCGGTG
CCCCGGTTTCCCAGGAAGACATGCAGGCGATGGTGCAACTCATACGCTACATGCACACCTACTGCCTCCCCCAGAG
GAAGCTGCCCCCACAGACCCCTGAGCCACTCCCCAAGGCCTGCAGCAACCCCTCCCAGCAGGTCAGATCCCGGCCC
TGGTCCCGGCACCACTCCAAAGCCTCCTGGGCTGAGTTCTCCATTCTGAGGGAACTTCTGGCTCAAGACGTGCTCT
GTGATGTCAGCAAACCCTACCGTCTGGCCACGCCTGTTTATGCCTCCCTCACACCTCGGTCAAGGCCCAGGCCCCCC
AAAGACAGTCAGGCCTCCCCTGGTCGCCCGTCCTCGGTGGAGGAGGTAAGGATCGCAGCTTCACCCAAGAGCACC
GGGCCCAGACCAAGCCTGCGCCCACTGCGGCTGGAGGTGAAAAGGGAGGTCCGCCGGCCTGCCAGACTGCAGCA
GCAGGAGGAGGAAGACGAGGAAGAAGAGGAGGAGGAAGAGGAAGAAGAAAAAGAGGAGGAGGAGGAGTGG
GGCAGGAAAAGGCCAGGCCGAGGCCTGCCATGGACGAAGCTGGGGAGGAAGCTGGAGAGCTCTGTGTGCCCCG
TGCGGCGTTCTCGGAGACTGAACCCTGAGCTGGGCCCCTGGCTGACATTTGCAGATGAGCCGCTGGTCCCCTCGG
AGCCCCAAGGTGCTCTGCCCTCACTGTGCCTGGCTCCCAAGGCCTACGACGTAGAGCGGGAGCTGGGCAGCCCCA
CGGACGAGGACAGTGGCCAAGACCAGCAGCTCCTACGGGGACCCCAGATCCCTGCCCTGGAGAGCCCCTGTGAG
AGTGGGTGTGGGGACATGGATGAGGACCCCAGCTGCCCGCAGCTCCCTCCCAGAGACTCTCCCAGGTGCCTCATG
CTGGCCTTGTCACAAAGCGACCCAACTTTTGGCAAGAAGAGCTTTGAGCAGACCTTGACAGTGGAGCTCTGTGGC
ACAGCAGGACTCACCCCACCCACCACACCACCGTACAAGCCCACAGAGGAGGATCCCTTCAAACCAGACATCAAG
CATAGTCTAGGCAAAGAAATAGCTCTCAGCCTCCCCTCCCCTGAGGGCCTCTCACTCAAGGCCACCCCAGGGGCTG
CCCACAAGCTGCCAAAGAAGCACCCAGAGCGAAGTGAGCTCCTGTCCCACCTGCGACATGCCACAGCCCAGCCAG
CCTCCCAGGCTGGCCAGAAGCGTCCCTTCTCCTGTTCCTTTGGAGACCATGACTACTGCCAGGTGCTCCGACCAGA
AGGCGTCCTGCAAAGGAAGGTGCTGAGGTCCTGGGAGCCGTCTGGGGTTCACCTTGAGGACTGGCCCCAGCAGG
GTGCCCCTTGGGCTGAGGCACAGGCCCCTGGCAGGGAGGAAGACAGAAGCTGTGATGCTGGCGCCCCACCCAAG
GACAGCACGCTGCTGAGAGACCATGAGATCCGTGCCAGCCTCACCAAACACTTTGGGCTGCTGGAGACCGCCCTG
GAGGAGGAAGACCTGGCCTCCTGCAAGAGCCCTGAGTATGACACTGTCTTTGAAGACAGCAGCAGCAGCAGCGG
CGAGAGCAGCTTCCTCCCAGAGGAGGAAGAGGAAGAAGGGGAGGAGGAGGAGGAGGACGATGAAGAAGAGG
ACTCAGGGGTCAGCCCCACTTGCTCTGACCACTGCCCCTACCAGAGCCCACCAAGCAAGGCCAACCGGCAGCTCTG
TTCCCGCAGCCGCTCAAGCTCTGGCTCTTCACCCTGCCACTCCTGGTCACCAGCCACTCGAAGGAACTTCAGATGTG
AGAGCAGAGGGCCGTGTTCAGACAGAACGCCAAGCATCCGGCACGCCAGGAAGCGGCGGGAAAAGGCCATTGG
GGAAGGCCGCGTGGTGTACATTCAAAATCTCTCCAGCGACATGAGCTCCCGAGAGCTGAAGAGGCGCTTTGAAGT
GTTTGGTGAGATTGAGGAGTGCGAGGTGCTGACAAGAAATAGGAGAGGCGAGAAGTACGGCTTCATCACCTACC
GGTGTTCTGAGCACGCGGCCCTCTCTTTGACAAAGGGCGCTGCCCTGAGGAAGCGCAACGAGCCCTCCTTCCAGC
TGAGCTACGGAGGGCTCCGGCACTTCTGCTGGCCCAGATACACTGACTACGATTCCAATTCAGAAGAGGCCCTTCC
TGCGTCAGGGAAAAGCAAGTATGAAGCCATGGATTTTGACAGCTTACTGAAAGAGGCCCAGCAGAGCCTGCATTG
A
NP_573570.3 (SEQ ID NO: 68)
MAGNDCGALLDEELSSFFLNYLADTQGGGSGEEQLYADFPELDLSQLDASDFDSATCFGELQWCPENSETEPNQYSPD
DSELFQIDSENEALLAELTKTLDDIPEDDVGLAAFPALDGGDALSCTSASPAPSSAPPSPAPEKPSAPAPEVDELSLLQKLLL
ATSYPTSSSDTQKEGTAWRQAGLRSKSQRPCVKADSTQDKKAPMMQSQSRSCTELHKHLTSAQCCLQDRGLQPPCLQ
SPRLPAKEDKEPGEDCPSPQPAPASPRDSLALGRADPGAPVSQEDMQAMVQLIRYMHTYCLPQRKLPPQTPEPLPKAC
SNPSQQVRSRPWSRHHSKASWAEFSILRELLAQDVLCDVSKPYRLATPVYASLTPRSRPRPPKDSQASPGRPSSVEEVRI
AASPKSTGPRPSLRPLRLEVKREVRRPARLQQQEEEDEEEEEEEEEEEKEEEEEWGRKRPGRGLPWTKLGRKLESSVCPV
RRSRRLNPELGPWLTFADEPLVPSEPQGALPSLCLAPKAYDVERELGSPTDEDSGQDQQLLRGPQIPALESPCESGCGD
MDEDPSCPQLPPRDSPRCLMLALSQSDPTFGKKSFEQTLTVELCGTAGLTPPTTPPYKPTEEDPFKPDIKHSLGKEIALSLP
SPEGLSLKATPGAAHKLPKKHPERSELLSHLRHATAQPASQAGQKRPFSCSFGDHDYCQVLRPEGVLQRKVLRSWEPSG
VHLEDWPQQGAPWAEAQAPGREEDRSCDAGAPPKDSTLLRDHEIRASLTKHFGLLETALEEEDLASCKSPEYDTVFEDS
SSSSGESSFLPEEEEEEGEEEEEDDEEEDSGVSPTCSDHCPYQSPPSKANRQLCSRSRSSSGSSPCHSWSPATRRNFRCES
RGPCSDRTPSIRHARKRREKAIGEGRVVYIQNLSSDMSSRELKRRFEVFGEIEECEVLTRNRRGEKYGFITYRCSEHAALSL
TKGAALRKRNEPSFQLSYGGLRHFCWPRYTDYDSNSEEALPASGKSKYEAMDFDSLLKEAQQSLH

1. A composition for treating a subject with a cardiac disorder or for reprogramming a mesenchymal stem cell (MSC) to an autologous induced cardiomyocyte (iCM), comprising a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B.

2. (canceled)

3. A method of treating a subject with a cardiac disorder,
by administering a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B to said subject; or
by administering an autologous mesenchymal stem cell that has been introduced with a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B to said subject.

4. A method of reprogramming a mesenchymal stem cell (MSC) to an autologous induced cardiomyocyte (iCM), by introducing a ribonucleotide or ribonucleotides or a deoxyribonucleotide or deoxyribonucleotides encoding at least two cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B into the MSC.

5. (canceled)

6. The composition of claim 1,
where the ribonucleotide or ribonucleotides or deoxyribonucleotide or deoxyribonucleotides encode at three cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B, or
where the ribonucleotide or ribonucleotides or deoxyribonucleotide or deoxyribonucleotides encode at least four cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B, or
where the ribonucleotide or ribonucleotides or deoxyribonucleotide or deoxyribonucleotides encode at least five cell fate determinants (CFD) selected from the group consisting of PBX2, ACTN2, POU2F1, HAND1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B.

7-8. (canceled)

9. The composition of claim 1, where the ribonucleotide or ribonucleotides or deoxyribonucleotide or deoxyribonucleotides encode POU2F1, HAND1, GATA4, NACA2, and TSHZ2.

10. The composition of claim 1, where the ribonucleotide or ribonucleotides or deoxyribonucleotide or deoxyribonucleotides encode GATA4, IKZF4, NACA2, and TSHZ2.

11. The composition of claim 1, where the ribonucleotide or ribonucleotides or deoxyribonucleotide or deoxyribonucleotides encode POU2F1, HAND1, GATA4, and HAND2.

12. The composition of claim 1, where the ribonucleotide or ribonucleotides or deoxyribonucleotide or deoxyribonucleotides encode GATA4, HAND2, and IKZF4.

13. The composition of claim 1, where the ribonucleotide or ribonucleotides or deoxyribonucleotide or deoxyribonucleotides encode POU2F1, GATA4, and TSHZ2.

14. The composition of claim 1, where the ribonucleotide or ribonucleotides or deoxyribonucleotide or deoxyribonucleotides encode HAND1, GATA4, IKZF4, and NACA2.

15. The composition of claim 1, where the ribonucleotide or ribonucleotides or deoxyribonucleotide or deoxyribonucleotides encode HAND1, GATA4, and NACA2.

16. The composition of claim 1, where the ribonucleotide or ribonucleotides or deoxyribonucleotide or deoxyribonucleotides encode POU2F1, HAND1, GATA4, IKZF4, and NACA2.

17. The composition of claim 1, where the ribonucleotide or ribonucleotides or deoxyribonucleotide or deoxyribonucleotides encode POU2F1, HAND1, GATA4, JUP, and TSHZ2.

18. The composition of claim 1, where the ribonucleotide or ribonucleotides or deoxyribonucleotide or deoxyribonucleotides encode ACTN2, POU2F1, HAND1, and GATA4.

19. The composition of claim 1,
where the ribonucleotide or ribonucleotides or deoxyribonucleotide or deoxyribonucleotides encode HAND1 and at least one of PBX2, ACTN2, POU2F1, TRIM24, GATA4, PBX1, ZBTB39, HAND2, IKZF4, NROB2, NACA2, SMYD1, JUP, NEUROD1, CKMT2, TSHZ2, MITF, MYOCD, and PPARGC1B, or
where the ribonucleotide or ribonucleotides or deoxyribonucleotide or deoxyribonucleotides encode HAND2 and at least one of PBX2, ACTN2, POU2F1, HAND1, TRIM24, GATA4, PBX1, ZBTB39, IKZF4, NROB2, NACA2, SMYD1, JUP, NEUROD1, CKMT2, TSHZ2, MITF, MYOCD, and PPARGC1B, or
where the ribonucleotide or ribonucleotides or deoxyribonucleotide or deoxyribonucleotides encode HAND1, GATA4, and at least one of PBX2, ACTN2, POU2F1, TRIM24, PBX1, ZBTB39, HAND2, IKZF4, NROB2, NACA2, SMYD1, JUP, NEUROD1, CKMT2, TSHZ2, MITF, MYOCD, and PPARGC1B, or
where the ribonucleotide or ribonucleotides or deoxyribonucleotide or deoxyribonucleotides encode HAND2, GATA4, and at least one of PBX2, ACTN2, POU2F1, HAND1, TRIM24, PBX1, ZBTB39, IKZF4, NROB2, NACA2, SMYD1, JUP, NEUROD1, CKMT2, TSHZ2, MITF, MYOCD, and PPARGC1B, or
where the ribonucleotide or ribonucleotides or deoxyribonucleotide or deoxyribonucleotides encode HAND1, HAND2, and at least one of PBX2, ACTN2, POU2F1, TRIM24, GATA4, PBX1, ZBTB39, IKZF4, NROB2, NACA2, SMYD1, JUP, NEUROD1, CKMT2, TSHZ2, MITF, MYOCD, and PPARGC1B, or
where the ribonucleotide or ribonucleotides or deoxyribonucleotide or deoxyribonucleotides encode HAND1, HAND2, and GATA4, or
where the ribonucleotide or ribonucleotides or deoxyribonucleotide or deoxyribonucleotides encode HAND1, HAND2, GATA4, and at least one of PBX2, ACTN2, POU2F1, TRIM24, PBX1, ZBTB39, IKZF4, NROB2, NACA2, SMYD1, JUP, NEUROD1, CKMT2, TSHZ2, MITF, MYOCD, and PPARGC1B.

20-25. (canceled)

26. The composition of claim 1, wherein the cardiac disorder is selected from the group consisting of myocardial infarction, coronary artery disease, ischemic cardiomyopathy, cardiac fibrosis, congestive heart failure (CHF), end-stage heart failure, cardiomyopathy, dilated cardiomyopathy, restrictive cardiomyopathy, and hypertrophic cardiomyopathy, viral cardiomyopathy, myocarditis, chemical-induced cardiomyopathy, post-partum cardiomyopathy, cardiomyopathy due to endocrine disorders, high cholesterol diseases, hemochromatosis and sarcoidosis.

27. Vector(s) comprising the ribonucleotide or ribonucleotides or deoxyribonucleotide or deoxyribonucleotides of claim 1.

28. The vector(s) of claim 27, wherein the vector(s) are viral vectors including retroviral systems such as MMLV, HIV-1, and ALV, adenoviral vectors, adeno-associated virus vectors, lentiviral vectors such as those based on HIV or FIV gag sequences; the poxvirus family such as vaccinia virus and the avian pox viruses, the alpha virus genus such as those derived from Sindbis and Semliki Forest Viruses, Venezuelan equine encephalitis virus, rhabdoviruses such as vesicular stomatitis virus, papillomaviruses, and baculoviruses, or nonviral vectors such as lipid-based vectors, polymeric vectors, dendrimer vectors, polypeptide vectors, and nanoparticles.

29-30. (canceled)

31. The method of claim 3, wherein the ribonucleotide or ribonucleotides or deoxyribonucleotide or deoxyribonucleotides are introduced by a vector or vectors.

32. The method of claim 31, wherein the vector(s) are viral vectors including retroviral systems such as MMLV, HIV-1, and ALV, adenoviral vectors, adeno-associated virus vectors, lentiviral vectors such as those based on HIV or FIV gag sequences, the poxvirus family such as vaccinia virus and the avian pox viruses, the alpha virus genus such as those derived from Sindbis and Semliki Forest Viruses, Venezuelan equine encephalitis virus, rhabdoviruses such as vesicular stomatitis virus, papillomaviruses, and baculoviruses, or nonviral vectors such as lipid-based vectors, polymeric vectors, dendrimer vectors, polypeptide vectors, and nanoparticles.

33-34. (canceled)


SEQUENCE LISTING (68 sequences)

SEQ ID NO: 1 - DNA, 1293 bp, Homo sapiens
  source 1..1293: mol_type = other DNA
atggacgaacggctactggggccgccccctccaggcgggggccgggggggcctgggattggtgagtggggagcctgggggccctggcgagcctcccggtggcggagaccccggtgggggtagcgggggggtcccgggaggccgagggaagcaagacatcggggacattctgcagcagataatgaccatcaccgaccagagcctggacgaggcccaggccaagaaacacgccctaaactgccaccgaatgaagcctgctctctttagcgtcctgtgtgaaatcaaggagaaaactggcctcagcattcggagctcccaggaggaggagccggtggacccacagctgatgcgcttggacaacatgcttctggcagagggtgtggctgggcccgagaaagggggcggctcagcagcagcagctgcagccgctgcagcctctggtggtggtgtgtcccctgacaactccatcgaacactcggactatcgcagcaaacttgcccagatccgtcacatataccactcggagctggagaagtatgagcaggcatgtaatgagttcacgacccatgtcatgaacctgctgagggagcagagccgcaccaggcccgtggcccccaaagagatggaacgcatggtgagcatcatccatcgaaagttcagcgccatccagatgcagctgaagcagagcacctgcgaggctgtgatgatcctgcgctcccgtttcctggatgccagacgaaagcgccgtaacttcagcaaacaggccactgaggtcctaaatgagtatttctactcccacctgagtaacccatatcctagtgaggaggccaaggaggagcttgccaagaagtgtggcatcaccgtgtctcaggtctccaactggtttggcaacaagaggattcgctataagaaaaacatcggaaagttccaagaggaggcaaacatctatgctgtcaagaccgccgtgtcagtcacccaggggggccacagccgcaccagctccccgacacccccttcctctgcaggctctggcggctctttcaatctctcaggatctggagacatgtttctggggatgcctgggctcaacggagattcctattctgcttcccaggtggaatcactccgacactcgatggggccagggggctatggggataacctcgggggaggccagatgtacagcccacgggaaatgagggcaaatggcagctggcaagaggctgtgaccccctcttcagtgacatccccaacggagggaccagggagtgttcactctgatacctccaactga

SEQ ID NO: 2 - protein, 430 aa, Homo sapiens
MDERLLGPPPPGGGRGGLGLVSGEPGGPGEPPGGGDPGGGSGGVPGGRGKQDIGDILQQIMTITDQSLDEAQAKKHALNCHRMKPALFSVLCEIKEKTGLSIRSSQEEEPVDPQLMRLDNMLLAEGVAGPEKGGGSAAAAAAAAASGGGVSPDNSIEHSDYRSKLAQIRHIYHSELEKYEQACNEFTTHVMNLLREQSRTRPVAPKEMERMVSIIHRKFSAIQMQLKQSTCEAVMILRSRFLDARRKRRNFSKQATEVLNEYFYSHLSNPYPSEEAKEELAKKCGITVSQVSNWFGNKRIRYKKNIGKFQEEANIYAVKTAVSVTQGGHSRTSSPTPPSSAGSGGSFNLSGSGDMFLGMPGLNGDSYSASQVESLRHSMGPGGYGDNLGGGQMYSPREMRANGSWQEAVTPSSVTSPTEGPGSVHSDTSN

SEQ ID NO: 3 - DNA, 2685 bp, Homo sapiens
  source 1..2685: mol_type = other DNA
atgaaccagatagagcccggcgtgcagtacaactacgtgtacgacgaggatgagtacatgatccaggaggaggagtgggaccgcgacctgctcctggacccagcctgggagaagcagcagaggaagaccttcactgcctggtgtaactcccacctaaggaaagccggcacccagattgagaacatcgaggaagacttcaggaatggccttaagctcatgctgcttttggaagtcatctcaggggaaaggctgcccaaacctgaccggggaaaaatgcggttccacaaaattgctaatgtcaacaaagctttggattacatagccagcaaaggggtgaaactggtgtccattggcgctgaagaaattgttgatggcaacgtgaaaatgaccctgggtatgatctggaccatcatccttcgctttgctattcaggatatttcggttgaagaaacatctgccaaagaaggtctgctgctttggtgtcagaggaaaactgctccttatagaaatgtgaacattcagaacttccatactagctggaaagatggccttggactctgtgccctcatccaccgacaccggcctgacctcattgactactcaaagcttaacaaggatgaccccataggaaatattaacctggccatggaaatcgctgagaagcacctggatattcctaaaatgttggatgctgaagacatcgtgaacacccctaaacccgatgaaagagccatcatgacgtacgtctcttgcttctaccacgcttttgcgggcgcggagcaggccgagacagcggctaacaggatatgtaaggttcttgctgtgaatcaagagaatgagaggctgatggaagaatatgagaggctagcgagtgagcttttggaatggattcgtcgcacgatcccctggctggagaaccggactcccgagaagaccatgcaagccatgcagaagaagctggaggacttccgggattaccgccggaagcacaagccacccaaggtgcaggagaaatgccagctggagatcaacttcaacacgctgcagaccaagctgcggatcagcaaccgtcctgccttcatgccctccgagggcaagatggtgtcggatattgctggtgcctggcagaggctggagcaggctgagaagggttacgaggagtggttgctcaatgagattcggagactggagcgcttggaacacctggctgagaagttcaggcagaaggcctcaacgcacgagacttgggcttatggcaaagagcagatcttgctgcagaaggattacgagtcggcgtcgctgacagaggtgcgggctctgctgcggaagcacgaggcgttcgagagcgacctggcagcgcaccaggaccgcgtggagcagatcgcagccatcgcgcaggagctcaatgaactggactatcacgacgctgtgaatgtcaatgatcggtgccagaaaatttgtgaccagtgggaccgactgggaacgcttactcagaagaggagagaagccctagagagaatggagaaattgctagaaaccattgatcagcttcacctggagtttgccaagagggctgctcctttcaacaattggatggagggcgctatggaggatctgcaagatatgttcattgtccacagcattgaggagatccagagtctgatcactgcgcatgagcagttcaaggccacgctgcccgaggcggacggagagcggcagtccatcatggccatccagaacgaggtggagaaggtgattcagagctacaacatcagaatcagctcaagcaacccgtacagcactgtcaccatggatgagctccggaccaagtgggacaaggtgaagcaactcgtgcccatccgcgatcaatccctgcaggaggagctggctcgccagcatgctaacgagcgtctgaggcgccagtttgctgcccaagccaatgccattgggccctggatccagaacaagatggaggagattgcccggagctccatccagatcacaggagccctggaagaccagatgaaccagctgaagcagtatgagcacaacatcatcaactataagaacaacatcgacaagctggagggagaccatcagctcatccaggaggcccttgtctttgacaacaagcacacgaactacacgatggagcacattcgtgttggatgggagctgctgctgacaaccatcgccagaaccatcaatgaggtggagactcagatcctgacgagagatgcgaagggcatcacccaggagcagatgaatgagttcagagcctccttcaaccactttgacaggaggaagaatggcctgatggatcatgaggatttcagagcctgcctgatttccatgggttatgacctgggtgaagccgaatttgcccgcattatgaccctggtagatcccaacgggcaaggcaccgtcaccttccaatccttcatcgacttcatgactagagagacggctgacaccgacactgccgagcaggtcatcgcctccttccggatcctggcttctgataagccatacatcctggcggaggagctgcgtcgggagctgcccccggatcaggcccagtactgcatcaagaggatgcccgcctactcgggcccaggcagtgtgcctggtgcactggattacgctgcgttctcttccgcactctacggggagagcgatctgtga

SEQ ID NO: 4 - protein, 894 aa, Homo sapiens
MNQIEPGVQYNYVYDEDEYMIQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHTSWKDGLGLCALIHRHRPDLIDYSKLNKDDPIGNINLAMEIAEKHLDIPKMLDAEDIVNTPKPDERAIMTYVSCFYHAFAGAEQAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHETWAYGKEQILLQKDYESASLTEVRALLRKHEAFESDLAAHQDRVEQIAAIAQELNELDYHDAVNVNDRCQKICDQWDRLGTLTQKRREALERMEKLLETIDQLHLEFAKRAAPFNNWMEGAMEDLQDMFIVHSIEEIQSLITAHEQFKATLPEADGERQSIMAIQNEVEKVIQSYNIRISSSNPYSTVTMDELRTKWDKVKQLVPIRDQSLQEELARQHANERLRRQFAAQANAIGPWIQNKMEEIARSSIQITGALEDQMNQLKQYEHNIINYKNNIDKLEGDHQLIQEALVFDNKHTNYTMEHIRVGWELLLTTIARTINEVETQILTRDAKGITQEQMNEFRASFNHFDRRKNGLMDHEDFRACLISMGYDLGEAEFARIMTLVDPNGQGTVTFQSFIDFMTRETADTDTAEQVIASFRILASDKPYILAEELRRELPPDQAQYCIKRMPAYSGPGSVPGALDYAAFSSALYGESDL

SEQ ID NO: 5 - DNA, 2685 bp, Homo sapiens
  source 1..2685: mol_type = other DNA
atgaaccagatagagcccggcgtgcagtacaactacgtgtacgacgaggatgagtacatgatccaggaggaggagtgggaccgcgacctgctcctggacccagcctgggagaagcagcagaggaagaccttcactgcctggtgtaactcccacctaaggaaagccggcacccagattgagaacatcgaggaagacttcaggaatggccttaagctcatgctgcttttggaagtcatctcaggggaaaggctgcccaaacctgaccggggaaaaatgcggttccacaaaattgctaatgtcaacaaagctttggattacatagccagcaaaggggtgaaactggtgtccattggcgctgaagaaattgttgatggcaacgtgaaaatgaccctgggtatgatctggaccatcatccttcgctttgctattcaggatatttcggttgaagaaacatctgccaaagaaggtctgctgctttggtgtcagaggaaaactgctccttatagaaatgtgaacattcagaacttccatactagctggaaagatggccttggactctgtgccctcatccaccgacaccggcctgacctcattgactactcaaagcttaacaaggatgaccccataggaaatattaacctggccatggaaatcgctgagaagcacctggatattcctaaaatgttggatgctgaagatttagtatacactgccagacccgatgaaagagccataatgacttatgtttcctgttactatcatgcttttgctggtgcacagaaggccgagacagcggctaacaggatatgtaaggttcttgctgtgaatcaagagaatgagaggctgatggaagaatatgagaggctagcgagtgagcttttggaatggattcgtcgcacgatcccctggctggagaaccggactcccgagaagaccatgcaagccatgcagaagaagctggaggacttccgggattaccgccggaagcacaagccacccaaggtgcaggagaaatgccagctggagatcaacttcaacacgctgcagaccaagctgcggatcagcaaccgtcctgccttcatgccctccgagggcaagatggtgtcggatattgctggtgcctggcagaggctggagcaggctgagaagggttacgaggagtggttgctcaatgagattcggagactggagcgcttggaacacctggctgagaagttcaggcagaaggcctcaacgcacgagacttgggcttatggcaaagagcagatcttgctgcagaaggattacgagtcggcgtcgctgacagaggtgcgggctctgctgcggaagcacgaggcgttcgagagcgacctggcagcgcaccaggaccgcgtggagcagatcgcagccatcgcgcaggagctcaatgaactggactatcacgacgctgtgaatgtcaatgatcggtgccagaaaatttgtgaccagtgggaccgactgggaacgcttactcagaagaggagagaagccctagagagaatggagaaattgctagaaaccattgatcagcttcacctggagtttgccaagagggctgctcctttcaacaattggatggagggcgctatggaggatctgcaagatatgttcattgtccacagcattgaggagatccagagtctgatcactgcgcatgagcagttcaaggccacgctgcccgaggcggacggagagcggcagtccatcatggccatccagaacgaggtggagaaggtgattcagagctacaacatcagaatcagctcaagcaacccgtacagcactgtcaccatggatgagctccggaccaagtgggacaaggtgaagcaactcgtgcccatccgcgatcaatccctgcaggaggagctggctcgccagcatgctaacgagcgtctgaggcgccagtttgctgcccaagccaatgccattgggccctggatccagaacaagatggaggagattgcccggagctccatccagatcacaggagccctggaagaccagatgaaccagctgaagcagtatgagcacaacatcatcaactataagaacaacatcgacaagctggagggagaccatcagctcatccaggaggcccttgtctttgacaacaagcacacgaactacacgatggagcacattcgtgttggatgggagctgctgctgacaaccatcgccagaaccatcaatgaggtggagactcagatcctgacgagagatgcgaagggcatcacccaggagcagatgaatgagttcagagcctccttcaaccactttgacaggaggaagaatggcctgatggatcatgaggatttcagagcctgcctgatttccatgggttatgacctgggtgaagccgaatttgcccgcattatgaccctggtagatcccaacgggcaaggcaccgtcaccttccaatccttcatcgacttcatgactagagagacggctgacaccgacactgccgagcaggtcatcgcctccttccggatcctggcttctgataagccatacatcctggcggaggagctgcgtcgggagctgcccccggatcaggcccagtactgcatcaagaggatgcccgcctactcgggcccaggcagtgtgcctggtgcactggattacgctgcgttctcttccgcactctacggggagagcgatctgtga

SEQ ID NO: 6 - protein, 894 aa, Homo sapiens
MNQIEPGVQYNYVYDEDEYMIQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHTSWKDGLGLCALIHRHRPDLIDYSKLNKDDPIGNINLAMEIAEKHLDIPKMLDAEDLVYTARPDERAIMTYVSCYYHAFAGAQKAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHETWAYGKEQILLQKDYESASLTEVRALLRKHEAFESDLAAHQDRVEQIAAIAQELNELDYHDAVNVNDRCQKICDQWDRLGTLTQKRREALERMEKLLETIDQLHLEFAKRAAPFNNWMEGAMEDLQDMFIVHSIEEIQSLITAHEQFKATLPEADGERQSIMAIQNEVEKVIQSYNIRISSSNPYSTVTMDELRTKWDKVKQLVPIRDQSLQEELARQHANERLRRQFAAQANAIGPWIQNKMEEIARSSIQITGALEDQMNQLKQYEHNIINYKNNIDKLEGDHQLIQEALVFDNKHTNYTMEHIRVGWELLLTTIARTINEVETQILTRDAKGITQEQMNEFRASFNHFDRRKNGLMDHEDFRACLISMGYDLGEAEFARIMTLVDPNGQGTVTFQSFIDFMTRETADTDTAEQVIASFRILASDKPYILAEELRRELPPDQAQYCIKRMPAYSGPGSVPGALDYAAFSSALYGESDL

SEQ ID NO: 7 - DNA, 2061 bp, Homo sapiens
  source 1..2061: mol_type = other DNA
atgacgtacgtctcttgcttctaccacgcttttgcgggcgcggagcaggttagacaaagtcttaaagcacactcagctctgtggaaggatccccctccagaaagttctacatgttcatatcaggagatgaggaggtcttcagtgaattcaagtgcaatggccgagacagcggctaacaggatatgtaaggttcttgctgtgaatcaagagaatgagaggctgatggaagaatatgagaggctagcgagtgagcttttggaatggattcgtcgcacgatcccctggctggagaaccggactcccgagaagaccatgcaagccatgcagaagaagctggaggacttccgggattaccgccggaagcacaagccacccaaggtgcaggagaaatgccagctggagatcaacttcaacacgctgcagaccaagctgcggatcagcaaccgtcctgccttcatgccctccgagggcaagatggtgtcggatattgctggtgcctggcagaggctggagcaggctgagaagggttacgaggagtggttgctcaatgagattcggagactggagcgcttggaacacctggctgagaagttcaggcagaaggcctcaacgcacgagacttgggcttatggcaaagagcagatcttgctgcagaaggattacgagtcggcgtcgctgacagaggtgcgggctctgctgcggaagcacgaggcgttcgagagcgacctggcagcgcaccaggaccgcgtggagcagatcgcagccatcgcgcaggagctcaatgaactggactatcacgacgctgtgaatgtcaatgatcggtgccagaaaatttgtgaccagtgggaccgactgggaacgcttactcagaagaggagagaagccctagagagaatggagaaattgctagaaaccattgatcagcttcacctggagtttgccaagagggctgctcctttcaacaattggatggagggcgctatggaggatctgcaagatatgttcattgtccacagcattgaggagatccagagtctgatcactgcgcatgagcagttcaaggccacgctgcccgaggcggacggagagcggcagtccatcatggccatccagaacgaggtggagaaggtgattcagagctacaacatcagaatcagctcaagcaacccgtacagcactgtcaccatggatgagctccggaccaagtgggacaaggtgaagcaactcgtgcccatccgcgatcaatccctgcaggaggagctggctcgccagcatgctaacgagcgtctgaggcgccagtttgctgcccaagccaatgccattgggccctggatccagaacaagatggaggagattgcccggagctccatccagatcacaggagccctggaagaccagatgaaccagctgaagcagtatgagcacaacatcatcaactataagaacaacatcgacaagctggagggagaccatcagctcatccaggaggcccttgtctttgacaacaagcacacgaactacacgatggagcacattcgtgttggatgggagctgctgctgacaaccatcgccagaaccatcaatgaggtggagactcagatcctgacgagagatgcgaagggcatcacccaggagcagatgaatgagttcagagcctccttcaaccactttgacaggaggaagaatggcctgatggatcatgaggatttcagagcctgcctgatttccatgggttatgacctgggtgaagccgaatttgcccgcattatgaccctggtagatcccaacgggcaaggcaccgtcaccttccaatccttcatcgacttcatgactagagagacggctgacaccgacactgccgagcaggtcatcgcctccttccggatcctggcttctgataagccatacatcctggcggaggagctgcgtcgggagctgcccccggatcaggcccagtactgcatcaagaggatgcccgcctactcgggcccaggcagtgtgcctggtgcactggattacgctgcgttctcttccgcactctacggggagagcgatctgtga

SEQ ID NO: 8 - protein, 686 aa, Homo sapiens
MTYVSCFYHAFAGAEQVRQSLKAHSALWKDPPPESSTCSYQEMRRSSVNSSAMAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHETWAYGKEQILLQKDYESASLTEVRALLRKHEAFESDLAAHQDRVEQIAAIAQELNELDYHDAVNVNDRCQKICDQWDRLGTLTQKRREALERMEKLLETIDQLHLEFAKRAAPFNNWMEGAMEDLQDMFIVHSIEEIQSLITAHEQFKATLPEADGERQSIMAIQNEVEKVIQSYNIRISSSNPYSTVTMDELRTKWDKVKQLVPIRDQSLQEELARQHANERLRRQFAAQANAIGPWIQNKMEEIARSSIQITGALEDQMNQLKQYEHNIINYKNNIDKLEGDHQLIQEALVFDNKHTNYTMEHIRVGWELLLTTIARTINEVETQILTRDAKGITQEQMNEFRASFNHFDRRKNGLMDHEDFRACLISMGYDLGEAEFARIMTLVDPNGQGTVTFQSFIDFMTRETADTDTAEQVIASFRILASDKPYILAEELRRELPPDQAQYCIKRMPAYSGPGSVPGALDYAAFSSALYGESDL

SEQ ID NO: 9 - DNA, 2301 bp, Homo sapiens
  source 1..2301: mol_type = other DNA
atggcggacggaggagcagcgagtcaagatgagagttcagccgcggcggcagcagcagcagactcaagaatgaacaatccgtcagaaaccagtaaaccatctatggagagtggagatggcaacacaggcacacaaaccaatggtctggactttcagaagcagcctgtgcctgtaggaggagcaatctcaacagcccaggcgcaggctttccttggacatctccatcaggtccaactcgctggaacaagtttacaggctgctgctcagtctttaaatgtacagtctaaatctaatgaagaatcgggggattcgcagcagccaagccagccttcccagcagccttcagtgcaggcagccattccccagacccagcttatgctagctggaggacagataactgggcttactttgacgcctgcccagcaacagttactactccagcaggcacaggcacaggcacagctgctggctgctgcagtgcagcagcactccgccagccagcagcacagtgctgctggagccaccatctccgcctctgctgccacgcccatgacgcagatccccctgtctcagcccatacagatcgcacaggatcttcaacaactgcaacagcttcaacagcagaatctcaacctgcaacagtttgtgttggtgcatccaaccaccaatttgcagccagcgcagtttatcatctcacagacgccccagggccagcagggtctcctgcaagcgcaaaatcttctaacgcaactacctcagcaaagccaagccaacctcctacagtcgcagccaagcatcaccctcacctcccagccagcaaccccaacacgcacaatagcagcaaccccaattcagacacttccacagagccagtcaacaccaaagcgaattgatactcccagcttggaggagcccagtgaccttgaggagcttgagcagtttgccaagaccttcaaacaaagacgaatcaaacttggattcactcagggtgatgttgggctcgctatggggaaactatatggaaatgacttcagccaaactaccatctctcgatttgaagccttgaacctcagctttaagaacatgtgcaagttgaagccacttttagagaagtggctaaatgatgcagagaacctctcatctgattcgtccctctccagcccaagtgccctgaattctccaggaattgagggcttgagccgtaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacccaccaagcagtggtgggaccagcagctcacctattaaagcaattttccccagcccaacttcactggtggcgaccacaccaagccttgtgactagcagtgcagcaactaccctcacagtcagccctgtcctccctctgaccagtgctgctgtgacgaatctttcagttacaggcacttcagacaccacctccaacaacacagcaaccgtgatttccacagcgcctccagcttcctcagcagtcacgtccccctctctgagtccctccccttctgcctcagcctccacctccgaggcatccagtgccagtgagaccagcacaacacagaccacctccactcctttgtcctcccctcttgggaccagccaggtgatggtgacagcatcaggtttgcaaacagcagcagctgctgcccttcaaggagctgcacagttgccagcaaatgccagtcttgctgccatggcagctgctgcaggactaaacccaagcctgatggcaccctcacagtttgcggctggaggtgccttactcagtctgaatccagggaccctgagcggtgctctcagcccagctctaatgagcaacagtacactggcaactattcaagctcttgcttctggtggctctcttccaataacatcacttgatgcaactgggaacctggtatttgccaatgcgggaggagcccccaacatcgtgactgcccctctgttcctgaaccctcagaacctctctctgctcaccagcaaccctgttagcttggtctctgccgccgcagcatctgcagggaactctgcacctgtagccagccttcacgccacctccacctctgctgagtccatccagaactctctcttcacagtggcctctgccagcggggctgcgtccaccaccaccaccgcctccaaggcacagtga

SEQ ID NO: 10 - protein, 766 aa, Homo sapiens
MADGGAASQDESSAAAAAAADSRMNNPSETSKPSMESGDGNTGTQTNGLDFQKQPVPVGGAISTAQAQAFLGHLHQVQLAGTSLQAAAQSLNVQSKSNEESGDSQQPSQPSQQPSVQAAIPQTQLMLAGGQITGLTLTPAQQQLLLQQAQAQAQLLAAAVQQHSASQQHSAAGATISASAATPMTQIPLSQPIQIAQDLQQLQQLQQQNLNLQQFVLVHPTTNLQPAQFIISQTPQGQQGLLQAQNLLTQLPQQSQANLLQSQPSITLTSQPATPTRTIAATPIQTLPQSQSTPKRIDTPSLEEPSDLEELEQFAKTFKQRRIKLGFTQGDVGLAMGKLYGNDFSQTTISRFEALNLSFKNMCKLKPLLEKWLNDAENLSSDSSLSSPSALNSPGIEGLSRRRKKRTSIETNIRVALEKSFLENQKPTSEEITMIADQLNMEKEVIRVWFCNRRQKEKRINPPSSGGTSSSPIKAIFPSPTSLVATTPSLVTSSAATTLTVSPVLPLTSAAVTNLSVTGTSDTTSNNTATVISTAPPASSAVTSPSLSPSPSASASTSEASSASETSTTQTTSTPLSSPLGTSQVMVTASGLQTAAAAALQGAAQLPANASLAAMAAAAGLNPSLMAPSQFAAGGALLSLNPGTLSGALSPALMSNSTLATIQALASGGSLPITSLDATGNLVFANAGGAPNIVTAPLFLNPQNLSLLTSNPVSLVSAAAASAGNSAPVASLHATSTSAESIQNSLFTVASASGAASTTTTASKAQ

SEQ ID NO: 11 - DNA, 2268 bp, Homo sapiens
  source 1..2268: mol_type = other DNA
atgctggactgcagtgactatgttctagactcaagaatgaacaatccgtcagaaaccagtaaaccatctatggagagtggagatggcaacacaggcacacaaaccaatggtctggactttcagaagcagcctgtgcctgtaggaggagcaatctcaacagcccaggcgcaggctttccttggacatctccatcaggtccaactcgctggaacaagtttacaggctgctgctcagtctttaaatgtacagtctaaatctaatgaagaatcgggggattcgcagcagccaagccagccttcccagcagccttcagtgcaggcagccattccccagacccagcttatgctagctggaggacagataactgggcttactttgacgcctgcccagcaacagttactactccagcaggcacaggcacaggcacagctgctggctgctgcagtgcagcagcactccgccagccagcagcacagtgctgctggagccaccatctccgcctctgctgccacgcccatgacgcagatccccctgtctcagcccatacagatcgcacaggatcttcaacaactgcaacagcttcaacagcagaatctcaacctgcaacagtttgtgttggtgcatccaaccaccaatttgcagccagcgcagtttatcatctcacagacgccccagggccagcagggtctcctgcaagcgcaaaatcttctaacgcaactacctcagcaaagccaagccaacctcctacagtcgcagccaagcatcaccctcacctcccagccagcaaccccaacacgcacaatagcagcaaccccaattcagacacttccacagagccagtcaacaccaaagcgaattgatactcccagcttggaggagcccagtgaccttgaggagcttgagcagtttgccaagaccttcaaacaaagacgaatcaaacttggattcactcagggtgatgttgggctcgctatggggaaactatatggaaatgacttcagccaaactaccatctctcgatttgaagccttgaacctcagctttaagaacatgtgcaagttgaagccacttttagagaagtggctaaatgatgcagagaacctctcatctgattcgtccctctccagcccaagtgccctgaattctccaggaattgagggcttgagccgtaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacccaccaagcagtggtgggaccagcagctcacctattaaagcaattttccccagcccaacttcactggtggcgaccacaccaagccttgtgactagcagtgcagcaactaccctcacagtcagccctgtcctccctctgaccagtgctgctgtgacgaatctttcagttacaggcacttcagacaccacctccaacaacacagcaaccgtgatttccacagcgcctccagcttcctcagcagtcacgtccccctctctgagtccctccccttctgcctcagcctccacctccgaggcatccagtgccagtgagaccagcacaacacagaccacctccactcctttgtcctcccctcttgggaccagccaggtgatggtgacagcatcaggtttgcaaacagcagcagctgctgcccttcaaggagctgcacagttgccagcaaatgccagtcttgctgccatggcagctgctgcaggactaaacccaagcctgatggcaccctcacagtttgcggctggaggtgccttactcagtctgaatccagggaccctgagcggtgctctcagcccagctctaatgagcaacagtacactggcaactattcaagctcttgcttctggtggctctcttccaataacatcacttgatgcaactgggaacctggtatttgccaatgcgggaggagcccccaacatcgtgactgcccctctgttcctgaaccctcagaacctctctctgctcaccagcaaccctgttagcttggtctctgccgccgcagcatctgcagggaactctgcacctgtagccagccttcacgccacctccacctctgctgagtccatccagaactctctcttcacagtggcctctgccagcggggctgcgtccaccaccaccaccgcctccaaggcacagtga

SEQ ID NO: 12 - protein, 755 aa, Homo sapiens
MLDCSDYVLDSRMNNPSETSKPSMESGDGNTGTQTNGLDFQKQPVPVGGAISTAQAQAFLGHLHQVQLAGTSLQAAAQSLNVQSKSNEESGDSQQPSQPSQQPSVQAAIPQTQLMLAGGQITGLTLTPAQQQLLLQQAQAQAQLLAAAVQQHSASQQHSAAGATISASAATPMTQIPLSQPIQIAQDLQQLQQLQQQNLNLQQFVLVHPTTNLQPAQFIISQTPQGQQGLLQAQNLLTQLPQQSQANLLQSQPSITLTSQPATPTRTIAATPIQTLPQSQSTPKRIDTPSLEEPSDLEELEQFAKTFKQRRIKLGFTQGDVGLAMGKLYGNDFSQTTISRFEALNLSFKNMCKLKPLLEKWLNDAENLSSDSSLSSPSALNSPGIEGLSRRRKKRTSIETNIRVALEKSFLENQKPTSEEITMIADQLNMEKEVIRVWFCNRRQKEKRINPPSSGGTSSSPIKAIFPSPTSLVATTPSLVTSSAATTLTVSPVLPLTSAAVTNLSVTGTSDTTSNNTATVISTAPPASSAVTSPSLSPSPSASASTSEASSASETSTTQTTSTPLSSPLGTSQVMVTASGLQTAAAAALQGAAQLPANASLAAMAAAAGLNPSLMAPSQFAAGGALLSLNPGTLSGALSPALMSNSTLATIQALASGGSLPITSLDATGNLVFANAGGAPNIVTAPLFLNPQNLSLLTSNPVSLVSAAAASAGNSAPVASLHATSTSAESIQNSLFTVASASGAASTTTTASKAQ

SEQ ID NO: 13 - DNA, 2112 bp, Homo sapiens
  source 1..2112: mol_type = other DNA
atggcggacggaggagcagcgagtcaagatgagagttcagccgcggcggcagcagcagcagactcaagaatgaacaatccgtcagaaaccagtaaaccatctatggagagtggagatggcaacacaggcacacaaaccaatggtctggactttcagaagcagcctgtgcctgtaggaggagcaatctcaacagcccaggcgcaggctttccttggacatctccatcaggtccaactcgctggaacaagtttacaggctgctgctcagtctttaaatgtacagtctaaatctaatgaagaatcgggggattcgcagcagccaagccagccttcccagcagccttcagtgcaggcagccattccccagacccagcttatgctagctggaggacagataactggggatcttcaacaactgcaacagcttcaacagcagaatctcaacctgcaacagtttgtgttggtgcatccaaccaccaatttgcagccagcgcagtttatcatctcacagacgccccagggccagcagggtctcctgcaagcgcaaaatcttctaacgcaactacctcagcaaagccaagccaacctcctacagtcgcagccaagcatcaccctcacctcccagccagcaaccccaacacgcacaatagcagcaaccccaattcagacacttccacagagccagtcaacaccaaagcgaattgatactcccagcttggaggagcccagtgaccttgaggagcttgagcagtttgccaagaccttcaaacaaagacgaatcaaacttggattcactcagggtgatgttgggctcgctatggggaaactatatggaaatgacttcagccaaactaccatctctcgatttgaagccttgaacctcagctttaagaacatgtgcaagttgaagccacttttagagaagtggctaaatgatgcagagaacctctcatctgattcgtccctctccagcccaagtgccctgaattctccaggaattgagggcttgagccgtaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacccaccaagcagtggtgggaccagcagctcacctattaaagcaattttccccagcccaacttcactggtggcgaccacaccaagccttgtgactagcagtgcagcaactaccctcacagtcagccctgtcctccctctgaccagtgctgctgtgacgaatctttcagttacaggcacttcagacaccacctccaacaacacagcaaccgtgatttccacagcgcctccagcttcctcagcagtcacgtccccctctctgagtccctccccttctgcctcagcctccacctccgaggcatccagtgccagtgagaccagcacaacacagaccacctccactcctttgtcctcccctcttgggaccagccaggtgatggtgacagcatcaggtttgcaaacagcagcagctgctgcccttcaaggagctgcacagttgccagcaaatgccagtcttgctgccatggcagctgctgcaggactaaacccaagcctgatggcaccctcacagtttgcggctggaggtgccttactcagtctgaatccagggaccctgagcggtgctctcagcccagctctaatgagcaacagtacactggcaactattcaagctcttgcttctggtggctctcttccaataacatcacttgatgcaactgggaacctggtatttgccaatgcgggaggagcccccaacatcgtgactgcccctctgttcctgaaccctcagaacctctctctgctcaccagcaaccctgttagcttggtctctgccgccgcagcatctgcagggaactctgcacctgtagccagccttcacgccacctccacctctgctgagtccatccagaactctctcttcacagtggcctctgccagcggggctgcgtccaccaccaccaccgcctccaaggcacagtga

SEQ ID NO: 14 - protein, 703 aa, Homo sapiens
MADGGAASQDESSAAAAAAADSRMNNPSETSKPSMESGDGNTGTQTNGLDFQKQPVPVGGAISTAQAQAFLGHLHQVQLAGTSLQAAAQSLNVQSKSNEESGDSQQPSQPSQQPSVQAAIPQTQLMLAGGQITGDLQQLQQLQQQNLNLQQFVLVHPTTNLQPAQFIISQTPQGQQGLLQAQNLLTQLPQQSQANLLQSQPSITLTSQPATPTRTIAATPIQTLPQSQSTPKRIDTPSLEEPSDLEELEQFAKTFKQRRIKLGFTQGDVGLAMGKLYGNDFSQTTISRFEALNLSFKNMCKLKPLLEKWLNDAENLSSDSSLSSPSALNSPGIEGLSRRRKKRTSIETNIRVALEKSFLENQKPTSEEITMIADQLNMEKEVIRVWFCNRRQKEKRINPPSSGGTSSSPIKAIFPSPTSLVATTPSLVTSSAATTLTVSPVLPLTSAAVTNLSVTGTSDTTSNNTATVISTAPPASSAVTSPSLSPSPSASASTSEASSASETSTTQTTSTPLSSPLGTSQVMVTASGLQTAAAAALQGAAQLPANASLAAMAAAAGLNPSLMAPSQFAAGGALLSLNPGTLSGALSPALMSNSTLATIQALASGGSLPITSLDATGNLVFANAGGAPNIVTAPLFLNPQNLSLLTSNPVSLVSAAAASAGNSAPVASLHATSTSAESIQNSLFTVASASGAASTTTTASKAQ

SEQ ID NO: 15 - DNA, 2226 bp, Homo sapiens
  source 1..2226: mol_type = other DNA
atgaagacaaggatgaagatctttgtgatgatccacttccacttaatgaatagcacacaaaccaatggtctggactttcagaagcagcctgtgcctgtaggaggagcaatctcaacagcccaggcgcaggctttccttggacatctccatcaggtccaactcgctggaacaagtttacaggctgctgctcagtctttaaatgtacagtctaaatctaatgaagaatcgggggattcgcagcagccaagccagccttcccagcagccttcagtgcaggcagccattccccagacccagcttatgctagctggaggacagataactgggcttactttgacgcctgcccagcaacagttactactccagcaggcacaggcacaggcacagctgctggctgctgcagtgcagcagcactccgccagccagcagcacagtgctgctggagccaccatctccgcctctgctgccacgcccatgacgcagatccccctgtctcagcccatacagatcgcacaggatcttcaacaactgcaacagcttcaacagcagaatctcaacctgcaacagtttgtgttggtgcatccaaccaccaatttgcagccagcgcagtttatcatctcacagacgccccagggccagcagggtctcctgcaagcgcaaaatcttctaacgcaactacctcagcaaagccaagccaacctcctacagtcgcagccaagcatcaccctcacctcccagccagcaaccccaacacgcacaatagcagcaaccccaattcagacacttccacagagccagtcaacaccaaagcgaattgatactcccagcttggaggagcccagtgaccttgaggagcttgagcagtttgccaagaccttcaaacaaagacgaatcaaacttggattcactcagggtgatgttgggctcgctatggggaaactatatggaaatgacttcagccaaactaccatctctcgatttgaagccttgaacctcagctttaagaacatgtgcaagttgaagccacttttagagaagtggctaaatgatgcagagaacctctcatctgattcgtccctctccagcccaagtgccctgaattctccaggaattgagggcttgagccgtaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacccaccaagcagtggtgggaccagcagctcacctattaaagcaattttccccagcccaacttcactggtggcgaccacaccaagccttgtgactagcagtgcagcaactaccctcacagtcagccctgtcctccctctgaccagtgctgctgtgacgaatctttcagttacaggcacttcagacaccacctccaacaacacagcaaccgtgatttccacagcgcctccagcttcctcagcagtcacgtccccctctctgagtccctccccttctgcctcagcctccacctccgaggcatccagtgccagtgagaccagcacaacacagaccacctccactcctttgtcctcccctcttgggaccagccaggtgatggtgacagcatcaggtttgcaaacagcagcagctgctgcccttcaaggagctgcacagttgccagcaaatgccagtcttgctgccatggcagctgctgcaggactaaacccaagcctgatggcaccctcacagtttgcggctggaggtgccttactcagtctgaatccagggaccctgagcggtgctctcagcccagctctaatgagcaacagtacactggcaactattcaagctcttgcttctggtggctctcttccaataacatcacttgatgcaactgggaacctggtatttgccaatgcgggaggagcccccaacatcgtgactgcccctctgttcctgaaccctcagaacctctctctgctcaccagcaaccctgttagcttggtctctgccgccgcagcatctgcagggaactctgcacctgtagccagccttcacgccacctccacctctgctgagtccatccagaactctctcttcacagtggcctctgccagcggggctgcgtccaccaccaccaccgcctccaaggcacagtga

SEQ ID NO: 16 - protein, 740 aa, Homo sapiens
MKTRMKIFVMIHFHLMNSTQTNGLDFQKQPVPVGGAISTAQAQAFLGHLHQVQLAGTSLQAAAQSLNVQSKSNEESGDSQQPSQPSQQPSVQAAIPQTQLMLAGGQITGLTLTPAQQQLLLQQAQAQAQLLAAAVQQHSAQQHSAAGATISASAATPMTQIPLSQPIQIAQDLQQLQQLQQQNLNLQQFVLVHPTTNLQPAQFIISQTPQGQQGLLQAQNLLTQLPQQSQANLLQSQPSITLTSQPATPTRTIAATPIQTLPQSQSTPKRIDTPSLEEPSDLEELEQFAKTFKQRRIKLGFTQGDVGLAMGKLYGNDFSQTTISRFEALNLSFKNMCKLKPLLEKWLNDAENLSSDSSLSSPSALNSPGIEGLSRRRKKRTSIETNIRVALEKSFLENQKPTSEEITMIADQLNMEKEVIRVWFCNRRQKEKRINPPSSGGTSSSPIKAIFPSPTSLVATTPSLVTSSAATTLTVSPVLPLTSAAVTNLSVTGTSDTTSNNTATVISTAPPASSAVTSPSLSPSPSASASTSEASSASETSTTQTTSTPLSSPLGTSQVMVTASGLQTAAAAALQGAAQLPANASLAAMAAAAGLNPSLMAPSQFAAGGALLSLNPGTLSGALSPALMSNSTLATIQALASGGSLPITSLDATGNLVFANAGGAPNIVTAPLFLNPQNLSLLTSNPVSLVSAAAASAGNSAPVASLHATSTSAESIQNSLFTVASASGAASTTTTASKAQ

SEQ ID NO: 17 - DNA, 648 bp, Homo sapiens
  source 1..648: mol_type = other DNA
atgaacctcgtgggcagctacgcacaccatcaccaccatcaccacccgcaccctgcgcaccccatgctccacgaacccttcctcttcggtccggcctcgcgctgtcatcaggaaaggccctacttccagagctggctgctgagcccggctgacgctgccccggacttccctgcgggcgggccgccgcccgcggccgctgcagccgccaccgcctatggtcctgacgccaggcctgggcagagccccgggcggctggaggcgcttggcggccgtcttggccggcggaaaggctcaggacccaagaaggagcggagacgcactgagagcattaacagcgcattcgcggagttgcgcgagtgcatccccaacgtgccggccgacaccaagctctccaagatcaagactctgcgcctagccaccagctacatcgcctacctgatggacgtgctggccaaggatgcacagtctggcgatcccgaggccttcaaggctgaactcaagaaggcggatggcggccgtgagagcaagcggaaaagggagctgcagcagcacgaaggttttcctcctgccctgggcccagtcgagaagaggattaaaggacgcaccggctggccgcagcaagtctgggcgctggagttaaaccagtga

SEQ ID NO: 18 - protein, 215 aa, Homo sapiens
MNLVGSYAHHHHHHHPHPAHPMLHEPFLFGPASRCHQERPYFQSWLLSPADAAPDFPAGGPPPAAAAAATAYGPDARPGQSPGRLEALGGRLGRRKGSGPKKERRRTESINSAFAELRECIPNVPADTKLSKIKTLRLATSYIAYLMDVLAKDAQSGDPEAFKAELKKADGGRESKRKRELQQHEGFPPALGPVEKRIKGRTGWPQQVWALELNQ

SEQ ID NO: 19 - DNA, 645 bp, Homo sapiens
  source 1..645: mol_type = other DNA
atgaacctcgtgggcagctacgcacaccatcaccaccatcaccacccgcaccctgcgcaccccatgctccacgaacccttcctcttcggtccggcctcgcgctgtcatcaggaaaggccctacttccagagctggctgctgagcccggctgacgctgccccggacttccctgcgggcgggccgccgcccgcggccgctgcagccgccaccgcctatggtcctgacgccaggcctgggcagagccccgggcggctggaggcgcttggcggccgtcttggccggcggaaaggctcaggacccaagaaggagcggagacgcactgagagcattaacagcgcattcgcggagttgcgcgagtgcatccccaacgtgccggccgacaccaagctctccaagatcaagactctgcgcctagccaccagctacatcgcctacctgatggacgtgctggccaaggatgcacagtctggcgatcccgaggccttcaaggctgaactcaagaaggcggatggcggccgtgagagcaagcggaaaagggagctgcagcacgaaggttttcctcctgccctgggcccagtcgagaagaggattaaaggacgcaccggctggccgcagcaagtctgggcgctggagttaaaccagtga

SEQ ID NO: 20 - protein, 214 aa, Homo sapiens
MNLVGSYAHHHHHHHPHPAHPMLHEPFLFGPASRCHQERPYFQSWLLSPADAAPDFPAGGPPPAAAAAATAYGPDARPGQSPGRLEALGGRLGRRKGSGPKKERRRTESINSAFAELRECIPNVPADTKLSKIKTLRLATSYIAYLMDVLAKDAQSGDPEAFKAELKKADGGRESKRKRELQHEGFPPALGPVEKRIKGRTGWPQQVWALELNQ

SEQ ID NO: 21 - DNA, 3051 bp, Homo sapiens
  source 1..3051: mol_type = other DNA
atggaggtggcggtggagaaggcggtggcggcggcggcagcggcctcggctgcggcctccggggggccctcggcggcgccgagcggggagaacgaggccgagagtcggcagggcccggactcggagcgcggcggcgaggcggcccggctcaacctgttggacacttgcgccgtgtgccaccagaacatccagagccgggcgcccaagctgctgccctgcctgcactctttctgccagcgctgcctgcccgcgccccagcgctacctcatgctgcccgcgcccatgctgggctcggccgagaccccgccacccgtccctgcccccggctcgccggtcagcggctcgtcgccgttcgccacccaagttggagtcattcgttgcccagtttgcagccaagaatgtgcagagagacacatcatagataacttttttgtgaaggacactactgaggttcccagcagtacagtagaaaagtcaaatcaggtatgtacaagctgtgaggacaacgcagaagccaatgggttttgtgtagagtgtgttgaatggctctgcaagacgtgtatcagagctcatcagagggtaaagttcacaaaagaccacactgtcagacagaaagaggaagtatctccagaggcagttggtgtcaccagccagcgaccagtgttttgtccttttcataaaaaggagcagctgaagctgtactgtgagacatgtgacaaactgacatgtcgagactgtcagttgttagaacataaagagcatagataccaatttatagaagaagcttttcagaatcagaaagtgatcatagatacactaatcaccaaactgatggaaaaaacaaaatacataaaattcacaggaaatcagatccaaaacagaattattgaagtaaatcaaaatcaaaagcaggtggaacaggatattaaagttgctatatttacactgatggtagaaataaataaaaaaggaaaagctctactgcatcagttagagagccttgcaaaggaccatcgcatgaaacttatgcaacaacaacaggaagtggctggactctctaaacaattggagcatgtcatgcatttttctaaatgggcagtttccagtggcagcagtacagcattactttatagcaaacgactgattacataccggttacggcacctccttcgtgcaaggtgtgatgcatccccagtgaccaacaacaccatccaatttcactgtgatcctagtttctgggctcaaaatatcatcaacttaggttctttagtaatcgaggataaagagagccagccacaaatgcctaagcagaatcctgtcgtggaacagaattcacagccaccaagtggtttatcatcaaaccagttatccaagttcccaacacagatcagcctagctcaattacggctccagcatatgcagcaacagcaaccgcctccacgtttgataaactttcagaatcacagccccaaacccaatggaccagttcttcctcctcatcctcaacaactgagatatccaccaaaccagaacataccacgacaagcaataaagccaaaccccctacagatggctttcttggctcaacaagccataaaacagtggcagatcagcagtggacagggaaccccatcaactaccaacagcacatcctctactccttccagccccacgattactagtgcagcaggatatgatggaaaggcttttggttcacctatgatcgatttgagctcaccagtgggagggtcttataatcttccctctcttccggatattgactgttcaagtactattatgctggacaatattgtgaggaaagatactaatatagatcatggccagccaagaccaccctcaaacagaacggtccagtcaccaaattcatcagtgccatctccaggccttgcaggacctgttactatgactagtgtacaccccccaatacgttcacctagtgcctccagcgttggaagccgaggaagctctggctcttccagcaaaccagcaggagctgactctacacacaaagtcccagtggtcatgctggagccaattcgaataaaacaagaaaacagtggaccaccggaaaattatgatttccctgttgttatagtgaagcaagaatcagatgaagaatctaggcctcaaaatgccaattatccaagaagcatactcacctccctgctcttaaatagcagtcagagctctacttctgaggagactgtgctaagatcagatgcccctgatagtacaggagatcaacctggacttcaccaggacaattcctcaaatggaaagtctgaatggttggatccttcccagaagtcacctcttcatgttggagagacaaggaaagaggatgaccccaatgaggactggtgtgcagtttgtcaaaacggaggggaactcctctgctgtgaaaagtgccccaaagtattccatctttcttgtcatgtgcccacattgacaaattttccaagtggagagtggatttgcactttctgccgagacttatctaaaccagaagttgaatatgattgtgatgctcccagtcacaactcagaaaaaaagaaaactgaaggccttgttaagttaacacctatagataaaaggaagtgtgagcgcctacttttatttctttactgccatgaaatgagcctggcttttcaagaccctgttcctctaactgtgcctgattattacaaaataattaaaaatccaatggatttgtcaaccatcaagaaaagactacaagaagattattccatgtactcaaaacctgaagattttgtagctgattttagattgatctttcaaaactgtgctgaattcaatgagcctgattcagaagtagccaatgctggtataaaacttgaaaattattttgaagaacttctaaagaacctctatccagaaaaaaggtttcccaaaccagaattcaggaatgaatcagaagataataaatttagtgatgattcagatgatgactttgtacagccccggaagaaacgcctcaaaagcattgaagaacgccagttgcttaaataa

SEQ ID NO: 22 - protein, 1016 aa, Homo sapiens
MEVAVEKAVAAAAAASAAASGGPSAAPSGENEAESRQGPDSERGGEAARLNLLDTCAVCHQNIQSRAPKLLPCLHSFCQRCLPAPQRYLMLPAPMLGSAETPPPVPAPGSPVSGSSPFATQVGVIRCPVCSQECAERHIIDNFFVKDTTEVPSSTVEKSNQVCTSCEDNAEANGFCVECVEWLCKTCIRAHQRVKFTKDHTVRQKEEVSPEAVGVTSQRPVFCPFHKKEQLKLYCETCDKLTCRDCQLLEHKEHRYQFIEEAFQNQKVIIDTLITKLMEKTKYIKFTGNQIQNRIIEVNQNQKQVEQDIKVAIFTLMVEINKKGKALLHQLESLAKDHRMKLMQQQQEVAGLSKQLEHVMHFSKWAVSSGSSTALLYSKRLITYRLRHLLRARCDASPVTNNTIQFHCDPSFWAQNIINLGSLVIEDKESQPQMPKQNPVVEQNSQPPSGLSSNQLSKFPTQISLAQLRLQHMQQQQPPPRLINFQNHSPKPNGPVLPPHPQQLRYPPNQNIPRQAIKPNPLQMAFLAQQAIKQWQISSGQGTPSTTNSTSSTPSSPTITSAAGYDGKAFGSPMIDLSSPVGGSYNLPSLPDIDCSSTIMLDNIVRKDTNIDHGQPRPPSNRTVQSPNSSVPSPGLAGPVTMTSVHPPIRSPSASSVGSRGSSGSSSKPAGADSTHKVPVVMLEPIRIKQENSGPPENYDFPVVIVKQESDEESRPQNANYPRSILTSLLLNSSQSSTSEETVLRSDAPDSTGDQPGLHQDNSSNGKSEWLDPSQKSPLHVGETRKEDDPNEDWCAVCQNGGELLCCEKCPKVFHLSCHVPTLTNFPSGEWICTFCRDLSKPEVEYDCDAPSHNSEKKKTEGLVKLTPIDKRKCERLLLFLYCHEMSLAFQDPVPLTVPDYYKIIKNPMDLSTIKKRLQEDYSMYSKPEDFVADFRLIFQNCAEFNEPDSEVANAGIKLENYFEELLKNLYPEKRFPKPEFRNESEDNKFSDDSDDDFVQPRKKRLKSIEERQLLK

SEQ ID NO: 23 - DNA, 3153 bp, Homo sapiens
  source 1..3153: mol_type = other DNA
atggaggtggcggtggagaaggcggtggcggcggcggcagcggcctcggctgcggcctccggggggccctcggcggcgccgagcggggagaacgaggccgagagtcggcagggcccggactcggagcgcggcggcgaggcggcccggctcaacctgttggacacttgcgccgtgtgccaccagaacatccagagccgggcgcccaagctgctgccctgcctgcactctttctgccagcgctgcctgcccgcgccccagcgctacctcatgctgcccgcgcccatgctgggctcggccgagaccccgccacccgtccctgcccccggctcgccggtcagcggctcgtcgccgttcgccacccaagttggagtcattcgttgcccagtttgcagccaagaatgtgcagagagacacatcatagataacttttttgtgaaggacactactgaggttcccagcagtacagtagaaaagtcaaatcaggtatgtacaagctgtgaggacaacgcagaagccaatgggttttgtgtagagtgtgttgaatggctctgcaagacgtgtatcagagctcatcagagggtaaagttcacaaaagaccacactgtcagacagaaagaggaagtatctccagaggcagttggtgtcaccagccagcgaccagtgttttgtccttttcataaaaaggagcagctgaagctgtactgtgagacatgtgacaaactgacatgtcgagactgtcagttgttagaacataaagagcatagataccaatttatagaagaagcttttcagaatcagaaagtgatcatagatacactaatcaccaaactgatggaaaaaacaaaatacataaaattcacaggaaatcagatccaaaacagaattattgaagtaaatcaaaatcaaaagcaggtggaacaggatattaaagttgctatatttacactgatggtagaaataaataaaaaaggaaaagctctactgcatcagttagagagccttgcaaaggaccatcgcatgaaacttatgcaacaacaacaggaagtggctggactctctaaacaattggagcatgtcatgcatttttctaaatgggcagtttccagtggcagcagtacagcattactttatagcaaacgactgattacataccggttacggcacctccttcgtgcaaggtgtgatgcatccccagtgaccaacaacaccatccaatttcactgtgatcctagtttctgggctcaaaatatcatcaacttaggttctttagtaatcgaggataaagagagccagccacaaatgcctaagcagaatcctgtcgtggaacagaattcacagccaccaagtggtttatcatcaaaccagttatccaagttcccaacacagatcagcctagctcaattacggctccagcatatgcagcaacaggtaatggctcagaggcaacaggtgcaacggaggccagcacctgtgggtttaccaaaccctagaatgcaggggcccatccagcaaccttccatctctcatcagcaaccgcctccacgtttgataaactttcagaatcacagccccaaacccaatggaccagttcttcctcctcatcctcaacaactgagatatccaccaaaccagaacataccacgacaagcaataaagccaaaccccctacagatggctttcttggctcaacaagccataaaacagtggcagatcagcagtggacagggaaccccatcaactaccaacagcacatcctctactccttccagccccacgattactagtgcagcaggatatgatggaaaggcttttggttcacctatgatcgatttgagctcaccagtgggagggtcttataatcttccctctcttccggatattgactgttcaagtactattatgctggacaatattgtgaggaaagatactaatatagatcatggccagccaagaccaccctcaaacagaacggtccagtcaccaaattcatcagtgccatctccaggccttgcaggacctgttactatgactagtgtacaccccccaatacgttcacctagtgcctccagcgttggaagccgaggaagctctggctcttccagcaaaccagcaggagctgactctacacacaaagtcccagtggtcatgctggagccaattcgaataaaacaagaaaacagtggaccaccggaaaattatgatttccctgttgttatagtgaagcaagaatcagatgaagaatctaggcctcaaaatgccaattatccaagaagcatactcacctccctgctcttaaatagcagtcagagctctacttctgaggagactgtgctaagatcagatgcccctgatagtacaggagatcaacctggacttcaccaggacaattcctcaaatggaaagtctgaatggttggatccttcccagaagtcacctcttcatgttggagagacaaggaaagaggatgaccccaatgaggactggtgtgcagtttgtcaaaacggaggggaactcctctgctgtgaaaagtgccccaaagtattccatctttcttgtcatgtgcccacattgacaaattttccaagtggagagtggatttgcactttctgccgagacttatctaaaccagaagttgaatatgattgtgatgctcccagtcacaactcagaaaaaaagaaaactgaaggccttgttaagttaacacctatagataaaaggaagtgtgagcgcctacttttatttctttactgccatgaaatgagcctggcttttcaagaccctgttcctctaactgtgcctgattattacaaaataattaaaaatccaatggatttgtcaaccatcaagaaaagactacaagaagattattccatgtactcaaaacctgaagattttgtagctgattttagattgatctttcaaaactgtgctgaattcaatgagcctgattcagaagtagccaatgctggtataaaacttgaaaattattttgaagaacttctaaagaacctctatccagaaaaaaggtttcccaaaccagaattcaggaatgaatcagaagataataaatttagtgatgattcagatgatgactttgtacagccccggaagaaacgcctcaaaagcattgaagaacgccagttgcttaaataa

SEQ ID NO: 24 - protein, 1050 aa, Homo sapiens
MEVAVEKAVAAAAAASAAASGGPSAAPSGENEAESRQGPDSERGGEAARLNLLDTCAVCHQNIQSRAPKLLPCLHSFCQRCLPAPQRYLMLPAPMLGSAETPPPVPAPGSPVSGSSPFATQVGVIRCPVCSQECAERHIIDNFFVKDTTEVPSSTVEKSNQVCTSCEDNAEANGFCVECVEWLCKTCIRAHQRVKFTKDHTVRQKEEVSPEAVGVTSQRPVFCPFHKKEQLKLYCETCDKLTCRDCQLLEHKEHRYQFIEEAFQNQKVIIDTLITKLMEKTKYIKFTGNQIQNRIIEVNQNQKQVEQDIKVAIFTLMVEINKKGKALLHQLESLAKDHRMKLMQQQQEVAGLSKQLEHVMHFSKWAVSSGSSTALLYSKRLITYRLRHLLRARCDASPVTNNTIQFHCDPSFWAQNIINLGSLVIEDKESQPQMPKQNPVVEQNSQPPSGLSSNQLSKFPTQISLAQLRLQHMQQQVMAQRQQVQRRPAPVGLPNPRMQGPIQQPSISHQQPPPRLINFQNHSPKPNGPVLPPHPQQLRYPPNQNIPRQAIKPNPLQMAFLAQQAIKQWQISSGQGTPSTTNSTSSTPSSPTITSAAGYDGKAFGSPMIDLSSPVGGSYNLPSLPDIDCSSTIMLDNIVRKDTNIDHGQPRPPSNRTVQSPNSSVPSPGLAGPVTMTSVHPPIRSPSASSVGSRGSSGSSSKPAGADSTHKVPVVMLEPIRIKQENSGPPENYDFPVVIVKQESDEESRPQNANYPRSILTSLLLNSSQSSTSEETVLRSDAPDSTGDQPGLHQDNSSNGKSEWLDPSQKSPLHVGETRKEDDPNEDWCAVCQNGGELLCCEKCPKVFHLSCHVPTLTNFPSGEWICTFCRDLSKPEVEYDCDAPSHNSEKKKTEGLVKLTPIDKRKCERLLLFLYCHEMSLAFQDPVPLTVPDYYKIIKNPMDLSTIKKRLQEDYSMYSKPEDFVADFRLIFQNCAEFNEPDSEVANAGIKLENYFEELLKNLYPEKRFPKPEFRNESEDNKFSDDSDDDFVQPRKKRLKSIEERQLLK

SEQ ID NO: 25 - DNA, 1329 bp, Homo sapiens
  source 1..1329: mol_type = other DNA
atgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 26 - protein, 442 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 27 - DNA, 1332 bp, Homo sapiens
  source 1..1332: mol_type = other DNA
atgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 28 - protein, 443 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLVDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 29 - DNA, 711 bp, Homo sapiens
  source 1..711: mol_type = other DNA
atgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 30 - protein, 236 aa, Homo sapiens
MFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 31 - DNA, 585 bp, Homo sapiens
  source 1..585: mol_type = other DNA
atgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 32 - protein, 194 aa, Homo sapiens
MFDDFSEGRECVNCGAMSTPLWRRDGTGHYLCNACGLYHKMNGINRPLIKPQRRLVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 33 - DNA, 1293 bp, Homo sapiens
  source 1..1293: mol_type = other DNA
atggacgagcagcccaggctgatgcattcccatgctggggtcgggatggccggacaccccggcctgtcccagcacttgcaggatggggccggagggaccgagggggagggcgggaggaagcaggacattggagacattttacagcaaattatgaccatcacagaccagagtttggatgaggcgcaggccagaaaacatgctttaaactgccacagaatgaagcctgccttgtttaatgtgttgtgtgaaatcaaagaaaaaacagttttgagtatccgaggagcccaggaggaggaacccacagacccccagctgatgcggctggacaacatgctgttagcggaaggcgtggcggggcctgagaagggcggagggtcggcggcagcggcggcagcggcggcggcttctggaggggcaggttcagacaactcagtggagcattcagattacagagccaaactctcacagatcagacaaatctaccatacggagctggagaaatacgagcaggcctgcaacgagttcaccacccacgtgatgaatctcctgcgagagcaaagccggaccaggcccatctccccaaaggagattgagcggatggtcagcatcatccaccgcaagttcagctccatccagatgcagctcaagcagagcacgtgcgaggcggtgatgatcctgcgttcccgatttctggatgcgcggcggaagagacggaatttcaacaagcaagcgacagaaatcctgaatgaatatttctattcccatctcagcaacccttaccccagtgaggaagccaaagaggagttagccaagaagtgtggcatcacagtctcccaggtatcaaactggtttggaaataagcgaatccggtacaagaagaacataggtaaatttcaagaggaagccaatatttatgctgccaaaacagctgtcactgctaccaatgtgtcagcccatggaagccaagctaactcgccctcaactcccaactcggctggttcttccagttcttttaacatgtcaaactctggagatttgttcatgagcgtgcagtcactcaatggggattcttaccaaggggcccaggttggagccaacgtgcaatcacaggtggatacccttcgccatgttatcagccagacaggaggatacagtgatggactcgcagccagtcagatgtacagtccgcagggcatcagtgctaatggaggttggcaggatgctactaccccttcatcagtgacctcccctacagaaggccctggcagtgttcactctgatacctccaactga

SEQ ID NO: 34 - protein, 430 aa, Homo sapiens
MDEQPRLMHSHAGVGMAGHPGLSQHLQDGAGGTEGEGGRKQDIGDILQQIMTITDQSLDEAQARKHALNCHRMKPALFNVLCEIKEKTVLSIRGAQEEEPTDPQLMRLDNMLLAEGVAGPEKGGGSAAAAAAAAASGGAGSDNSVEHSDYRAKLSQIRQIYHTELEKYEQACNEFTTHVMNLLREQSRTRPISPKEIERMVSIIHRKFSSIQMQLKQSTCEAVMILRSRFLDARRKRRNFNKQATEILNEYFYSHLSNPYPSEEAKEELAKKCGITVSQVSNWFGNKRIRYKKNIGKFQEEANIYAAKTAVTATNVSAHGSQANSPSTPNSAGSSSSFNMSNSGDLFMSVQSLNGDSYQGAQVGANVQSQVDTLRHVISQTGGYSDGLAASQMYSPQGISANGGWQDATTPSSVTSPTEGPGSVHSDTSN

SEQ ID NO: 35 - DNA, 2138 bp, Homo sapiens
  source 1..2138: mol_type = other DNA
atgggcatgaggatcaaactgcaaagcaccaaccaccccaacaacctgctgaaggaactcaacaagtgccggctctcagagaccatgtgcgacgtcaccattgtggtggggagccgctccttcccggcccacaaggctgtgctggcctgtgcagctggctacttccagaacctcttcctgaatactgggcttgatgctgccaggacctatgtggtggacttcatcacccctgccaactttgagaaggttctgagctttgtctacacttcagaactcttcacagacctgatcaatgttggggtcatctacgaggtagctgagcgtctgggtatggaggacctcctccaggcctgtcactctacctttcctgatctggagagcactgccagggccaagcccctgaccagcaccagtgagagccactctggtaccctgagttgtccttcggcagaacctgcccatccccttggagaactccgaggtggtggggctaccttggtgctgatagaaactatgtgttgcccagtgatgctggagggagctataaagaggaagagaagaatgttgccagtgacgctaaccatagcctgcatctgccgcaaccgcccccaccaccgccaaagacagaagaccatgacacccctgctcccttcacgtccattcctagcatgatgacccagccactcctaggcactgtcagcacgggcatccagaccagcacgagctcctgccagccatacaaagttcaaagcaatggagacttcagtaaaaacagcttcctcacccctgacaatgcagtagacattaccactgggaccaactcctgtctgagcaatagtgagcactccaaagatcctggctttgggcagatggatgagctccagctcgaggacctgggggatgatgacttgcagtttgaagaccctgctgaggatataggcacaactgaggaggtgattgagctgagtgatgacagtgaggatgagttggcttttggagagaatgacaatcgggagaataaggccatgccctgccaggtgtgcaagaaagttctagagcccaacattcaactgatccggcagcatgctcgggaccatgtggacctgctgacgggcaactgcaaggtctgcgagacccacttccaggaccgaaactcccgggtaactcatgtcctgtcccacattggtattttccttttctcctgcgacatgtgtgaaactaagttctttacccagtggcagctgacccttcaccgacgggatggaatatttgagaacaacatcattgtccaccccaacgatcccctgccagggaagctgggtctcttttcaggggcagcctccccagagctgaaatgcgctgcctgtgggaaagtattggccaaagatttccatgtggtccggggccacatccttgaccatctaaacttgaagggccaggcctgcagtgtctgcgaccagcgtcaccttaacctctgcagcctcatgtggcacacgctgtcccatctcggcatctcagtcttctcctgttctgtctgtgcgaacagctttgtggactggcatcttctagagaagcacatggctgtgcaccaaagtctggaagacgccctcttccactgccgcttgtgcagccagagcttcaagtcagaggctgcctatcgctaccacgtcagccagcacaaatgcaacagtggccttgatgcacggcctggttttgggctgcagcacccagctctccagaagcggaagctgccagcagaggagtttctgggtgaagagctggcgctgcagggccaacctgggaacagcaagtatagctgcaaggtctgtggcaaaagatttgcccacacaagcgaattcaactaccaccggcggatccacacgggggagaagccataccaatgtaaggtgtgccacaagttctttcgaggccgctcgaccatcaagtgccacctaaagacacactcgggggccctcatgtaccgctgcacagtctgtgggcactacagttccacccttaacctcatgagcaaacatgttggtgtgcacaaaggcagcctcccccctgacttcaccatcgagcagaccttcatgtacatcatccattccaaagaggcggataagaacccggacagttga

SEQ ID NO: 36 - protein, 712 aa, Homo sapiens
MGMRIKLQSTNHPNNLLKELNKCRLSETMCDVTIVVGSRSFPAHKAVLACAAGYFQNLFLNTGLDAARTYVVDFITPANFEKVLSFVYTSELFTDLINVGVIYEVAERLGMEDLLQACHSTFPDLESTARAKPLTSTSESHSGTLSCPSAEPAHPLGELRGGGDYLGADRNYVLPSDAGGSYKEEEKNVASDANHSLHLPQPPPPPPKTEDHDTPAPFTSIPSMMTQPLLGTVSTGIQTSTSSCQPYKVQSNGDFSKNSFLTPDNAVDITTGTNSCLSNSEHSKDPGFGQMDELQLEDLGDDDLQFEDPAEDIGTTEEVIELSDDSEDELAFGENDNRENKAMPCQVCKKVLEPNIQLIRQHARDHVDLLTGNCKVCETHFQDRNSRVTHVLSHIGIFLFSCDMCETKFFTQWQLTLHRRDGIFENNIIVHPNDPLPGKLGLFSGAASPELKCAACGKVLAKDFHVVRGHILDHLNLKGQACSVCDQRHLNLCSLMWHTLSHLGISVFSCSVCANSFVDWHLLEKHMAVHQSLEDALFHCRLCSQSFKSEAAYRYHVSQHKCNSGLDARPGFGLQHPALQKRKLPAEEFLGEELALQGQPGNSKYSCKVCGKRFAHTSEFNYHRRIHTGEKPYQCKVCHKFFRGRSTIKCHLKTHSGALMYRCTVCGHYSSTLNLMSKHVGVHKGSLPPDFTIEQTFMYIIHSKEADKNPDS

SEQ ID NO: 37 - DNA, 654 bp, Homo sapiens
  source 1..654: mol_type = other DNA
atgagtctggtaggtggttttccccaccacccggtggtgcaccacgagggctacccgtttgccgccgccgccgccgcagctgccgccgccgccgccagccgctgcagccatgaggagaacccctacttccatggctggctcatcggccaccccgagatgtcgccccccgactacagcatggccctgtcctacagccccgagtatgccagcggcgccgccggcctggaccactcccattacgggggggtgccgccgggcgccgggcccccgggcctgggggggccgcgcccggtgaagcgccgaggcaccgccaaccgcaaggagcggcgcaggactcagagcatcaacagcgccttcgccgaactgcgcgagtgcatccccaacgtacccgccgacaccaaactctccaaaatcaagaccctgcgcctggccaccagctacatcgcctacctcatggacctgctggccaaggacgaccagaatggcgaggcggaggccttcaaggcagagatcaagaagaccgacgtgaaagaggagaagaggaagaaggagctgaacgaaatcttgaaaagcacagtgagcagcaacgacaagaaaaccaaaggccggacgggctggccgcagcacgtctgggccctggagctcaagcagtga

SEQ ID NO: 38 - protein, 217 aa, Homo sapiens
MSLVGGFPHHPVVHHEGYPFAAAAAAAAAAAASRCSHEENPYFHGWLIGHPEMSPPDYSMALSYSPEYASGAAGLDHSHYGGVPPGAGPPGLGGPRPVKRRGTANRKERRRTQSINSAFAELRECIPNVPADTKLSKIKTLRLATSYIAYLMDLLAKDDQNGEAEAFKAEIKKTDVKEEKRKKELNEILKSTVSSNDKKTKGRTGWPQHVWALELKQ

SEQ ID NO: 39 - DNA, 1617 bp, Homo sapiens
  source 1..1617: mol_type = other DNA
atggacatagaagactgcaatggccgctcctatgtgtctggtagcggggactcatctctggagaaggagttcctcggggccccagtggggccctcggtgagcacccccaacagccagcactcttctcctagccgctcactcagtgccaactccatcaaggtggagatgtacagcgatgaggagtcaagcagactgctggggccagatgagcggctcctggaaaaggacgacagcgtgattgtggaagattcattgtctgagcccctgggctactgtgatgggagtgggccagagcctcactcccctgggggcatccggctgcccaatggcaagctcaagtgtgacgtctgcggcatggtctgtattggacccaacgtgctcatggtgcacaagcgcagtcacactggtgaaaggcccttccattgcaaccagtgtggtgcctccttcacccagaaggggaacctgctgcgccacatcaagctgcactctggggagaagccctttaaatgtcccttctgcaactatgcctgccgccggcgtgatgcactcactggtcacctccgcacacactcagtctcctctcccacagtgggcaagccctacaagtgtaactactgtggccggagctacaaacagcagagtaccctggaggagcacaaggagcggtgccataactacctacagagtctcagcactgaagcccaagctttggctggccaaccaggtgacgaaatacgtgacctggagatggtgccagactccatgctgcactcatcctctgagcggccaactttcatcgatcgtctggccaatagcctcaccaaacgcaagcgttccacaccccagaagtttgtaggcgaaaagcagatgcgcttcagcctctcagacctcccctatgatgtgaactcgggtggctatgaaaaggatgtggagttggtggcacaccacagcctagagcctggctttggaagttccctggcctttgtgggtgcagagcatctgcgtcccctccgccttccacccaccaattgcatctcagaactcacgcctgtcatcagctctgtctacacccagatgcagcccctccctggtcgactggagcttccaggatcccgagaagcaggtgagggacctgaggacctggctgatggaggtcccctcctctaccggccccgaggccccctgactgaccctggggcatcccccagcaatggctgccaggactccacagacacagaaagcaaccacgaagatcgggttgcgggggtggtatccctccctcagggtcccccaccccagccacctcccaccattgtggtgggccggcacagtcctgcctacgccaaagaggaccccaagccacaggaggggttattgcggggcaccccaggcccctccaaggaagtgcttcgggtggtgggcgagagtggtgagcctgtgaaggccttcaagtgtgagcactgccgtatcctcttcctggaccacgtcatgttcactatccacatgggctgccatggcttcagagacccttttgagtgcaacatctgtggttatcacagccaggaccggtacgaattctcttcccacattgtccggggggagcataaggtgggctag

SEQ ID NO: 40 - protein, 538 aa, Homo sapiens
MDIEDCNGRSYVSGSGDSSLEKEFLGAPVGPSVSTPNSQHSSPSRSLSANSIKVEMYSDEESSRLLGPDERLLEKDDSVIVEDSLSEPLGYCDGSGPEPHSPGGIRLPNGKLKCDVCGMVCIGPNVLMVHKRSHTGERPFHCNQCGASFTQKGNLLRHIKLHSGEKPFKCPFCNYACRRRDALTGHLRTHSVSSPTVGKPYKCNYCGRSYKQQSTLEEHKERCHNYLQSLSTEAQALAGQPGDEIRDLEMVPDSMLHSSSERPTFIDRLANSLTKRKRSTPQKFVGEKQMRFSLSDLPYDVNSGGYEKDVELVAHHSLEPGFGSSLAFVGAEHLRPLRLPPTNCISELTPVISSVYTQMQPLPGRLELPGSREAGEGPEDLADGGPLLYRPRGPLTDPGASPSNGCQDSTDTESNHEDRVAGVVSLPQGPPPQPPPTIVVGRHSPAYAKEDPKPQEGLLRGTPGPSKEVLRVVGESGEPVKAFKCEHCRILFLDHVMFTIHMGCHGFRDPFECNICGYHSQDRYEFSSHIVRGEHKVG

SEQ ID NO: 41 - DNA, 774 bp, Homo sapiens
  source 1..774: mol_type = other DNA
atgagcaccagccaaccaggggcctgcccatgccagggagctgcaagccgccccgccattctctacgcacttctgagctccagcctcaaggctgtcccccgaccccgtagccgctgcctatgtaggcagcaccggcccgtccagctatgtgcacctcatcgcacctgccgggaggccttggatgttctggccaagacagtggccttcctcaggaacctgccatccttctggcagctgcctccccaggaccagcggcggctgctgcagggttgctggggccccctcttcctgcttgggttggcccaagatgctgtgacctttgaggtggctgaggccccggtgcccagcatactcaagaagattctgctggaggagcccagcagcagtggaggcagtggccaactgccagacagaccccagccctccctggctgcggtgcagtggcttcaatgctgtctggagtccttctggagcctggagcttagccccaaggaatatgcctgcctgaaagggaccatcctcttcaaccccgatgtgccaggcctccaagccgcctcccacattgggcacctgcagcaggaggctcactgggtgctgtgtgaagtcctggaaccctggtgcccagcagcccaaggccgcctgacccgtgtcctcctcacggcctccaccctcaagtccattccgaccagcctgcttggggacctcttctttcgccctatcattggagatgttgacatcgctggccttcttggggacatgcttttgctcaggtga

SEQ ID NO: 42 - protein, 257 aa, Homo sapiens
MSTSQPGACPCQGAASRPAILYALLSSSLKAVPRPRSRCLCRQHRPVQLCAPHRTCREALDVLAKTVAFLRNLPSFWQLPPQDQRRLLQGCWGPLFLLGLAQDAVTFEVAEAPVPSILKKILLEEPSSSGGSGQLPDRPQPSLAAVQWLQCCLESFWSLELSPKEYACLKGTILFNPDVPGLQAASHIGHLQQEAHWVLCEVLEPWCPAAQGRLTRVLLTASTLKSIPTSLLGDLFFRPIIGDVDIAGLLGDMLLLR

SEQ ID NO: 43 - DNA, 648 bp, Homo sapiens
  source 1..648: mol_type = other DNA
atgccgggcgaagccacagaaaccgtccctgctacagagcaggagttgccgcagtcccaggctgagacagggtctggaacagcatctgatagtggtgaatcagtaccagggattgaagaacaggattccacccagaccaccacacaaaaagcctggctggtggcagcagctgaaattgatgaagaaccagtcggtaaagcaaaacagagtcggagtgaaaagagggcacggaaggctatgtccaaactgggtcttctacaggttacaggagttactagagtcactatctggaaatctaagaatatcctctttgtcatcacaaaactggacgtctacaagagccctgcttcggatgcctacatagtttttggggaagccaagatccaagatttatctcagcaagcacaactagcagctgcggagaaattcagagttcaaggtgaagctgtcggaaacattcaagaaaacacacagactccaactgtacaagaggagagtgaagaggaagaggtcgatgaaacaggtgtagaagttaaagacgtgaaattggtcatgtcacaagcaaatgtgtcgagagcaaaggcagtccgagctctgaagaacaacagtaatgatattgtaaatgcgattatggaattaacagtgtaa

SEQ ID NO: 44 - protein, 215 aa, Homo sapiens
MPGEATETVPATEQELPQSQAETGSGTASDSGESVPGIEEQDSTQTTTQKAWLVAAAEIDEEPVGKAKQSRSEKRARKAMSKLGLLQVTGVTRVTIWKSKNILFVITKLDVYKSPASDAYIVFGEAKIQDLSQQAQLAAAEKFRVQGEAVGNIQENTQTPTVQEESEEEEVDETGVEVKDVKLVMSQANVSRAKAVRALKNNSNDIVNAIMELTV

SEQ ID NO: 45 - DNA, 1473 bp, Homo sapiens
  source 1..1473: mol_type = other DNA
atgacaatagggagaatggagaacgtggaggtcttcaccgctgagggcaaaggaaggggtctgaaggccaccaaggagttctgggctgcagatatcatctttgctgagcgggcttattccgcagtggtttttgacagccttgttaattttgtgtgccacacctgcttcaagaggcaggagaagctccatcgctgtgggcagtgcaagtttgcccattactgcgaccgcacctgccagaaggatgcttggctgaaccacaagaatgaatgttcggccatcaagagatatgggaaggtgcccaatgagaacatcaggctggcggcgcgcatcatgtggcgggtggagagagaaggcaccgggctcacggagggctgcctggtgtccgtggacgacttgcagaaccacgtggagcactttggggaggaggagcagaaggacctgcgggtggacgtggacacattcttgcagtactggccgccgcagagccagcagttcagcatgcagtacatctcgcacatcttcggagtgattaactgcaacggttttactctcagtgatcagagaggcctgcaggccgtgggcgtaggcatcttccccaacctgggcctggtgaaccatgactgttggcccaactgtactgtcatatttaacaatggcaatcatgaggcagtgaaatccatgtttcatacccagatgagaattgagctccgggccctaggcaagatctcagaaggagaggagctgactgtgtcctatattgacttcctcaacgttagtgaagaacgcaagaggcagctgaagaagcagtactactttgactgcacatgtgaacactgccagaaaaaactgaaggatgacctcttcctgggggtgaaagacaaccccaagccctctcaggaagtggtgaaggagatgatacaattctccaaggatacattggaaaagatagacaaggctcgttccgagggtttgtatcatgaggttgtgaaattatgccgggagtgcctggagaagcaggagccagtgtttgctgacaccaacatctacatgctgcggatgctgagcattgtttcggaggtcctttcctacctccaggcctttgaggaggcctcgttctatgccaggaggatggtggacggctatatgaagctctaccaccccaacaatgcccaactgggcatggccgtgatgcgggcagggctgaccaactggcatgctggtaacattgaggtggggcacgggatgatctgcaaagcctatgccattctcctggtgacacacggaccctcccaccccatcactaaggacttagaggccatgcgggtgcagacggagatggagctacgcatgttccgccagaacgaattcatgtactacaagatgcgcgaggctgccctgaacaaccagcccatgcaggtcatggccgagcccagcaatgagccatccccagctctgttccacaagaagcaatga

SEQ ID NO: 46 - protein, 490 aa, Homo sapiens
MTIGRMENVEVFTAEGKGRGLKATKEFWAADIIFAERAYSAVVFDSLVNFVCHTCFKRQEKLHRCGQCKFAHYCDRTCQKDAWLNHKNECSAIKRYGKVPNENIRLAARIMWRVEREGTGLTEGCLVSVDDLQNHVEHFGEEEQKDLRVDVDTFLQYWPPQSQQFSMQYISHIFGVINCNGFTLSDQRGLQAVGVGIFPNLGLVNHDCWPNCTVIFNNGNHEAVKSMFHTQMRIELRALGKISEGEELTVSYIDFLNVSEERKRQLKKQYYFDCTCEHCQKKLKDDLFLGVKDNPKPSQEVVKEMIQFSKDTLEKIDKARSEGLYHEVVKLCRECLEKQEPVFADTNIYMLRMLSIVSEVLSYLQAFEEASFYARRMVDGYMKLYHPNNAQLGMAVMRAGLTNWHAGNIEVGHGMICKAYAILLVTHGPSHPITKDLEAMRVQTEMELRMFRQNEFMYYKMREAALNNQPMQVMAEPSNEPSPALFHKKQ

SEQ ID NO: 47 - DNA, 1434 bp, Homo sapiens
  source 1..1434: mol_type = other DNA
atgacaatagggagaatggagaacgtggaggtcttcaccgctgagggcaaaggaaggggtctgaaggccaccaaggagttctgggctgcagatatcatctttgctgagcgggcttattccgcagtggtttttgacagccttgttaattttgtgtgccacacctgcttcaagaggcaggagaagctccatcgctgtgggcagtgcaagtttgcccattactgcgaccgcacctgccagaaggatgcttggctgaaccacaagaatgaatgttcggccatcaagagatatgggaaggtgcccaatgagaacatcaggctggcggcgcgcatcatgtggcgggtggagagagaaggcaccgggctcacggagggctgcctggtgtccgtggacgacttgcagaaccacgtggagcactttggggaggaggagcagaaggacctgcgggtggacgtggacacattcttgcagtactggccgccgcagagccagcagttcagcatgcagtacatctcgcacatcttcggagtgattaactgcaacggttttactctcagtgatcagagaggcctgcaggccgtgggcgtaggcatcttccccaacctgggcctggtgaaccatgactgttggcccaactgtactgtcatatttaacaatggcaaaattgagctccgggccctaggcaagatctcagaaggagaggagctgactgtgtcctatattgacttcctcaacgttagtgaagaacgcaagaggcagctgaagaagcagtactactttgactgcacatgtgaacactgccagaaaaaactgaaggatgacctcttcctgggggtgaaagacaaccccaagccctctcaggaagtggtgaaggagatgatacaattctccaaggatacattggaaaagatagacaaggctcgttccgagggtttgtatcatgaggttgtgaaattatgccgggagtgcctggagaagcaggagccagtgtttgctgacaccaacatctacatgctgcggatgctgagcattgtttcggaggtcctttcctacctccaggcctttgaggaggcctcgttctatgccaggaggatggtggacggctatatgaagctctaccaccccaacaatgcccaactgggcatggccgtgatgcgggcagggctgaccaactggcatgctggtaacattgaggtggggcacgggatgatctgcaaagcctatgccattctcctggtgacacacggaccctcccaccccatcactaaggacttagaggccatgcgggtgcagacggagatggagctacgcatgttccgccagaacgaattcatgtactacaagatgcgcgaggctgccctgaacaaccagcccatgcaggtcatggccgagcccagcaatgagccatccccagctctgttccacaagaagcaatga

SEQ ID NO: 48 - protein, 477 aa, Homo sapiens
MTIGRMENVEVFTAEGKGRGLKATKEFWAADIIFAERAYSAVVFDSLVNFVCHTCFKRQEKLHRCGQCKFAHYCDRTCQKDAWLNHKNECSAIKRYGKVPNENIRLAARIMWRVEREGTGLTEGCLVSVDDLQNHVEHFGEEEQKDLRVDVDTFLQYWPPQSQQFSMQYISHIFGVINCNGFTLSDQRGLQAVGVGIFPNLGLVNHDCWPNCTVIFNNGKIELRALGKISEGEELTVSYIDFLNVSEERKRQLKKQYYFDCTCEHCQKKLKDDLFLGVKDNPKPSQEVVKEMIQFSKDTLEKIDKARSEGLYHEVVKLCRECLEKQEPVFADTNIYMLRMLSIVSEVLSYLQAFEEASFYARRMVDGYMKLYHPNNAQLGMAVMRAGLTNWHAGNIEVGHGMICKAYAILLVTHGPSHPITKDLEAMRVQTEMELRMFRQNEFMYYKMREAALNNQPMQVMAEPSNEPSPALFHKKQ

SEQ ID NO: 49 - DNA, 2238 bp, Homo sapiens
  source 1..2238: mol_type = other DNA
atggaggtgatgaacctgatggagcagcctatcaaggtgactgagtggcagcagacatacacctacgactcgggtatccactcgggcgccaacacctgcgtgccctccgtcagcagcaagggcatcatggaggaggatgaggcctgcgggcgccagtacacgctcaagaaaaccaccacttacacccagggggtgccccccagccaaggtgatctggagtaccagatgtccacaacagccagggccaaacgggtgcgggaggccatgtgccctggtgtgtcaggcgaggacagctcgcttctgctggccacccaggtggaggggcaggccaccaacctgcagcgactggccgagccgtcccagctgctcaagtcggccattgtgcatctcatcaactaccaggacgatgccgagctggccactcgcgccctgcccgagctcaccaaactgctcaacgacgaggacccggtggtggtgaccaaggcggccatgattgtgaaccagctgtcgaagaaggaggcgtcgcggcgggccctgatgggctcgccccagctggtggccgctgtcgtgcgtaccatgcagaataccagcgacctggacacagcccgctgcaccaccagcatcctgcacaacctctcccaccaccgggaggggctgctcgccatcttcaagtcgggtggcatccctgctctggtccgcatgctcagctcccctgtggagtcggtcctgttctatgccatcaccacgctgcacaacctgctcctgtaccaggagggcgccaagatggccgtgcgcctggccgacgggctgcaaaagatggtgcccctgctcaacaagaacaaccccaagttcctggccatcaccaccgactgcctgcagctcctggcctacggcaaccaggagagcaagctgatcatcctggccaatggtgggccccaggccctcgtgcagatcatgcgtaactacagttatgaaaagctgctctggaccaccagtcgtgtgctcaaggtgctatccgtgtgtcccagcaataagcctgccattgtggaggctggtgggatgcaggccctgggcaagcacctgaccagcaacagcccccgcctggtgcagaactgcctgtggaccctgcgcaacctctcagatgtggccaccaagcaggagggcctggagagtgtgctgaagattctggtgaatcagctgagtgtggatgacgtcaacgtcctcacctgtgccacgggcacactctccaacctgacatgcaacaacagcaagaacaagacgctggtgacacagaacagcggtgtggaggctctcatccatgccatcctgcgtgctggtgacaaggacgacatcacggagcctgccgtctgcgctctgcgccacctcactagccgccaccctgaggccgagatggcccagaactctgtgcgtctcaactatggcatcccagccatcgtgaagctgctcaaccagcccaaccagtggccactggtcaaggcaaccatcggcttgatcaggaatctggccctgtgcccagccaaccatgccccgctgcaggaggcagcggtcatcccccgcctcgtccaactgctggtgaaggcccaccaggatgcccagcgccacgtagctgcaggcacacagcagccctacacggatggtgtgaggatggaggagattgtggagggctgcaccggagcactgcacatcctcgcccgggaccccatgaaccgcatggagatcttccggctcaacaccattcccctgtttgtgcagctcctgtactcgtcggtggagaacatccagcgcgtggctgccggggtgctgtgtgagctggcccaggacaaggaggcggccgacgccattgatgcagagggggcctcggccccactcatggagttgctgcactcccgcaacgagggcactgccacctacgctgctgccgtcctgttccgcatctccgaggacaagaacccagactaccggaagcgcgtgtccgtggagctcaccaactccctcttcaagcatgacccggctgcctgggaggctgcccagagcatgattcccatcaatgagccctatggagatgacatggatgccacctaccgccccatgtactccagcgatgtgccccttgacccgctggagatgcacatggacatggatggagactaccccatcgacacctacagcgacggcctcaggcccccgtaccccactgcagaccacatgctggcctag

SEQ ID NO: 50 - protein, 745 aa, Homo sapiens
MEVMNLMEQPIKVTEWQQTYTYDSGIHSGANTCVPSVSSKGIMEEDEACGRQYTLKKTTTYTQGVPPSQGDLEYQMSTTARAKRVREAMCPGVSGEDSSLLLATQVEGQATNLQRLAEPSQLLKSAIVHLINYQDDAELATRALPELTKLLNDEDPVVVTKAAMIVNQLSKKEASRRALMGSPQLVAAVVRTMQNTSDLDTARCTTSILHNLSHHREGLLAIFKSGGIPALVRMLSSPVESVLFYAITTLHNLLLYQEGAKMAVRLADGLQKMVPLLNKNNPKFLAITTDCLQLLAYGNQESKLIILANGGPQALVQIMRNYSYEKLLWTTSRVLKVLSVCPSNKPAIVEAGGMQALGKHLTSNSPRLVQNCLWTLRNLSDVATKQEGLESVLKILVNQLSVDDVNVLTCATGTLSNLTCNNSKNKTLVTQNSGVEALIHAILRAGDKDDITEPAVCALRHLTSRHPEAEMAQNSVRLNYGIPAIVKLLNQPNQWPLVKATIGLIRNLALCPANHAPLQEAAVIPRLVQLLVKAHQDAQRHVAAGTQQPYTDGVRMEEIVEGCTGALHILARDPMNRMEIFRLNTIPLFVQLLYSSVENIQRVAAGVLCELAQDKEAADAIDAEGASAPLMELLHSRNEGTATYAAAVLFRISEDKNPDYRKRVSVELTNSLFKHDPAAWEAAQSMIPINEPYGDDMDATYRPMYSSDVPLDPLEMHMDMDGDYPIDTYSDGLRPPYPTADHMLA

SEQ ID NO: 51 - DNA, 1071 bp, Homo sapiens
  source 1..1071: mol_type = other DNA
atgaccaaatcgtacagcgagagtgggctgatgggcgagcctcagccccaaggtcctccaagctggacagacgagtgtctcagttctcaggacgaggagcacgaggcagacaagaaggaggacgacctcgaaaccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

SEQ ID NO: 52 - protein, 356 aa, Homo sapiens
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 53 - DNA, 1260 bp, Homo sapiens
  source 1..1260: mol_type = other DNA
atggccagtatcttttctaagttgctaactggccgcaatgcttctctgctgtttgctaccatgggcaccagtgtcctgaccaccgggtacctgctgaaccggcagaaagtgtgtgccgaggtccgggagcagcctaggctatttcctccaagcgcagactacccagacctgcgcaagcacaacaactgcatggccgagtgcctcacccccgccatttatgccaagcttcgcaacaaggtgacacccaacggctacacgctggaccagtgcatccagactggagtggacaaccctggccaccccttcataaagactgtgggcatggtggctggtgacgaggagtcctatgaggtgtttgctgacctttttgaccccgtcatcaaactaagacacaacggctatgaccccagggtgatgaagcacacaacggatctggatgcatcaaagatcacccaagggcagttcgacgagcattacgtgctgtcttctcgggtgcgcactggccgcagcatccgtgggctgagcctgcctccagcctgcacccgggccgagcgaagggaggtagagaacgtggccatcactgccctggagggcctcaagggggacctggctggccgctactacaagctgtccgagatgacggagcaggaccagcagcggctcatcgatgaccactttctgtttgataagccagtgtcccctttattaacatgtgctgggatggcccgtgactggccagatgccaggggaatctggcataattatgataagacatttctcatctggataaatgaggaggatcacaccagggtaatctcaatggaaaaaggaggcaatatgaaacgagtatttgagcgattctgtcgtggactaaaagaagtagaacggttaatccaagaacgaggctgggagttcatgtggaatgagcgcctaggatacattttgacctgtccttcgaaccttggaacaggactacgagctggtgtccacgttaggatcccaaagctcagcaaggacccacgcttttctaagatcctggaaaacctaagactccagaagcgtggcacaggtggtgtggacactgccgcggtcgcagatgtgtacgacatttccaacatagatagaattggtcgatcagaggttgagcttgttcagatagtcatcgatggagtcaattacctggtggattgtgaaaagaagttggagagaggccaagatattaaggtgccaccccctctgcctcagtttggcaaaaagtaa

SEQ ID NO: 54 - protein, 419 aa, Homo sapiens
MASIFSKLLTGRNASLLFATMGTSVLTTGYLLNRQKVCAEVREQPRLFPPSADYPDLRKHNNCMAECLTPAIYAKLRNKVTPNGYTLDQCIQTGVDNPGHPFIKTVGMVAGDEESYEVFADLFDPVIKLRHNGYDPRVMKHTTDLDASKITQGQFDEHYVLSSRVRTGRSIRGLSLPPACTRAERREVENVAITALEGLKGDLAGRYYKLSEMTEQDQQRLIDDHFLFDKPVSPLLTCAGMARDWPDARGIWHNYDKTFLIWINEEDHTRVISMEKGGNMKRVFERFCRGLKEVERLIQERGWEFMWNERLGYILTCPSNLGTGLRAGVHVRIPKLSKDPRFSKILENLRLQKRGTGGVDTAAVADVYDISNIDRIGRSEVELVQIVIDGVNYLVDCEKKLERGQDIKVPPPLPQFGKK

SEQ ID NO: 55 - DNA, 3105 bp, Homo sapiens
  source 1..3105: mol_type = other DNA
atgccgaggagaaaacagcaggcacccaagcgggcggcaggctacgcccaggaggaacagctgaaagaagaggaggaaataaaagaagaggaggaggaggaggacagcggttcagtagctcaactgcagggtggcaatgacacagggacggacgaggagctagaaacgggcccagagcaaaaaggctgcttcagctaccagaactctccaggaagtcatttgtccaatcaggatgccgagaacgagtctctgctgagtgacgccagtgatcaggtgtcggacatcaagagtgtctgcggcagagatgcctcagacaagaaagcacacactcacgtcaggcttccaaacgaagcacacaattgcatggataaaatgaccgctgtctacgccaacatcctgtcggattcctactggtcaggcctgggccttggcttcaagctgtccaatagtgagaggaggaactgtgacacccgaaacggcagcaacaagagtgattttgattggcaccaagacgctctgtccaaaagcctgcagcagaacttgccttctcggtccgtctcgaaacccagcctgttcagctcggtgcagttgtaccgacagagcagcaagatgtgcgggactgtgttcacaggggccagcagattccgatgccgacagtgcagcgcggcctatgacaccctagtcgagctgactgtgcacatgaatgaaacgggccactatcaagatgacaaccgcaaaaaggacaagctcagacccacgagctattcaaagcccaggaaaagggctttccaggatatggacaaagaggatgctcaaaaggttctgaaatgtatgttttgtggcgactcctttgattccctccaagatttgagcgtccacatgattaaaacaaaacattaccaaaaagtgcctttgaaggagccagtcccaaccatttcctcgaaaatggtcaccccggctaagaaacgcgtttttgatgtcaatcggccgtgttcccccgattcaaccacaggatcttttgcagattctttttcttctcagaagaacgccaacttgcagttgtcctccaacaaccgctatggctaccaaaatggagccagctacacctggcagtttgaggcctgcaagtcccagatcttaaagtgcatggagtgtgggagctcccatgacaccttgcagcagctcaccacccacatgatggtcacaggtcactttctcaaggtcaccagctctgcctccaagaaagggaagcagctggtattagacccgttagcagtggagaaaatgcagtcgttgtctgaggccccaaacagtgattctctggctcccaagccatccagtaactcagcatcagattgtacagcctctacaactgagttaaagaaagagagtaaaaaagaaaggccagaggaaaccagcaaggatgagaaagtcgtgaaaagcgaggactatgaagatcctctacaaaaacctttagaccctacaatcaaatatcaatacctaagggaggaagacttggaagatggctcaaagggtggaggggacattttgaaatctttggaaaatactgtcaccacagccatcaacaaagcccaaaacggggcccccagctggagtgcctaccccagcatccacgcagcctaccagctgtctgagggcaccaagccgcctttgcctatgggatcccaggtactgcagatccggcctaatctcaccaacaagctgaggcccattgcaccaaagtggaaagtgatgccactggtttctatgcccacacacctggccccttacactcaagtcaagaaagagtcagaagacaaagatgaagcggtgaaggagtgtgggaaagaaagtccccacgaagaggcctcatctttcagccacagtgagggcgattctttccgcaaaagtgaaacacctccagaagccaaaaagaccgagctgggtcccctgaaggaggaggagaagctgatgaaagagggcagcgagaaggagaaaccccagcccctggagcccacatctgctctgagcaatgggtgcgccctcgccaaccacgccccggccctgccatgcatcaacccactcagcgccctgcagtccgtcctgaacaatcacttgggcaaagccacggagcccttgcgctcaccttcctgctccagcccaagttcaagcacaatttccatgttccacaagtcgaatctcaatgtcatggacaagccggtcttgagtcctgcctccacaaggtcagccagcgtgtccaggcgctacctgtttgagaacagcgatcagcccattgacctgaccaagtccaaaagcaagaaagccgagtcctcgcaagcacaatcttgtatgtccccacctcagaagcacgctctgtctgacatcgccgacatggtcaaagtcctccccaaagccaccaccccaaagccagcctcctcctccagggtcccccccatgaagctggaaatggatgtcaggcgctttgaggatgtctccagtgaagtctcaactttgcataaaagaaaaggccggcagtccaactggaatcctcagcatcttctgattctacaagcccagtttgcctcgagcctcttccagacatcagagggcaaatacctgctgtctgatctgggcccacaagagcgtatgcaaatctctaagtttacgggactctcaatgaccactatcagtcactggctggccaacgtcaagtaccagcttaggaaaacgggcgggacaaaatttctgaaaaacatggacaaaggccaccccatcttttattgcagtgactgtgcctcccagttcagaaccccttctacctacatcagtcacttagaatctcacctgggtttccaaatgaaggacatgacccgcttgtcagtggaccagcaaagcaaggtggagcaagagatctcccgggtatcgtcggctcagaggtctccagaaacaatagctgccgaagaggacacagactctaaattcaagtgtaagttgtgctgtcggacatttgtgagcaaacatgcggtaaaactccacctaagcaaaacgcacagcaagtcacccgaacaccattcacagtttgtaacagacgtggatgaagaatag

SEQ ID NO: 56 - protein, 1034 aa, Homo sapiens
MPRRKQQAPKRAAGYAQEEQLKEEEEIKEEEEEEDSGSVAQLQGGNDTGTDEELETGPEQKGCFSYQNSPGSHLSNQDAENESLLSDASDQVSDIKSVCGRDASDKKAHTHVRLPNEAHNCMDKMTAVYANILSDSYWSGLGLGFKLSNSERRNCDTRNGSNKSDFDWHQDALSKSLQQNLPSRSVSKPSLFSSVQLYRQSSKMCGTVFTGASRFRCRQCSAAYDTLVELTVHMNETGHYQDDNRKKDKLRPTSYSKPRKRAFQDMDKEDAQKVLKCMFCGDSFDSLQDLSVHMIKTKHYQKVPLKEPVPTISSKMVTPAKKRVFDVNRPCSPDSTTGSFADSFSSQKNANLQLSSNNRYGYQNGASYTWQFEACKSQILKCMECGSSHDTLQQLTTHMMVTGHFLKVTSSASKKGKQLVLDPLAVEKMQSLSEAPNSDSLAPKPSSNSASDCTASTTELKKESKKERPEETSKDEKVVKSEDYEDPLQKPLDPTIKYQYLREEDLEDGSKGGGDILKSLENTVTTAINKAQNGAPSWSAYPSIHAAYQLSEGTKPPLPMGSQVLQIRPNLTNKLRPIAPKWKVMPLVSMPTHLAPYTQVKKESEDKDEAVKECGKESPHEEASSFSHSEGDSFRKSETPPEAKKTELGPLKEEEKLMKEGSEKEKPQPLEPTSALSNGCALANHAPALPCINPLSALQSVLNNHLGKATEPLRSPSCSSPSSSTISMFHKSNLNVMDKPVLSPASTRSASVSRRYLFENSDQPIDLTKSKSKKAESSQAQSCMSPPQKHALSDIADMVKVLPKATTPKPASSSRVPPMKLEMDVRRFEDVSSEVSTLHKRKGRQSNWNPQHLLILQAQFASSLFQTSEGKYLLSDLGPQERMQISKFTGLSMTTISHWLANVKYQLRKTGGTKFLKNMDKGHPIFYCSDCASQFRTPSTYISHLESHLGFQMKDMTRLSVDQQSKVEQEISRVSSAQRSPETIAAEEDTDSKFKCKLCCRTFVSKHAVKLHLSKTHSKSPEHHSQFVTDVDEE

SEQ ID NO: 57 - DNA, 3096 bp, Homo sapiens
  source 1..3096: mol_type = other DNA
atgatggctgctgcgttgctccattatacaggctacgcccaggaggaacagctgaaagaagaggaggaaataaaagaagaggaggaggaggaggacagcggttcagtagctcaactgcagggtggcaatgacacagggacggacgaggagctagaaacgggcccagagcaaaaaggctgcttcagctaccagaactctccaggaagtcatttgtccaatcaggatgccgagaacgagtctctgctgagtgacgccagtgatcaggtgtcggacatcaagagtgtctgcggcagagatgcctcagacaagaaagcacacactcacgtcaggcttccaaacgaagcacacaattgcatggataaaatgaccgctgtctacgccaacatcctgtcggattcctactggtcaggcctgggccttggcttcaagctgtccaatagtgagaggaggaactgtgacacccgaaacggcagcaacaagagtgattttgattggcaccaagacgctctgtccaaaagcctgcagcagaacttgccttctcggtccgtctcgaaacccagcctgttcagctcggtgcagttgtaccgacagagcagcaagatgtgcgggactgtgttcacaggggccagcagattccgatgccgacagtgcagcgcggcctatgacaccctagtcgagctgactgtgcacatgaatgaaacgggccactatcaagatgacaaccgcaaaaaggacaagctcagacccacgagctattcaaagcccaggaaaagggctttccaggatatggacaaagaggatgctcaaaaggttctgaaatgtatgttttgtggcgactcctttgattccctccaagatttgagcgtccacatgattaaaacaaaacattaccaaaaagtgcctttgaaggagccagtcccaaccatttcctcgaaaatggtcaccccggctaagaaacgcgtttttgatgtcaatcggccgtgttcccccgattcaaccacaggatcttttgcagattctttttcttctcagaagaacgccaacttgcagttgtcctccaacaaccgctatggctaccaaaatggagccagctacacctggcagtttgaggcctgcaagtcccagatcttaaagtgcatggagtgtgggagctcccatgacaccttgcagcagctcaccacccacatgatggtcacaggtcactttctcaaggtcaccagctctgcctccaagaaagggaagcagctggtattagacccgttagcagtggagaaaatgcagtcgttgtctgaggccccaaacagtgattctctggctcccaagccatccagtaactcagcatcagattgtacagcctctacaactgagttaaagaaagagagtaaaaaagaaaggccagaggaaaccagcaaggatgagaaagtcgtgaaaagcgaggactatgaagatcctctacaaaaacctttagaccctacaatcaaatatcaatacctaagggaggaagacttggaagatggctcaaagggtggaggggacattttgaaatctttggaaaatactgtcaccacagccatcaacaaagcccaaaacggggcccccagctggagtgcctaccccagcatccacgcagcctaccagctgtctgagggcaccaagccgcctttgcctatgggatcccaggtactgcagatccggcctaatctcaccaacaagctgaggcccattgcaccaaagtggaaagtgatgccactggtttctatgcccacacacctggccccttacactcaagtcaagaaagagtcagaagacaaagatgaagcggtgaaggagtgtgggaaagaaagtccccacgaagaggcctcatctttcagccacagtgagggcgattctttccgcaaaagtgaaacacctccagaagccaaaaagaccgagctgggtcccctgaaggaggaggagaagctgatgaaagagggcagcgagaaggagaaaccccagcccctggagcccacatctgctctgagcaatgggtgcgccctcgccaaccacgccccggccctgccatgcatcaacccactcagcgccctgcagtccgtcctgaacaatcacttgggcaaagccacggagcccttgcgctcaccttcctgctccagcccaagttcaagcacaatttccatgttccacaagtcgaatctcaatgtcatggacaagccggtcttgagtcctgcctccacaaggtcagccagcgtgtccaggcgctacctgtttgagaacagcgatcagcccattgacctgaccaagtccaaaagcaagaaagccgagtcctcgcaagcacaatcttgtatgtccccacctcagaagcacgctctgtctgacatcgccgacatggtcaaagtcctccccaaagccaccaccccaaagccagcctcctcctccagggtcccccccatgaagctggaaatggatgtcaggcgctttgaggatgtctccagtgaagtctcaactttgcataaaagaaaaggccggcagtccaactggaatcctcagcatcttctgattctacaagcccagtttgcctcgagcctcttccagacatcagagggcaaatacctgctgtctgatctgggcccacaagagcgtatgcaaatctctaagtttacgggactctcaatgaccactatcagtcactggctggccaacgtcaagtaccagcttaggaaaacgggcgggacaaaatttctgaaaaacatggacaaaggccaccccatcttttattgcagtgactgtgcctcccagttcagaaccccttctacctacatcagtcacttagaatctcacctgggtttccaaatgaaggacatgacccgcttgtcagtggaccagcaaagcaaggtggagcaagagatctcccgggtatcgtcggctcagaggtctccagaaacaatagctgccgaagaggacacagactctaaattcaagtgtaagttgtgctgtcggacatttgtgagcaaacatgcggtaaaactccacctaagcaaaacgcacagcaagtcacccgaacaccattcacagtttgtaacagacgtggatgaagaatag

SEQ ID NO: 58 - protein, 1031 aa, Homo sapiens
MMAAALLHYTGYAQEEQLKEEEEIKEEEEEEDSGSVAQLQGGNDTGTDEELETGPEQKGCFSYQNSPGSHLSNQDAENESLLSDASDQVSDIKSVCGRDASDKKAHTHVRLPNEAHNCMDKMTAVYANILSDSYWSGLGLGFKLSNSERRNCDTRNGSNKSDFDWHQDALSKSLQQNLPSRSVSKPSLFSSVQLYRQSSKMCGTVFTGASRFRCRQCSAAYDTLVELTVHMNETGHYQDDNRKKDKLRPTSYSKPRKRAFQDMDKEDAQKVLKCMFCGDSFDSLQDLSVHMIKTKHYQKVPLKEPVPTISSKMVTPAKKRVFDVNRPCSPDSTTGSFADSFSSQKNANLQLSSNNRYGYQNGASYTWQFEACKSQILKCMECGSSHDTLQQLTTHMMVTGHFLKVTSSASKKGKQLVLDPLAVEKMQSLSEAPNSDSLAPKPSSNSASDCTASTTELKKESKKERPEETSKDEKVVKSEDYEDPLQKPLDPTIKYQYLREEDLEDGSKGGGDILKSLENTVTTAINKAQNGAPSWSAYPSIHAAYQLSEGTKPPLPMGSQVLQIRPNLTNKLRPIAPKWKVMPLVSMPTHLAPYTQVKKESEDKDEAVKECGKESPHEEASSFSHSEGDSFRKSETPPEAKKTELGPLKEEEKLMKEGSEKEKPQPLEPTSALSNGCALANHAPALPCINPLSALQSVLNNHLGKATEPLRSPSCSSPSSSTISMFHKSNLNVMDKPVLSPASTRSASVSRRYLFENSDQPIDLTKSKSKKAESSQAQSCMSPPQKHALSDIADMVKVLPKATTPKPASSSRVPPMKLEMDVRRFEDVSSEVSTLHKRKGRQSNWNPQHLLILQAQFASSLFQTSEGKYLLSDLGPQERMQISKFTGLSMTTISHWLANVKYQLRKTGGTKFLKNMDKGHPIFYCSDCASQFRTPSTYISHLESHLGFQMKDMTRLSVDQQSKVEQEISRVSSAQRSPETIAAEEDTDSKFKCKLCCRTFVSKHAVKLHLSKTHSKSPEHHSQFVTDVDEE

SEQ ID NO: 59 - DNA, 1563 bp, Homo sapiens
  source 1..1563: mol_type = other DNA
atgcagtccgaatcggggatcgtgccggatttcgaagtcggggaggagtttcatgaagagcccaaaacctattacgaactcaaaagtcaaccgctgaagagcagcagttccgccgagcatcctggggcctccaagcctccgataagctcctccagtatgacatcacgcatcttgctacgccagcaactcatgcgtgagcagatgcaggagcaggagcgcagggagcagcagcagaagctgcaggcggcccagttcatgcaacagagagtgcccgtgagtcagacaccagccataaacgtcagtgtgcccaccacccttccctctgccacgcaggtgccgatggaagtccttaaggtgcagacccacctcgaaaaccccaccaagtaccacatacagcaagcccaacggcagcaggtaaagcagtacctttctaccactttagcaaataaacatgccaaccaagtcctgagcttgccatgtccaaaccagcctggcgatcatgtcatgccaccggtgccggggagcagcgcacccaacagccccatggctatgcttacgcttaactccaactgtgaaaaagagggattttataagtttgaagagcaaaacagggcagagagcgagtgcccaggcatgaacacacattcacgagcgtcctgtatgcagatggatgatgtaatcgatgacatcattagcctagaatcaagttataatgaggaaatcttgggcttgatggatcctgctttgcaaatggcaaatacgttgcctgtctcgggaaacttgattgatctttatggaaaccaaggtctgcccccaccaggcctcaccatcagcaactcctgtccagccaaccttcccaacataaaaagggagctcacagagtctgaagcaagagcactggccaaagagaggcagaaaaaggacaatcacaacctgattgaacgaagaagaagatttaacataaatgaccgcattaaagaactaggtactttgattcccaagtcaaatgatccagacatgcgctggaacaagggaaccatcttaaaagcatccgtggactatatccgaaagttgcaacgagaacagcaacgcgcaaaagaacttgaaaaccgacagaagaaactggagcacgccaaccggcatttgttgctcagaatacaggaacttgaaatgcaggctcgagctcatggactttcccttattccatccacgggtctctgctctccagatttggtgaatcggatcatcaagcaagaacccgttcttgagaactgcagccaagacctccttcagcatcatgcagacctaacctgtacaacaactctcgatctcacggatggcaccatcaccttcaacaacaacctcggaactgggactgaggccaaccaagcctatagtgtccccacaaaaatgggatccaaactggaagacatcctgatggacgacaccctttctcccgtcggtgtcactgatccactcctttcctcagtgtcccccggagcttccaaaacaagcagccggaggagcagtatgagcatggaagagacggagcacacttgttag

SEQ ID NO: 60 - protein, 520 aa, Homo sapiens
MQSESGIVPDFEVGEEFHEEPKTYYELKSQPLKSSSSAEHPGASKPPISSSSMTSRILLRQQLMREQMQEQERREQQQKLQAAQFMQQRVPVSQTPAINVSVPTTLPSATQVPMEVLKVQTHLENPTKYHIQQAQRQQVKQYLSTTLANKHANQVLSLPCPNQPGDHVMPPVPGSSAPNSPMAMLTLNSNCEKEGFYKFEEQNRAESECPGMNTHSRASCMQMDDVIDDIISLESSYNEEILGLMDPALQMANTLPVSGNLIDLYGNQGLPPPGLTISNSCPANLPNIKRELTESEARALAKERQKKDNHNLIERRRRFNINDRIKELGTLIPKSNDPDMRWNKGTILKASVDYIRKLQREQQRAKELENRQKKLEHANRHLLLRIQELEMQARAHGLSLIPSTGLCSPDLVNRIIKQEPVLENCSQDLLQHHADLTCTTTLDLTDGTITFNNNLGTGTEANQAYSVPTKMGSKLEDILMDDTLSPVGVTDPLLSSVSPGASKTSSRRSSMSMEETEHTC

SEQ ID NO: 61 - DNA, 2961 bp, Homo sapiens
  source 1..2961: mol_type = other DNA
atgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccagggccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtag

SEQ ID NO: 62 - protein, 986 aa, Homo sapiens
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 63 - DNA, 2817 bp, Homo sapiens
  source 1..2817: mol_type = other DNA
atgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccagggccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtag

SEQ ID NO: 64 - protein, 938 aa, Homo sapiens
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 65 - DNA, 2724 bp, Homo sapiens
  source 1..2724: mol_type = other DNA
atgcacatactccaagcttccactgcagagaggtccattccaactgctcagatgaagctgaaaagagcccgactcgccgatgatctcaatgaaaaaattgctctacgaccagggccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtag

SEQ ID NO: 66 - protein, 907 aa, Homo sapiens
MHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 67 - DNA, 3072 bp, Homo sapiens
  source 1..3072: mol_type = other DNA
atggcggggaacgactgcggcgcgctgctggacgaagagctctcctccttcttcctcaactatctcgctgacacgcagggtggagggtccggggaggagcaactctatgctgactttccagaacttgacctctcccagctggatgccagcgactttgactcggccacctgctttggggagctgcagtggtgcccagagaactcagagactgaacccaaccagtacagccccgatgactccgagctcttccagattgacagtgagaatgaggccctcctggcagagctcaccaagaccctggatgacatccctgaagatgacgtgggtctggctgccttcccagccctggatggtggagacgctctatcatgcacctcagcttcgcctgccccctcatctgcaccccccagccctgccccggagaagccctcggccccagcccctgaggtggacgagctctcactgctgcagaagctcctcctggccacatcctacccaacatcaagctctgacacccagaaggaagggaccgcctggcgccaggcaggcctcagatctaaaagtcaacggccttgtgttaaggcggacagcacccaagacaagaaggctcccatgatgcagtctcagagccgaagttgtacagaactacataagcacctcacctcggcacagtgctgcctgcaggatcggggtctgcagccaccatgcctccagagtccccggctccctgccaaggaggacaaggagccgggtgaggactgcccgagcccccagccagctccagcctctccccgggactccctagctctgggcagggcagaccccggtgccccggtttcccaggaagacatgcaggcgatggtgcaactcatacgctacatgcacacctactgcctcccccagaggaagctgcccccacagacccctgagccactccccaaggcctgcagcaacccctcccagcaggtcagatcccggccctggtcccggcaccactccaaagcctcctgggctgagttctccattctgagggaacttctggctcaagacgtgctctgtgatgtcagcaaaccctaccgtctggccacgcctgtttatgcctccctcacacctcggtcaaggcccaggccccccaaagacagtcaggcctcccctggtcgcccgtcctcggtggaggaggtaaggatcgcagcttcacccaagagcaccgggcccagaccaagcctgcgcccactgcggctggaggtgaaaagggaggtccgccggcctgccagactgcagcagcaggaggaggaagacgaggaagaagaggaggaggaagaggaagaagaaaaagaggaggaggaggagtggggcaggaaaaggccaggccgaggcctgccatggacgaagctggggaggaagctggagagctctgtgtgccccgtgcggcgttctcggagactgaaccctgagctgggcccctggctgacatttgcagatgagccgctggtcccctcggagccccaaggtgctctgccctcactgtgcctggctcccaaggcctacgacgtagagcgggagctgggcagccccacggacgaggacagtggccaagaccagcagctcctacggggaccccagatccctgccctggagagcccctgtgagagtgggtgtggggacatggatgaggaccccagctgcccgcagctccctcccagagactctcccaggtgcctcatgctggccttgtcacaaagcgacccaacttttggcaagaagagctttgagcagaccttgacagtggagctctgtggcacagcaggactcaccccacccaccacaccaccgtacaagcccacagaggaggatcccttcaaaccagacatcaagcatagtctaggcaaagaaatagctctcagcctcccctcccctgagggcctctcactcaaggccaccccaggggctgcccacaagctgccaaagaagcacccagagcgaagtgagctcctgtcccacctgcgacatgccacagcccagccagcctcccaggctggccagaagcgtcccttctcctgttcctttggagaccatgactactgccaggtgctccgaccagaaggcgtcctgcaaaggaaggtgctgaggtcctgggagccgtctggggttcaccttgaggactggccccagcagggtgccccttgggctgaggcacaggcccctggcagggaggaagacagaagctgtgatgctggcgccccacccaaggacagcacgctgctgagagaccatgagatccgtgccagcctcaccaaacactttgggctgctggagaccgccctggaggaggaagacctggcctcctgcaagagccctgagtatgacactgtctttgaagacagcagcagcagcagcggcgagagcagcttcctcccagaggaggaagaggaagaaggggaggaggaggaggaggacgatgaagaagaggactcaggggtcagccccacttgctctgaccactgcccctaccagagcccaccaagcaaggccaaccggcagctctgttcccgcagccgctcaagctctggctcttcaccctgccactcctggtcaccagccactcgaaggaacttcagatgtgagagcagagggccgtgttcagacagaacgccaagcatccggcacgccaggaagcggcgggaaaaggccattggggaaggccgcgtggtgtacattcaaaatctctccagcgacatgagctcccgagagctgaagaggcgctttgaagtgtttggtgagattgaggagtgcgaggtgctgacaagaaataggagaggcgagaagtacggcttcatcacctaccggtgttctgagcacgcggccctctctttgacaaagggcgctgccctgaggaagcgcaacgagccctccttccagctgagctacggagggctccggcacttctgctggcccagatacactgactacgattccaattcagaagaggcccttcctgcgtcagggaaaagcaagtatgaagccatggattttgacagcttactgaaagaggcccagcagagcctgcattga

SEQ ID NO: 68 - protein, 1023 aa, Homo sapiens
MAGNDCGALLDEELSSFFLNYLADTQGGGSGEEQLYADFPELDLSQLDASDFDSATCFGELQWCPENSETEPNQYSPDDSELFQIDSENEALLAELTKTLDDIPEDDVGLAAFPALDGGDALSCTSASPAPSSAPPSPAPEKPSAPAPEVDELSLLQKLLLATSYPTSSSDTQKEGTAWRQAGLRSKSQRPCVKADSTQDKKAPMMQSQSRSCTELHKHLTSAQCCLQDRGLQPPCLQSPRLPAKEDKEPGEDCPSPQPAPASPRDSLALGRADPGAPVSQEDMQAMVQLIRYMHTYCLPQRKLPPQTPEPLPKACSNPSQQVRSRPWSRHHSKASWAEFSILRELLAQDVLCDVSKPYRLATPVYASLTPRSRPRPPKDSQASPGRPSSVEEVRIAASPKSTGPRPSLRPLRLEVKREVRRPARLQQQEEEDEEEEEEEEEEEKEEEEEWGRKRPGRGLPWTKLGRKLESSVCPVRRSRRLNPELGPWLTFADEPLVPSEPQGALPSLCLAPKAYDVERELGSPTDEDSGQDQQLLRGPQIPALESPCESGCGDMDEDPSCPQLPPRDSPRCLMLALSQSDPTFGKKSFEQTLTVELCGTAGLTPPTTPPYKPTEEDPFKPDIKHSLGKEIALSLPSPEGLSLKATPGAAHKLPKKHPERSELLSHLRHATAQPASQAGQKRPFSCSFGDHDYCQVLRPEGVLQRKVLRSWEPSGVHLEDWPQQGAPWAEAQAPGREEDRSCDAGAPPKDSTLLRDHEIRASLTKHFGLLETALEEEDLASCKSPEYDTVFEDSSSSSGESSFLPEEEEEEGEEEEEDDEEEDSGVSPTCSDHCPYQSPPSKANRQLCSRSRSSSGSSPCHSWSPATRRNFRCESRGPCSDRTPSIRHARKRREKAIGEGRVVYIQNLSSDMSSRELKRRFEVFGEIEECEVLTRNRRGEKYGFITYRCSEHAALSLTKGAALRKRNEPSFQLSYGGLRHFCWPRYTDYDSNSEEALPASGKSKYEAMDFDSLLKEAQQSLH